(19) United States
(12) Patent Application Publication
(10) Pub. No.: US 2025/0326765 A1
(43) Pub. Date: Oct. 23, 2025

(54) PDE4B INHIBITOR AND USE THEREOF

(71) Applicant: XIZANG HAISCO PHARMACEUTICAL CO., LTD., Tibet (CN)

(72) Inventors: Yao LI, Tibet (CN); Lei CHEN, Tibet (CN); Zongjun SHI, Tibet (CN); Lei REN, Tibet (CN); Shuai HUANG, Tibet (CN); Shuang YANG, Tibet (CN); Tiancheng HE, Tibet (CN); Jie WANG, Tibet (CN); Fengkai CHENG, Tibet (CN); Chen ZHANG, Tibet (CN); Pangke YAN, Tibet (CN)

(73) Assignee: XIZANG HAISCO PHARMACEUTICAL CO., LTD., Tibet (CN)

(21) Appl. No.: 18/870,606

(22) PCT Filed: Jun. 2, 2023
(86) PCT No.: PCT/CN2023/097944

§ 371 Date: Nov. 29, 2024

(30) Foreign Application Priority Data
Jun. 2, 2022 (CN) 202210625926.4
Jun. 22, 2022 (CN) 202210714663.4
Jul. 19, 2022 (CN) 202210848130.5
Aug. 9, 2022 (CN) 202210952925.0
Nov. 2, 2022 (CN) 202211362508.7
Nov. 24, 2022 (CN) 202211482574.8
Jan. 12, 2023 (CN) 202310038878.3
Feb. 24, 2023 (CN) 202310162024.6
Mar. 20, 2023 (CN) 202310271298.9
Apr. 6, 2023 (CN) 202310357142.2
Apr. 23, 2023 (CN) 202310440428.7

(51) Int. Cl. C07D 495/04; A61K 31/519; A61K 31/5383; A61K 31/55; A61K 31/675; A61K 31/69; A61K 31/695; C07D 519/00; C07F 5/02; C07F 7/08; C07F 9/6561

(52) U.S. Cl. CPC C07D 495/04; A61K 31/519; A61K 31/5383; A61K 31/55; A61K 31/675; A61K 31/69; A61K 31/695; C07D 519/00; C07F 5/025; C07F 7/0816; C07F 9/6561

(57) ABSTRACT

Disclosed are a compound as represented by formula I; a stereoisomer or a pharmaceutically acceptable salt thereof, or a pharmaceutical composition containing same; and the use thereof as a PDE4B inhibitor in the preparation of a drug for the treatment of related diseases. Each group as shown in formula (I) is as defined in the description.

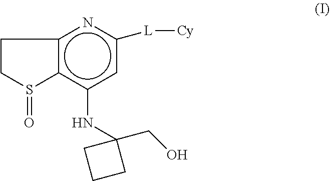

### CROSS-REFERENCE TO RELATED APPLICATIONS

This application is a 35 U.S.C. § 371 National Stage of International Patent Application No. PCT/CN2023/097944, filed Jun. 2, 2023, designating the United States, which claims priority to and the benefits of Chinese Patent Application No. 202210625926.4, filed Jun. 2, 2022, Chinese Patent Application No. 202210714663.4, filed Jun. 22, 2022, Chinese Patent Application No. 202210848130.5, filed Jul. 19, 2022, Chinese Patent Application No. 202210952925.0, filed Aug. 9, 2022, Chinese Patent Application No. 202211362508.7, filed Nov. 2, 2022, Chinese Patent Application No. 202211482574.8, filed Nov. 24, 2022, Chinese Patent Application No. 202310038878.3, filed Jan. 12, 2023, Chinese Patent Application No. 202310162024.6, filed Feb. 24, 2023, Chinese Patent Application No. 202310271298.9, filed Mar. 20, 2023, Chinese Patent Application No. 202310357142.2, filed Apr. 6, 2023, and Chinese Patent Application No. 202310440428.7, filed Apr. 23, 2023, the disclosures of which are incorporated herein in their entirety by reference, and priority is claimed to each of the foregoing.

### TECHNICAL FIELD

The present invention belongs to the field of medicine and in particular relates to a small molecule compound and a stereoisomer or pharmaceutically acceptable salt thereof, which have a selective inhibitory activity against PDE4B, and the use thereof in the preparation of a medicament for treating a related disease.

### BACKGROUND

PDE4 inhibitors produce antidepressant effects in humans and animals by enhancing cAMP signaling in the brain. PDE4 inhibitors also play an important role in the treatment of other central nervous system diseases, including diseases such as Alzheimer's disease, Parkinson's disease, schizophrenia, stroke, and Huntington's chorea.

In addition, the research and development of PDE4 inhibitors have also made great progress in the treatment of respiratory diseases such as asthma and chronic obstructive pulmonary disease. The principle of research and development of such drugs stems from the role of PDE4 in inhibiting the function of a range of inflammatory cells and resident cells, which is believed to be associated with the pathogenesis of such diseases. A large number of clinical studies have shown that cyclic adenosine monophosphate (cAMP) can block the proliferation and chemotaxis of inflammatory cells and inhibit the release of inflammatory and cytotoxic mediators in lungs. In addition, PDE4 is especially abundant in immune cells, inflammatory cells, and smooth muscle cells.

PDE4 inhibitors play an anti-inflammatory role mainly by inhibiting the hydrolysis function of PDE4 so as to increase the cAMP level in vivo, thereby inhibiting the release of inflammatory factors and at the same time promoting the production of anti-inflammatory mediators. Roflumilast is used clinically for the treatment of COPD with a significant anti-inflammatory effect and can inhibit the release of inflammatory mediators such as TNF-α, interleukins, and chemokines from monocytes, macrophages, T cells, etc. However, such inhibitors generally have serious side effects such as nausea and vomiting, which limits the clinical application of PDE4 inhibitors. A large number of studies have shown that phosphodiesterase 4 subtype B (PDE4B), which is associated with inflammatory responses in the human body, participates in the release of various inflammatory mediators in vivo, while subtype D is closely related to the production of side effects such as nausea and vomiting, which provides a new idea for finding PDE4 inhibitors with low side effects, i.e., designing PDE4B inhibitors that may have reduced side effects, thereby promoting further clinical application.

Phosphodiesterase 4 is highly selective for cAMP and has four subtypes, namely PDE4A, 4B, 4C, and 4D, with at least 25 splice variants. The protein sequences of the catalytic domains of the four subtypes of PDE4 are highly homologous; therefore, inhibitors that act on the catalytic domain do not exhibit subtype selectivity. Most of the classical PDE4 inhibitors have been reported to act on the catalytic domain. A new mode of action of PDE4 inhibitors has been reported in recent years, in which the inhibitor acts on both the catalytic domain and a regulatory sequence, so that the regulatory sequence can stabilize the closed conformation of the protein, thereby preventing cAMP from entering, thus exerting an inhibitory effect. However, it has been found from research that there are amino acid differences between PDE4B and 4D in this regulatory sequence, so designing inhibitors based on such differences can produce subtype selectivity. Therefore, based on the difference of the two amino acids PDE4B Leu674/PDE4D Gln594 in CR3 (Conserved Region 3) of the downstream regulatory sequence, it is expected to achieve the selectivity to subtype B and reduce the side effects of the inhibitors while maintaining the activity.

A selective PDE4B inhibitor with a good activity, a high safety, less side effects, and superior pharmacokinetics has been found, which has good clinical development prospects and can be used for treating COPD, cancer, or other proliferative diseases or conditions.

### SUMMARY OF THE INVENTION

The present invention provides a small molecule compound with PDE4B inhibitory activity, or a stereoisomer or pharmaceutically acceptable salt thereof, wherein the compound is as represented by formula (I),

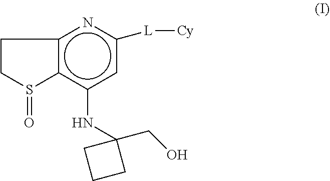

wherein Cy is selected from Cy1, Cy2, a 5-membered monocyclic heteroaryl, or an 8- to 10-membered bicyclic heteroaryl, wherein the monocyclic heteroaryl and the bicyclic heteroaryl are optionally further substituted with 1-3 R; in some embodiments, Cy is selected from Cy1 or Cy2; in some embodiments, Cy is selected from Cy1; in some embodiments, Cy is selected from 5-membered monocyclic heteroaryl, 5-membered heteroaryl fused benzene ring, 5-membered heteroaryl fused 5-membered heteroaryl, 5-membered heteroaryl fused 6-membered heteroaryl, 5-membered heteroaryl fused 4-membered cycloalkyl, 5-membered heteroaryl fused 5-membered cycloalkyl, 5-membered heteroaryl fused 6-membered cycloalkyl, 5-membered heteroaryl fused 4-membered heterocycloalkyl, 5-membered heteroaryl fused 5-membered heterocycloalkyl, 5-membered heteroaryl fused 6-membered heterocycloalkyl, 6-membered heteroaryl fused benzene ring, 6-membered heteroaryl fused 5-membered heteroaryl, 6-membered heteroaryl fused 6-membered heteroaryl, 6-membered heteroaryl fused 4-membered cycloalkyl, 6-membered heteroaryl fused 5-membered cycloalkyl, 6-membered heteroaryl fused 6-membered cycloalkyl, 6-membered heteroaryl fused 4-membered heterocycloalkyl, 6-membered heteroaryl fused 5-membered heterocycloalkyl, and 6-membered heteroaryl fused 6-membered heterocycloalkyl; in some embodiments, Cy is selected from the following structures:

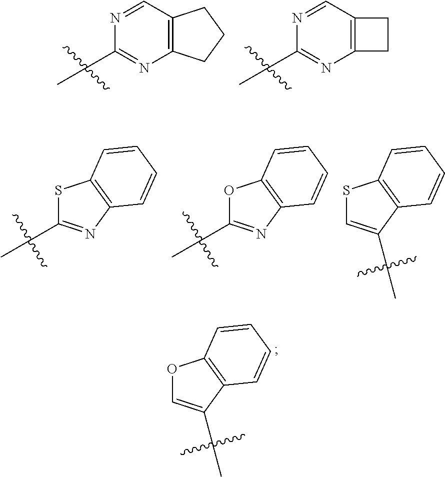

in some embodiments, Cy is selected from the following structures:

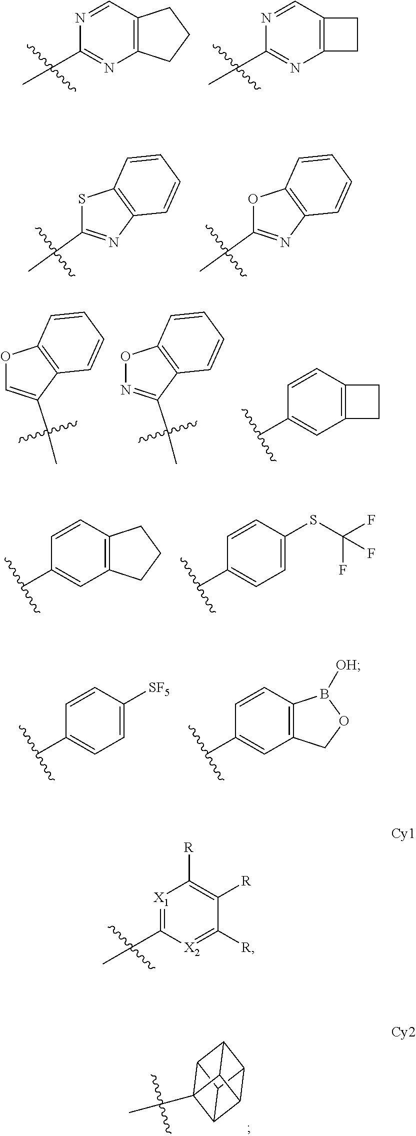

Cy2 is optionally substituted with 1-3 R; in some embodiments, Cy2 is unsubstituted;
L is -(L₁)n-(L₂)m-;
in all the solutions of the present invention, the site of attachment of L to Cy is arbitrary; in some embodiments, the L₂ end is a site of attachment to Cy; in some embodiments, the L₁ end is a site of attachment to Cy;
L₁ is —NR₁—, —CR₂═N—, —CR₂═CR₂—, or —CR₂R₂—;
in all the solutions of the present invention, the site of attachment of L₁ is arbitrary, for example, N atom and C atom in —CR₂═N— connected to L₂ can both be the site of attachment to L₂;

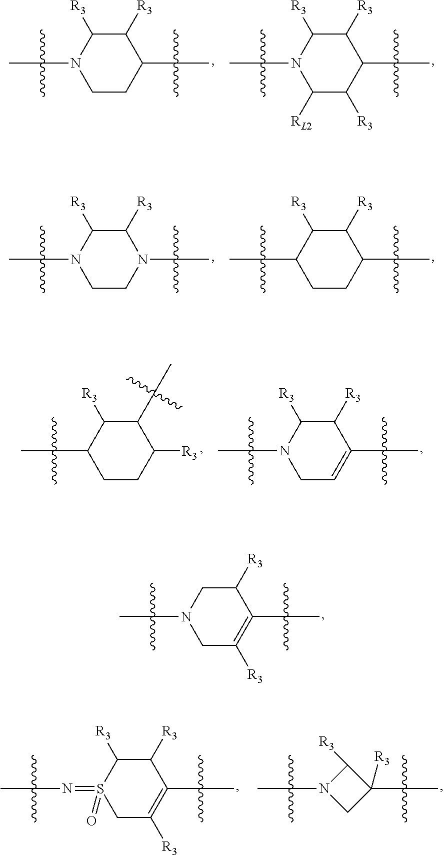

C₁₋₄ alkylene, C₂₋₄ alkenylene, C₂₋₄ alkynylene, CO, O, NR_{L2}, C₃₋₅ cycloalkyl, 6- to 9-membered aryl, 5- or 6-membered heteroaryl containing 1-3 heteroatoms selected from N, S and O, 5-membered monoheterocyclic alkyl containing 1-3 heteroatoms selected from N, S and O, 7- or 8-membered monoheterocyclic alkyl containing 1-3 heteroatoms selected from N, S and O, 5- to 10-membered fused-heterocyclic alkyl containing 1-3 heteroatoms selected from N, S and O, 6- to 10-membered bridged-heterocyclic alkyl containing 1-3 heteroatoms selected from N, S and O, or 7- to 12-membered spiro-heterocyclic alkyl containing 1-3 heteroatoms selected from N, S and O, wherein the alkylene, alkenylene, alkynylene, cycloalkyl, aryl, heteroaryl, monoheterocyclic alkyl, fused-heterocyclic alkyl, bridged-heterocyclic alkyl, and spiro-heterocyclic alkyl are optionally substituted with 1-3 groups selected from halogen, ═O, CN, C₁₋₄ alkyl, C₁₋₄ alkoxy, OH, and NH₂; in some embodiments, L₂

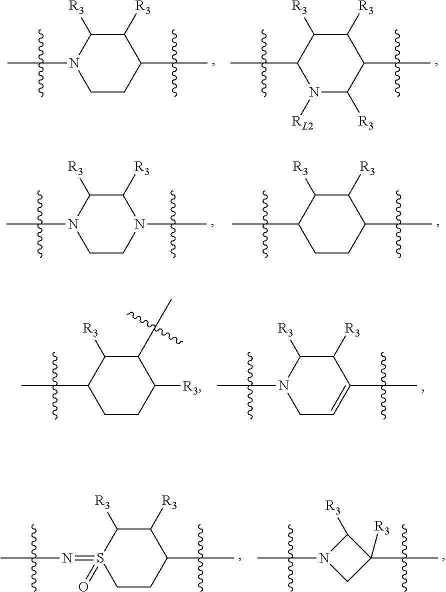

C₁₋₄ alkylene, C₂₋₄ alkenylene, C₂₋₄ alkynylene, CO, O, NR_{L2}, C₃₋₅ cycloalkyl, 6- to 9-membered aryl, 5- or 6-membered heteroaryl containing 1-3 heteroatoms selected from N, S and O, 5-membered monoheterocyclic alkyl containing 1-3 heteroatoms selected from N, S and O, 7- or 8-membered monoheterocyclic alkyl containing 1-3 heteroatoms selected from N, S and O, 5- to 10-membered fused-heterocyclic alkyl containing 1-3 heteroatoms selected from N, S and O, 6- to 10-membered bridged-heterocyclic alkyl containing 1-3 heteroatoms selected from N, S and O, or 7- to 12-membered spiro-heterocyclic alkyl containing 1-3 heteroatoms selected from N, S and O, wherein the alkylene, alkenylene, alkynylene, cycloalkyl, aryl, heteroaryl, monoheterocyclic alkyl, fused-heterocyclic alkyl, bridged-heterocyclic alkyl, and spiro-heterocyclic alkyl are optionally substituted with 1-3 groups selected from halogen, ═O, CN, C₁₋₄ alkyl, C₁₋₄ alkoxy, OH, and NH₂; in some embodiments, each L₂ is independently selected from CO, O, NR_{L2}, C₁₋₄ alkylene, C₂₋₄ alkenylene, C₂₋₄ alkynylene,

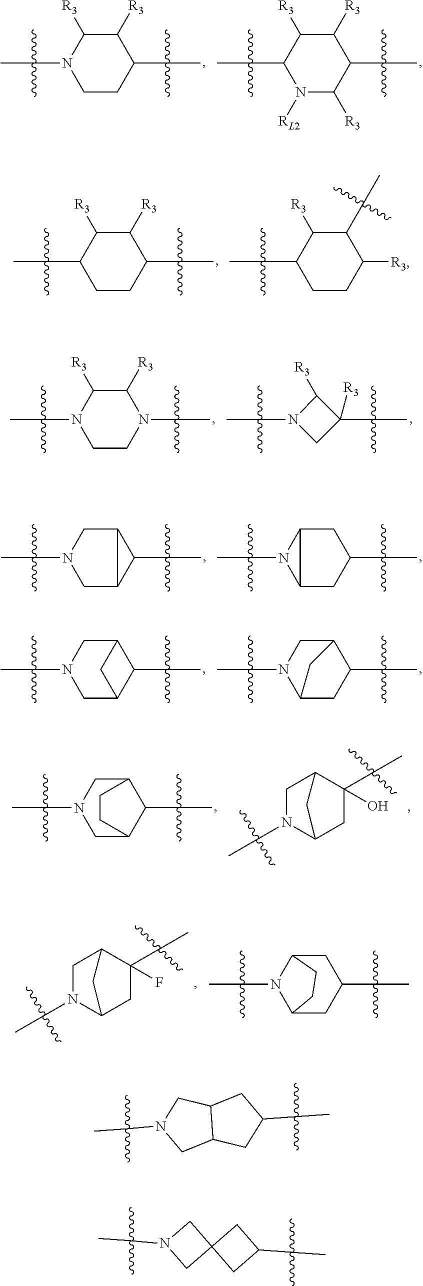

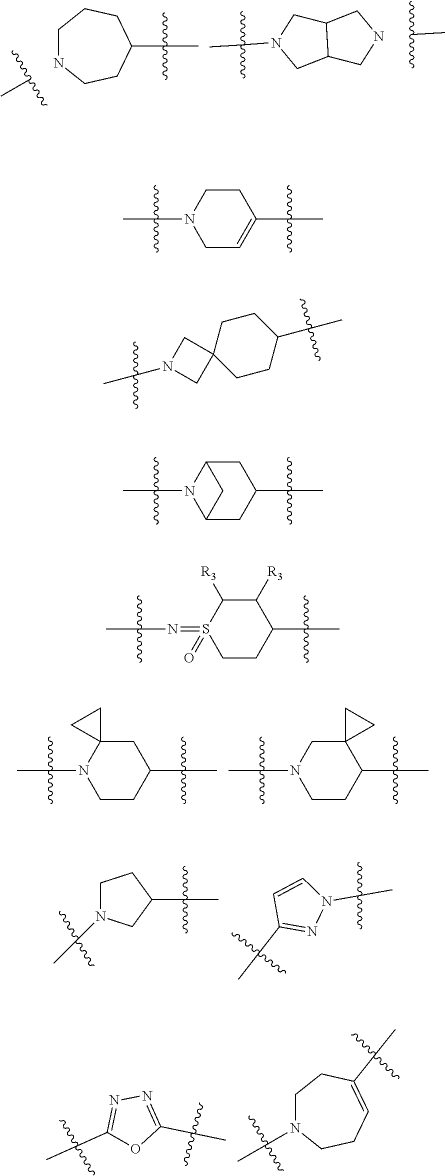

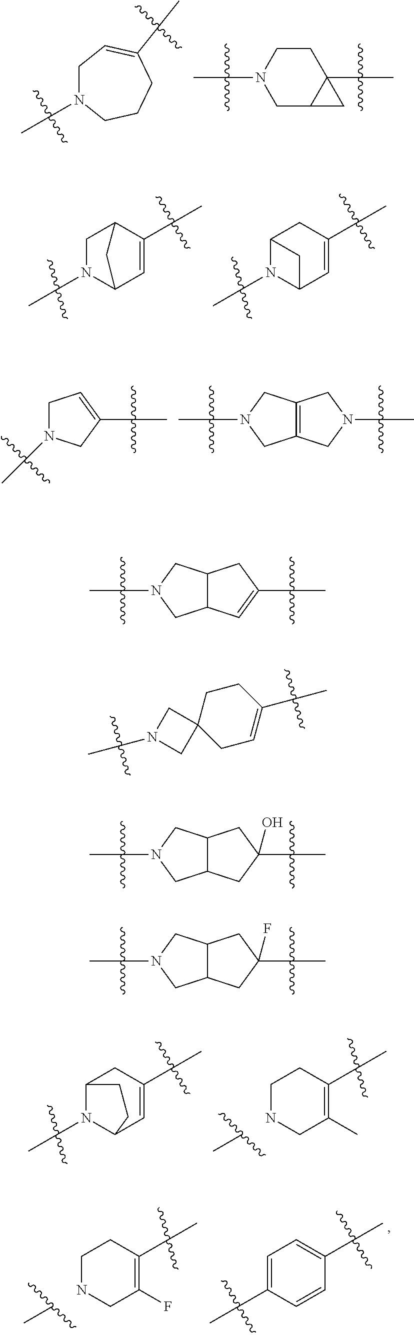

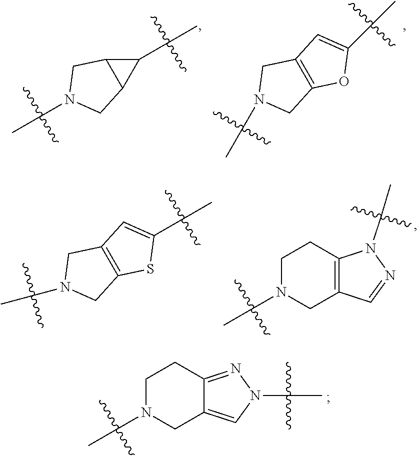 [SMILES: *C1C2CN(*)CC12 ; *c1cc2c(o1)CN(*)C2 ; *c1cc2c(s1)CN(*)C2 ; *N1CCc2c(cnn2*)C1 ; *N1CCc2nn(*)cc2C1]

in some embodiments, each L₂ is independently selected from CO, O, NR_{L2}, C₁₋₄ alkylene, C₂₋₄ alkenylene, C₂₋₄ alkynylene,

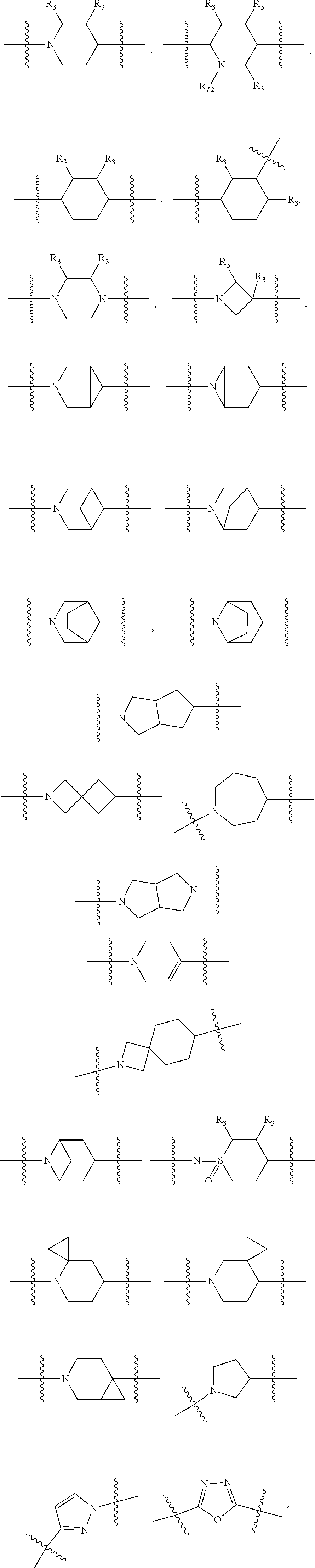

in some embodiments, each L₂ may also be independently selected from CO, O, NR_{L2}, C₁₋₄ alkylene, C₂₋₄ alkenylene, C₂₋₄ alkynylene,

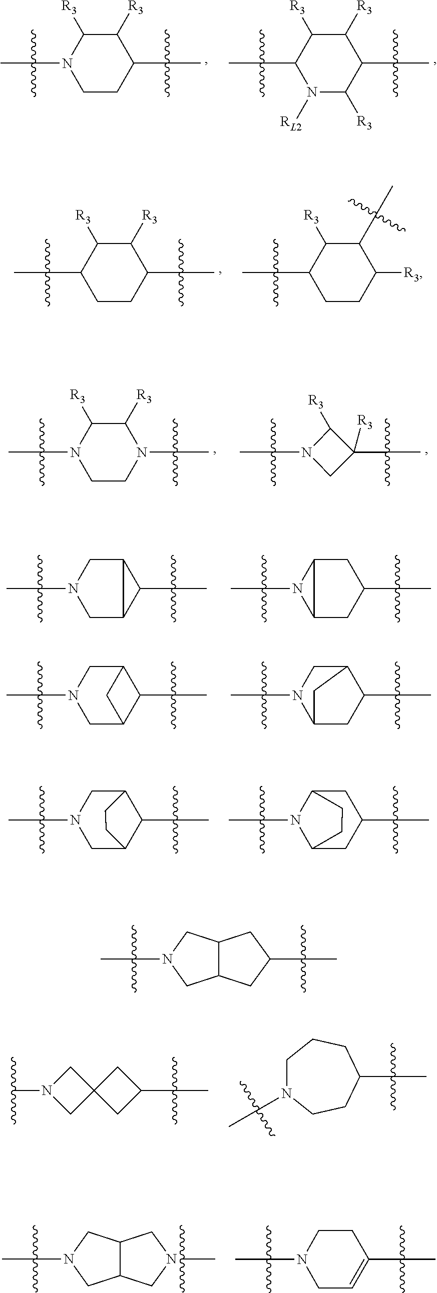

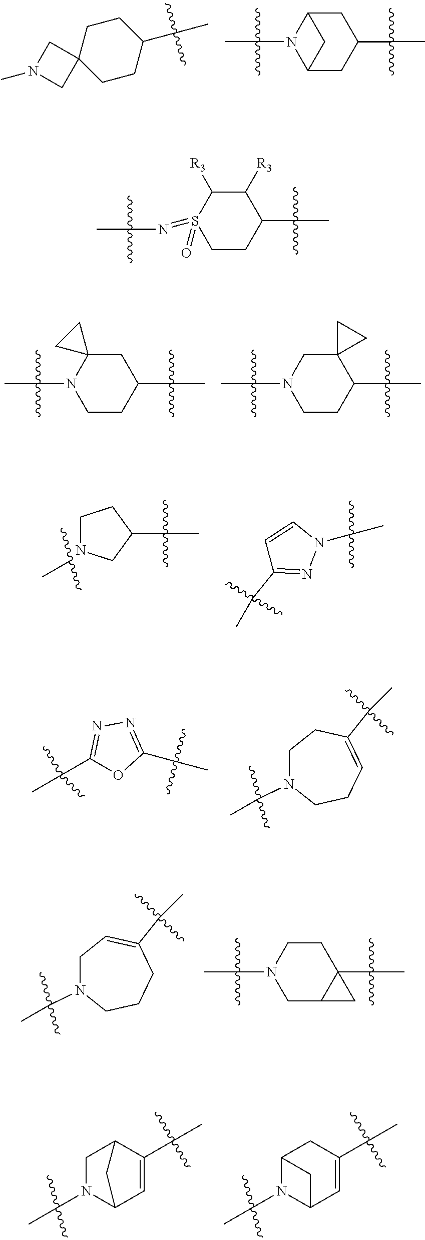

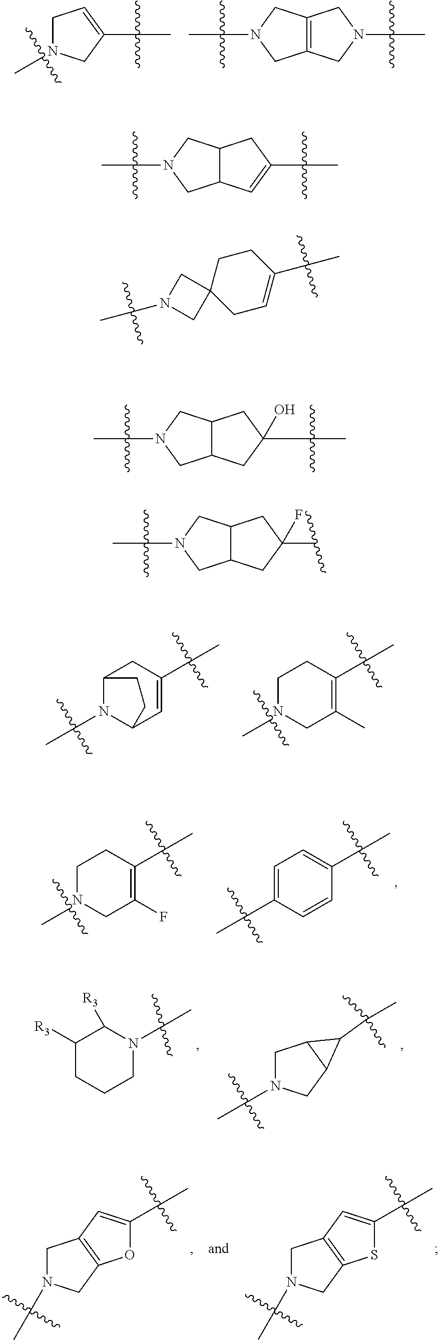

in some embodiments, each L₂ is independently selected from CO, O, NR_{L2}, C₁₋₄ alkylene, C₂₋₄ alkenylene, C₂₋₄ alkynylene,

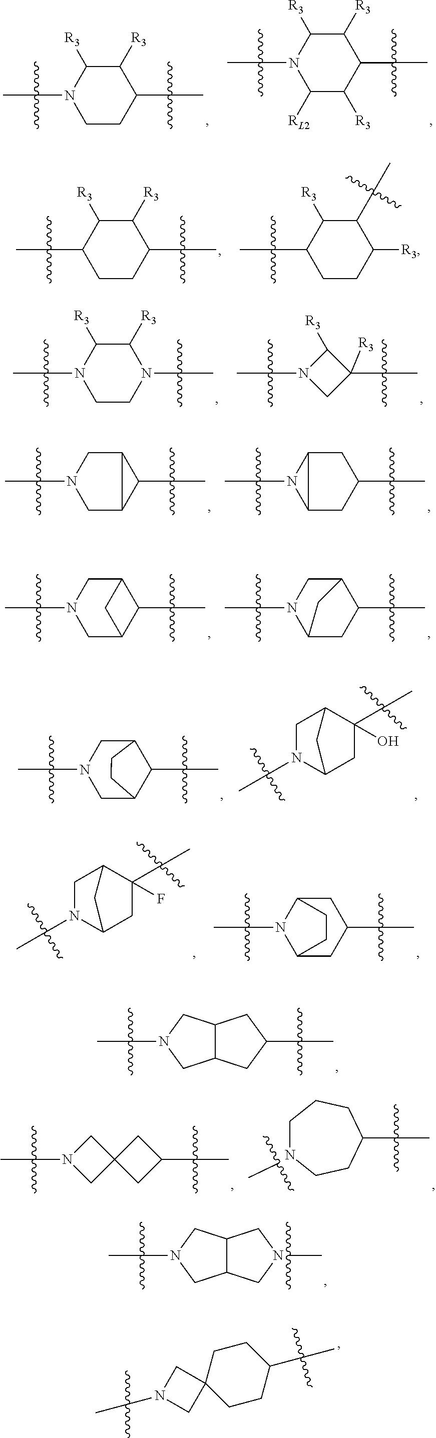

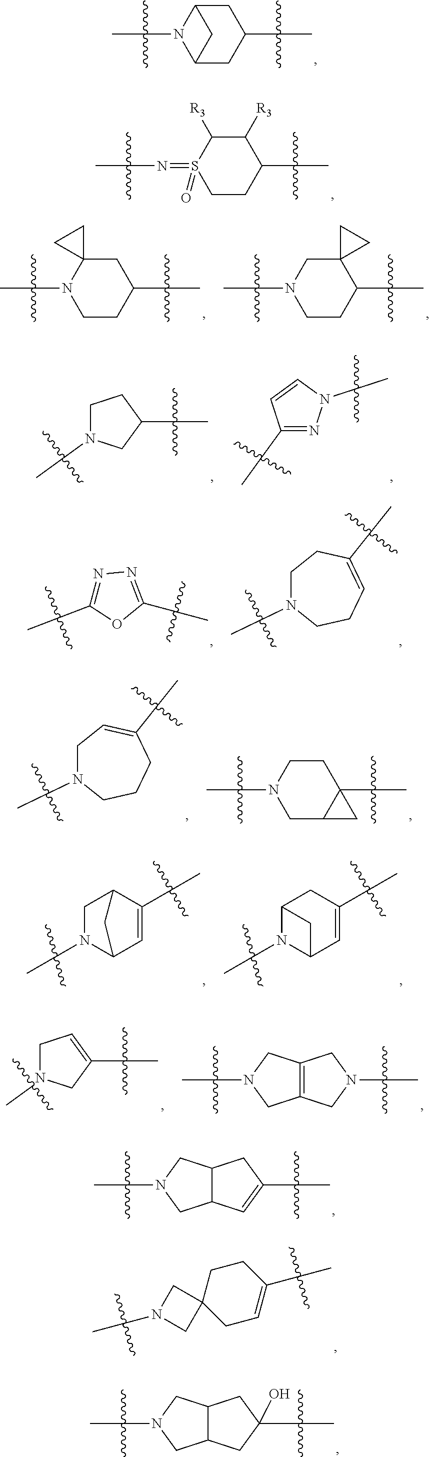

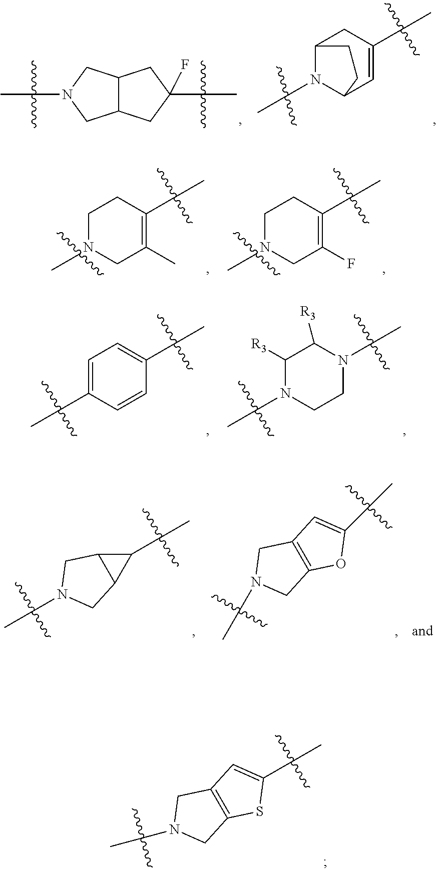

in all the solutions of the present invention, the sites of attachment of L₂ to the left and right groups are arbitrary, for example, in L₂

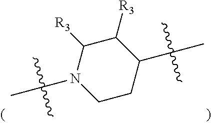

as one end, both N atom and C atom can be used as sites of attachment to Cy;
R_{L2} is H, C₁₋₄ alkyl, or C₃₋₆ cycloalkyl; in some embodiments, R_{L2} is H, C₁₋₄ alkyl, or C₃₋₆ cycloalkyl; in some embodiments, R_{L2} is H, methyl, ethyl, propyl, or isopropyl;
L is selected from one of the following structures:

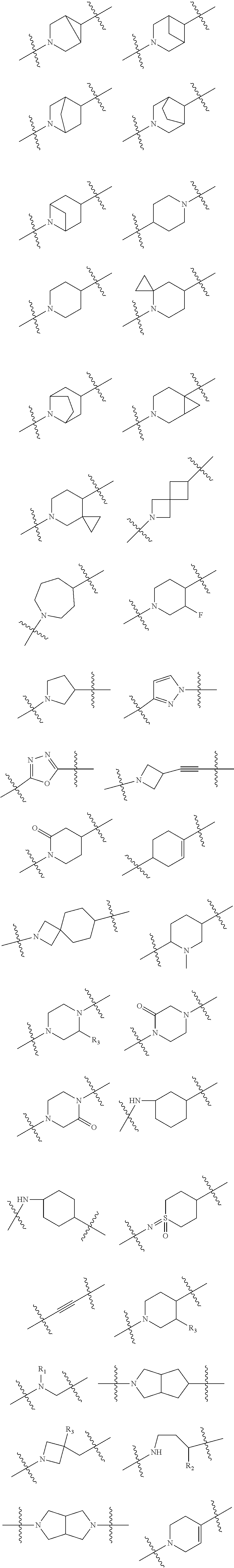

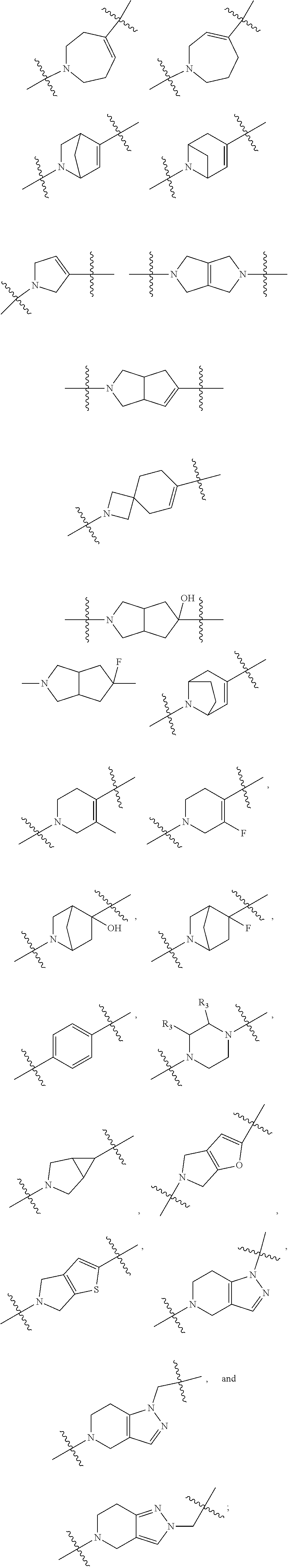

in some embodiments, L is selected from

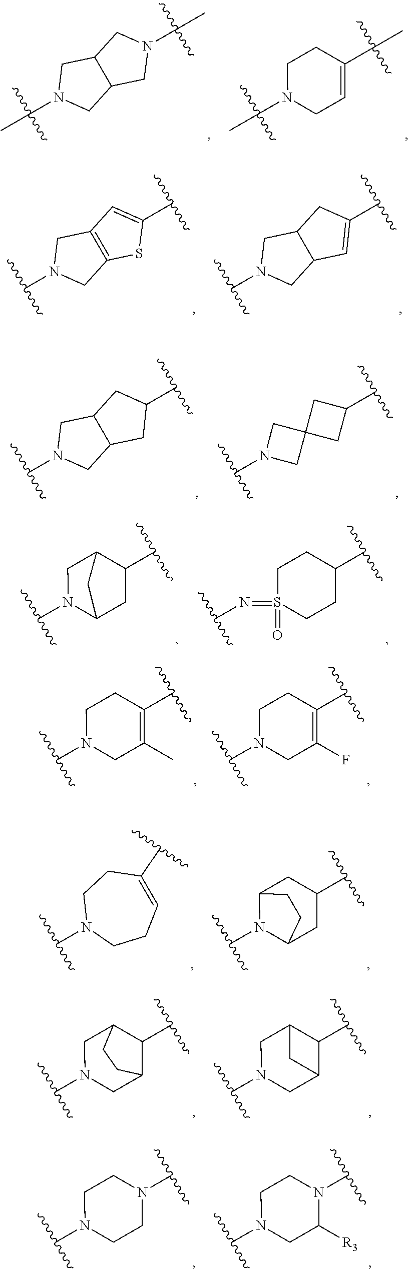

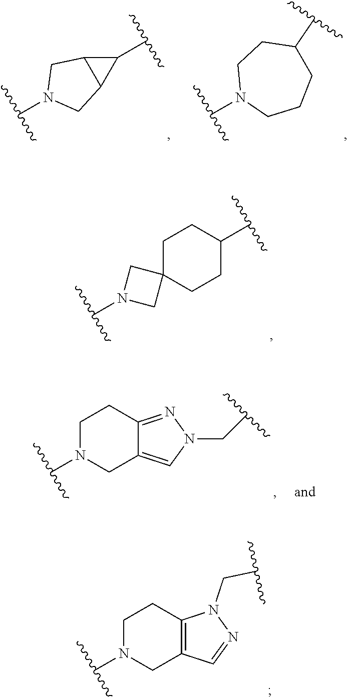 [SMILES: *C1C2CN(*)CC12 ; *C1CCCN(*)CC1 ; *C1CCC2(CC1)CN(*)C2 ; *Cn1cc2c(n1)CCN(*)C2 ; *Cn1ncc2c1CCN(*)C2]

in some embodiments, L is selected from

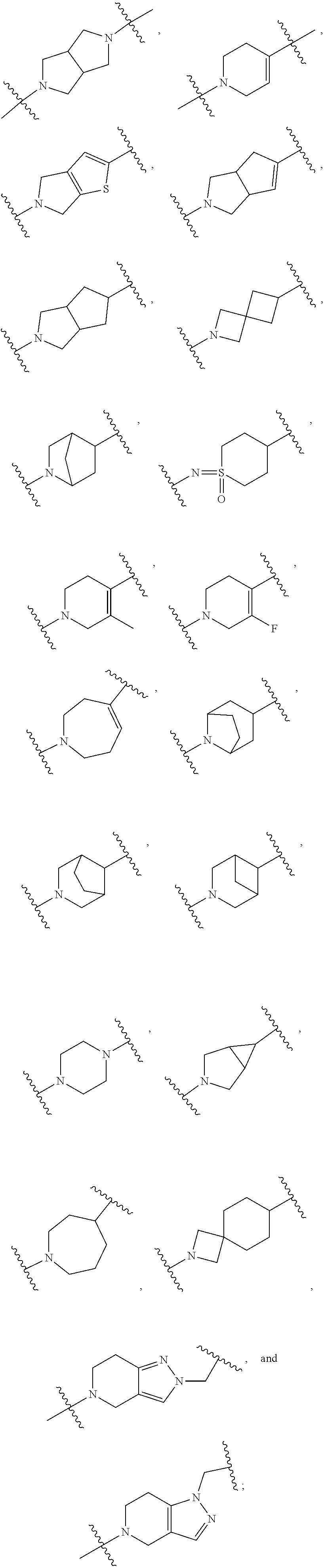

in some embodiments, L is selected from

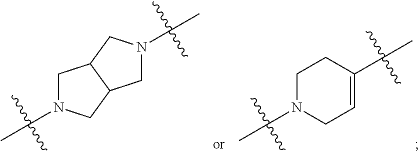 [SMILES: *N1CC2CN(*)CC2C1 ; *C1=CCN(*)CC1]

in some embodiments, L is selected from

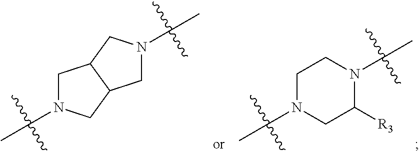

in some embodiments,

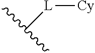

is selected from the following structures:

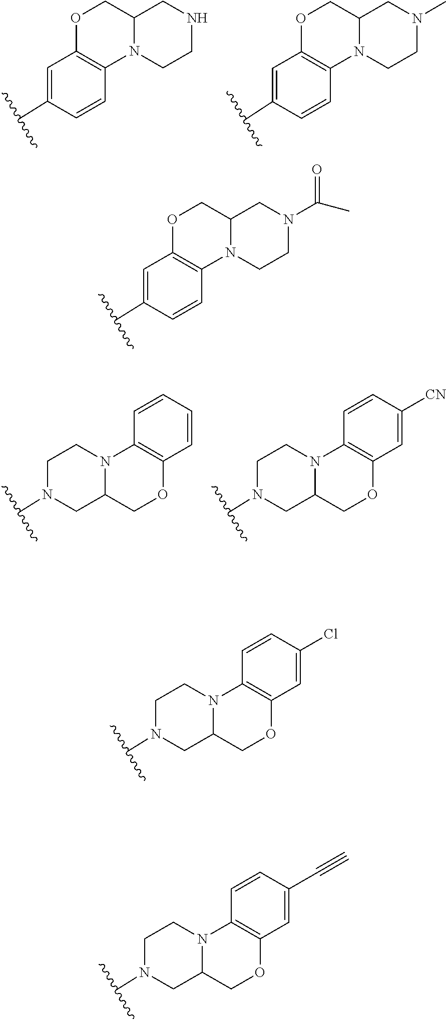

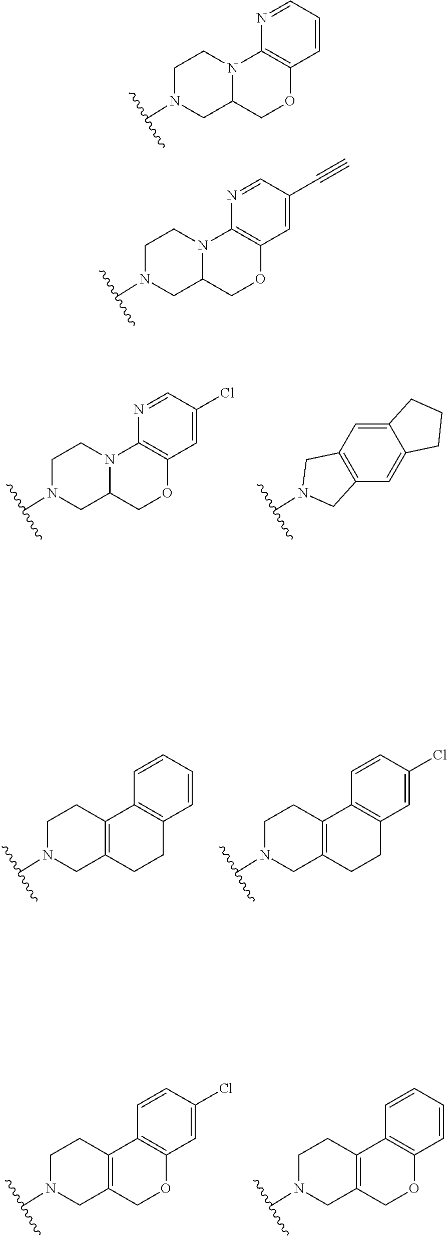

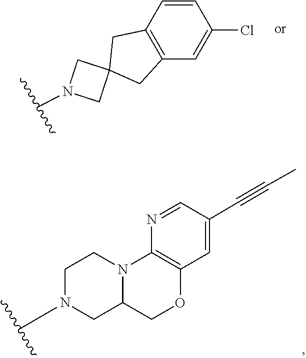 [SMILES: *N1CC2(Cc3ccc(Cl)cc3C2)C1 ; "or" ; *N1CCN2c3ncc(C#CC)cc3OCC2C1]

or selected from

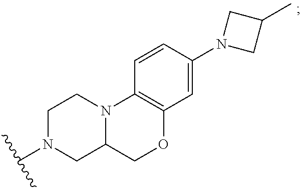 [SMILES: *N1CCN2c3ccc(N4CC(C)C4)cc3OCC2C1]

X₁ and X₂ are independently CR_{X1} or N; in some embodiments, X₁ and X₂ are CR_{X1}; in some embodiments, X₁ and X₂ are N;
each R_{X1} is independently H, halogen, C₁₋₄ alkyl, C₁₋₄ haloalkyl, C₂₋₄ alkenyl, C₂₋₄ alkynyl, or —SO₂NH₂, or R_{X1} and R₁, R_{X1} and R₂, or R_{X1} and R₃, together with the atoms to which they are attached, form C₃₋₈ cycloalkyl, 5- to 10-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂; in some embodiments, each R_{X1} is independently H, halogen, C₁₋₄ alkyl, or C₁₋₄ haloalkyl, or R_{X1} and R₁, R_{X1} and R₂, or R_{X1} and R₃ together with the atoms to which they are attached form C₃₋₆cycloalkyl, 5- to 7-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂; in some embodiments, each R_{X1} is independently H, halogen, C₁₋₄ alkyl, or C₁₋₄ haloalkyl, or R_{X1} and R₁, R_{X1} and R₂, or R_{X1} and R₃ together with the atoms to which they are attached form C₃₋₆ cycloalkyl, 5- to 7-membered heterocycloalkyl containing 1-3 heteroatoms selected from N, S and 0, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂; in some embodiments, each R_{X1} is independently H, F, Cl, Br, I, methyl, ethyl, propyl, or halomethyl, haloethyl, or halopropyl, or R_{X1} and R₁, R_{X1} and R₂, or R_{X1} and R₃ together with the atoms to which they are attached form C₅ cycloalkyl, C₆ cycloalkyl, 5-membered heterocycloalkyl or 6-membered heterocycloalkyl containing 1-3 heteroatoms selected from N, S and O, pyrrolyl, imidazolyl, furyl, pyranyl, morpholinyl, etc., which are optionally substituted with 1-3 groups selected from halogen, ═O, CN, C₁₋₄ alkyl, C₁₋₄ alkoxy, OH, and NH₂; in some embodiments, each R_{X1} is independently H, F, Cl, methyl, or ethyl, or R_{X1} and R₃ together with the atoms to which they are attached form C₅ cycloalkyl, C₆ cycloalkyl, 5-membered heterocycloalkyl containing 1-3 heteroatoms selected from N, S and O, or 6-membered heterocycloalkyl containing 1-3 heteroatoms selected from N, S and O;
each R is independently H, halogen, cyano, C₁₋₄ alkyl, C₂₋₄ alkenyl, C₂₋₄ alkynyl, C₃₋₈ cycloalkyl, 4- to 7-membered heterocycloalkyl containing 1-3 heteroatoms selected from N, S, O, Si and P, —SO₂NH₂, —NHSO₂NH₂, —NHCONH₂, —NHCOC₁₋₄ alkyl, —CONHC₁₋₄ alkyl, —NHSO₂C₁₋₄ alkyl, —SO₂NHC₁₋₄ alkyl, SO₂C₁₋₄ alkyl, —P(O)(C₁₋₄ alkyl)₂, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, —C(O)C₁₋₄ alkyl, and NH₂; or two adjacent R, or R and R_{X1} together with the atoms to which they are attached form C₃₋₈ cycloalkyl, 5- to 10-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, —C(O)C₁₋₄ alkyl, and NH₂; in some embodiments, each R is independently H, halogen, cyano, C₁₋₄ alkyl, C₂₋₄ alkenyl, C₂₋₄ alkynyl, C₃₋₈ cycloalkyl, 4- to 7-membered heterocycloalkyl containing 1-3 heteroatoms selected from N, S, O, Si and P, —SO₂NH₂, —NHSO₂NH₂, —NHCONH₂, —NHCOC₁₋₄ alkyl, —CONHC₁₋₄ alkyl, —NHSO₂C₁₋₄ alkyl, —SO₂NHC₁₋₄ alkyl, SO₂C₁₋₄ alkyl, —P(O)(C₁₋₄ alkyl)₂, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, and NH₂; or two adjacent R, or R and R_{X1} together with the atoms to which they are attached form C₃₋₈ cycloalkyl, 5- to 10-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂; in some embodiments, each R is independently H, halogen, cyano, C₁₋₄ alkyl, C₂₋₄ alkenyl, C₂₋₄ alkynyl, C₃₋₆ cycloalkyl, 4- to 6-membered heterocycloalkyl containing 1-3 heteroatoms selected from N, S, O, Si and P, —SO₂NH₂, —NHSO₂NH₂, —NHSO₂C₁₋₄ alkyl, —SO₂NHC₁₋₄ alkyl, SO₂C₁₋₄ alkyl, —P(O)(C₁₋₂alkyl)₂, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, and NH₂; or two adjacent R, or R and R_{X1} together with the atoms to which they are attached form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂; in some embodiments, each R is independently H, halogen, cyano, C₁₋₄ alkyl, C₂₋₄ alkynyl, C₃₋₆ cycloalkyl, 4- to 6-membered heterocycloalkyl containing 1-3 heteroatoms selected from N, S, O, Si and P, —SO₂NH₂, —NHSO₂C₁₋₄ alkyl, —SO₂NHC₁₋₄ alkyl, SO₂C₁₋₄ alkyl, —P(O)(CH₃)₂, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, and NH₂; or two adjacent R, or R and R_{X1} together with the atoms to which they are attached form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂; in some embodiments, each R is independently H, halogen, C₁₋₄ alkyl, C₂₋₄ alkenyl, C₂₋₄ alkynyl, —SO₂NH₂, —NHSO₂NH₂, —NHCONH₂, —NHCOC₁₋₄ alkyl, —CONHC₁₋₄ alkyl, —NHSO₂C₁₋₄ alkyl, —SO₂NHC₁₋₄ alkyl, SO₂C₁₋₄ alkyl, SCF₃, SF₅, or C₁₋₄ haloalkyl, or two adjacent R, or R and R_{X1} together with the atoms to which they are attached form C₃₋₈ cycloalkyl, 5- to 10-membered heterocycloalkyl containing 1-3 heteroatoms selected from N, S, O, P and B, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂ or —C(O)C₁₋₄ alkyl; in some embodiments, each R is independently H, halogen, C₁₋₄ alkyl, C₂₋₄ alkenyl, C₂₋₄ alkynyl, —SO₂NH₂, —NHSO₂NH₂, —NHSO₂C₁₋₄ alkyl, —SO₂NHC₁₋₄ alkyl, SO₂C₁₋₄ alkyl, SCF₃, SF₅, or C₁₋₄ haloalkyl, or two adjacent R, or R and R_{X1} together with the atoms to which they are attached form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂; in some embodiments, each R is independently H, halogen, C₁₋₄ alkyl, C₂₋₄ alkynyl, —SO₂NH₂, —NHSO₂C₁₋₄ alkyl, —SO₂NHC₁₋₄ alkyl, SO₂C₁₋₄ alkyl, SCF₃, SF₅, or C₁₋₄ haloalkyl, or two adjacent R, or R and R_{X1} together with the atoms to which they are attached form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂; in some embodiments, each R is independently H, F, Cl, Br, I, methyl, ethyl, propyl, methenyl, ethynyl, propynyl, —SO₂NH₂, —NHSO₂C₁₋₄ alkyl (such as methyl, ethyl, or propyl), —SO₂NHC₁₋₄ alkyl (such as methyl, ethyl, or propyl), SO₂C₁₋₄ alkyl (such as methyl, ethyl, or propyl), SCF₃, SF₅, or C₁₋₄ haloalkyl (such as methyl, ethyl, or propyl), or two adjacent R, or R and R_{X1} together with the atoms to which they are attached form cyclobutenyl, cyclopentenyl, cyclohexenyl, cyclopentadienyl, cyclohexadienyl, etc., which are optionally substituted with 1-3 groups selected from halogen, ═O, CN, C₁₋₄ alkyl, C₁₋₄ alkoxy, OH, and NH₂, or —C(O)C₁₋₄ alkyl; R₁ is H, C₁₋₄ alkyl, C₁₋₄ haloalkyl, or C₃₋₆ cycloalkyl; in some embodiments, R₁ is H, C₁₋₄ alkyl, or C₁₋₄ haloalkyl; in some embodiments, R₁ is H, methyl, ethyl, propyl, isopropyl, or halogenated methyl, ethyl, propyl, or isopropyl, wherein the halogen includes F, Cl, Br, and I; R₂ is H, C₁₋₄ alkyl, C₁₋₄ haloalkyl, C₁₋₄ alkoxy, —NHC₁₋₄ alkyl, or C₃₋₆ cycloalkyl; in some embodiments, R₂ is H, C₁₋₄ alkyl, C₁₋₄ haloalkyl, or C₁₋₄ alkoxy; in some embodiments, R₂ is H, C₁₋₄ alkyl, or C₁₋₄ haloalkyl; in some embodiments, R₂ is H, methyl, ethyl, or propyl; R₃ is H, halogen, ═O, CN, C₁₋₄ alkyl, or C₁₋₄ alkoxy; in some embodiments, R₃ is H, F, Cl, ═O, CN, C₁₋₃ alkyl, or C₁₋₃ alkoxy; in some embodiments, R₃ is H, halogen, ═O, CN, or C₁₋₄ alkyl; in some embodiments, R₃ is H, halogen, ═O, CN, methyl, ethyl, or propyl; in some embodiments, R₃ is H, F, Cl, ═O, CN, or C₁₋₃ alkyl;
n is 0, 1, 2, 3, or 4; in some embodiments, n is 0, 1, 2, or 3; in some embodiments, n is 0, 1, or 2;
m is 0, 1, 2, or 3; in some embodiments, m is 0, 1, or 2; in some embodiments, n is 0 or 1;
provided that
the compound is not

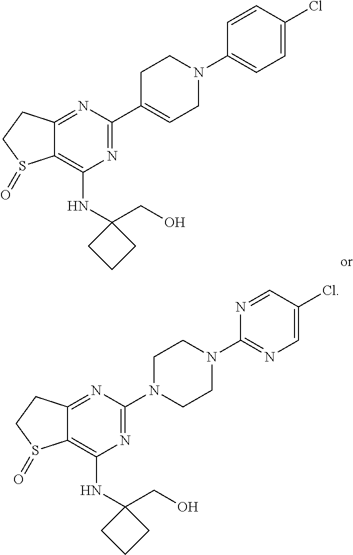 [SMILES: O=S1CCc2nc(C3=CCN(c4ccc(Cl)cc4)CC3)nc(NC3(CO)CCC3)c21 ; O=S1CCc2nc(N3CCN(c4ncc(Cl)cn4)CC3)nc(NC3(CO)CCC3)c21]

Specific solution 1 provided by the present invention relates to a compound represented by formula I or a stereoisomer or pharmaceutically acceptable salt thereof,

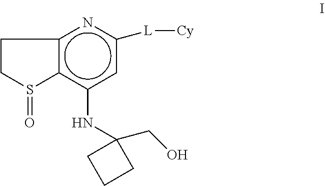

wherein Cy is selected from Cy1, Cy2, a 5-membered monocyclic heteroaryl, or an 8- to 10-membered bicyclic heteroaryl, wherein the monocyclic heteroaryl and the bicyclic heteroaryl are optionally further substituted with 1-3 R; furthermore, Cy is selected from Cy1 or Cy2;

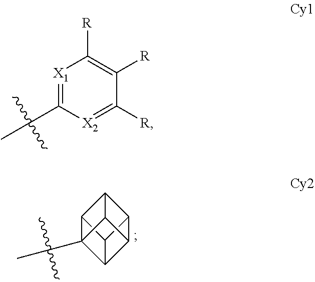

Cy2 is optionally substituted with 1-3 R;
L is -(L₁)n-(L₂)m-;
L₁ is —NR₁—, —CR₂═N—, —CR₂═CR₂—, or —CR₂R₂—;

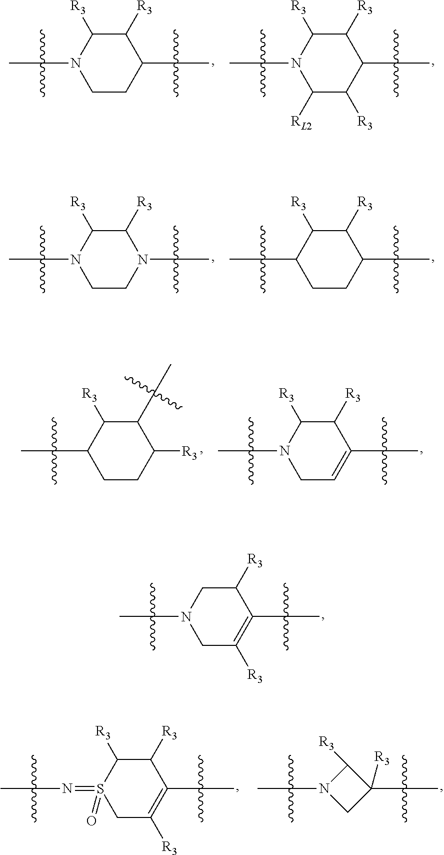

C₁₋₄ alkylene, C₂₋₄ alkenylene, C₂₋₄ alkynylene, CO, O, NR_{L2}, C₃₋₅ cycloalkyl, 6- to 9-membered aryl, 5- or 6-membered heteroaryl containing 1-3 heteroatoms selected from N, S and O, 5-membered monoheterocyclic alkyl containing 1-3 heteroatoms selected from N, S and O, 7- or 8-membered monoheterocyclic alkyl containing 1-3 heteroatoms selected from N, S and O, 5- to 10-membered fused-heterocyclic alkyl containing 1-3 heteroatoms selected from N, S and O, 6- to 10-membered bridged-heterocyclic alkyl containing 1-3 heteroatoms selected from N, S and O, or 7- to 12-membered spiro-heterocyclic alkyl containing 1-3 heteroatoms selected from N, S and O, wherein the alkylene, alkenylene, alkynylene, cycloalkyl, aryl, heteroaryl, monoheterocyclic alkyl, fused-heterocyclic alkyl, bridged-heterocyclic alkyl, and spiro-heterocyclic alkyl are optionally substituted with 1-3 groups selected from halogen, ═O, CN, C₁₋₄ alkyl, C₁₋₄ alkoxy, OH, and NH₂;
R_{L2} is H, C₁₋₄ alkyl, or C₃₋₆ cycloalkyl;
X₁ and X₂ are independently CR_{X1} or N;
each R_{X1} is independently H, halogen, C₁₋₄ alkyl, C₁₋₄ haloalkyl, C₂₋₄ alkenyl, C₂₋₄ alkynyl, or —SO₂NH₂, or R_{X1} and R₁, R_{X1} and R₂, or R_{X1} and R₃, together with the atoms to which they are attached, form C₃₋₈ cycloalkyl, 5- to 10-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂; each R is independently H, halogen, cyano, C₁₋₄ alkyl, C₂₋₄ alkenyl, C₂₋₄ alkynyl, C₃₋₈ cycloalkyl, 4- to 7-membered heterocycloalkyl containing 1-3 heteroatoms selected from N, S, O, Si and P, —SO₂NH₂, —NHSO₂NH₂, —NHCONH₂, —NHCOC₁₋₄ alkyl, —CONHC₁₋₄ alkyl, —NHSO₂C₁₋₄ alkyl, —SO₂NHC₁₋₄ alkyl, SO₂C₁₋₄ alkyl, —P(O)(C₁₋₄ alkyl)₂, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, —C(O)C₁₋₄ alkyl, and NH₂; or two adjacent R, or R and R_{X1} together with the atoms to which they are attached form C₃₋₈ cycloalkyl, 5- to 10-membered heterocycloalkyl containing 1-3 heteroatoms selected from N, S, O, P and B, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂, or —C(O)C₁₋₄ alkyl; or each R is independently H, halogen, C₁₋₄ alkyl, C₂₋₄ alkenyl, C₂₋₄ alkynyl, —SO₂NH₂, —NHSO₂NH₂, —NHCONH₂, —NHCOC₁₋₄ alkyl, —CONHC₁₋₄ alkyl, —NHSO₂C₁₋₄ alkyl, —SO₂NHC₁₋₄ alkyl, SO₂C₁₋₄ alkyl, SCF₃, SF₅, or C₁₋₄ haloalkyl, or two adjacent R, or R and R_{X1} together with the atoms to which they are attached form C₃₋₈ cycloalkyl, 5- to 10-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂, or —C(O)C₁₋₄ alkyl;
R₁ is H, C₁₋₄ alkyl, C₁₋₄ haloalkyl, or C₃₋₆ cycloalkyl;
R₂ is H, C₁₋₄ alkyl, C₁₋₄ haloalkyl, C₁₋₄ alkoxy, —NHC₁₋₄ alkyl, or C₃₋₆ cycloalkyl;
R₃ is H, halogen, ═O, CN, C₁₋₄ alkyl, or C₁₋₄ alkoxy;
n is 0, 1, 2, 3, or 4;
m is 0, 1, 2, or 3;
provided that
the compound is not

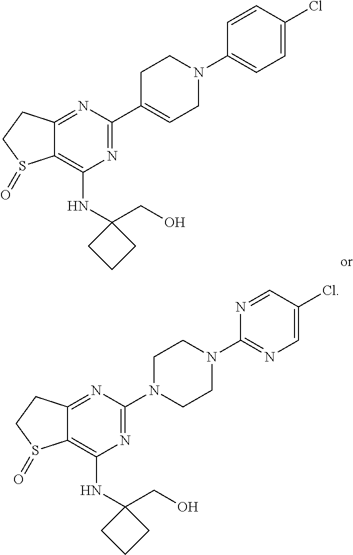 [SMILES: O=S1CCc2nc(C3=CCN(c4ccc(Cl)cc4)CC3)nc(NC3(CO)CCC3)c21 ; O=S1CCc2nc(N3CCN(c4ncc(Cl)cn4)CC3)nc(NC3(CO)CCC3)c21]

Furthermore, A compound as represented by formula (I) or a stereoisomer or pharmaceutically acceptable salt thereof,

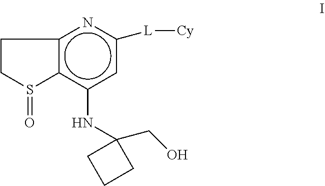

wherein Cy is selected from Cy1 or Cy2;

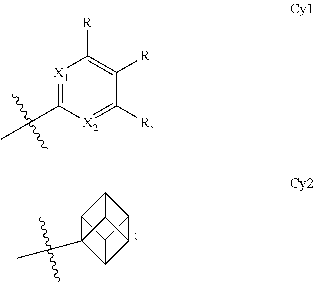

Cy2 is optionally substituted with 1-3 R;
L is -(L₁)n-(L₂)m-;
L₁ is —NR₁—, —CR₂═N—, —CR₂═CR₂—, or —CR₂R₂—;
L₂ is

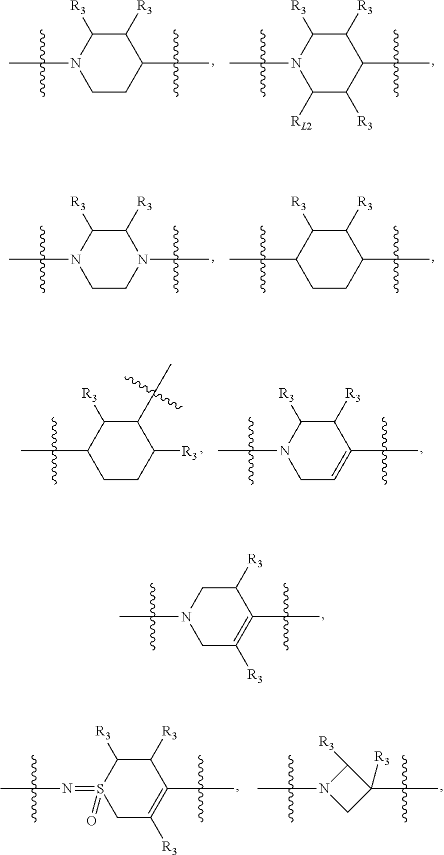

C₁₋₄ alkylene, C₂₋₄ alkenylene, C₂₋₄ alkynylene, CO, O, NR_{L2}, C₃₋₅ cycloalkyl, 5- or 6-membered heteroaryl containing 1-3 heteroatoms selected from N, S and O, 5-membered monoheterocyclic alkyl containing 1-3 heteroatoms selected from N, S and O, 7- or 8-membered monoheterocyclic alkyl containing 1-3 heteroatoms selected from N, S and O, 5- to 10-membered fused-heterocyclic alkyl containing 1-3 heteroatoms selected from N, S and O, 6-to 10-membered bridged-heterocyclic alkyl containing 1-3 heteroatoms selected from N, S and O, or 7- to 12-membered spiro-heterocyclic alkyl containing 1-3 heteroatoms selected from N, S and O, wherein the alkylene, alkenylene, alkynylene, cycloalkyl, heteroaryl, monoheterocyclic alkyl, fused-heterocyclic alkyl, bridged-heterocyclic alkyl, and spiro-heterocyclic alkyl are optionally substituted with 1-3 groups selected from halogen, ═O, CN, C₁₋₄ alkyl, C₁₋₄ alkoxy, OH, and NH₂; or L₂ is

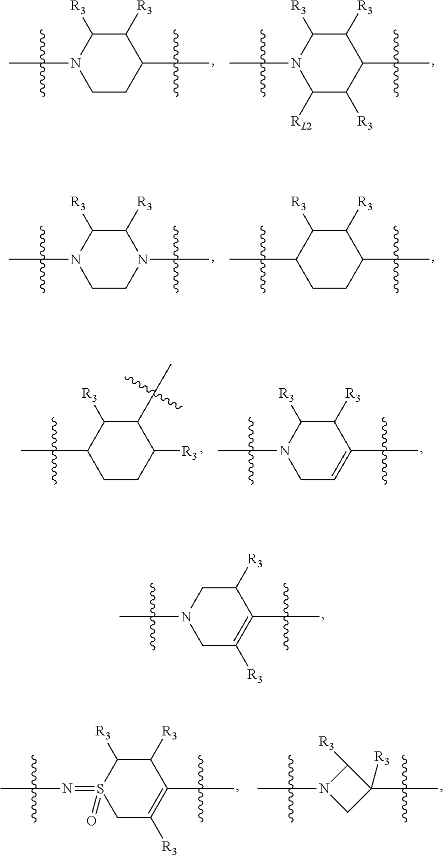

C₁₋₄ alkylene, C₂₋₄ alkenylene, C₂₋₄ alkynylene, CO, O, NR_{L2}, C₃₋₅ cycloalkyl, 5- or 6-membered heteroaryl containing 1-3 heteroatoms selected from N, S and O, 5-membered monoheterocyclic alkyl containing 1-3 heteroatoms selected from N, S and O, 7- or 8-membered monoheterocyclic alkyl containing 1-3 heteroatoms selected from N, S and O, 5- to 10-membered fused-heterocyclic alkyl containing 1-3 heteroatoms selected from N, S and O, 6- to 10-membered bridged-heterocyclic alkyl containing 1-3 heteroatoms selected from N, S and O, or 7- to 12-membered spiro-heterocyclic alkyl containing 1-3 heteroatoms selected from N, S and O, wherein the alkylene, alkenylene, alkynylene, cycloalkyl, heteroaryl, monoheterocyclic alkyl, fused-heterocyclic alkyl, bridged-heterocyclic alkyl, and spiro-heterocyclic alkyl are optionally substituted with 1-3 groups selected from halogen, ═O, CN, C₁₋₄ alkyl, C₁₋₄ alkoxy, OH, and NH₂;
R_{L2} is H, C₁₋₄ alkyl, or C₃₋₆ cycloalkyl;
X₁ and X₂ are independently CR_{X1} or N;
each R_{X1} is independently H, halogen, C₁₋₄ alkyl, C₁₋₄ haloalkyl, C₂₋₄ alkenyl, C₂₋₄ alkynyl, or —SO₂NH₂, or R_{X1} and R₁, R_{X1} and R₂, or R_{X1} and R₃, together with the atoms to which they are attached, form C₃₋₈ cycloalkyl, 5- to 10-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂; each R is independently H, halogen, C₁₋₄ alkyl, C₂₋₄ alkenyl, C₂₋₄ alkynyl, —SO₂NH₂, —NHSO₂NH₂, —NHCONH₂, —NHCOC₁₋₄ alkyl, —CONHC₁₋₄ alkyl, —NHSO₂C₁₋₄ alkyl, —SO₂NHC₁₋₄ alkyl, SO₂C₁₋₄ alkyl, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, —C(O)C₁₋₄ alkyl, and NH₂; or two adjacent R, or R and R_{X1} together with the atoms to which they are attached form C₃₋₈ cycloalkyl, 5- to 10-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂, or —C(O)C₁₋₄ alkyl;
R₁ is H, C₁₋₄ alkyl, C₁₋₄ haloalkyl, or C₃₋₆ cycloalkyl;
R₂ is H, C₁₋₄ alkyl, C₁₋₄ haloalkyl, C₁₋₄ alkoxy, —NHC₁₋₄ alkyl, or C₃₋₆ cycloalkyl;
R₃ is H, halogen, ═O, CN, C₁₋₄ alkyl, or C₁₋₄ alkoxy;
n is 0, 1, 2, 3, or 4;
m is 0, 1, 2, or 3;
provided that
the compound is not

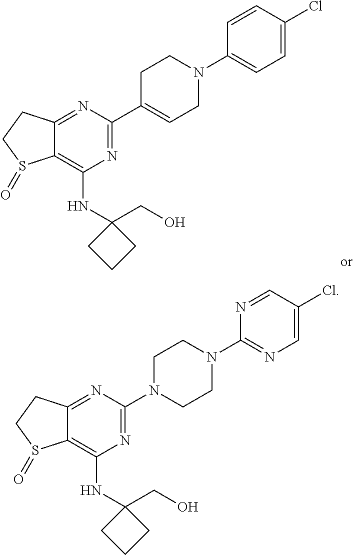 [SMILES: O=S1CCc2nc(C3=CCN(c4ccc(Cl)cc4)CC3)nc(NC3(CO)CCC3)c21 ; O=S1CCc2nc(N3CCN(c4ncc(Cl)cn4)CC3)nc(NC3(CO)CCC3)c21]

Specific solution 2 of the present invention relates to the compound or the stereoisomer or pharmaceutically acceptable salt thereof according to solution 1,
wherein each L₂ is independently selected from CO, O, NR_{L2}, C₁₋₄ alkylene, C₂₋₄ alkenylene, C₂₋₄ alkynylene,

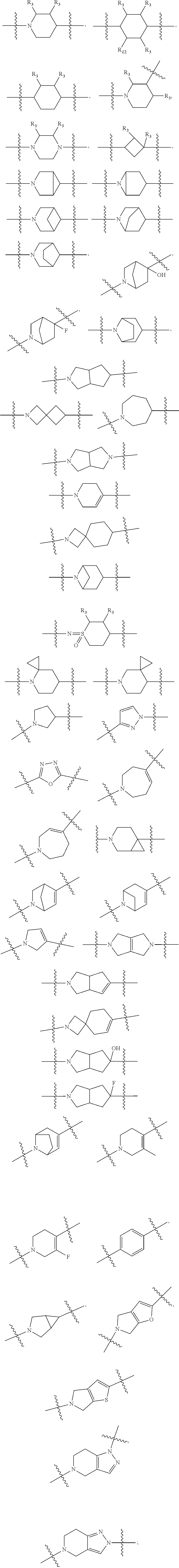

or each L₂ is independently selected from CO, O, NR_{L2}, C₁₋₄ alkylene, C₂₋₄ alkenylene, C₂₋₄ alkynylene

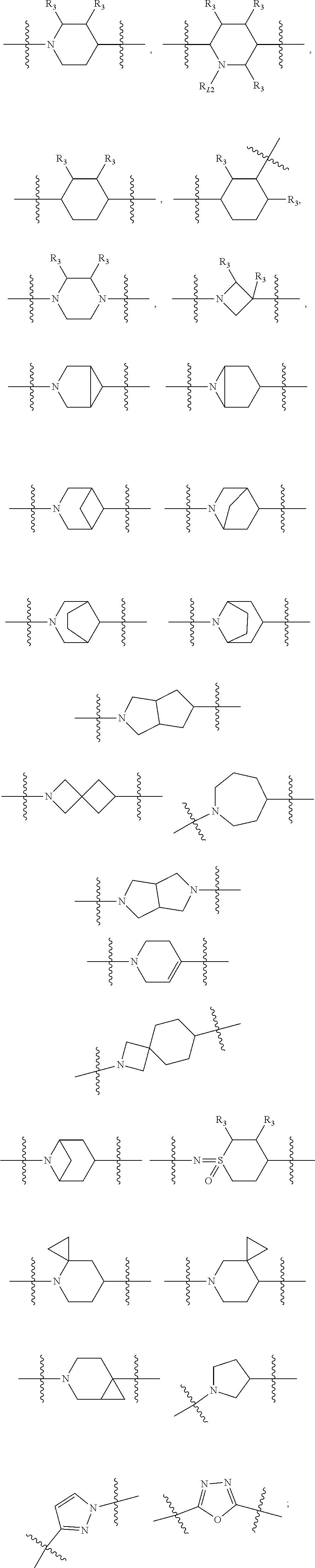

or each L₂ is independently selected from CO, O, NR_{L2}, C₁₋₄ alkylene, C₂₋₄ alkenylene, C₂₋₄ alkynylene,

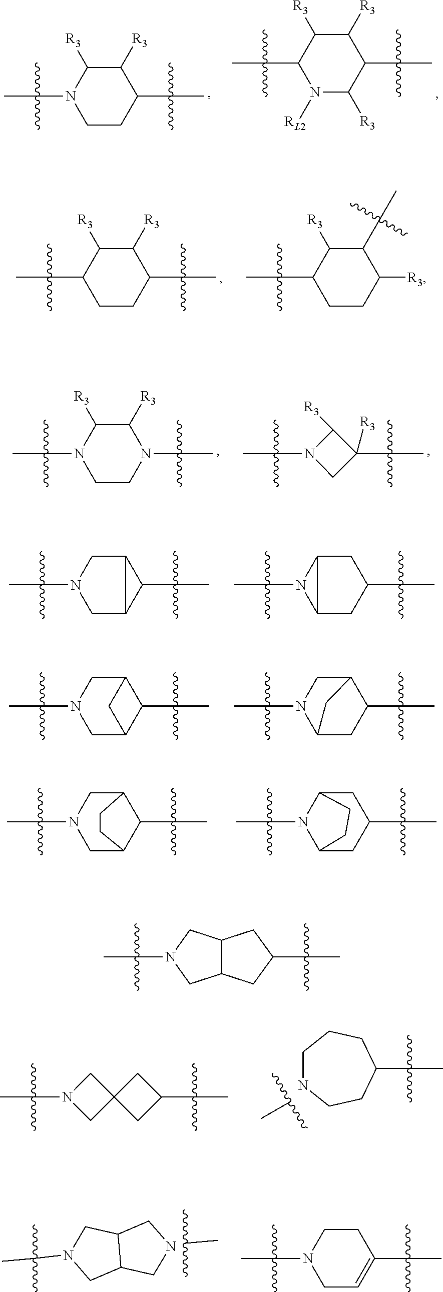

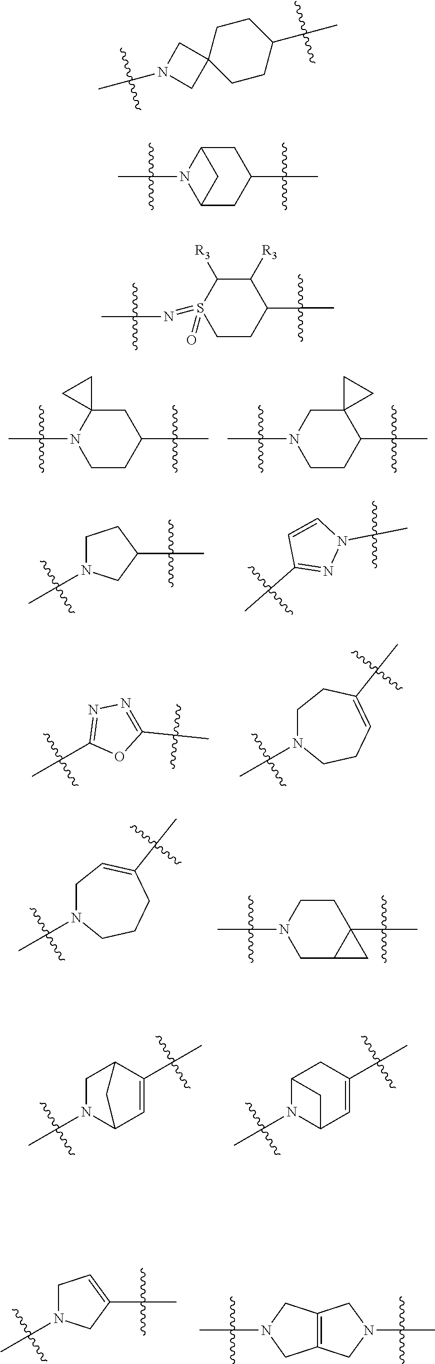

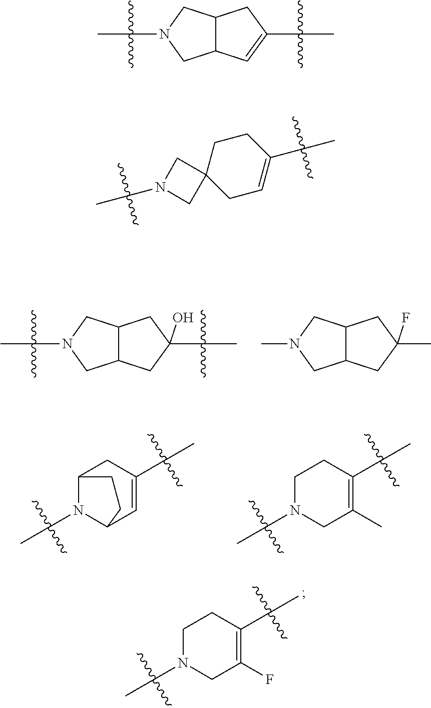

or each L₂ is independently selected from CO, O, NR_{L2}, C₁₋₄ alkylene, C₂₋₄ alkenylene, C₂₋₄alkynylene,

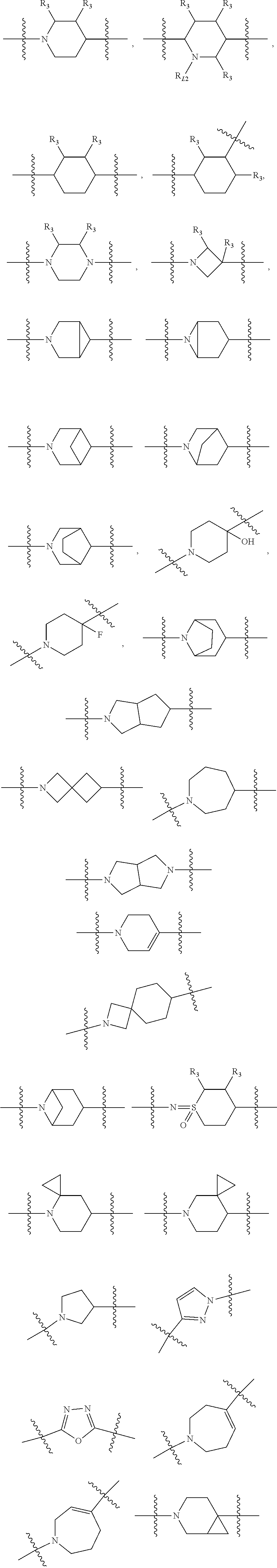

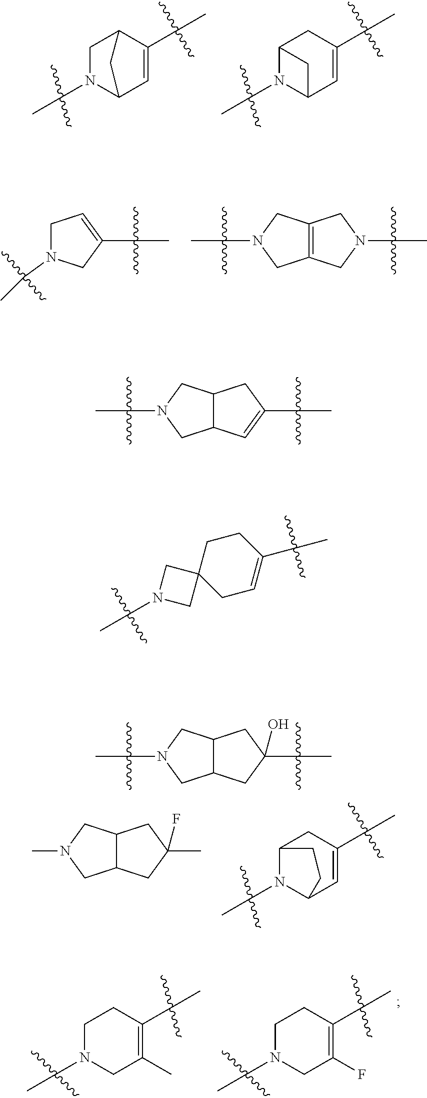

or each L₂ is independently selected from CO, O, NR_{L2}, C₁₋₄ alkylene, C₂₋₄ alkenylene, C₂₋₄ alkynylene

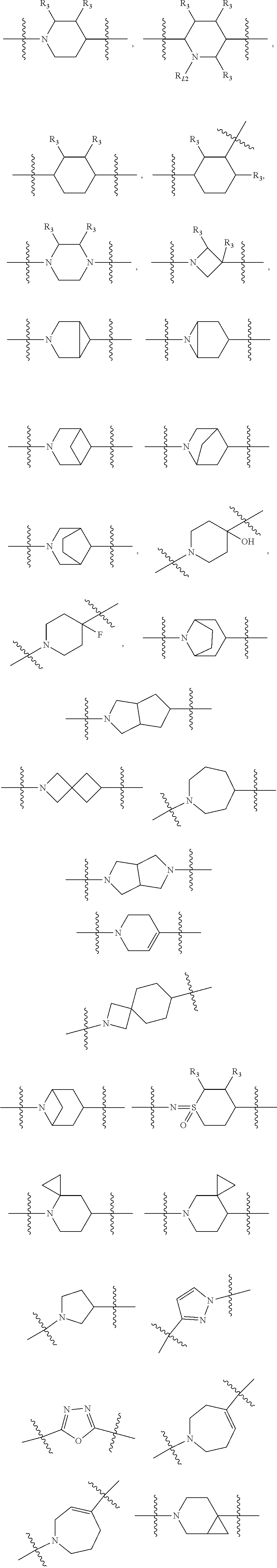

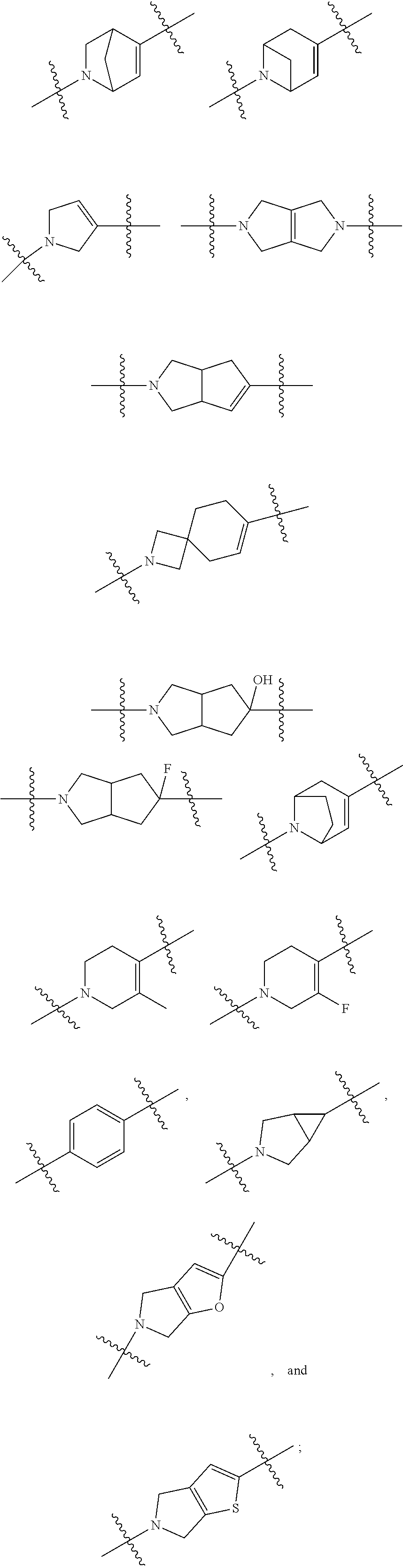

R_{L2} is H, C₁₋₄ alkyl, or C₃₋₆ cycloalkyl;
each R_{X1} is independently H, halogen, C₁₋₄ alkyl, or C₁₋₄ haloalkyl, or R_{X1} and R₁, R_{X1} and R₂, or R_{X1} and R₃ together with the atoms to which they are attached form C₃₋₆ cycloalkyl, 5- to 7-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
each R is independently H, halogen, cyano, C₁₋₄ alkyl, C₂₋₄ alkenyl, C₂₋₄ alkynyl, C₃₋₆ cycloalkyl, 4- to 6-membered heterocycloalkyl containing 1-3 heteroatoms selected from N, S, O, Si and P, —SO₂NH₂, —NHSO₂NH₂, —NHSO₂C₁₋₄ alkyl, —SO₂NHC₁₋₄ alkyl, SO₂C₁₋₄ alkyl, —P(O)(C₁ 2 alkyl)₂, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, and NH₂; or two adjacent R, or R and R_{X1} together with the atoms to which they are attached form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂; or each R is independently H, halogen, C₁₋₄ alkyl, C₂₋₄ alkenyl, C₂₋₄ alkynyl, —SO₂NH₂, —NHSO₂NH₂, —NHSO₂C₁₋₄ alkyl, —SO₂NHC₁₋₄ alkyl, SO₂C₁₋₄ alkyl, SCF₃, SF₅, or C₁₋₄ haloalkyl, or two adjacent R, or R and R_{X1} together with the atoms to which they are attached form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂; or two adjacent R, or R and R_{X1} together with the atoms to which they are attached form 5- to 10-membered heterocycloalkyl containing 1-3 heteroatoms selected from N, S, O, P and B, wherein the heterocycloalkyl is optionally substituted with 1-3 groups selected from halogen, ═O, CN, C₁₋₄ alkyl, C₁₋₄ alkoxy, OH, and NH₂;
R₁ is H, C₁₋₄ alkyl, or C₁₋₄ haloalkyl;
R₂ is H, C₁₋₄ alkyl, C₁₋₄ haloalkyl, or C₁₋₄ alkoxy;
R₃ is H, halogen, ═O, CN, or C₁₋₄ alkyl;
n is 0, 1, 2, 3, or 4;
m is 0, 1, or 2.

Solution 3 of the present invention relates to the compound or the stereoisomer or pharmaceutically acceptable salt thereof as in solution 1 or 2, wherein the compound has the structure of the following formula II,

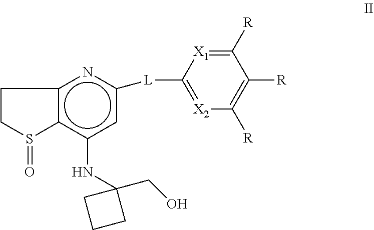

wherein L is selected from one of the following structures:

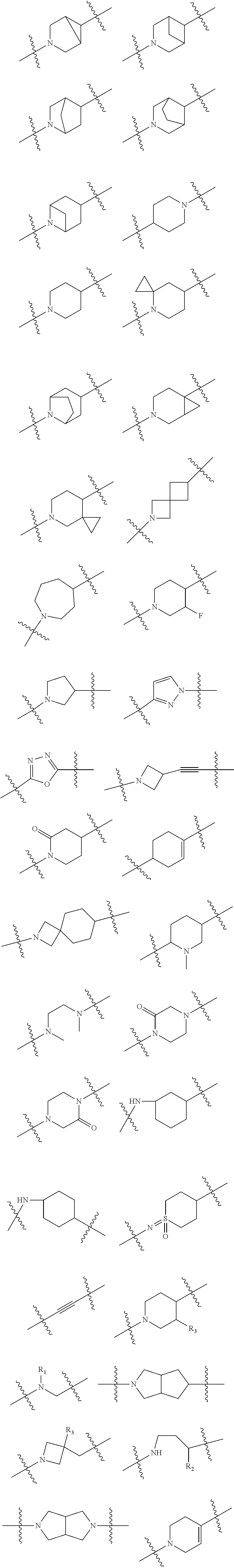

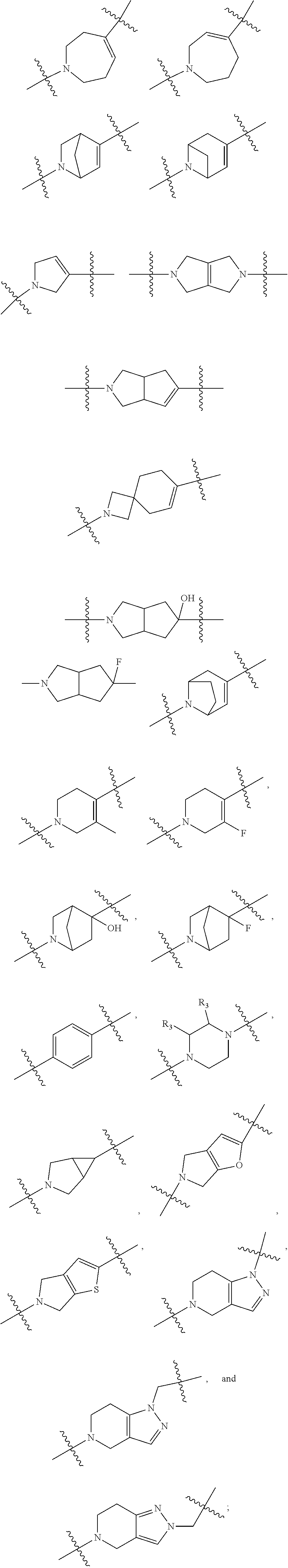

or L is selected from one of the following structures:

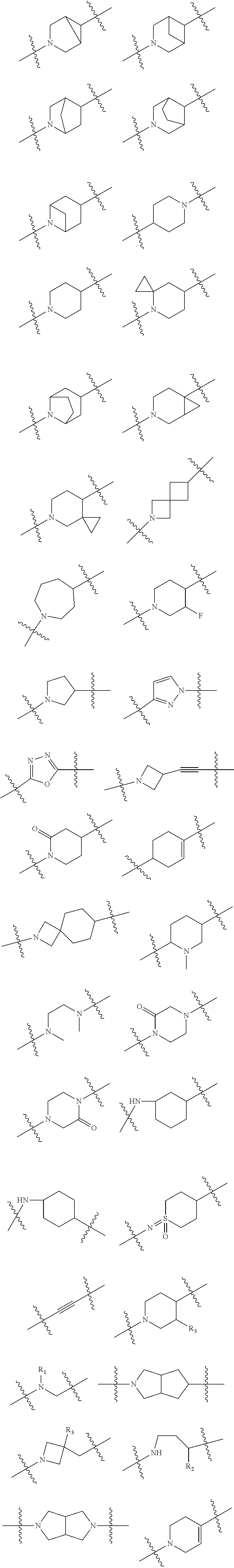

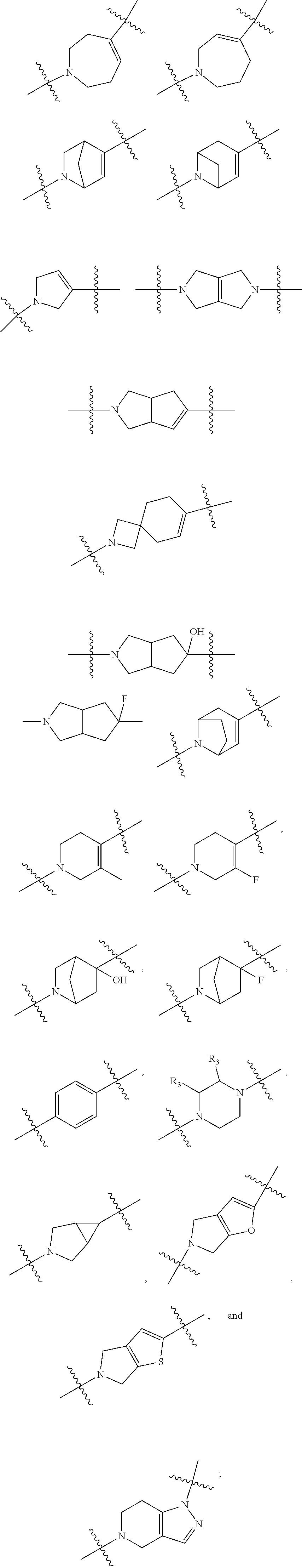

or L is selected from one of the following structures:

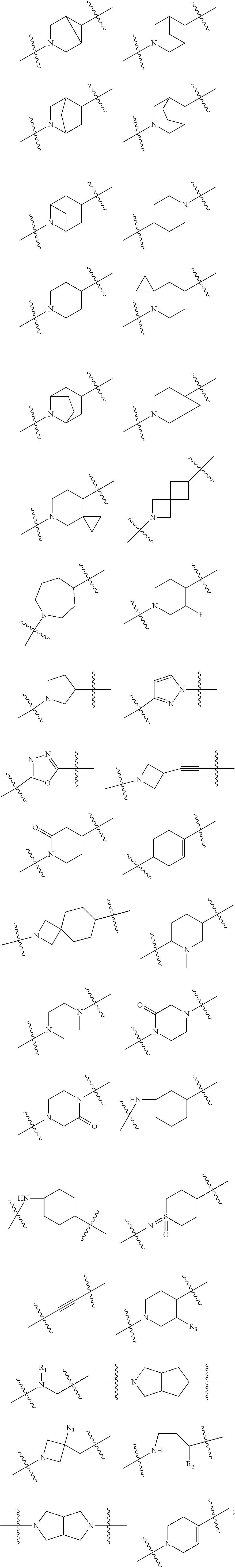

or L is selected from one of the following structures:

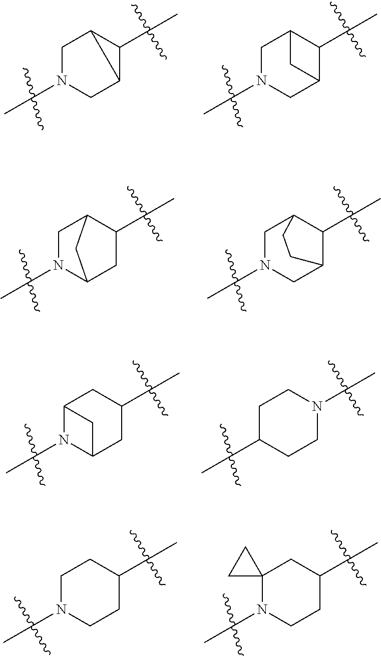

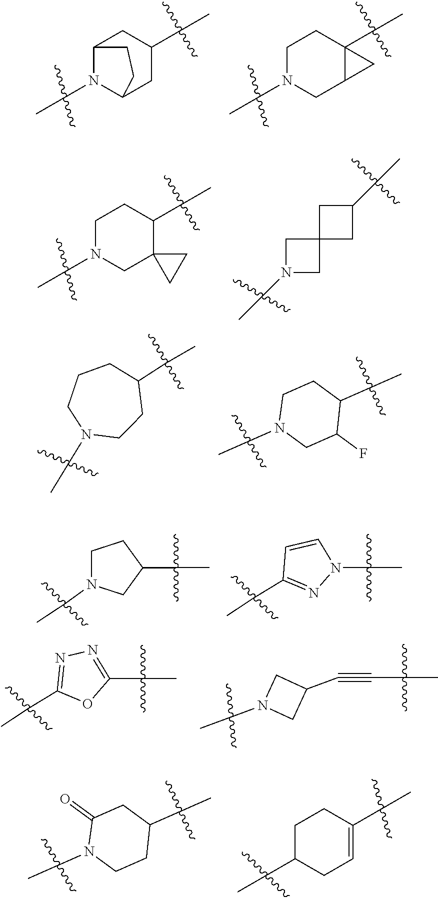

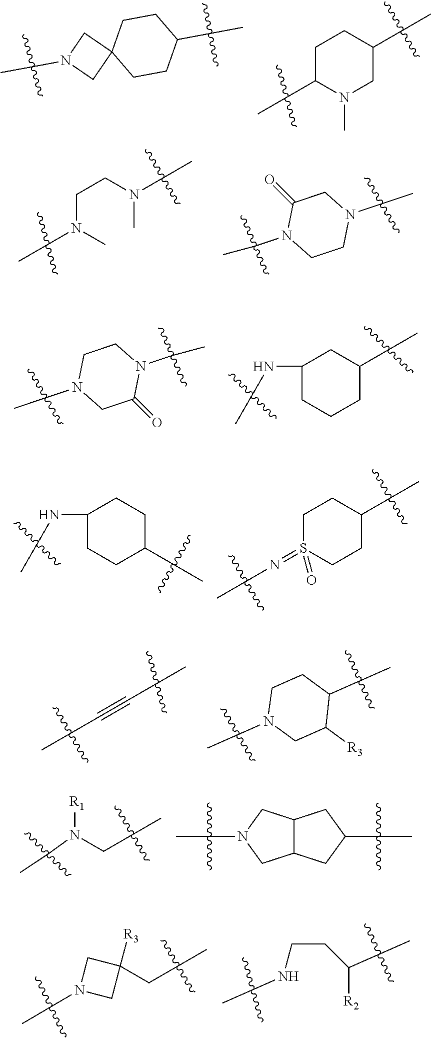

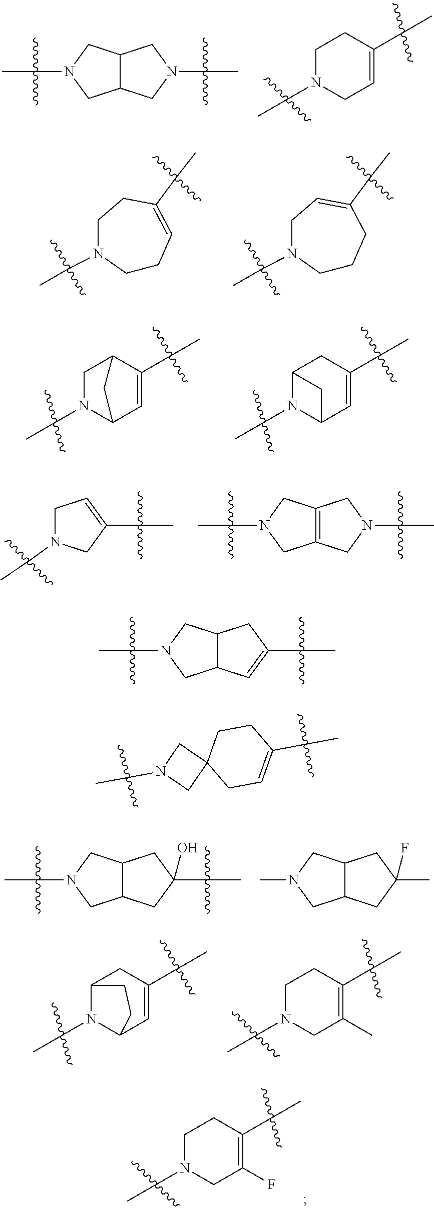

or L is selected from one of the following structures:

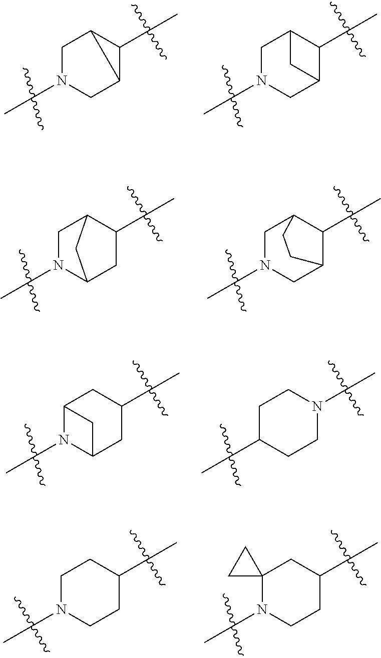

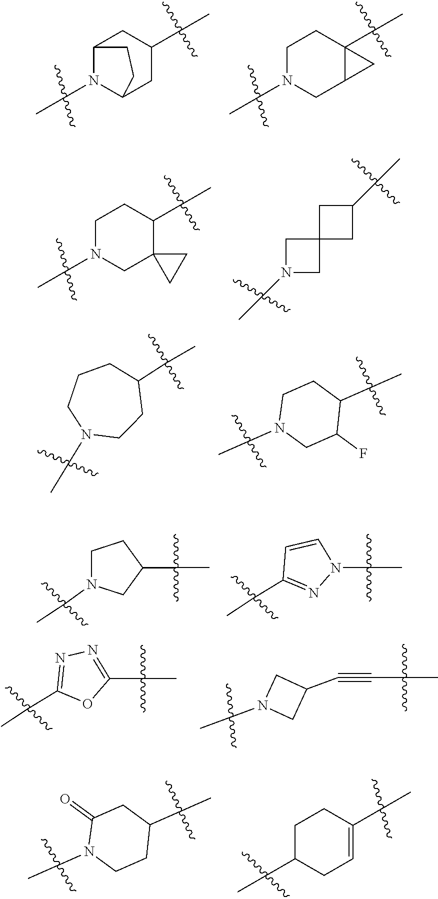

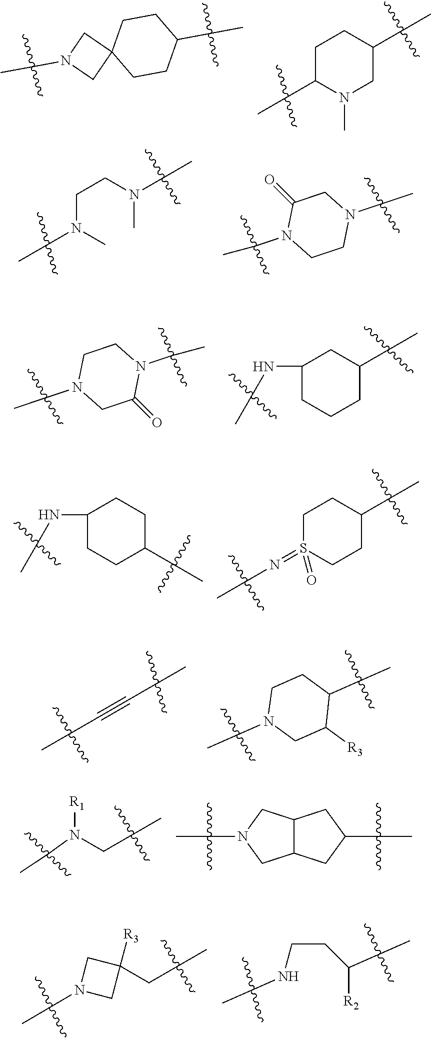

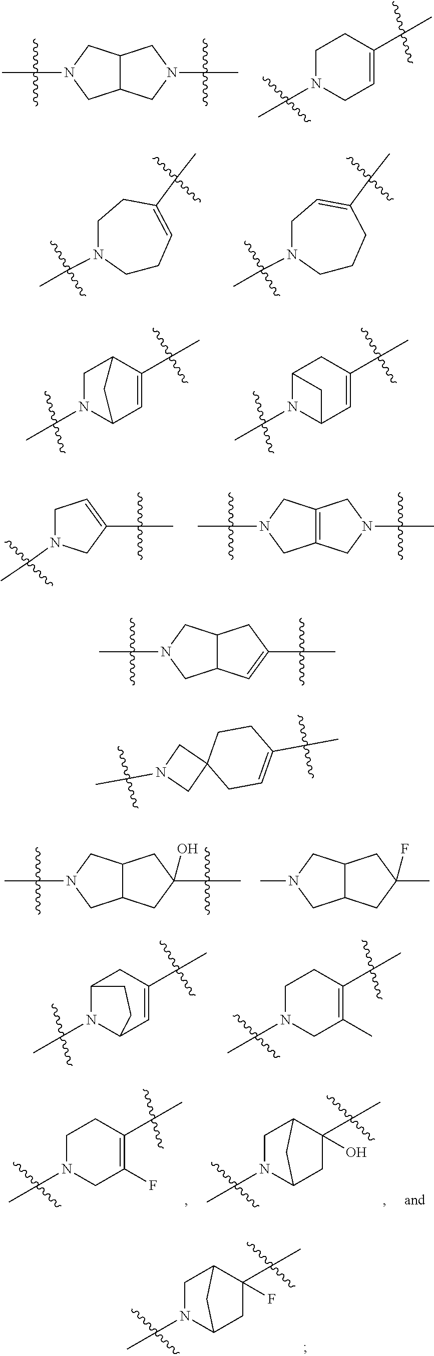

or L is selected from one of the following structures:

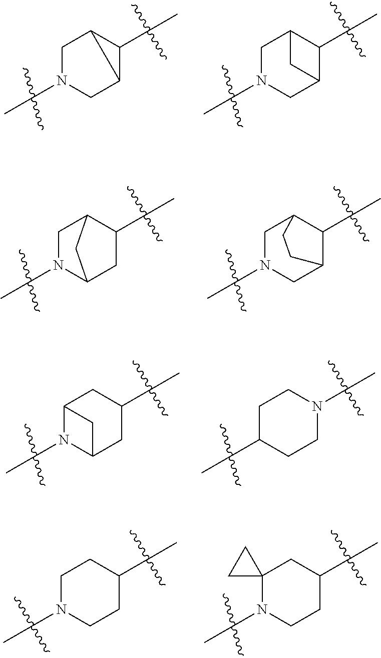

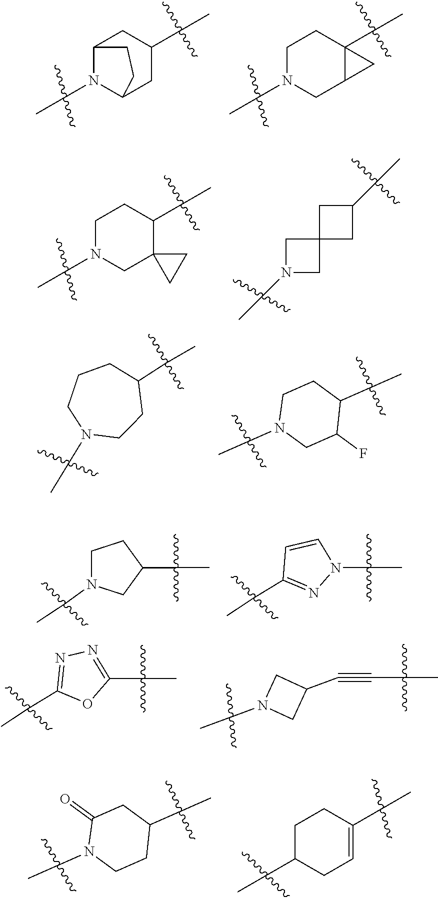

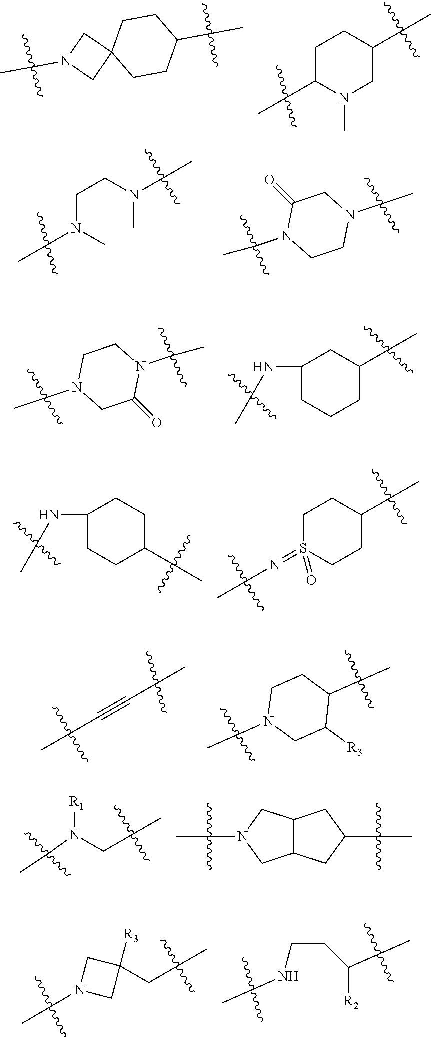

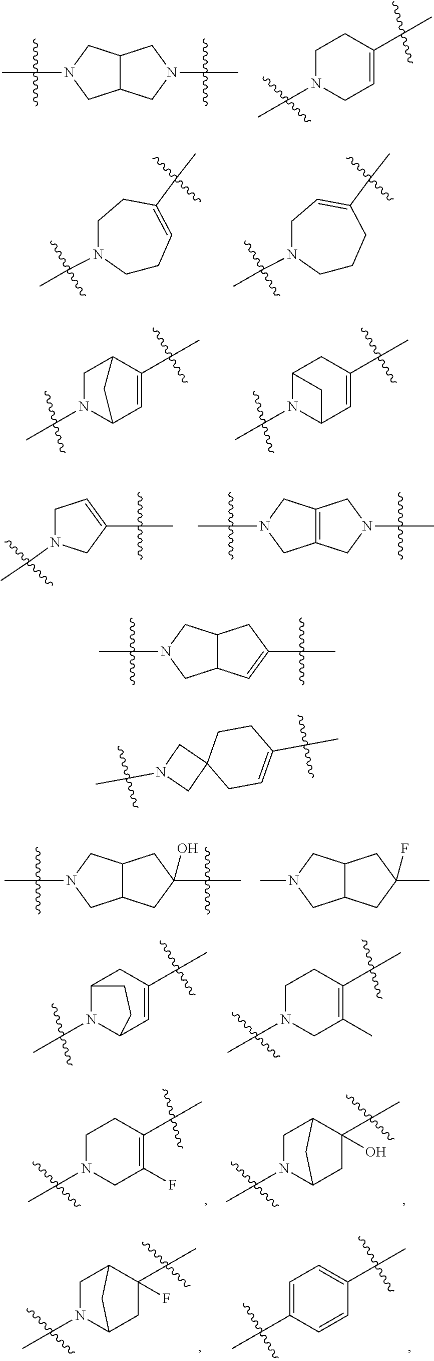

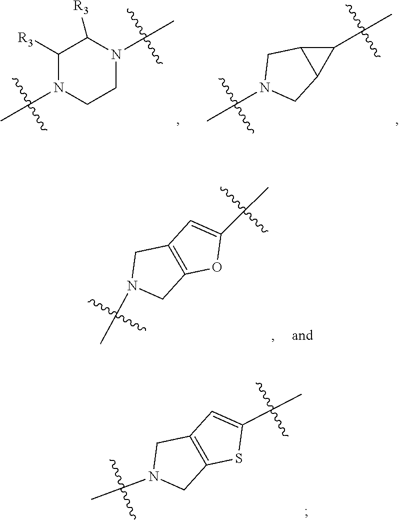

X₁ and X₂ are independently CR_{X1} or N;
each R_{X1} is independently H, halogen, C₁₋₄ alkyl, or C₁₋₄ haloalkyl, or Rx, and R₁, R_{X1} and R₂, or R_{X1} and R₃ together with the atoms to which they are attached form C₃₋₆ cycloalkyl, 5- to 7-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
each R is independently H, halogen, cyano, C₁₋₄ alkyl, C₂₋₄ alkynyl, C₃₋₆ cycloalkyl, 4- to 6-membered heterocycloalkyl containing 1-3 heteroatoms selected from N, S, O, Si and P, —SO₂NH₂, —NHSO₂C₁₋₄ alkyl, —SO₂NHC₁₋₄ alkyl, SO₂C₁₋₄ alkyl, —P(O)(CH₃)₂, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, and NH₂; or two adjacent R, or R and R_{X1} together with the atoms to which they are attached form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂; or each R is independently H, halogen, C₁₋₄ alkyl, C₂₋₄ alkynyl, —SO₂NH₂, —NHSO₂C₁₋₄ alkyl, —SO₂NHC₁₋₄ alkyl, SO₂C₁₋₄ alkyl, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, and NH₂; or two adjacent R, or R and R_{X1} together with the atoms to which they are attached form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂; or two adjacent R, or R and R_{X1} together with the atoms to which they are attached form 5- to 10-membered heterocycloalkyl containing 1-3 heteroatoms selected from N, S, O, P and B, wherein the heterocycloalkyl is optionally substituted with 1-3 groups selected from halogen, ═O, CN, C₁₋₄ alkyl, C₁₋₄ alkoxy, OH, and NH₂;
R₁ is H, or C₁₋₄ alkyl, or C₁₋₄ haloalkyl;
R₂ is H, C₁₋₄ alkyl, or C₁₋₄ haloalkyl; and
R₃ is H, halogen, ═O, CN, or C₁₋₄ alkyl.

Solution 4 of the present invention relates to the compound or the stereoisomer or pharmaceutically acceptable salt thereof according to solution 1 or 2, wherein Cy is selected from 5-membered monocyclic heteroaryl, 5-membered heteroaryl fused benzene ring, 5-membered heteroaryl fused 5-membered heteroaryl, 5-membered heteroaryl fused 6-membered heteroaryl, 5-membered heteroaryl fused 4-membered cycloalkyl, 5-membered heteroaryl fused 5-membered cycloalkyl, 5-membered heteroaryl fused 6-membered cycloalkyl, 5-membered heteroaryl fused 4-membered heterocycloalkyl, 5-membered heteroaryl fused 5-membered heterocycloalkyl, 5-membered heteroaryl fused 6-membered heterocycloalkyl, 6-membered heteroaryl fused benzene ring, 6-membered heteroaryl fused 5-membered heteroaryl, 6-membered heteroaryl fused 6-membered heteroaryl, 6-membered heteroaryl fused 4-membered cycloalkyl, 6-membered heteroaryl fused 5-membered cycloalkyl, 6-membered heteroaryl fused 6-membered cycloalkyl, 6-membered heteroaryl fused 4-membered heterocycloalkyl, 6-membered heteroaryl fused 5-membered heterocycloalkyl, and 6-membered heteroaryl fused 6-membered heterocycloalkyl;
or Cy is selected from the following structures that are optionally further substituted with 1-3 R:

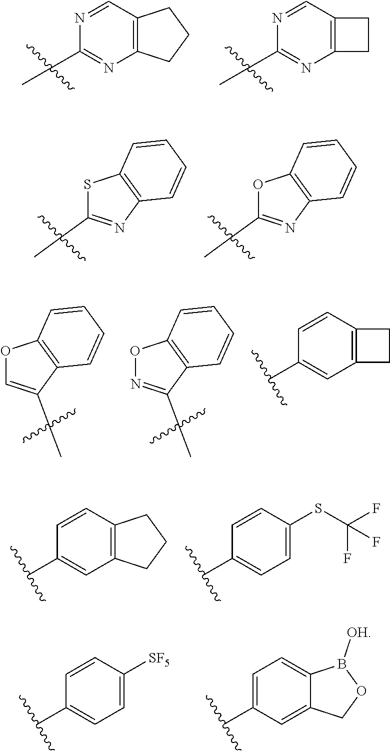

Furthermore, in the compound or the stereoisomer or pharmaceutically acceptable salt thereof according to solution 1 or 2,
Cy is selected from 5-membered monocyclic heteroaryl, 5-membered heteroaryl fused benzene ring, 5-membered heteroaryl fused 5-membered heteroaryl, 5-membered heteroaryl fused 6-membered heteroaryl, 5-membered heteroaryl fused 4-membered cycloalkyl, 5-membered heteroaryl fused 5-membered cycloalkyl, 5-membered heteroaryl fused 6-membered cycloalkyl, 5-membered heteroaryl fused 4-membered heterocycloalkyl, 5-membered heteroaryl fused 5-membered heterocycloalkyl, 5-membered heteroaryl fused 6-membered heterocycloalkyl, 6-membered heteroaryl fused benzene ring, 6-membered heteroaryl fused 5-membered heteroaryl, 6-membered heteroaryl fused 6-membered heteroaryl, 6-membered heteroaryl fused 4-membered cycloalkyl, 6-membered heteroaryl fused 5-membered cycloalkyl, 6-membered heteroaryl fused 6-membered cycloalkyl, 6-membered heteroaryl fused 4-membered heterocycloalkyl, 6-membered heteroaryl fused 5-membered heterocycloalkyl, and 6-membered heteroaryl fused 6-membered heterocycloalkyl; or

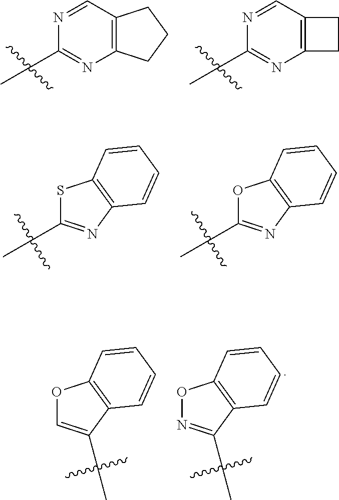

Solution 5 of the present invention relates to the compound or the stereoisomer or pharmaceutically acceptable salt thereof according to solution 3, wherein
L is selected from

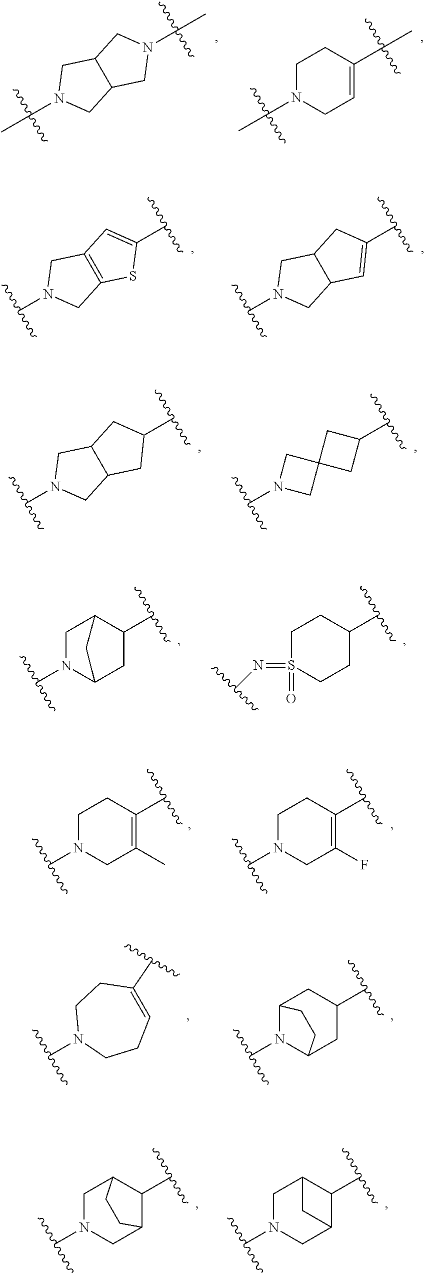

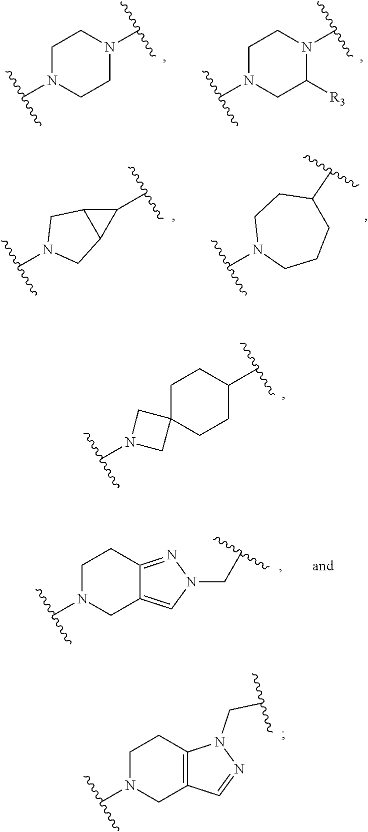

X₁ and X₂ are independently CR_{X1} or N;
each R_{X1} is independently H, F, Cl, methyl, or ethyl, or Rx, and R₃ together with the atoms to which they are attached form C₅ cycloalkyl, C₆ cycloalkyl, 5-membered heterocycloalkyl containing 1-3 heteroatoms selected from N, S and O, or 6-membered heterocycloalkyl containing 1-3 heteroatoms selected from N, S and O;
R₃ is H, F, Cl, ═O, CN, or C₁₋₃ alkyl; and each R is independently H, F, Cl, methyl, ethyl, acetylene, or propyne, or two adjacent R together with the atoms to which they are attached form cyclopropenyl, cyclobutenyl, cyclopentenyl, or cyclohexenyl, wherein the cyclopropenyl, cyclobutenyl, cyclopentenyl, and cyclohexenyl are optionally substituted with 1-3 groups selected from F, Cl, ═O, CN, methyl, ethyl, methoxy, ethoxy, OH, and NH₂;
furthermore, in the compound or the stereoisomer or pharmaceutically acceptable salt thereof according to solution 3, L is selected from

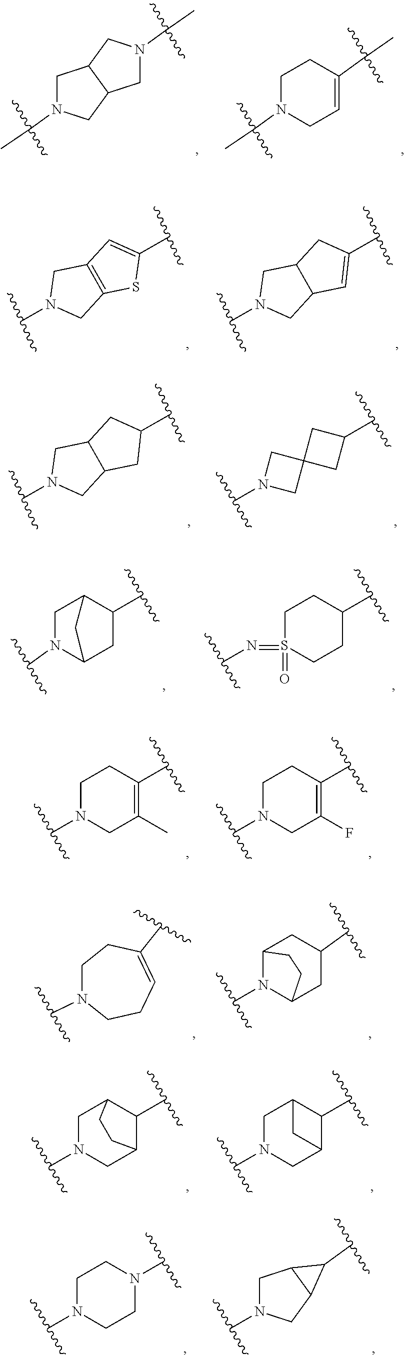

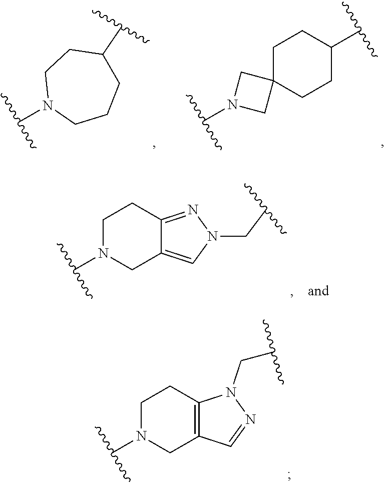 [SMILES: *C1CCCN(*)CC1 ; *C1CCC2(CC1)CN(*)C2 ; *Cn1cc2c(n1)CCN(*)C2 ; *Cn1ncc2c1CCN(*)C2]

X₁ and X₂ are independently CR_{X1} or N;
each R_{X1} is independently H, F, Cl, methyl, or ethyl; and
each R is independently H, F, Cl, methyl, ethyl, acetylene, or propyne, or two adjacent R together with the atoms to which they are attached form cyclopropenyl, cyclobutenyl, cyclopentenyl, or cyclohexenyl, wherein the cyclopropenyl, cyclobutenyl, cyclopentenyl, and cyclohexenyl are optionally substituted with 1-3 groups selected from F, Cl, ═O, CN, methyl, ethyl, methoxy, ethoxy, OH, and NH₂.

Furthermore, in the compound or the stereoisomer or pharmaceutically acceptable salt thereof according to solution 3,

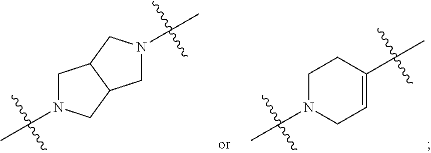 [SMILES: *N1CC2CN(*)CC2C1 ; *C1=CCN(*)CC1]

L is selected from or
X₁ and X₂ are independently CR_{X1} or N;
each R_{X1} is independently H, F, Cl, methyl, or ethyl; and
each R is independently H, F, Cl, methyl, ethyl, acetylene, or propyne, or two adjacent R together with the atoms to which they are attached form cyclopropenyl, cyclobutenyl, cyclopentenyl, or cyclohexenyl, wherein the cyclopropenyl, cyclobutenyl, cyclopentenyl, and cyclohexenyl are optionally substituted with 1-3 groups selected from F, Cl, ═O, CN, methyl, ethyl, methoxy, ethoxy, OH, and NH₂.

Solution 6 of the present invention relates to the compound or the stereoisomer or pharmaceutically acceptable salt thereof according to solution 1, wherein

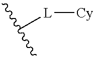

is selected from the following structures:

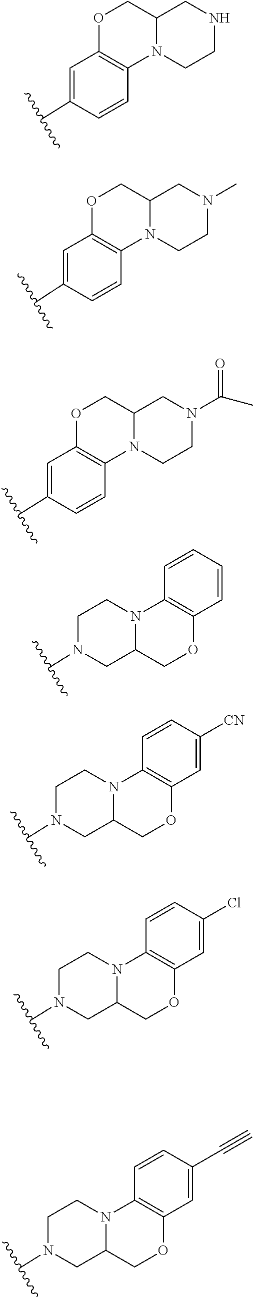

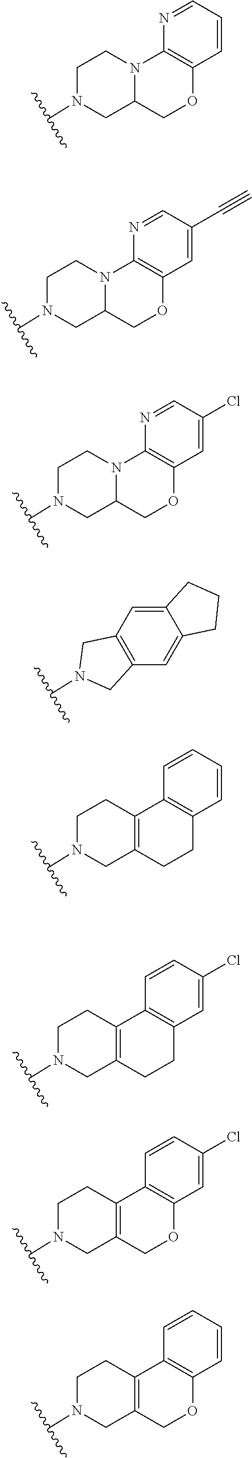

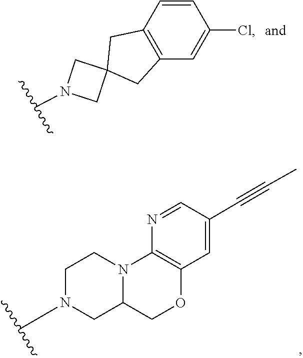 [SMILES: *N1CC2(Cc3ccc(Cl)cc3C2)C1 ; "and" ; *N1CCN2c3ncc(C#CC)cc3OCC2C1]

or selected from

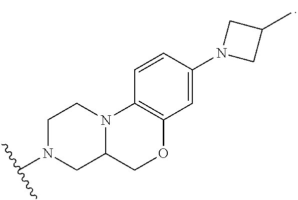 [SMILES: *N1CCN2c3ccc(N4CC(C)C4)cc3OCC2C1]

In solution 7 of the present invention, the compound of the present invention is selected from a compound with one of the structures in Table 1 below, or a stereoisomer or pharmaceutically acceptable salt thereof:

TABLE 1

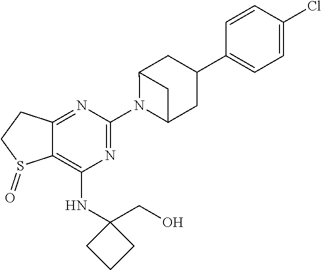 [SMILES: O=S1CCc2nc(N3C4CC(c5ccc(Cl)cc5)CC3C4)nc(NC3(CO)CCC3)c21]

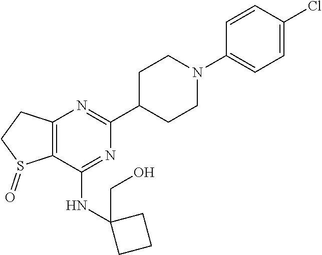 [SMILES: O=S1CCc2nc(C3CCN(c4ccc(Cl)cc4)CC3)nc(NC3(CO)CCC3)c21]

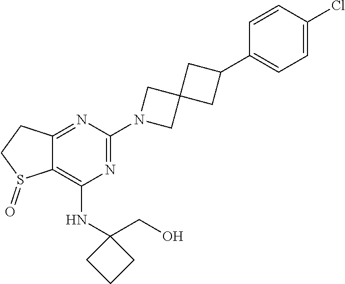 [SMILES: O=S1CCc2nc(N3CC4(CC(c5ccc(Cl)cc5)C4)C3)nc(NC3(CO)CCC3)c21]

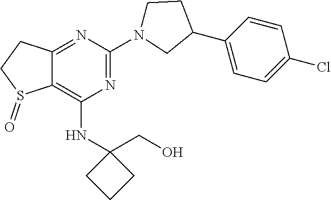 [SMILES: O=S1CCc2nc(N3CCC(c4ccc(Cl)cc4)C3)nc(NC3(CO)CCC3)c21]

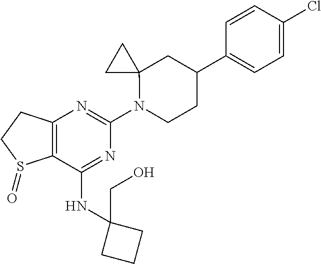 [SMILES: O=S1CCc2nc(N3CCC(c4ccc(Cl)cc4)CC34CC4)nc(NC3(CO)CCC3)c21]

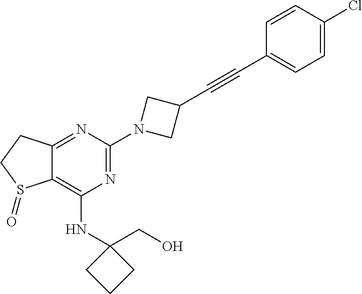 [SMILES: O=S1CCc2nc(N3CC(C#Cc4ccc(Cl)cc4)C3)nc(NC3(CO)CCC3)c21]

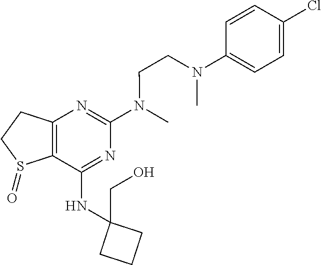 [SMILES: CN(CCN(C)c1nc2c(c(NC3(CO)CCC3)n1)S(=O)CC2)c1ccc(Cl)cc1]

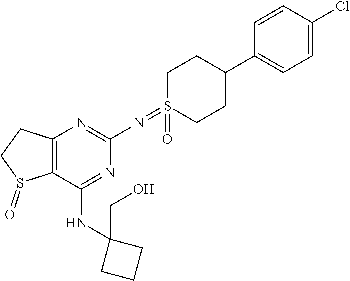 [SMILES: O=S1CCc2nc(N=S3(=O)CCC(c4ccc(Cl)cc4)CC3)nc(NC3(CO)CCC3)c21]

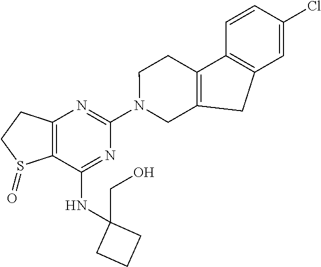 [SMILES: O=S1CCc2nc(N3CCC4=C(Cc5cc(Cl)ccc54)C3)nc(NC3(CO)CCC3)c21]

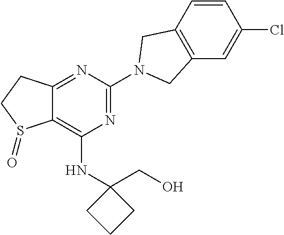 [SMILES: O=S1CCc2nc(N3Cc4ccc(Cl)cc4C3)nc(NC3(CO)CCC3)c21]

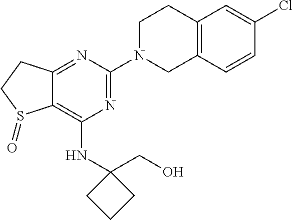 [SMILES: O=S1CCc2nc(N3CCc4cc(Cl)ccc4C3)nc(NC3(CO)CCC3)c21]

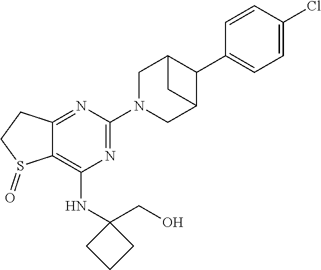 [SMILES: O=S1CCc2nc(N3CC4CC(C3)C4c3ccc(Cl)cc3)nc(NC3(CO)CCC3)c21]

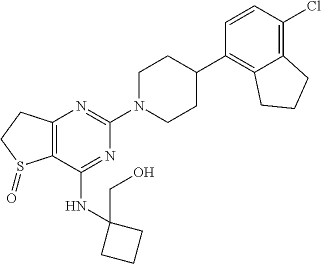 [SMILES: O=S1CCc2nc(N3CCC(c4ccc(Cl)c5c4CCC5)CC3)nc(NC3(CO)CCC3)c21]

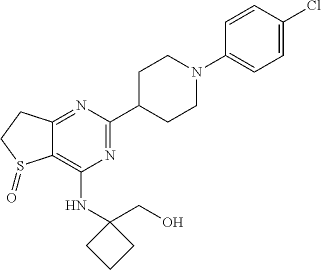 [SMILES: O=S1CCc2nc(C3CCN(c4ccc(Cl)cc4)CC3)nc(NC3(CO)CCC3)c21]

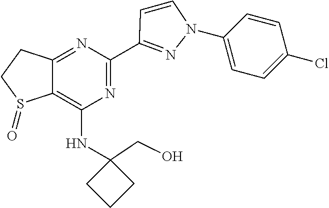 [SMILES: O=S1CCc2nc(-c3ccn(-c4ccc(Cl)cc4)n3)nc(NC3(CO)CCC3)c21]

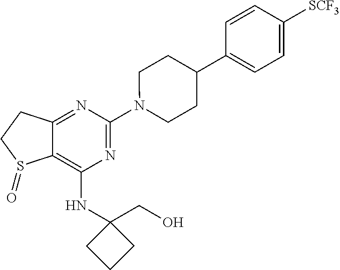

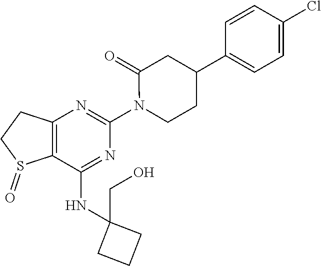 [SMILES: O=C1CC(c2ccc(Cl)cc2)CCN1c1nc2c(c(NC3(CO)CCC3)n1)S(=O)CC2]

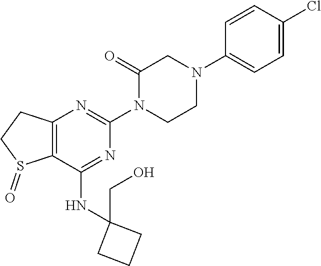 [SMILES: O=C1CN(c2ccc(Cl)cc2)CCN1c1nc2c(c(NC3(CO)CCC3)n1)S(=O)CC2]

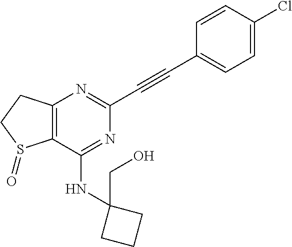 [SMILES: O=S1CCc2nc(C#Cc3ccc(Cl)cc3)nc(NC3(CO)CCC3)c21]

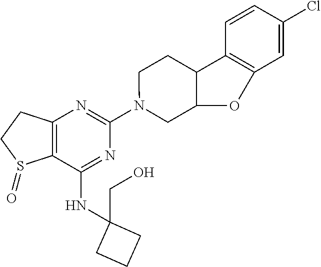 [SMILES: O=S1CCc2nc(N3CCC4c5ccc(Cl)cc5OC4C3)nc(NC3(CO)CCC3)c21]

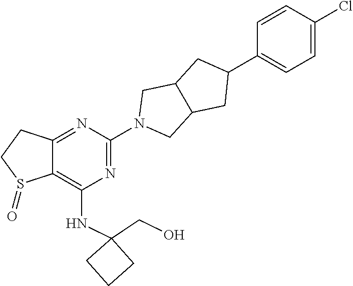 [SMILES: O=S1CCc2nc(N3CC4CC(c5ccc(Cl)cc5)CC4C3)nc(NC3(CO)CCC3)c21]

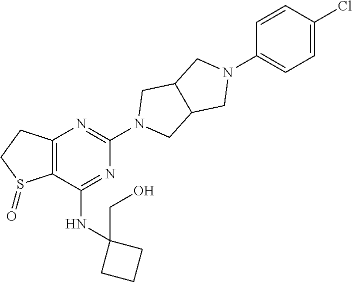 [SMILES: O=S1CCc2nc(N3CC4CN(c5ccc(Cl)cc5)CC4C3)nc(NC3(CO)CCC3)c21]

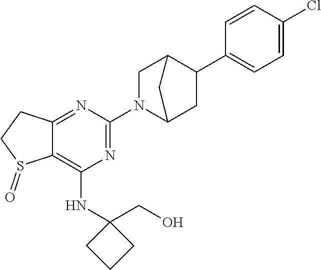 [SMILES: O=S1CCc2nc(N3CC4CC3CC4c3ccc(Cl)cc3)nc(NC3(CO)CCC3)c21]

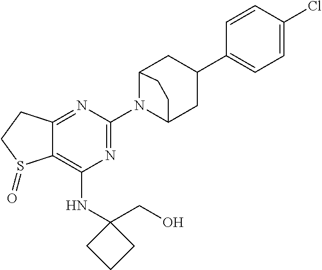 [SMILES: O=S1CCc2nc(N3C4CCC3CC(c3ccc(Cl)cc3)C4)nc(NC3(CO)CCC3)c21]

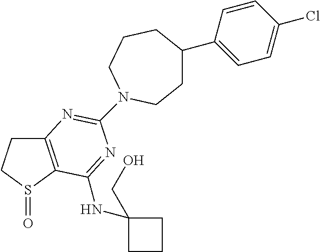 [SMILES: O=S1CCc2nc(N3CCCC(c4ccc(Cl)cc4)CC3)nc(NC3(CO)CCC3)c21]

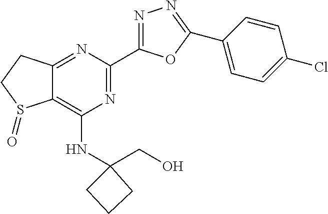 [SMILES: O=S1CCc2nc(-c3nnc(-c4ccc(Cl)cc4)o3)nc(NC3(CO)CCC3)c21]

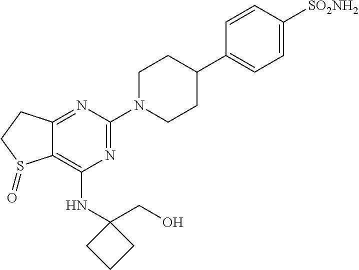 [SMILES: NS(=O)(=O)c1ccc(C2CCN(c3nc4c(c(NC5(CO)CCC5)n3)S(=O)CC4)CC2)cc1]

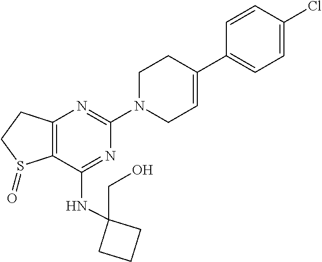 [SMILES: O=S1CCc2nc(N3CC=C(c4ccc(Cl)cc4)CC3)nc(NC3(CO)CCC3)c21]

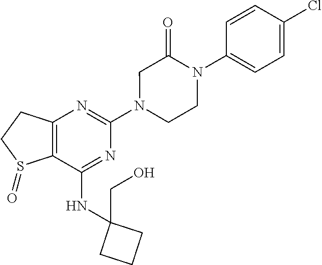 [SMILES: O=C1CN(c2nc3c(c(NC4(CO)CCC4)n2)S(=O)CC3)CCN1c1ccc(Cl)cc1]

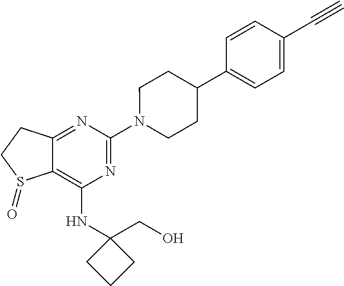 [SMILES: C#Cc1ccc(C2CCN(c3nc4c(c(NC5(CO)CCC5)n3)S(=O)CC4)CC2)cc1]

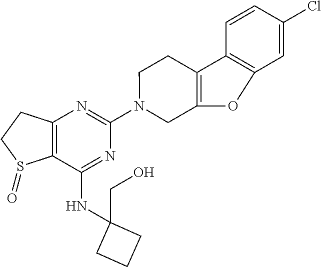 [SMILES: O=S1CCc2nc(N3CCc4c(oc5cc(Cl)ccc45)C3)nc(NC3(CO)CCC3)c21]

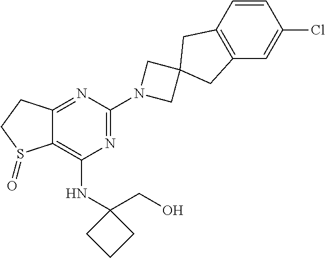 [SMILES: O=S1CCc2nc(N3CC4(Cc5ccc(Cl)cc5C4)C3)nc(NC3(CO)CCC3)c21]

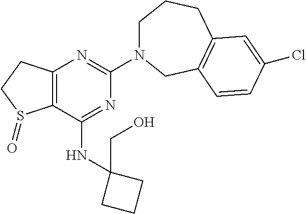 [SMILES: O=S1CCc2nc(N3CCCc4cc(Cl)ccc4C3)nc(NC3(CO)CCC3)c21]

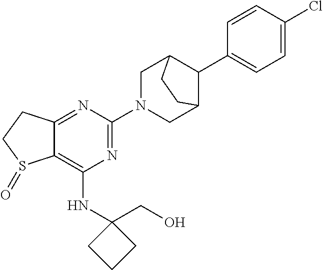 [SMILES: O=S1CCc2nc(N3CC4CCC(C3)C4c3ccc(Cl)cc3)nc(NC3(CO)CCC3)c21]

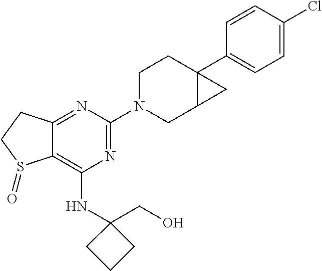 [SMILES: O=S1CCc2nc(N3CCC4(c5ccc(Cl)cc5)CC4C3)nc(NC3(CO)CCC3)c21]

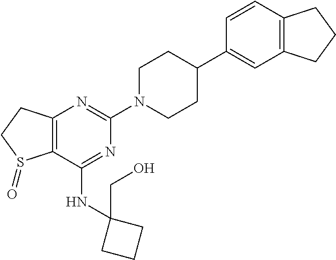 [SMILES: O=S1CCc2nc(N3CCC(c4ccc5c(c4)CCC5)CC3)nc(NC3(CO)CCC3)c21]

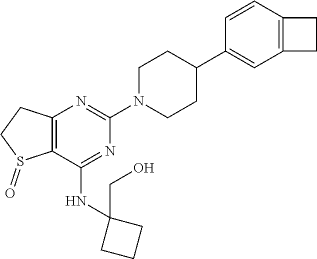 [SMILES: O=S1CCc2nc(N3CCC(c4ccc5c(c4)CC5)CC3)nc(NC3(CO)CCC3)c21]

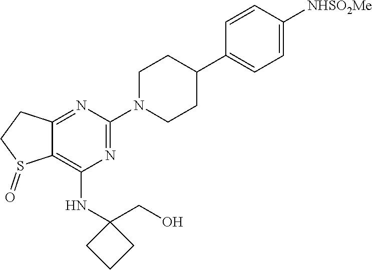

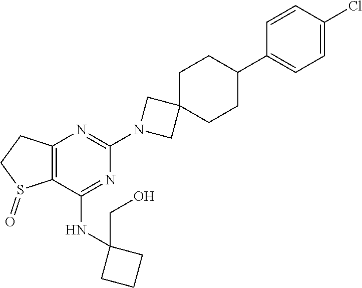 [SMILES: O=S1CCc2nc(N3CC4(CCC(c5ccc(Cl)cc5)CC4)C3)nc(NC3(CO)CCC3)c21]

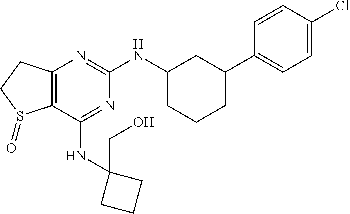 [SMILES: O=S1CCc2nc(NC3CCCC(c4ccc(Cl)cc4)C3)nc(NC3(CO)CCC3)c21]

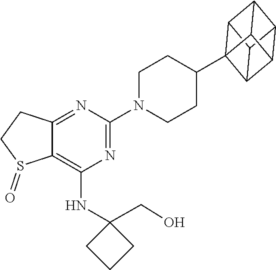 [SMILES: O=S1CCc2nc(N3CCC(C45C6C7C8C6C4C8C75)CC3)nc(NC3(CO)CCC3)c21]

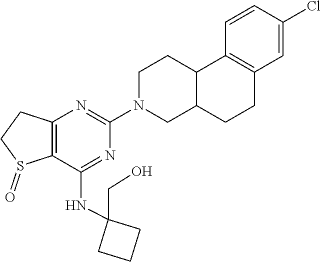 [SMILES: O=S1CCc2nc(N3CCC4c5ccc(Cl)cc5CCC4C3)nc(NC3(CO)CCC3)c21]

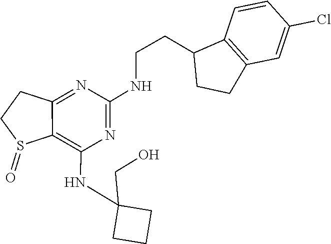 [SMILES: O=S1CCc2nc(NCCC3CCc4cc(Cl)ccc43)nc(NC3(CO)CCC3)c21]

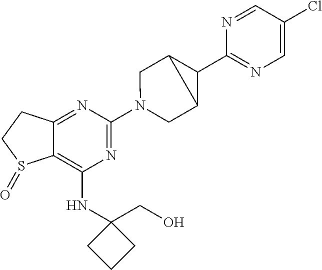 [SMILES: O=S1CCc2nc(N3CC4C(C3)C4c3ncc(Cl)cn3)nc(NC3(CO)CCC3)c21]

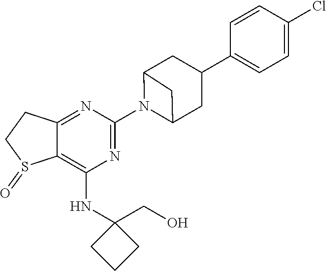 [SMILES: O=S1CCc2nc(N3C4CC(c5ccc(Cl)cc5)CC3C4)nc(NC3(CO)CCC3)c21]

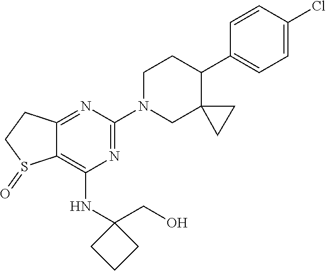 [SMILES: O=S1CCc2nc(N3CCC(c4ccc(Cl)cc4)C4(CC4)C3)nc(NC3(CO)CCC3)c21]

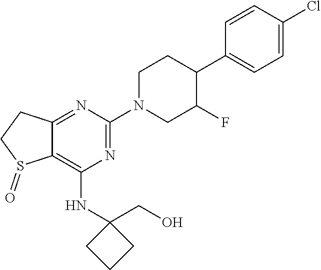 [SMILES: O=S1CCc2nc(N3CCC(c4ccc(Cl)cc4)C(F)C3)nc(NC3(CO)CCC3)c21]

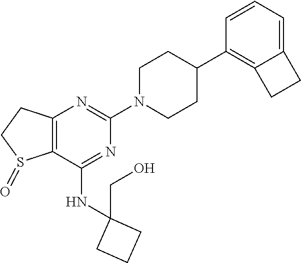 [SMILES: O=S1CCc2nc(N3CCC(c4cccc5c4CC5)CC3)nc(NC3(CO)CCC3)c21]

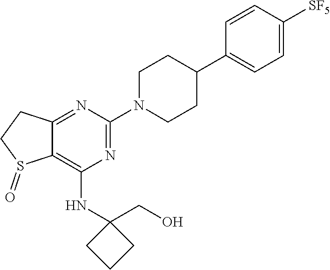

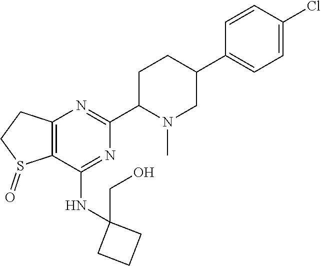 [SMILES: CN1CC(c2ccc(Cl)cc2)CCC1c1nc2c(c(NC3(CO)CCC3)n1)S(=O)CC2]

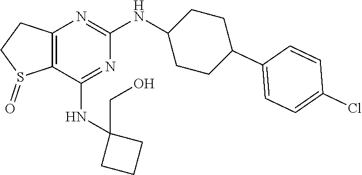 [SMILES: O=S1CCc2nc(NC3CCC(c4ccc(Cl)cc4)CC3)nc(NC3(CO)CCC3)c21]

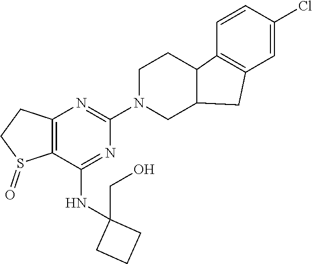 [SMILES: O=S1CCc2nc(N3CCC4c5ccc(Cl)cc5CC4C3)nc(NC3(CO)CCC3)c21]

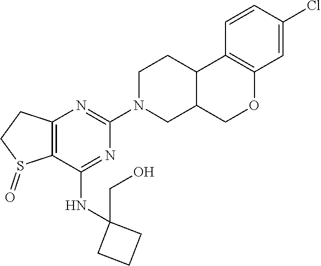 [SMILES: O=S1CCc2nc(N3CCC4c5ccc(Cl)cc5OCC4C3)nc(NC3(CO)CCC3)c21]

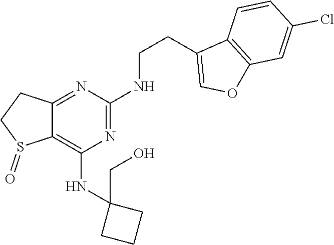 [SMILES: O=S1CCc2nc(NCCc3coc4cc(Cl)ccc34)nc(NC3(CO)CCC3)c21]

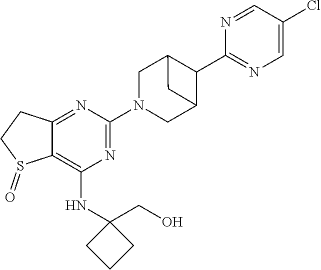 [SMILES: O=S1CCc2nc(N3CC4CC(C3)C4c3ncc(Cl)cn3)nc(NC3(CO)CCC3)c21]

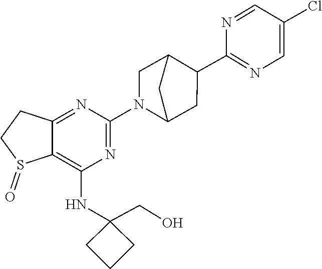 [SMILES: O=S1CCc2nc(N3CC4CC3CC4c3ncc(Cl)cn3)nc(NC3(CO)CCC3)c21]

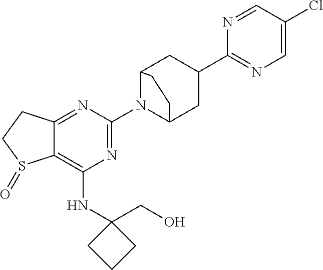 [SMILES: O=S1CCc2nc(N3C4CCC3CC(c3ncc(Cl)cn3)C4)nc(NC3(CO)CCC3)c21]

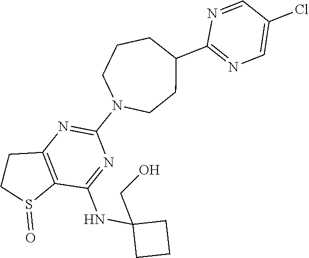 [SMILES: O=S1CCc2nc(N3CCCC(c4ncc(Cl)cn4)CC3)nc(NC3(CO)CCC3)c21]

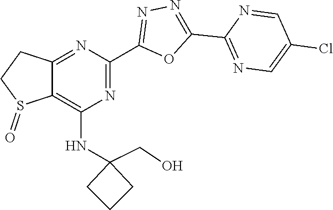 [SMILES: O=S1CCc2nc(-c3nnc(-c4ncc(Cl)cn4)o3)nc(NC3(CO)CCC3)c21]

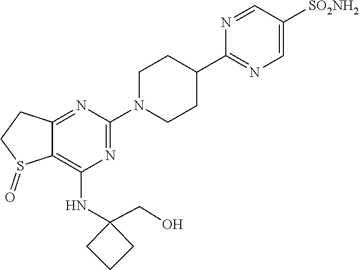 [SMILES: NS(=O)(=O)c1cnc(C2CCN(c3nc4c(c(NC5(CO)CCC5)n3)S(=O)CC4)CC2)nc1]

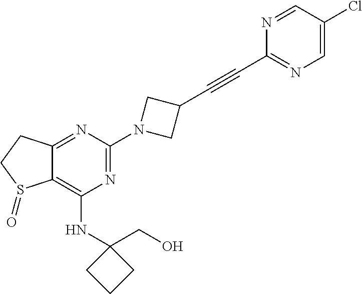 [SMILES: O=S1CCc2nc(N3CC(C#Cc4ncc(Cl)cn4)C3)nc(NC3(CO)CCC3)c21]

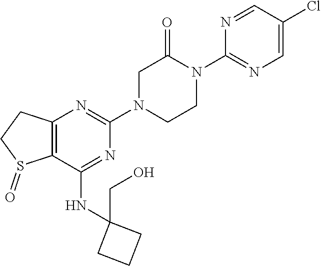 [SMILES: O=C1CN(c2nc3c(c(NC4(CO)CCC4)n2)S(=O)CC3)CCN1c1ncc(Cl)cn1]

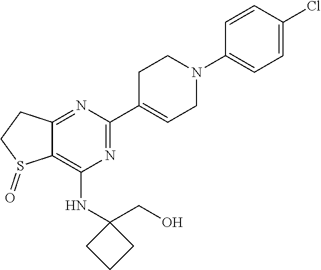 [SMILES: O=S1CCc2nc(C3=CCN(c4ccc(Cl)cc4)CC3)nc(NC3(CO)CCC3)c21]

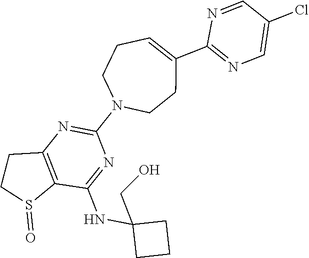 [SMILES: O=S1CCc2nc(N3CCC=C(c4ncc(Cl)cn4)CC3)nc(NC3(CO)CCC3)c21]

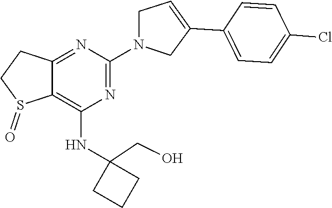 [SMILES: O=S1CCc2nc(N3CC=C(c4ccc(Cl)cc4)C3)nc(NC3(CO)CCC3)c21]

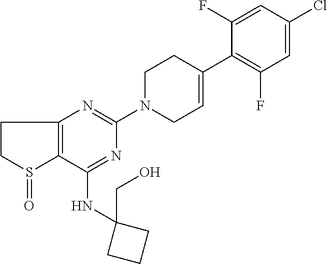 [SMILES: O=S1CCc2nc(N3CC=C(c4c(F)cc(Cl)cc4F)CC3)nc(NC3(CO)CCC3)c21]

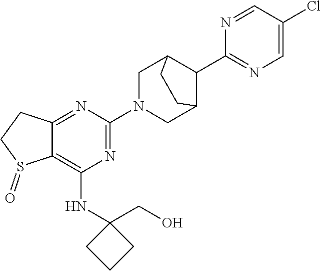 [SMILES: O=S1CCc2nc(N3CC4CCC(C3)C4c3ncc(Cl)cn3)nc(NC3(CO)CCC3)c21]

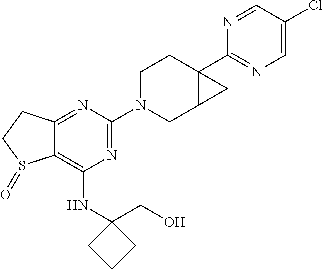 [SMILES: O=S1CCc2nc(N3CCC4(c5ncc(Cl)cn5)CC4C3)nc(NC3(CO)CCC3)c21]

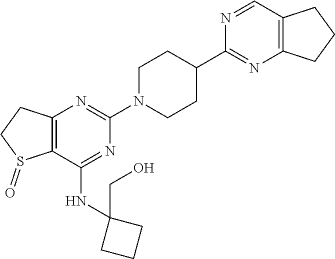 [SMILES: O=S1CCc2nc(N3CCC(c4ncc5c(n4)CCC5)CC3)nc(NC3(CO)CCC3)c21]

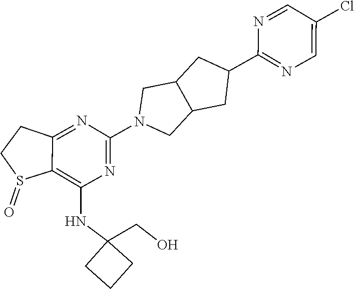 [SMILES: O=S1CCc2nc(N3CC4CC(c5ncc(Cl)cn5)CC4C3)nc(NC3(CO)CCC3)c21]

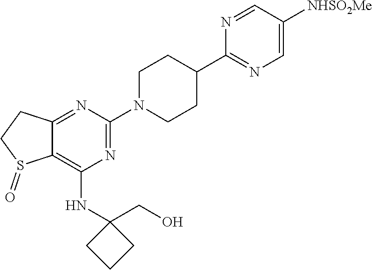

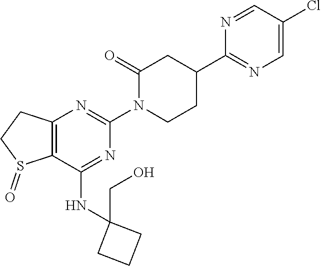 [SMILES: O=C1CC(c2ncc(Cl)cn2)CCN1c1nc2c(c(NC3(CO)CCC3)n1)S(=O)CC2]

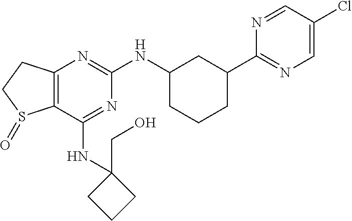 [SMILES: O=S1CCc2nc(NC3CCCC(c4ncc(Cl)cn4)C3)nc(NC3(CO)CCC3)c21]

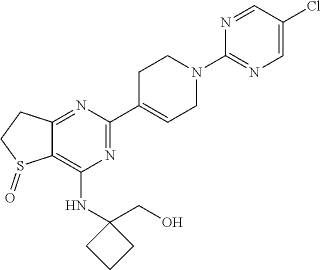 [SMILES: O=S1CCc2nc(C3=CCN(c4ncc(Cl)cn4)CC3)nc(NC3(CO)CCC3)c21]

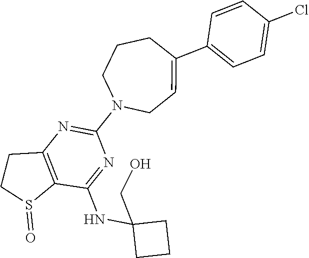 [SMILES: O=S1CCc2nc(N3CC=C(c4ccc(Cl)cc4)CCC3)nc(NC3(CO)CCC3)c21]

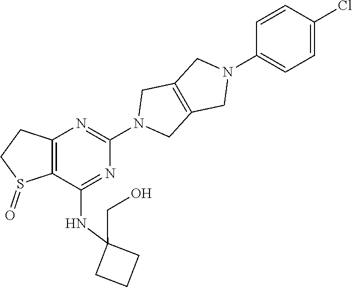 [SMILES: O=S1CCc2nc(N3CC4=C(CN(c5ccc(Cl)cc5)C4)C3)nc(NC3(CO)CCC3)c21]

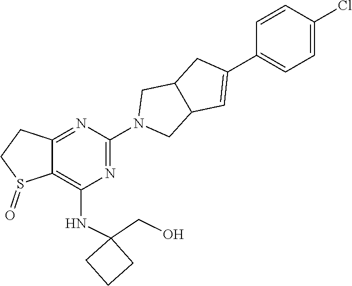 [SMILES: O=S1CCc2nc(N3CC4C=C(c5ccc(Cl)cc5)CC4C3)nc(NC3(CO)CCC3)c21]

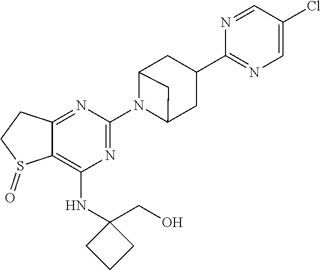 [SMILES: O=S1CCc2nc(N3C4CC(c5ncc(Cl)cn5)CC3C4)nc(NC3(CO)CCC3)c21]

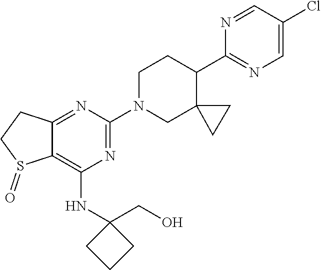 [SMILES: O=S1CCc2nc(N3CCC(c4ncc(Cl)cn4)C4(CC4)C3)nc(NC3(CO)CCC3)c21]

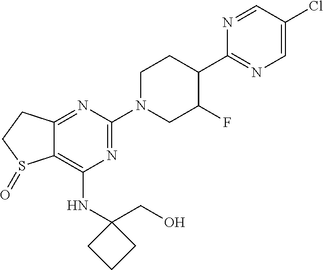 [SMILES: O=S1CCc2nc(N3CCC(c4ncc(Cl)cn4)C(F)C3)nc(NC3(CO)CCC3)c21]

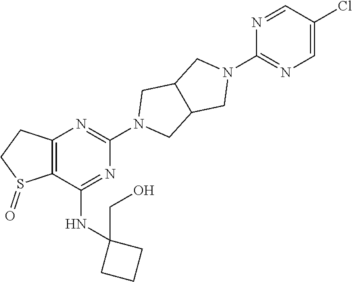 [SMILES: O=S1CCc2nc(N3CC4CN(c5ncc(Cl)cn5)CC4C3)nc(NC3(CO)CCC3)c21]

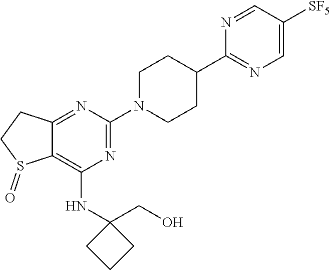

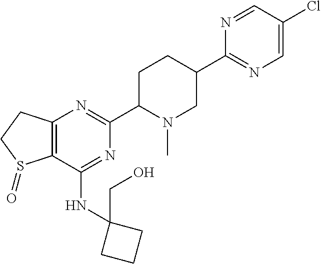 [SMILES: CN1CC(c2ncc(Cl)cn2)CCC1c1nc2c(c(NC3(CO)CCC3)n1)S(=O)CC2]

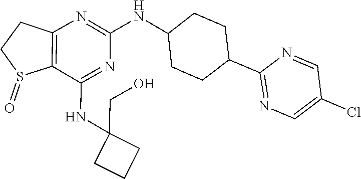 [SMILES: O=S1CCc2nc(NC3CCC(c4ncc(Cl)cn4)CC3)nc(NC3(CO)CCC3)c21]

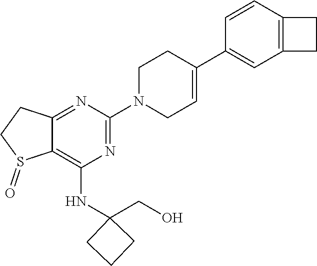 [SMILES: O=S1CCc2nc(N3CC=C(c4ccc5c(c4)CC5)CC3)nc(NC3(CO)CCC3)c21]

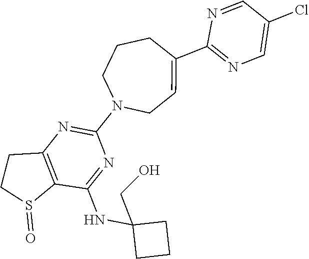 [SMILES: O=S1CCc2nc(N3CC=C(c4ncc(Cl)cn4)CCC3)nc(NC3(CO)CCC3)c21]

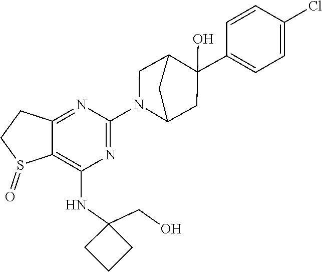 [SMILES: O=S1CCc2nc(N3CC4CC3CC4(O)c3ccc(Cl)cc3)nc(NC3(CO)CCC3)c21]

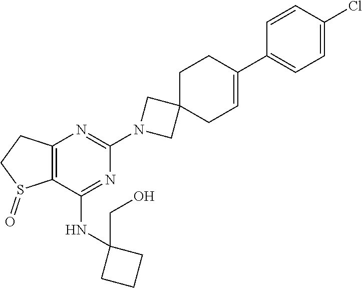 [SMILES: O=S1CCc2nc(N3CC4(CC=C(c5ccc(Cl)cc5)CC4)C3)nc(NC3(CO)CCC3)c21]

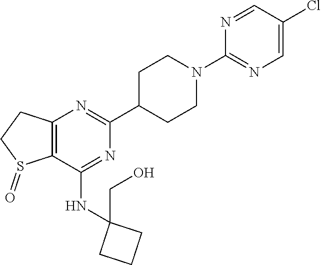 [SMILES: O=S1CCc2nc(C3CCN(c4ncc(Cl)cn4)CC3)nc(NC3(CO)CCC3)c21]

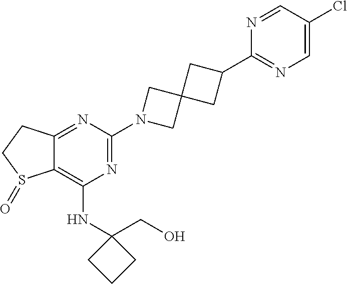 [SMILES: O=S1CCc2nc(N3CC4(CC(c5ncc(Cl)cn5)C4)C3)nc(NC3(CO)CCC3)c21]

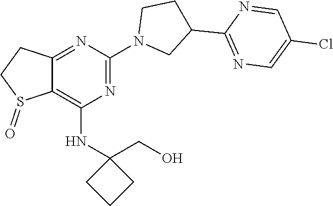 [SMILES: O=S1CCc2nc(N3CCC(c4ncc(Cl)cn4)C3)nc(NC3(CO)CCC3)c21]

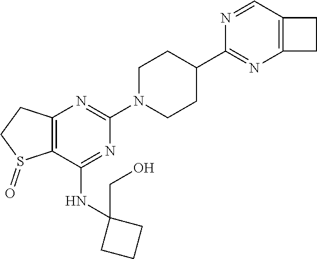 [SMILES: O=S1CCc2nc(N3CCC(c4ncc5c(n4)CC5)CC3)nc(NC3(CO)CCC3)c21]

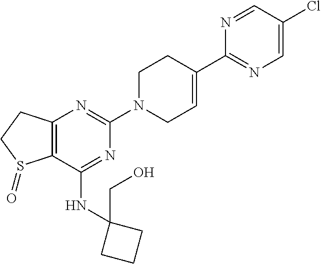 [SMILES: O=S1CCc2nc(N3CC=C(c4ncc(Cl)cn4)CC3)nc(NC3(CO)CCC3)c21]

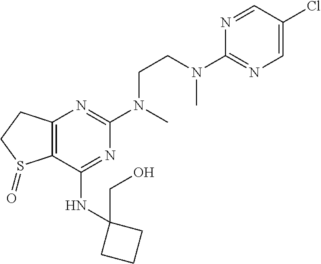 [SMILES: CN(CCN(C)c1nc2c(c(NC3(CO)CCC3)n1)S(=O)CC2)c1ncc(Cl)cn1]

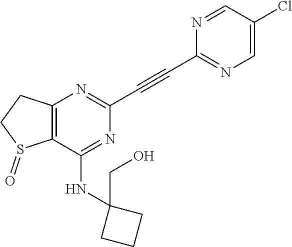 [SMILES: O=S1CCc2nc(C#Cc3ncc(Cl)cn3)nc(NC3(CO)CCC3)c21]

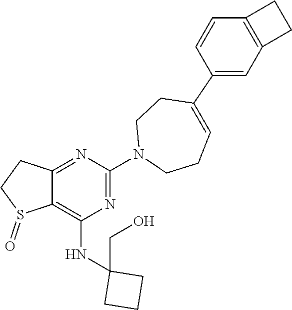 [SMILES: O=S1CCc2nc(N3CCC=C(c4ccc5c(c4)CC5)CC3)nc(NC3(CO)CCC3)c21]

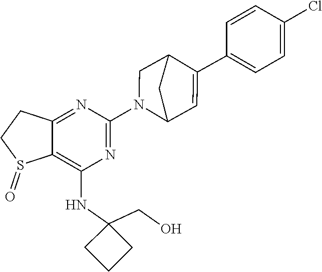 [SMILES: O=S1CCc2nc(N3CC4CC3C=C4c3ccc(Cl)cc3)nc(NC3(CO)CCC3)c21]

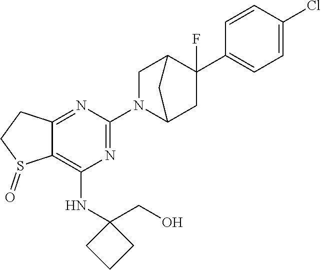 [SMILES: O=S1CCc2nc(N3CC4CC3CC4(F)c3ccc(Cl)cc3)nc(NC3(CO)CCC3)c21]

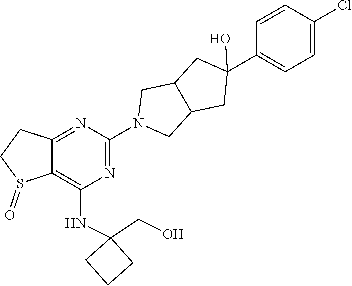 [SMILES: O=S1CCc2nc(N3CC4CC(O)(c5ccc(Cl)cc5)CC4C3)nc(NC3(CO)CCC3)c21]

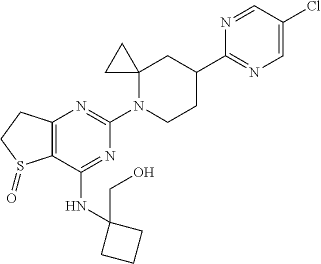 [SMILES: O=S1CCc2nc(N3CCC(c4ncc(Cl)cn4)CC34CC4)nc(NC3(CO)CCC3)c21]

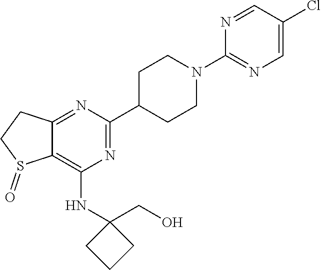 [SMILES: O=S1CCc2nc(C3CCN(c4ncc(Cl)cn4)CC3)nc(NC3(CO)CCC3)c21]

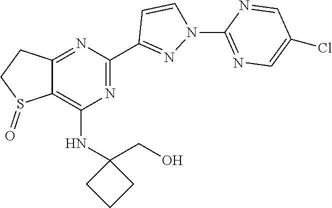 [SMILES: O=S1CCc2nc(-c3ccn(-c4ncc(Cl)cn4)n3)nc(NC3(CO)CCC3)c21]

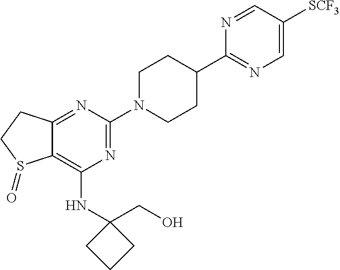

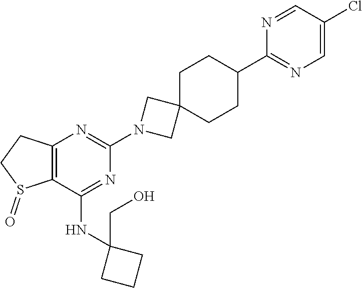 [SMILES: O=S1CCc2nc(N3CC4(CCC(c5ncc(Cl)cn5)CC4)C3)nc(NC3(CO)CCC3)c21]

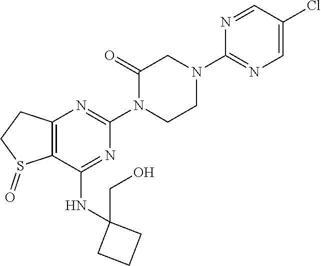 [SMILES: O=C1CN(c2ncc(Cl)cn2)CCN1c1nc2c(c(NC3(CO)CCC3)n1)S(=O)CC2]

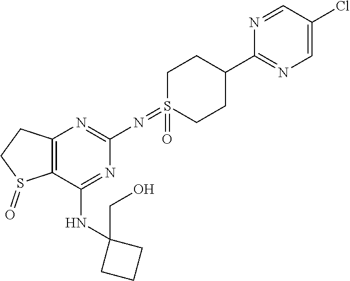 [SMILES: O=S1CCc2nc(N=S3(=O)CCC(c4ncc(Cl)cn4)CC3)nc(NC3(CO)CCC3)c21]

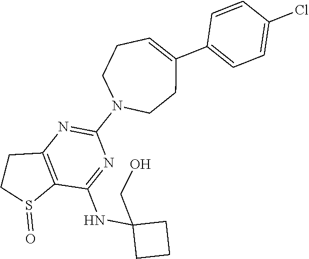 [SMILES: O=S1CCc2nc(N3CCC=C(c4ccc(Cl)cc4)CC3)nc(NC3(CO)CCC3)c21]

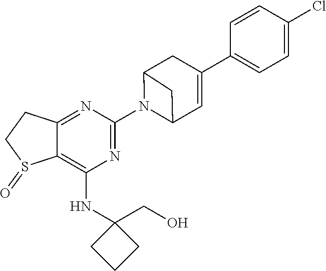 [SMILES: O=S1CCc2nc(N3C4C=C(c5ccc(Cl)cc5)CC3C4)nc(NC3(CO)CCC3)c21]

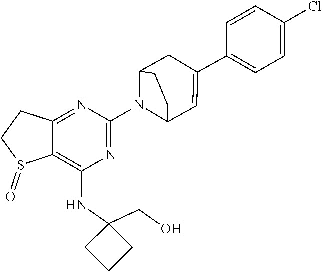 [SMILES: O=S1CCc2nc(N3C4C=C(c5ccc(Cl)cc5)CC3CC4)nc(NC3(CO)CCC3)c21]

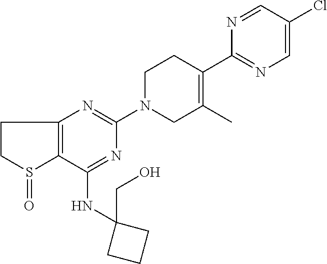 [SMILES: CC1=C(c2ncc(Cl)cn2)CCN(c2nc3c(c(NC4(CO)CCC4)n2)S(=O)CC3)C1]

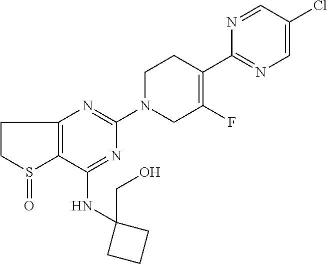 [SMILES: O=S1CCc2nc(N3CCC(c4ncc(Cl)cn4)=C(F)C3)nc(NC3(CO)CCC3)c21]

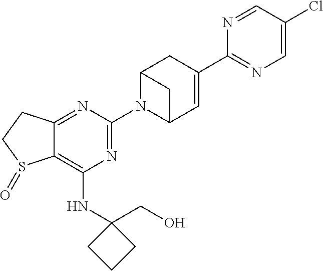 [SMILES: O=S1CCc2nc(N3C4C=C(c5ncc(Cl)cn5)CC3C4)nc(NC3(CO)CCC3)c21]

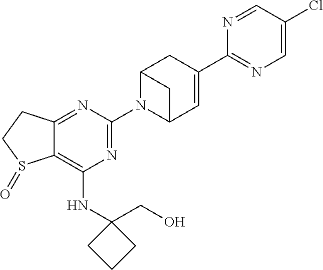 [SMILES: O=S1CCc2nc(N3C4C=C(c5ncc(Cl)cn5)CC3C4)nc(NC3(CO)CCC3)c21]

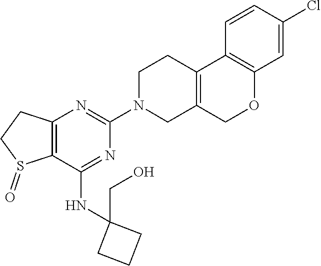 [SMILES: O=S1CCc2nc(N3CCC4=C(COc5cc(Cl)ccc54)C3)nc(NC3(CO)CCC3)c21]

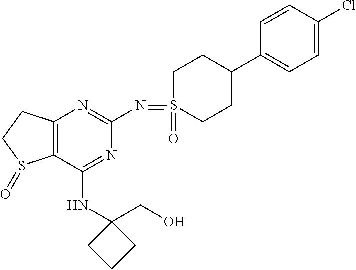 [SMILES: O=S1CCc2nc(N=S3(=O)CCC(c4ccc(Cl)cc4)CC3)nc(NC3(CO)CCC3)c21]

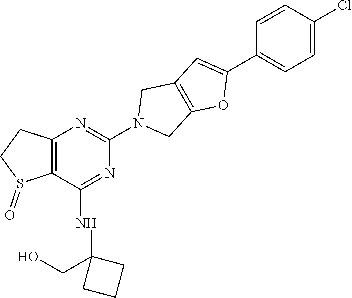 [SMILES: O=S1CCc2nc(N3Cc4cc(-c5ccc(Cl)cc5)oc4C3)nc(NC3(CO)CCC3)c21]

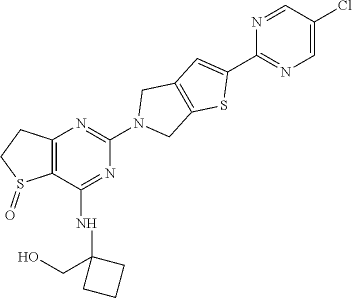 [SMILES: O=S1CCc2nc(N3Cc4cc(-c5ncc(Cl)cn5)sc4C3)nc(NC3(CO)CCC3)c21]

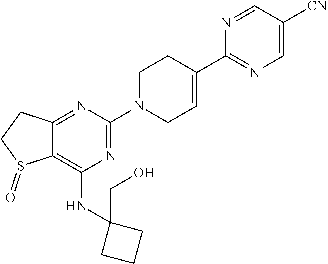 [SMILES: N#Cc1cnc(C2=CCN(c3nc4c(c(NC5(CO)CCC5)n3)S(=O)CC4)CC2)nc1]

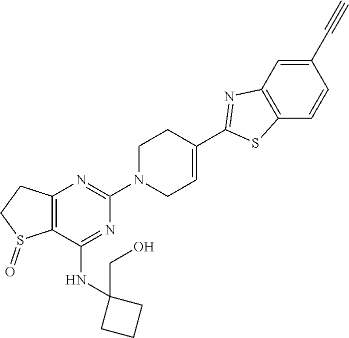 [SMILES: C#Cc1ccc2sc(C3=CCN(c4nc5c(c(NC6(CO)CCC6)n4)S(=O)CC5)CC3)nc2c1]

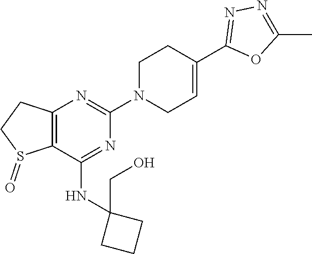 [SMILES: Cc1nnc(C2=CCN(c3nc4c(c(NC5(CO)CCC5)n3)S(=O)CC4)CC2)o1]

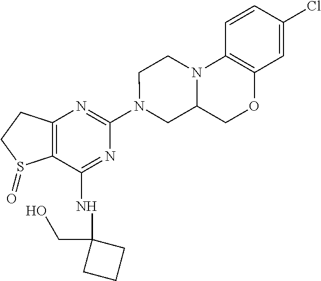 [SMILES: O=S1CCc2nc(N3CCN4c5ccc(Cl)cc5OCC4C3)nc(NC3(CO)CCC3)c21]

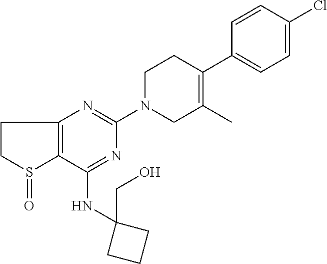 [SMILES: CC1=C(c2ccc(Cl)cc2)CCN(c2nc3c(c(NC4(CO)CCC4)n2)S(=O)CC3)C1]

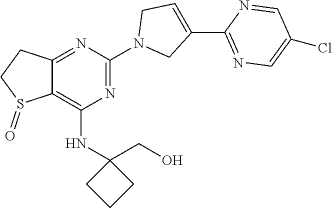 [SMILES: O=S1CCc2nc(N3CC=C(c4ncc(Cl)cn4)C3)nc(NC3(CO)CCC3)c21]

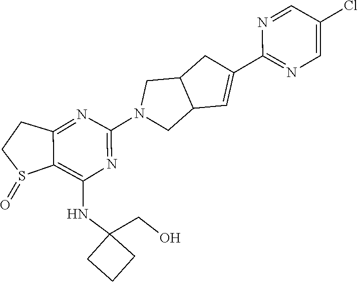 [SMILES: O=S1CCc2nc(N3CC4C=C(c5ncc(Cl)cn5)CC4C3)nc(NC3(CO)CCC3)c21]

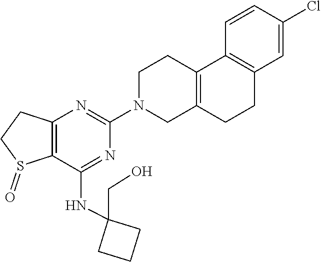 [SMILES: O=S1CCc2nc(N3CCC4=C(CCc5cc(Cl)ccc54)C3)nc(NC3(CO)CCC3)c21]

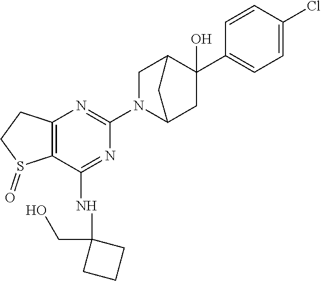 [SMILES: O=S1CCc2nc(N3CC4CC3CC4(O)c3ccc(Cl)cc3)nc(NC3(CO)CCC3)c21]

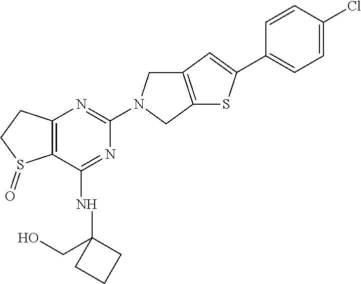 [SMILES: O=S1CCc2nc(N3Cc4cc(-c5ccc(Cl)cc5)sc4C3)nc(NC3(CO)CCC3)c21]

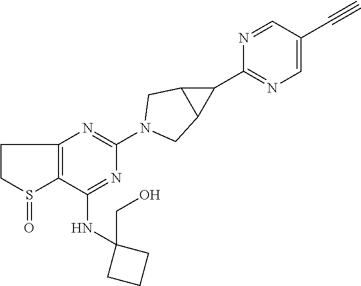 [SMILES: C#Cc1cnc(C2C3CN(c4nc5c(c(NC6(CO)CCC6)n4)S(=O)CC5)CC32)nc1]

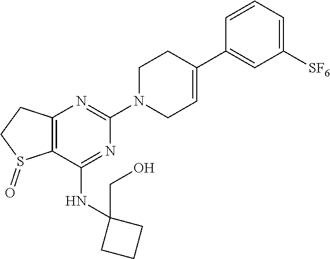

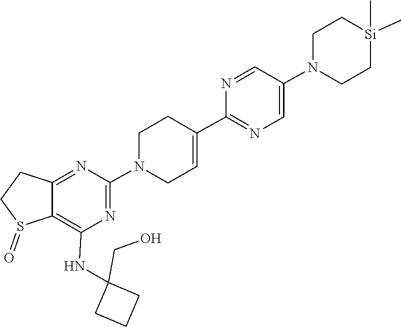 [SMILES: C[Si]1(C)CCN(c2cnc(C3=CCN(c4nc5c(c(NC6(CO)CCC6)n4)S(=O)CC5)CC3)nc2)CC1]

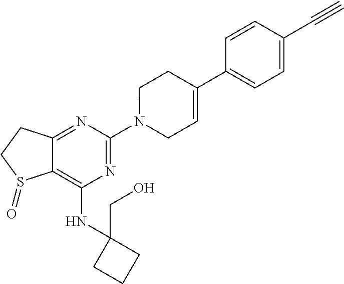 [SMILES: C#Cc1ccc(C2=CCN(c3nc4c(c(NC5(CO)CCC5)n3)S(=O)CC4)CC2)cc1]

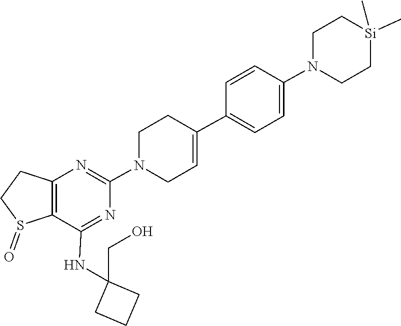 [SMILES: C[Si]1(C)CCN(c2ccc(C3=CCN(c4nc5c(c(NC6(CO)CCC6)n4)S(=O)CC5)CC3)cc2)CC1]

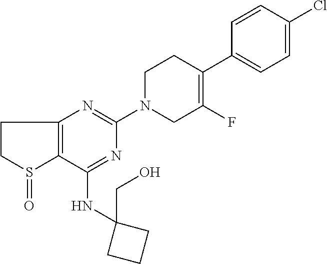 [SMILES: O=S1CCc2nc(N3CCC(c4ccc(Cl)cc4)=C(F)C3)nc(NC3(CO)CCC3)c21]

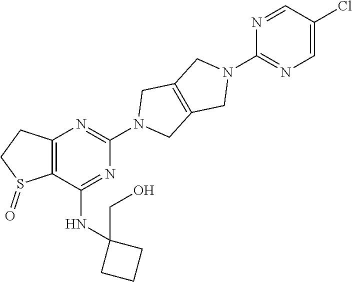 [SMILES: O=S1CCc2nc(N3CC4=C(CN(c5ncc(Cl)cn5)C4)C3)nc(NC3(CO)CCC3)c21]

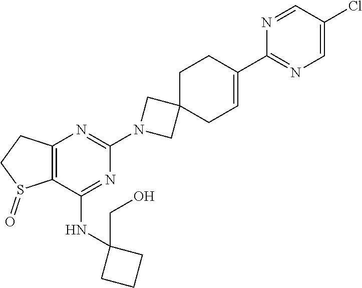 [SMILES: O=S1CCc2nc(N3CC4(CC=C(c5ncc(Cl)cn5)CC4)C3)nc(NC3(CO)CCC3)c21]

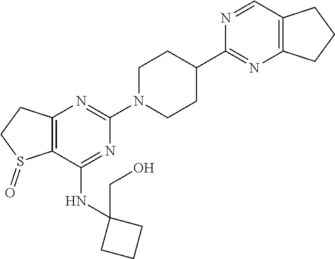 [SMILES: O=S1CCc2nc(N3CCC(c4ncc5c(n4)CCC5)CC3)nc(NC3(CO)CCC3)c21]

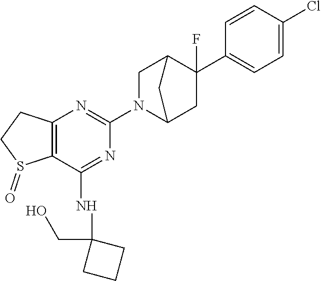 [SMILES: O=S1CCc2nc(N3CC4CC3CC4(F)c3ccc(Cl)cc3)nc(NC3(CO)CCC3)c21]

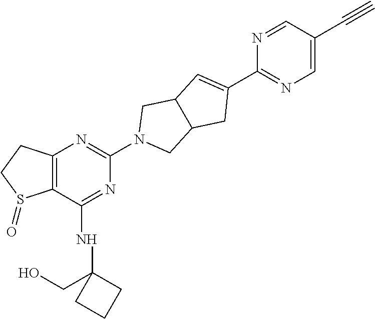 [SMILES: C#Cc1cnc(C2=CC3CN(c4nc5c(c(NC6(CO)CCC6)n4)S(=O)CC5)CC3C2)nc1]

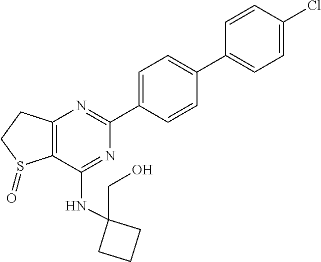 [SMILES: O=S1CCc2nc(-c3ccc(-c4ccc(Cl)cc4)cc3)nc(NC3(CO)CCC3)c21]

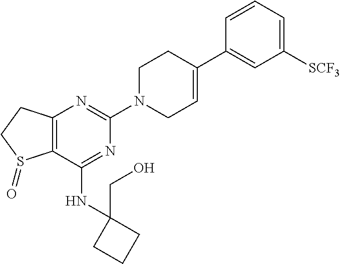

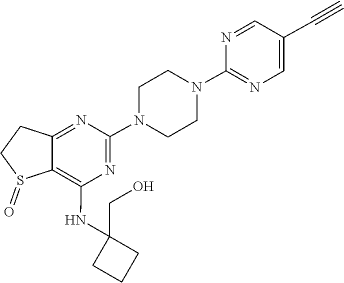 [SMILES: C#Cc1cnc(N2CCN(c3nc4c(c(NC5(CO)CCC5)n3)S(=O)CC4)CC2)nc1]

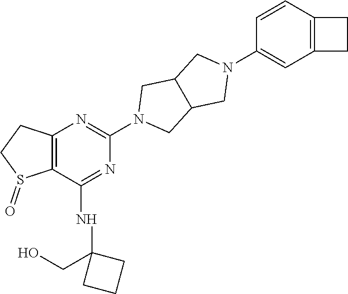 [SMILES: O=S1CCc2nc(N3CC4CN(c5ccc6c(c5)CC6)CC4C3)nc(NC3(CO)CCC3)c21]

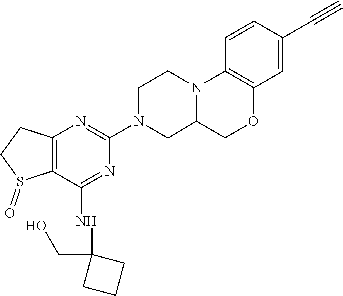 [SMILES: C#Cc1ccc2c(c1)OCC1CN(c3nc4c(c(NC5(CO)CCC5)n3)S(=O)CC4)CCN21]

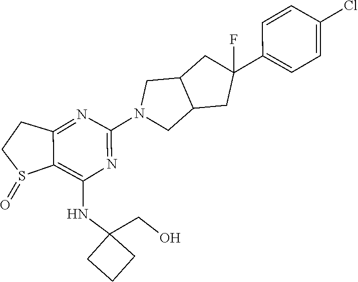 [SMILES: O=S1CCc2nc(N3CC4CC(F)(c5ccc(Cl)cc5)CC4C3)nc(NC3(CO)CCC3)c21]

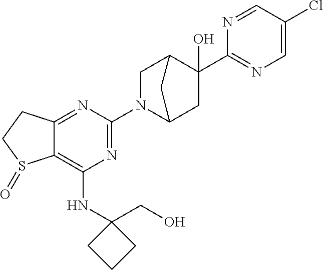 [SMILES: O=S1CCc2nc(N3CC4CC3CC4(O)c3ncc(Cl)cn3)nc(NC3(CO)CCC3)c21]

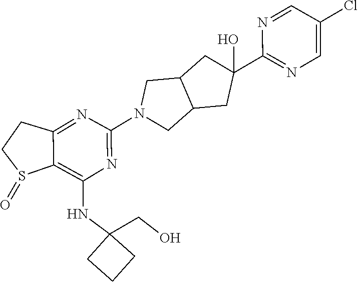 [SMILES: O=S1CCc2nc(N3CC4CC(O)(c5ncc(Cl)cn5)CC4C3)nc(NC3(CO)CCC3)c21]

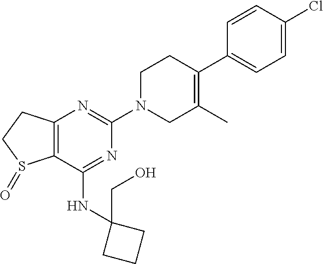 [SMILES: CC1=C(c2ccc(Cl)cc2)CCN(c2nc3c(c(NC4(CO)CCC4)n2)S(=O)CC3)C1]

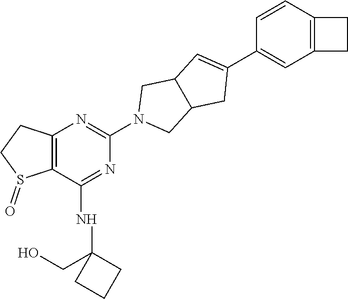 [SMILES: O=S1CCc2nc(N3CC4C=C(c5ccc6c(c5)CC6)CC4C3)nc(NC3(CO)CCC3)c21]

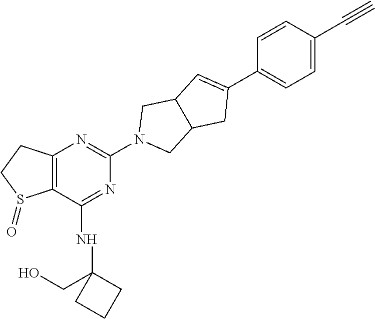 [SMILES: C#Cc1ccc(C2=CC3CN(c4nc5c(c(NC6(CO)CCC6)n4)S(=O)CC5)CC3C2)cc1]

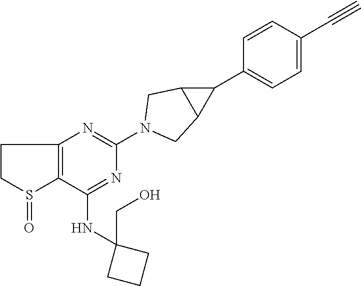 [SMILES: C#Cc1ccc(C2C3CN(c4nc5c(c(NC6(CO)CCC6)n4)S(=O)CC5)CC32)cc1]

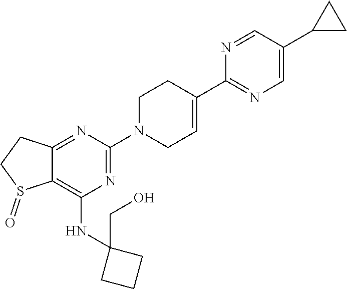 [SMILES: O=S1CCc2nc(N3CC=C(c4ncc(C5CC5)cn4)CC3)nc(NC3(CO)CCC3)c21]

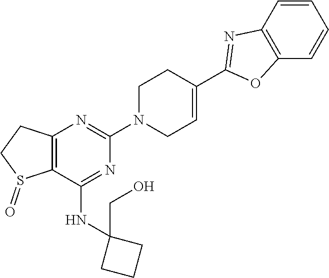 [SMILES: O=S1CCc2nc(N3CC=C(c4nc5ccccc5o4)CC3)nc(NC3(CO)CCC3)c21]

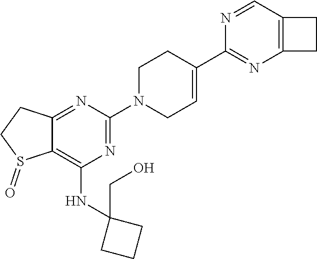 [SMILES: O=S1CCc2nc(N3CC=C(c4ncc5c(n4)CC5)CC3)nc(NC3(CO)CCC3)c21]

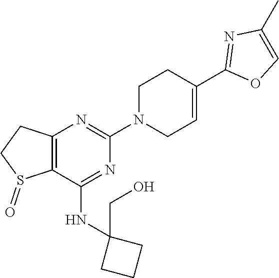 [SMILES: Cc1coc(C2=CCN(c3nc4c(c(NC5(CO)CCC5)n3)S(=O)CC4)CC2)n1]

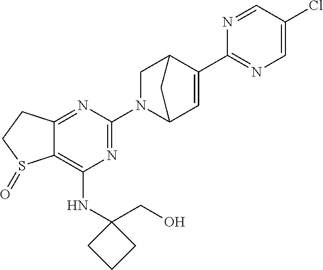 [SMILES: O=S1CCc2nc(N3CC4CC3C=C4c3ncc(Cl)cn3)nc(NC3(CO)CCC3)c21]

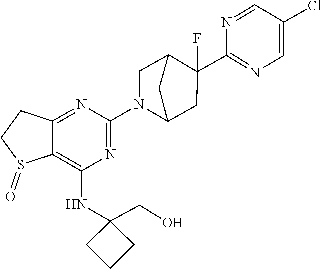 [SMILES: O=S1CCc2nc(N3CC4CC3CC4(F)c3ncc(Cl)cn3)nc(NC3(CO)CCC3)c21]

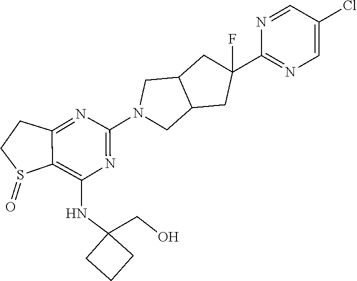 [SMILES: O=S1CCc2nc(N3CC4CC(F)(c5ncc(Cl)cn5)CC4C3)nc(NC3(CO)CCC3)c21]

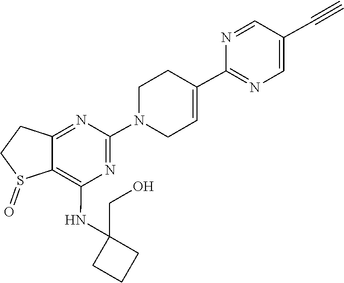 [SMILES: C#Cc1cnc(C2=CCN(c3nc4c(c(NC5(CO)CCC5)n3)S(=O)CC4)CC2)nc1]

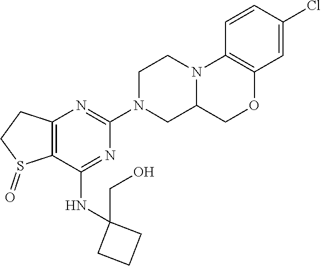 [SMILES: O=S1CCc2nc(N3CCN4c5ccc(Cl)cc5OCC4C3)nc(NC3(CO)CCC3)c21]

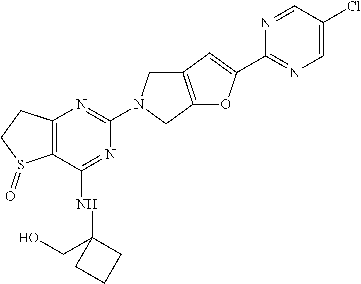 [SMILES: O=S1CCc2nc(N3Cc4cc(-c5ncc(Cl)cn5)oc4C3)nc(NC3(CO)CCC3)c21]

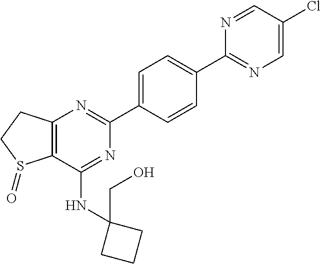 [SMILES: O=S1CCc2nc(-c3ccc(-c4ncc(Cl)cn4)cc3)nc(NC3(CO)CCC3)c21]

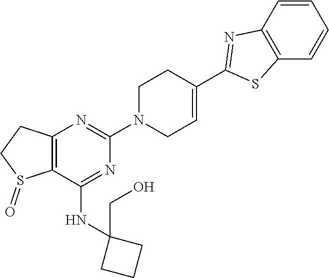 [SMILES: O=S1CCc2nc(N3CC=C(c4nc5ccccc5s4)CC3)nc(NC3(CO)CCC3)c21]

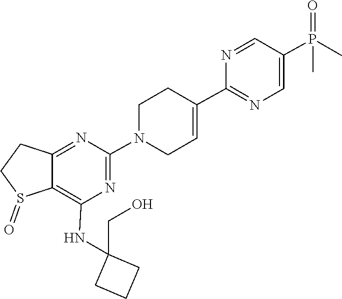 [SMILES: CP(C)(=O)c1cnc(C2=CCN(c3nc4c(c(NC5(CO)CCC5)n3)S(=O)CC4)CC2)nc1]

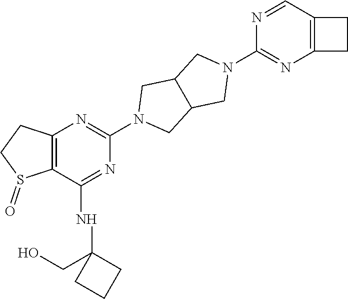 [SMILES: O=S1CCc2nc(N3CC4CN(c5ncc6c(n5)CC6)CC4C3)nc(NC3(CO)CCC3)c21]

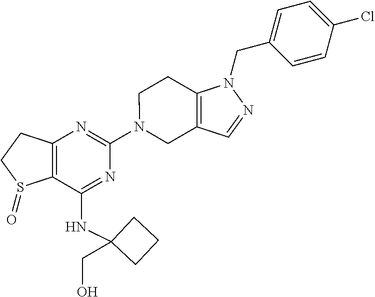 [SMILES: O=S1CCc2nc(N3CCc4c(cnn4Cc4ccc(Cl)cc4)C3)nc(NC3(CO)CCC3)c21]

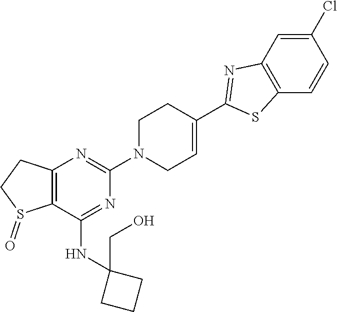 [SMILES: O=S1CCc2nc(N3CC=C(c4nc5cc(Cl)ccc5s4)CC3)nc(NC3(CO)CCC3)c21]

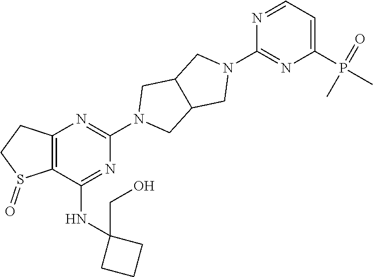 [SMILES: CP(C)(=O)c1ccnc(N2CC3CN(c4nc5c(c(NC6(CO)CCC6)n4)S(=O)CC5)CC3C2)n1]

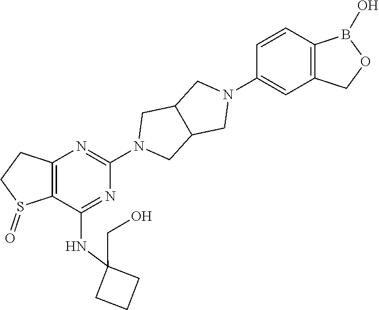 [SMILES: O=S1CCc2nc(N3CC4CN(c5ccc6c(c5)COB6O)CC4C3)nc(NC3(CO)CCC3)c21]

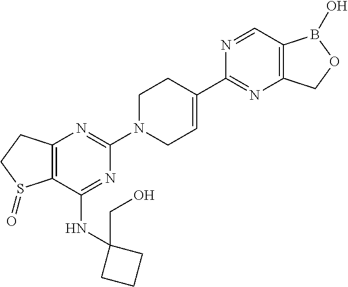 [SMILES: O=S1CCc2nc(N3CC=C(c4ncc5c(n4)COB5O)CC3)nc(NC3(CO)CCC3)c21]

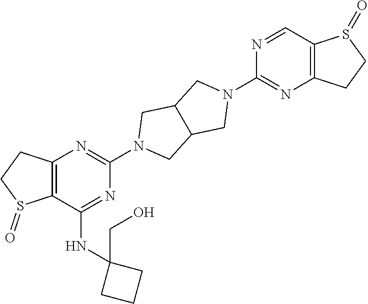 [SMILES: O=S1CCc2nc(N3CC4CN(c5nc6c(c(NC7(CO)CCC7)n5)S(=O)CC6)CC4C3)ncc21]

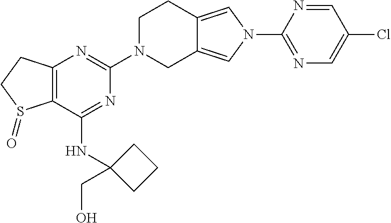 [SMILES: O=S1CCc2nc(N3CCc4cn(-c5ncc(Cl)cn5)cc4C3)nc(NC3(CO)CCC3)c21]

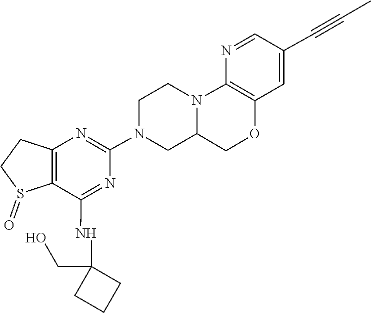 [SMILES: CC#Cc1cnc2c(c1)OCC1CN(c3nc4c(c(NC5(CO)CCC5)n3)S(=O)CC4)CCN21]

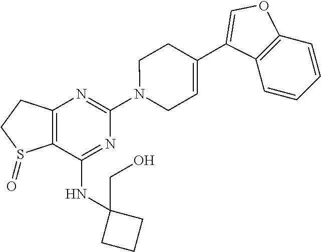 [SMILES: O=S1CCc2nc(N3CC=C(c4coc5ccccc45)CC3)nc(NC3(CO)CCC3)c21]

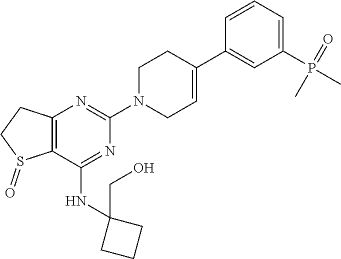 [SMILES: CP(C)(=O)c1cccc(C2=CCN(c3nc4c(c(NC5(CO)CCC5)n3)S(=O)CC4)CC2)c1]

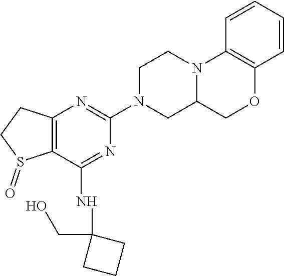 [SMILES: O=S1CCc2nc(N3CCN4c5ccccc5OCC4C3)nc(NC3(CO)CCC3)c21]

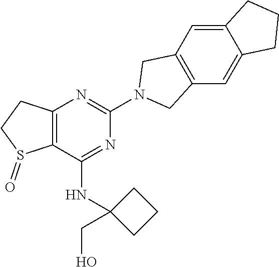 [SMILES: O=S1CCc2nc(N3Cc4cc5c(cc4C3)CCC5)nc(NC3(CO)CCC3)c21]

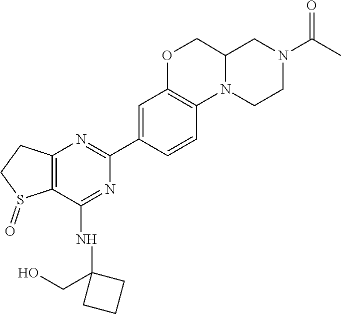 [SMILES: CC(=O)N1CCN2c3ccc(-c4nc5c(c(NC6(CO)CCC6)n4)S(=O)CC5)cc3OCC2C1]

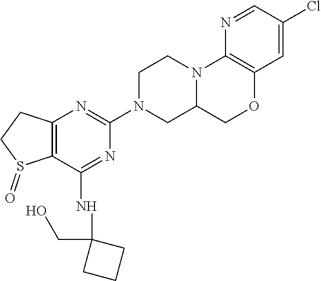 [SMILES: O=S1CCc2nc(N3CCN4c5ncc(Cl)cc5OCC4C3)nc(NC3(CO)CCC3)c21]

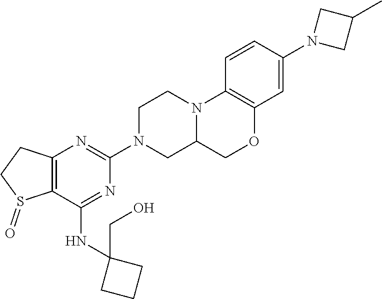 [SMILES: CC1CN(c2ccc3c(c2)OCC2CN(c4nc5c(c(NC6(CO)CCC6)n4)S(=O)CC5)CCN32)C1]

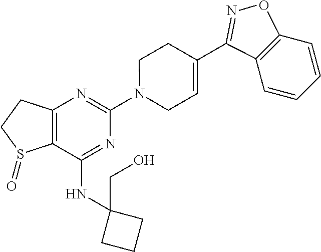 [SMILES: O=S1CCc2nc(N3CC=C(c4noc5ccccc45)CC3)nc(NC3(CO)CCC3)c21]

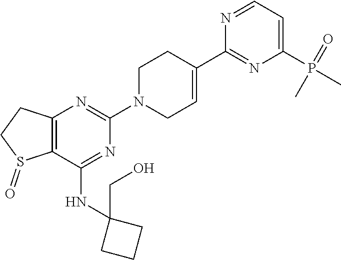 [SMILES: CP(C)(=O)c1ccnc(C2=CCN(c3nc4c(c(NC5(CO)CCC5)n3)S(=O)CC4)CC2)n1]

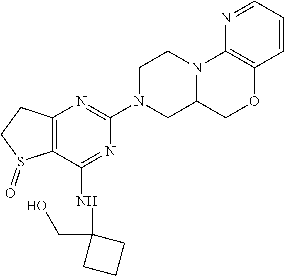 [SMILES: O=S1CCc2nc(N3CCN4c5ncccc5OCC4C3)nc(NC3(CO)CCC3)c21]

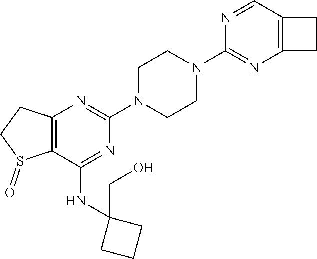 [SMILES: O=S1CCc2nc(N3CCN(c4ncc5c(n4)CC5)CC3)nc(NC3(CO)CCC3)c21]

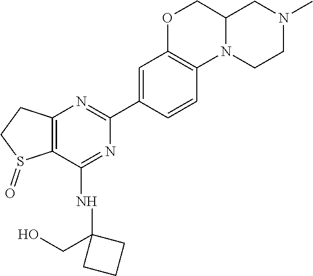 [SMILES: CN1CCN2c3ccc(-c4nc5c(c(NC6(CO)CCC6)n4)S(=O)CC5)cc3OCC2C1]

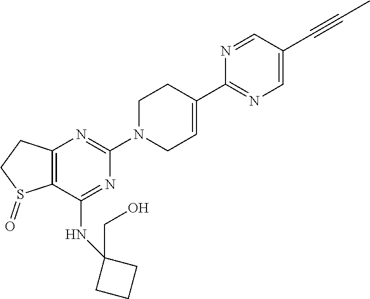 [SMILES: CC#Cc1cnc(C2=CCN(c3nc4c(c(NC5(CO)CCC5)n3)S(=O)CC4)CC2)nc1]

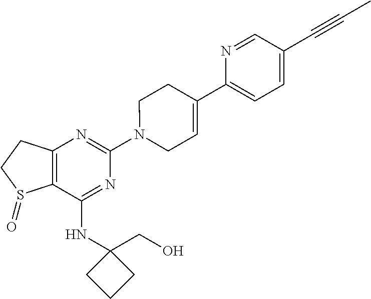 [SMILES: CC#Cc1ccc(C2=CCN(c3nc4c(c(NC5(CO)CCC5)n3)S(=O)CC4)CC2)nc1]

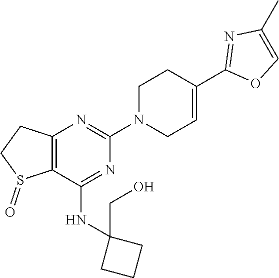 [SMILES: Cc1coc(C2=CCN(c3nc4c(c(NC5(CO)CCC5)n3)S(=O)CC4)CC2)n1]

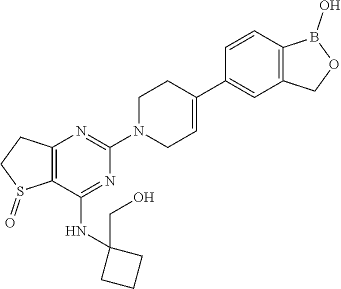 [SMILES: O=S1CCc2nc(N3CC=C(c4ccc5c(c4)COB5O)CC3)nc(NC3(CO)CCC3)c21]

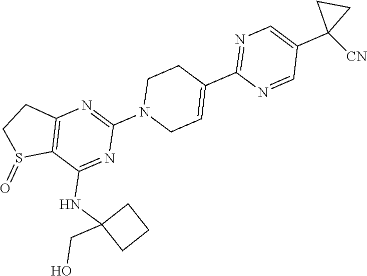 [SMILES: N#CC1(c2cnc(C3=CCN(c4nc5c(c(NC6(CO)CCC6)n4)S(=O)CC5)CC3)nc2)CC1]

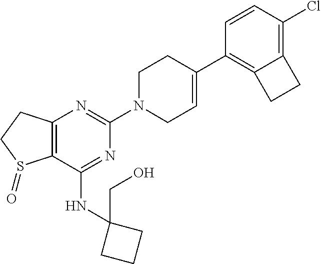 [SMILES: O=S1CCc2nc(N3CC=C(c4ccc(Cl)c5c4CC5)CC3)nc(NC3(CO)CCC3)c21]

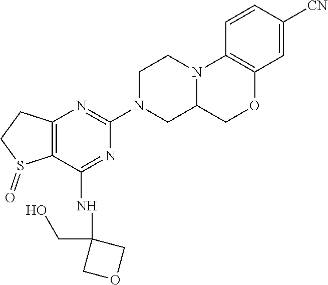 [SMILES: N#Cc1ccc2c(c1)OCC1CN(c3nc4c(c(NC5(CO)COC5)n3)S(=O)CC4)CCN21]

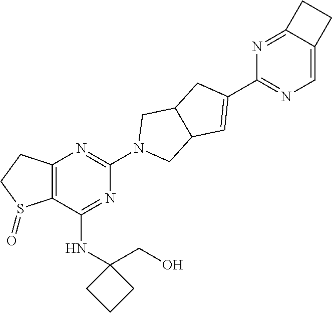 [SMILES: O=S1CCc2nc(N3CC4C=C(c5ncc6c(n5)CC6)CC4C3)nc(NC3(CO)CCC3)c21]

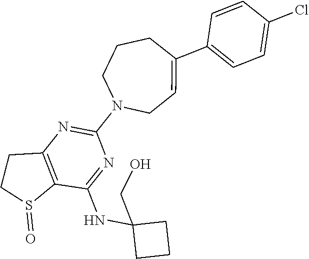 [SMILES: O=S1CCc2nc(N3CC=C(c4ccc(Cl)cc4)CCC3)nc(NC3(CO)CCC3)c21]

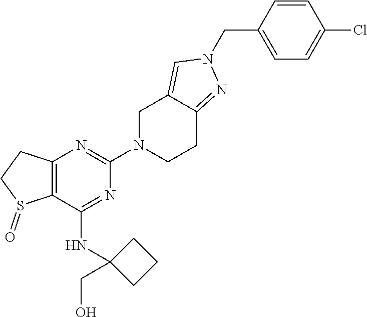 [SMILES: O=S1CCc2nc(N3CCc4nn(Cc5ccc(Cl)cc5)cc4C3)nc(NC3(CO)CCC3)c21]

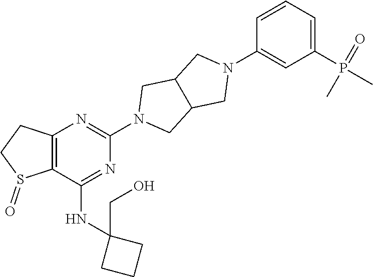 [SMILES: CP(C)(=O)c1cccc(N2CC3CN(c4nc5c(c(NC6(CO)CCC6)n4)S(=O)CC5)CC3C2)c1]

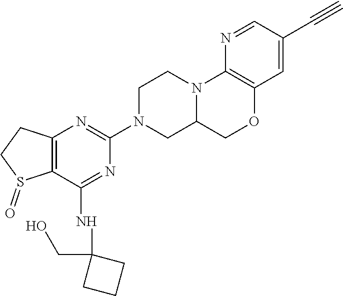 [SMILES: C#Cc1cnc2c(c1)OCC1CN(c3nc4c(c(NC5(CO)CCC5)n3)S(=O)CC4)CCN21]

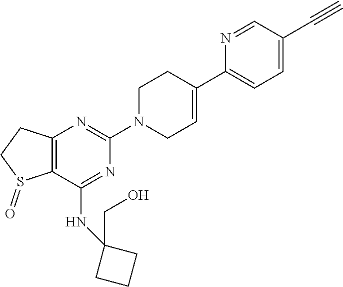 [SMILES: C#Cc1ccc(C2=CCN(c3nc4c(c(NC5(CO)CCC5)n3)S(=O)CC4)CC2)nc1]

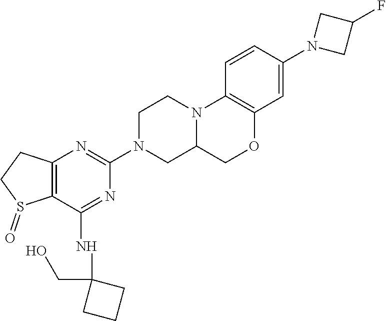 [SMILES: O=S1CCc2nc(N3CCN4c5ccc(N6CC(F)C6)cc5OCC4C3)nc(NC3(CO)CCC3)c21]

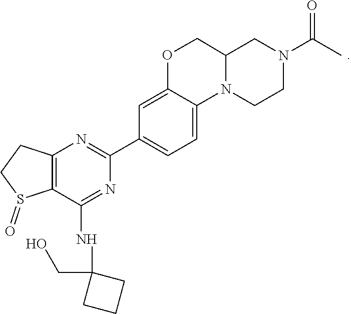 [SMILES: CC(=O)N1CCN2c3ccc(-c4nc5c(c(NC6(CO)CCC6)n4)S(=O)CC5)cc3OCC2C1]

In solution 8 of the present invention, the compound of the present invention is selected from a compound with one of the structures in Table 2 below, or a stereoisomer or pharmaceutically acceptable salt thereof,

TABLE 2

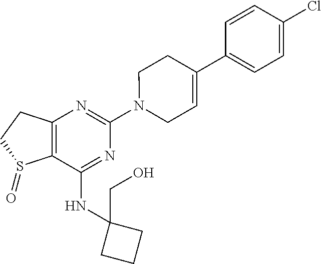 [SMILES: O=[S@@]1CCc2nc(N3CC=C(c4ccc(Cl)cc4)CC3)nc(NC3(CO)CCC3)c21]

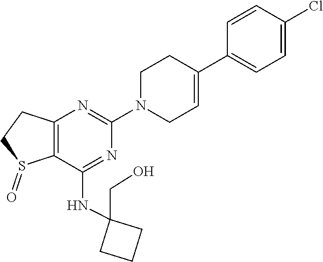 [SMILES: O=[S@]1CCc2nc(N3CC=C(c4ccc(Cl)cc4)CC3)nc(NC3(CO)CCC3)c21]

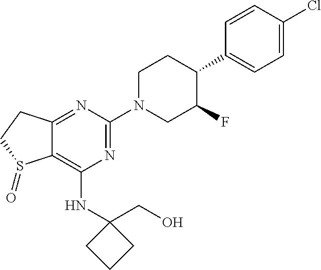 [SMILES: O=[S@@]1CCc2nc(N3CC[C@H](c4ccc(Cl)cc4)[C@@H](F)C3)nc(NC3(CO)CCC3)c21]

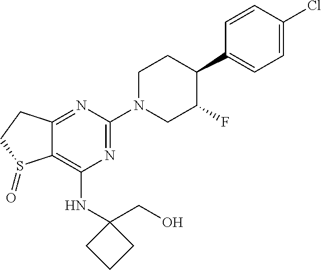 [SMILES: O=[S@@]1CCc2nc(N3CC[C@@H](c4ccc(Cl)cc4)[C@H](F)C3)nc(NC3(CO)CCC3)c21]

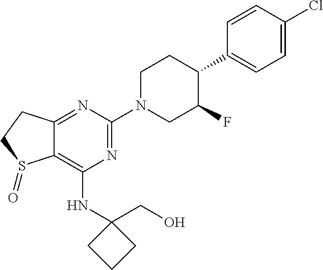 [SMILES: O=[S@]1CCc2nc(N3CC[C@H](c4ccc(Cl)cc4)[C@@H](F)C3)nc(NC3(CO)CCC3)c21]

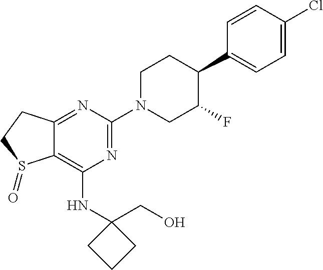 [SMILES: O=[S@]1CCc2nc(N3CC[C@@H](c4ccc(Cl)cc4)[C@H](F)C3)nc(NC3(CO)CCC3)c21]

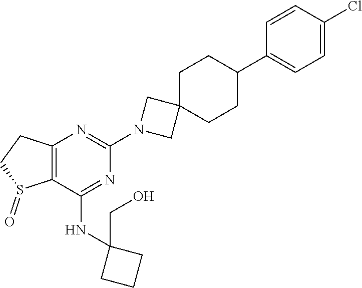

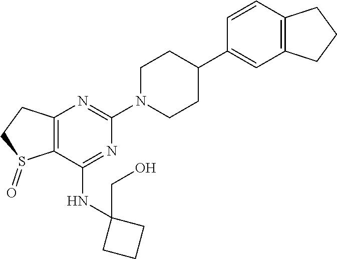 [SMILES: O=[S@]1CCc2nc(N3CCC(c4ccc5c(c4)CCC5)CC3)nc(NC3(CO)CCC3)c21]

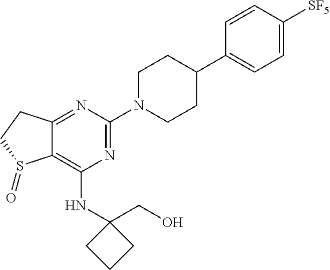

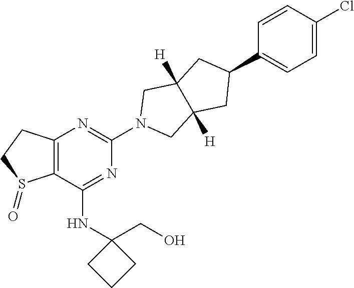 [SMILES: O=[S@]1CCc2nc(N3C[C@H]4C[C@H](c5ccc(Cl)cc5)C[C@H]4C3)nc(NC3(CO)CCC3)c21]

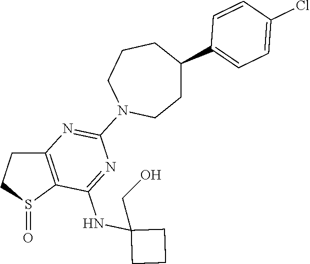 [SMILES: O=[S@]1CCc2nc(N3CCC[C@@H](c4ccc(Cl)cc4)CC3)nc(NC3(CO)CCC3)c21]

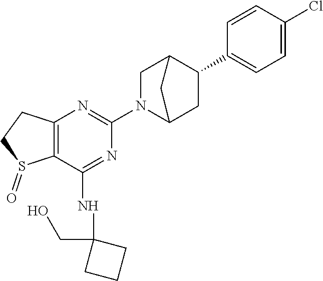 [SMILES: O=[S@]1CCc2nc(N3CC4CC3C[C@H]4c3ccc(Cl)cc3)nc(NC3(CO)CCC3)c21]

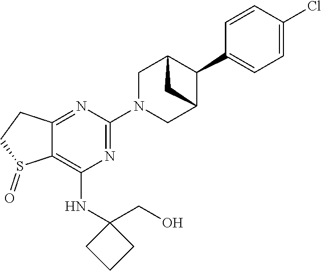 [SMILES: O=[S@@]1CCc2nc(N3C[C@H]4C[C@@H](C3)[C@@H]4c3ccc(Cl)cc3)nc(NC3(CO)CCC3)c21]

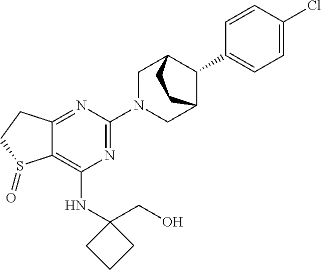

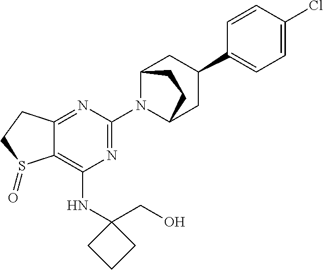

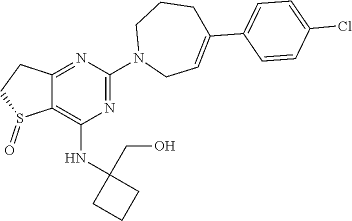 [SMILES: O=[S@@]1CCc2nc(N3CC=C(c4ccc(Cl)cc4)CCC3)nc(NC3(CO)CCC3)c21]

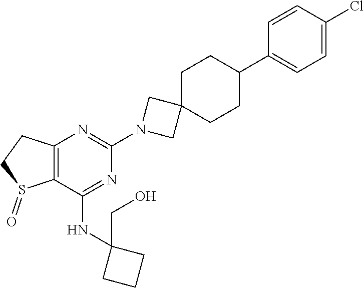

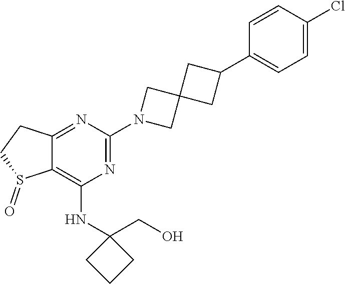 [SMILES: O=[S@@]1CCc2nc(N3CC4(CC(c5ccc(Cl)cc5)C4)C3)nc(NC3(CO)CCC3)c21]

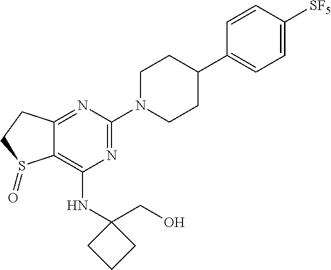

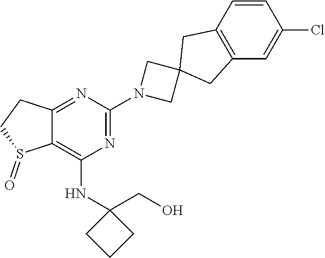 [SMILES: O=[S@@]1CCc2nc(N3CC4(Cc5ccc(Cl)cc5C4)C3)nc(NC3(CO)CCC3)c21]

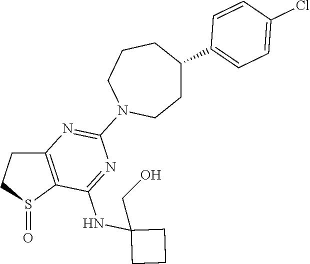 [SMILES: O=[S@]1CCc2nc(N3CCC[C@H](c4ccc(Cl)cc4)CC3)nc(NC3(CO)CCC3)c21]

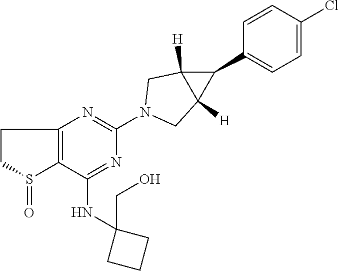 [SMILES: O=[S@@]1CCc2nc(N3C[C@@H]4[C@H](C3)[C@H]4c3ccc(Cl)cc3)nc(NC3(CO)CCC3)c21]

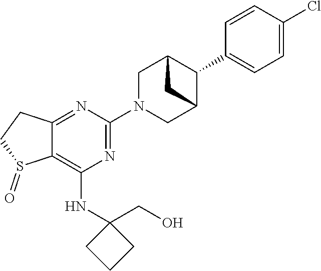 [SMILES: O=[S@@]1CCc2nc(N3C[C@H]4C[C@@H](C3)[C@H]4c3ccc(Cl)cc3)nc(NC3(CO)CCC3)c21]

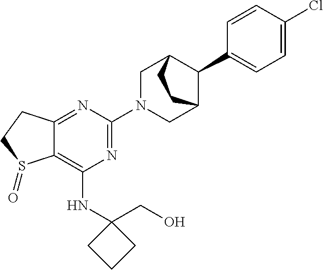

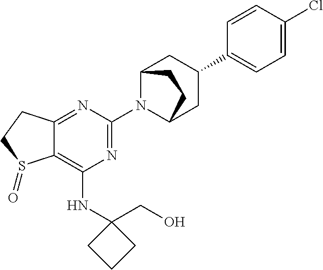

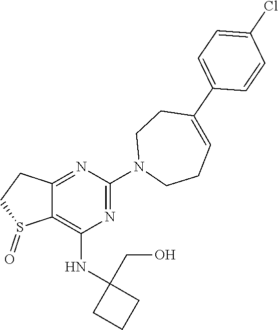 [SMILES: O=[S@@]1CCc2nc(N3CCC=C(c4ccc(Cl)cc4)CC3)nc(NC3(CO)CCC3)c21]

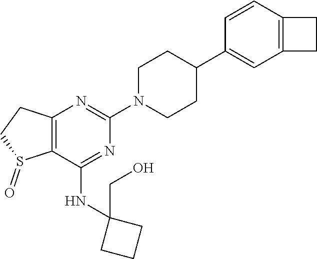 [SMILES: O=[S@@]1CCc2nc(N3CCC(c4ccc5c(c4)CC5)CC3)nc(NC3(CO)CCC3)c21]

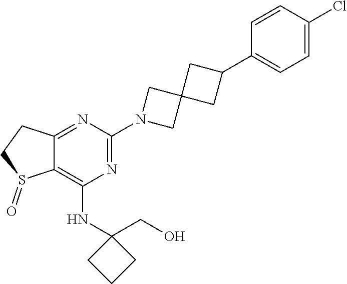 [SMILES: O=[S@]1CCc2nc(N3CC4(CC(c5ccc(Cl)cc5)C4)C3)nc(NC3(CO)CCC3)c21]

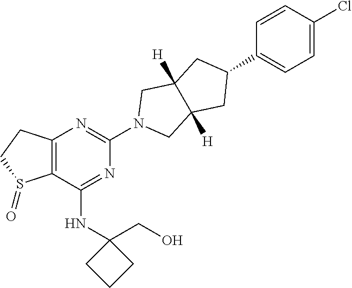 [SMILES: O=[S@@]1CCc2nc(N3C[C@H]4C[C@@H](c5ccc(Cl)cc5)C[C@H]4C3)nc(NC3(CO)CCC3)c21]

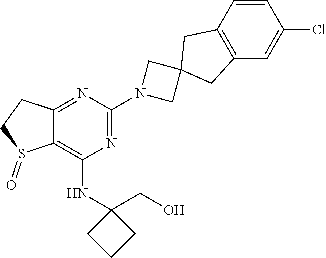 [SMILES: O=[S@]1CCc2nc(N3CC4(Cc5ccc(Cl)cc5C4)C3)nc(NC3(CO)CCC3)c21]

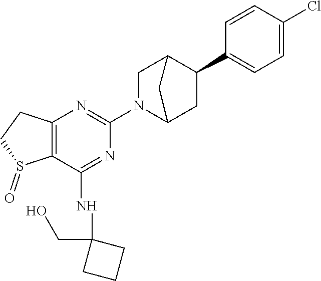 [SMILES: O=[S@@]1CCc2nc(N3CC4CC3C[C@@H]4c3ccc(Cl)cc3)nc(NC3(CO)CCC3)c21]

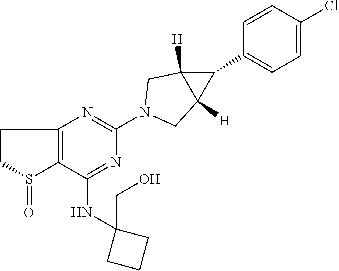 [SMILES: O=[S@@]1CCc2nc(N3C[C@@H]4[C@H](C3)[C@@H]4c3ccc(Cl)cc3)nc(NC3(CO)CCC3)c21]

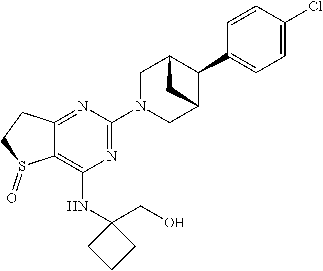 [SMILES: O=[S@]1CCc2nc(N3C[C@H]4C[C@@H](C3)[C@@H]4c3ccc(Cl)cc3)nc(NC3(CO)CCC3)c21]

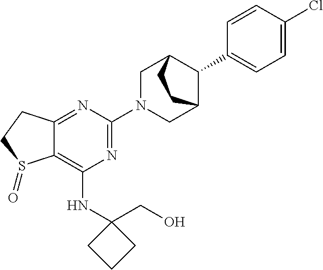

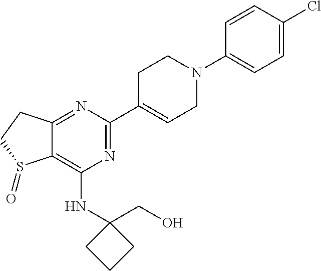 [SMILES: O=[S@@]1CCc2nc(C3=CCN(c4ccc(Cl)cc4)CC3)nc(NC3(CO)CCC3)c21]

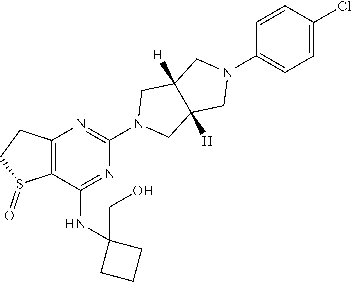 [SMILES: O=[S@@]1CCc2nc(N3C[C@H]4CN(c5ccc(Cl)cc5)C[C@H]4C3)nc(NC3(CO)CCC3)c21]

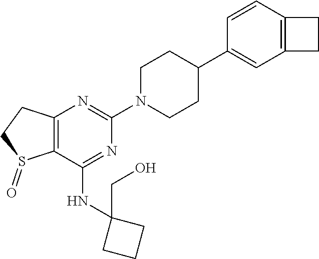 [SMILES: O=[S@]1CCc2nc(N3CCC(c4ccc5c(c4)CC5)CC3)nc(NC3(CO)CCC3)c21]

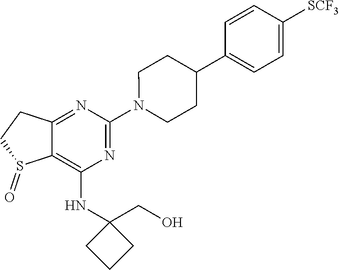

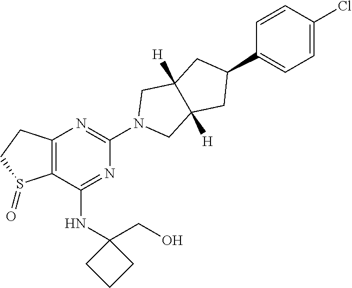 [SMILES: O=[S@@]1CCc2nc(N3C[C@H]4C[C@H](c5ccc(Cl)cc5)C[C@H]4C3)nc(NC3(CO)CCC3)c21]

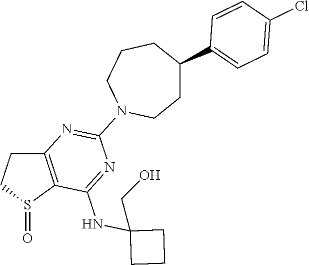 [SMILES: O=[S@@]1CCc2nc(N3CCC[C@@H](c4ccc(Cl)cc4)CC3)nc(NC3(CO)CCC3)c21]

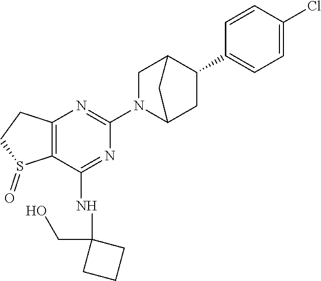 [SMILES: O=[S@@]1CCc2nc(N3CC4CC3C[C@H]4c3ccc(Cl)cc3)nc(NC3(CO)CCC3)c21]

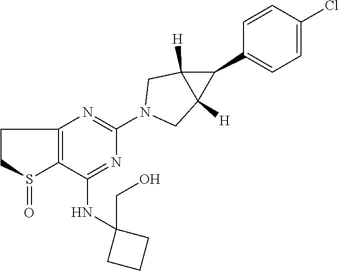 [SMILES: O=[S@]1CCc2nc(N3C[C@@H]4[C@H](C3)[C@H]4c3ccc(Cl)cc3)nc(NC3(CO)CCC3)c21]

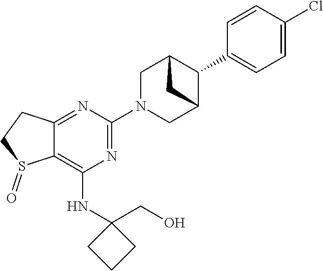 [SMILES: O=[S@]1CCc2nc(N3C[C@H]4C[C@@H](C3)[C@H]4c3ccc(Cl)cc3)nc(NC3(CO)CCC3)c21]

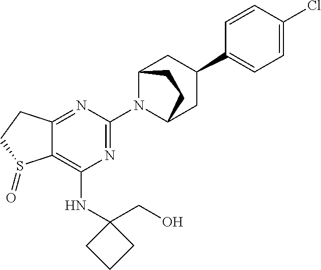

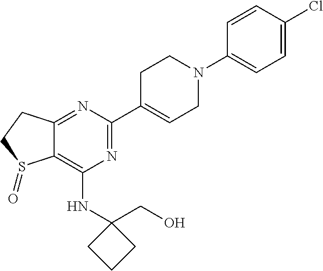 [SMILES: O=[S@]1CCc2nc(C3=CCN(c4ccc(Cl)cc4)CC3)nc(NC3(CO)CCC3)c21]

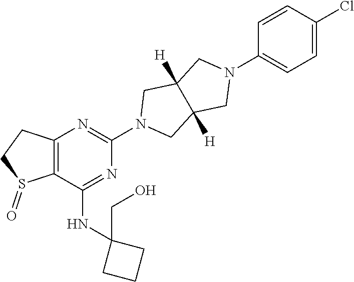 [SMILES: O=[S@]1CCc2nc(N3C[C@H]4CN(c5ccc(Cl)cc5)C[C@H]4C3)nc(NC3(CO)CCC3)c21]

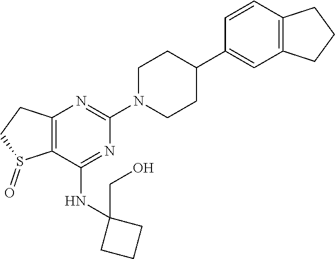 [SMILES: O=[S@@]1CCc2nc(N3CCC(c4ccc5c(c4)CCC5)CC3)nc(NC3(CO)CCC3)c21]

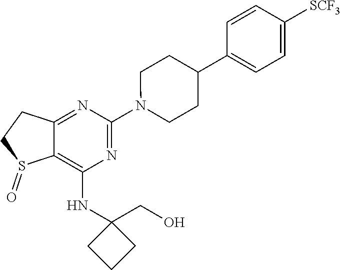

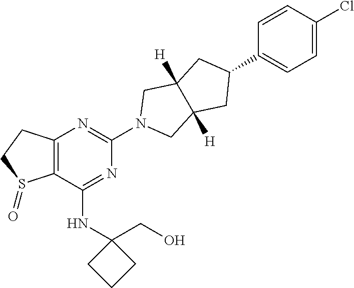 [SMILES: O=[S@]1CCc2nc(N3C[C@H]4C[C@@H](c5ccc(Cl)cc5)C[C@H]4C3)nc(NC3(CO)CCC3)c21]

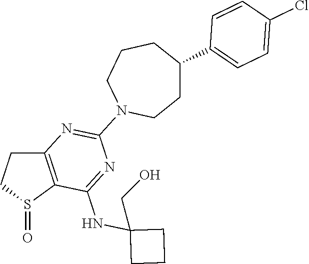 [SMILES: O=[S@@]1CCc2nc(N3CCC[C@H](c4ccc(Cl)cc4)CC3)nc(NC3(CO)CCC3)c21]

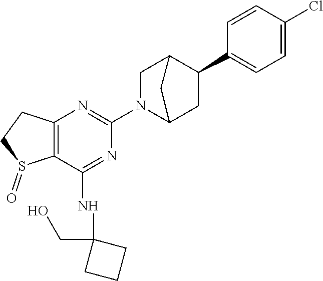 [SMILES: O=[S@]1CCc2nc(N3CC4CC3C[C@@H]4c3ccc(Cl)cc3)nc(NC3(CO)CCC3)c21]

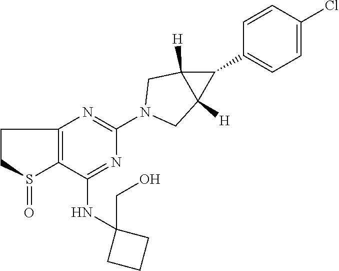 [SMILES: O=[S@]1CCc2nc(N3C[C@@H]4[C@H](C3)[C@@H]4c3ccc(Cl)cc3)nc(NC3(CO)CCC3)c21]

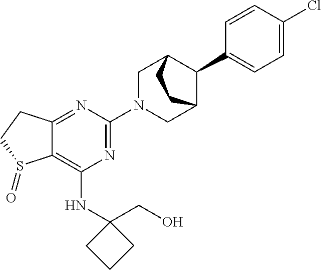

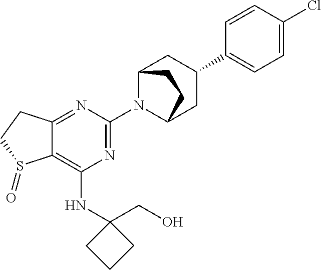

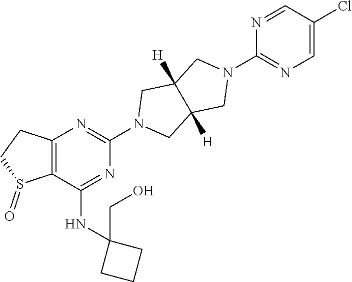 [SMILES: O=[S@@]1CCc2nc(N3C[C@H]4CN(c5ncc(Cl)cn5)C[C@H]4C3)nc(NC3(CO)CCC3)c21]

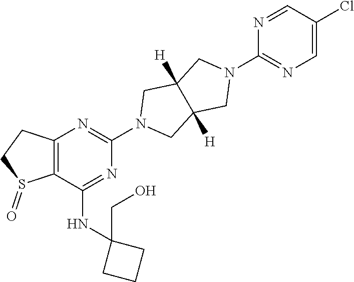 [SMILES: O=[S@]1CCc2nc(N3C[C@H]4CN(c5ncc(Cl)cn5)C[C@H]4C3)nc(NC3(CO)CCC3)c21]

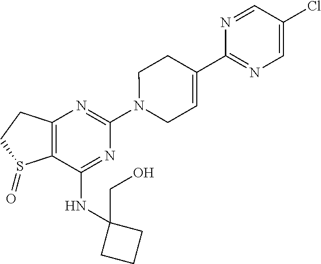 [SMILES: O=[S@@]1CCc2nc(N3CC=C(c4ncc(Cl)cn4)CC3)nc(NC3(CO)CCC3)c21]

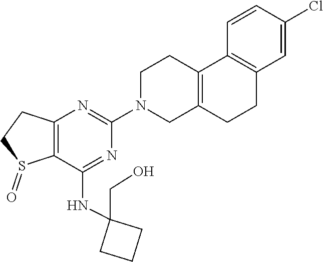 [SMILES: O=[S@]1CCc2nc(N3CCC4=C(CCc5cc(Cl)ccc54)C3)nc(NC3(CO)CCC3)c21]

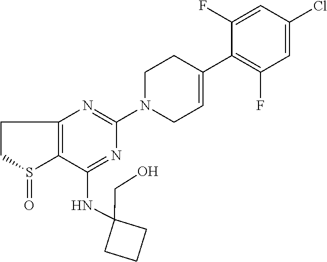 [SMILES: O=[S@@]1CCc2nc(N3CC=C(c4c(F)cc(Cl)cc4F)CC3)nc(NC3(CO)CCC3)c21]

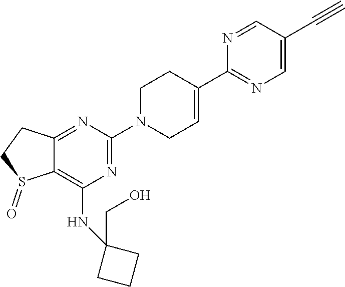 [SMILES: C#Cc1cnc(C2=CCN(c3nc4c(c(NC5(CO)CCC5)n3)[S@@](=O)CC4)CC2)nc1]

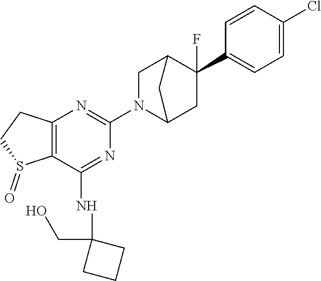 [SMILES: O=[S@@]1CCc2nc(N3CC4CC3C[C@]4(F)c3ccc(Cl)cc3)nc(NC3(CO)CCC3)c21]

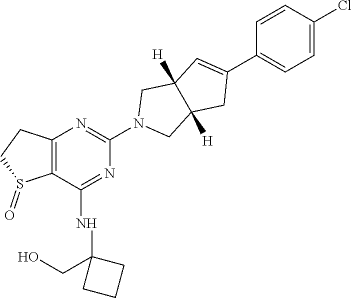 [SMILES: O=[S@@]1CCc2nc(N3C[C@H]4CC(c5ccc(Cl)cc5)=C[C@H]4C3)nc(NC3(CO)CCC3)c21]

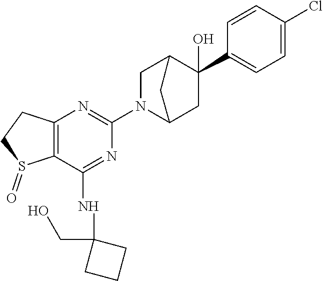 [SMILES: O=[S@]1CCc2nc(N3CC4CC3C[C@]4(O)c3ccc(Cl)cc3)nc(NC3(CO)CCC3)c21]

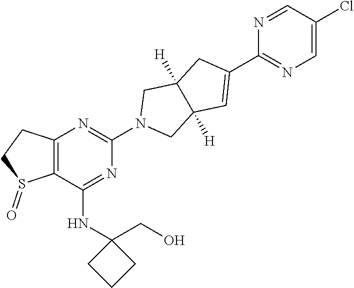 [SMILES: O=[S@]1CCc2nc(N3C[C@H]4CC(c5ncc(Cl)cn5)=C[C@H]4C3)nc(NC3(CO)CCC3)c21]

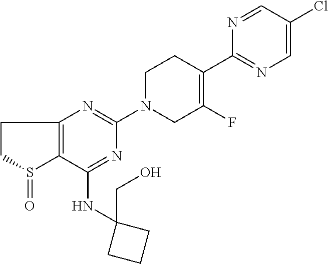 [SMILES: O=[S@@]1CCc2nc(N3CCC(c4ncc(Cl)cn4)=C(F)C3)nc(NC3(CO)CCC3)c21]

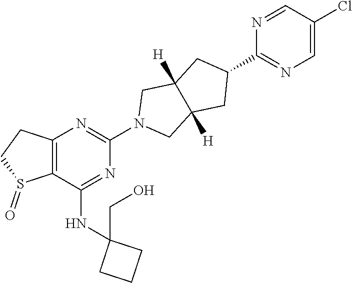 [SMILES: O=[S@@]1CCc2nc(N3C[C@H]4C[C@@H](c5ncc(Cl)cn5)C[C@H]4C3)nc(NC3(CO)CCC3)c21]

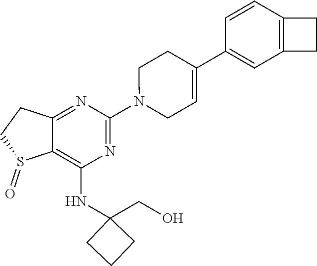 [SMILES: O=[S@@]1CCc2nc(N3CC=C(c4ccc5c(c4)CC5)CC3)nc(NC3(CO)CCC3)c21]

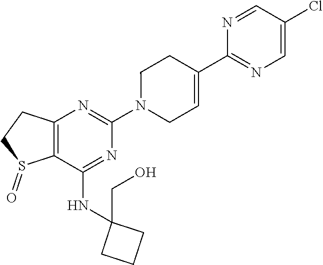 [SMILES: O=[S@]1CCc2nc(N3CC=C(c4ncc(Cl)cn4)CC3)nc(NC3(CO)CCC3)c21]

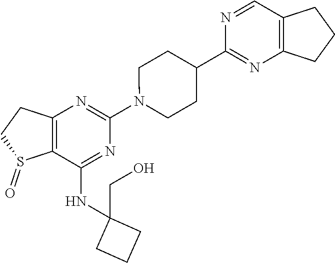 [SMILES: O=[S@@]1CCc2nc(N3CCC(c4ncc5c(n4)CCC5)CC3)nc(NC3(CO)CCC3)c21]

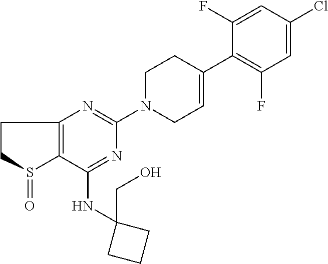 [SMILES: O=[S@]1CCc2nc(N3CC=C(c4c(F)cc(Cl)cc4F)CC3)nc(NC3(CO)CCC3)c21]

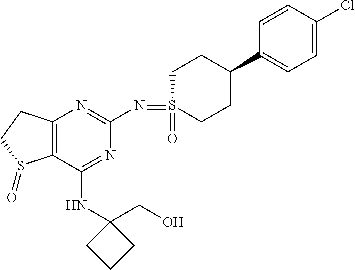 [SMILES: O=[S@@]1CCc2nc(N=[S@]3(=O)CC[C@H](c4ccc(Cl)cc4)CC3)nc(NC3(CO)CCC3)c21]

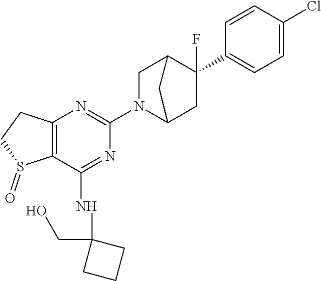 [SMILES: O=[S@@]1CCc2nc(N3CC4CC3C[C@@]4(F)c3ccc(Cl)cc3)nc(NC3(CO)CCC3)c21]

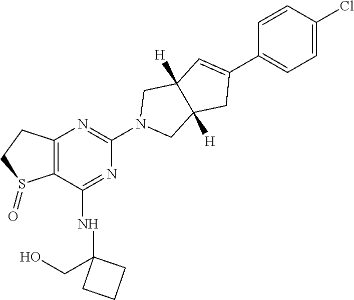 [SMILES: O=[S@]1CCc2nc(N3C[C@H]4CC(c5ccc(Cl)cc5)=C[C@H]4C3)nc(NC3(CO)CCC3)c21]

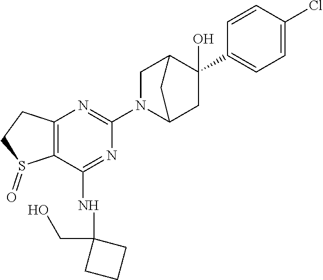 [SMILES: O=[S@]1CCc2nc(N3CC4CC3C[C@@]4(O)c3ccc(Cl)cc3)nc(NC3(CO)CCC3)c21]

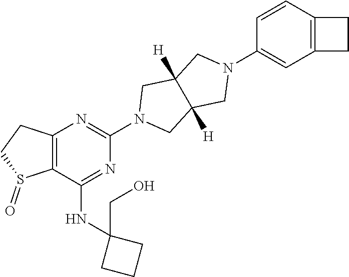 [SMILES: O=[S@@]1CCc2nc(N3C[C@H]4CN(c5ccc6c(c5)CC6)C[C@H]4C3)nc(NC3(CO)CCC3)c21]

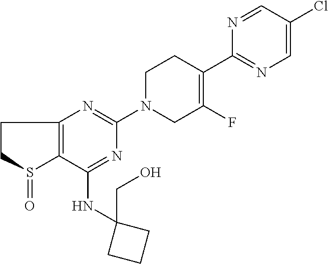 [SMILES: O=[S@]1CCc2nc(N3CCC(c4ncc(Cl)cn4)=C(F)C3)nc(NC3(CO)CCC3)c21]

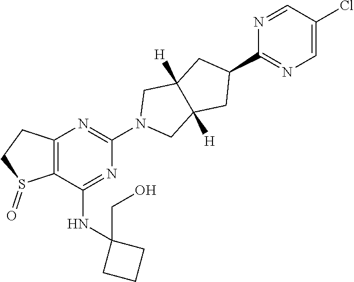 [SMILES: O=[S@]1CCc2nc(N3C[C@H]4C[C@H](c5ncc(Cl)cn5)C[C@H]4C3)nc(NC3(CO)CCC3)c21]

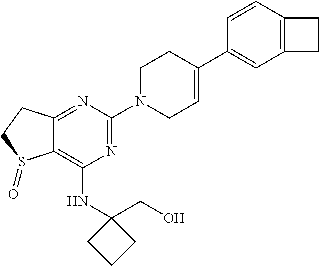 [SMILES: O=[S@]1CCc2nc(N3CC=C(c4ccc5c(c4)CC5)CC3)nc(NC3(CO)CCC3)c21]

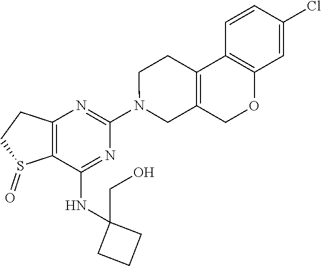 [SMILES: O=[S@@]1CCc2nc(N3CCC4=C(COc5cc(Cl)ccc54)C3)nc(NC3(CO)CCC3)c21]

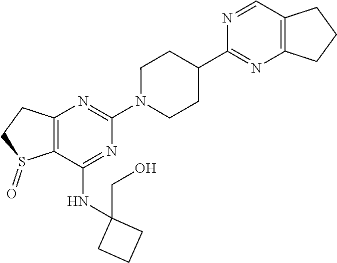 [SMILES: O=[S@]1CCc2nc(N3CCC(c4ncc5c(n4)CCC5)CC3)nc(NC3(CO)CCC3)c21]

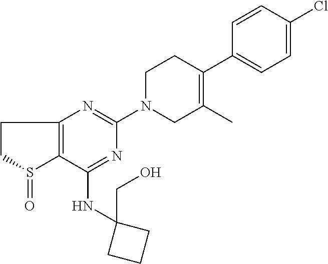 [SMILES: CC1=C(c2ccc(Cl)cc2)CCN(c2nc3c(c(NC4(CO)CCC4)n2)[S@](=O)CC3)C1]

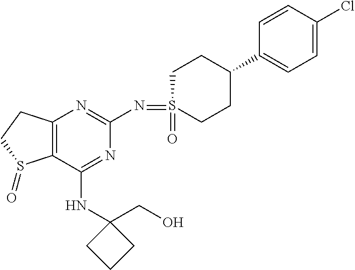 [SMILES: O=[S@@]1CCc2nc(N=[S@]3(=O)CC[C@@H](c4ccc(Cl)cc4)CC3)nc(NC3(CO)CCC3)c21]

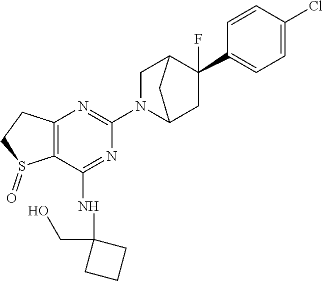 [SMILES: O=[S@]1CCc2nc(N3CC4CC3C[C@]4(F)c3ccc(Cl)cc3)nc(NC3(CO)CCC3)c21]

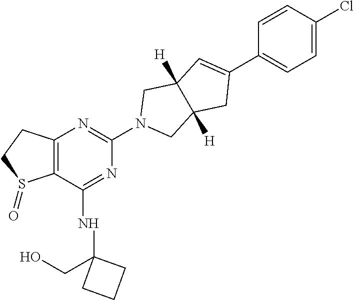 [SMILES: O=[S@]1CCc2nc(N3C[C@H]4CC(c5ccc(Cl)cc5)=C[C@H]4C3)nc(NC3(CO)CCC3)c21]

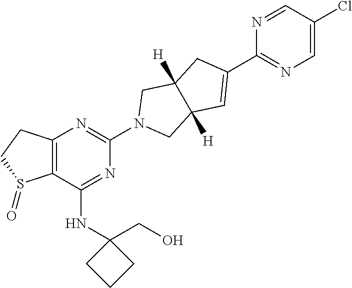 [SMILES: O=[S@@]1CCc2nc(N3C[C@@H]4CC(c5ncc(Cl)cn5)=C[C@@H]4C3)nc(NC3(CO)CCC3)c21]

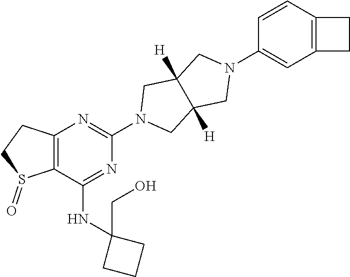 [SMILES: O=[S@]1CCc2nc(N3C[C@H]4CN(c5ccc6c(c5)CC6)C[C@H]4C3)nc(NC3(CO)CCC3)c21]

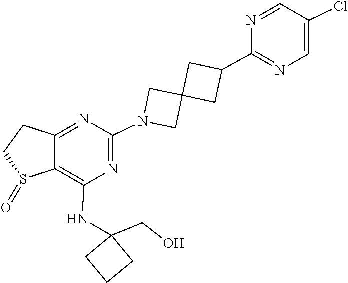

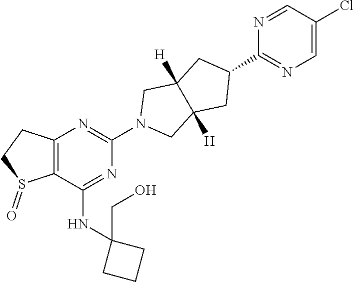 [SMILES: O=[S@]1CCc2nc(N3C[C@H]4C[C@@H](c5ncc(Cl)cn5)C[C@H]4C3)nc(NC3(CO)CCC3)c21]

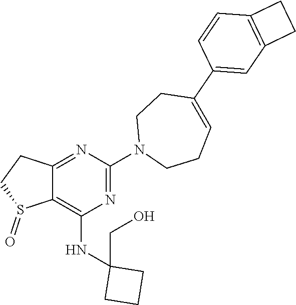 [SMILES: O=[S@@]1CCc2nc(N3CCC=C(c4ccc5c(c4)CC5)CC3)nc(NC3(CO)CCC3)c21]

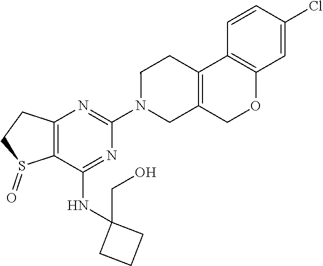 [SMILES: O=[S@]1CCc2nc(N3CCC4=C(COc5cc(Cl)ccc54)C3)nc(NC3(CO)CCC3)c21]

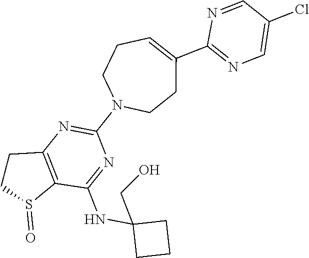 [SMILES: O=[S@@]1CCc2nc(N3CCC=C(c4ncc(Cl)cn4)CC3)nc(NC3(CO)CCC3)c21]

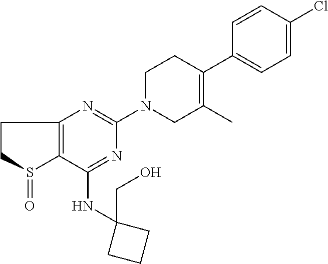 [SMILES: CC1=C(c2ccc(Cl)cc2)CCN(c2nc3c(c(NC4(CO)CCC4)n2)[S@@](=O)CC3)C1]

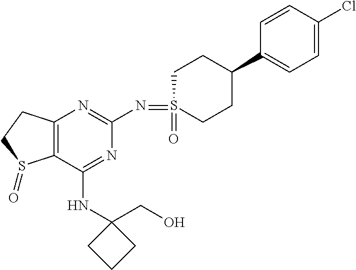 [SMILES: O=[S@]1CCc2nc(N=[S@]3(=O)CC[C@H](c4ccc(Cl)cc4)CC3)nc(NC3(CO)CCC3)c21]

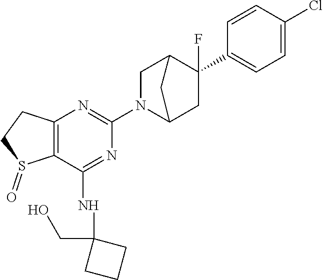 [SMILES: O=[S@]1CCc2nc(N3CC4CC3C[C@@]4(F)c3ccc(Cl)cc3)nc(NC3(CO)CCC3)c21]

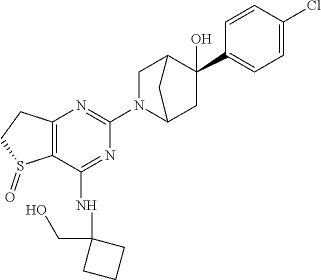 [SMILES: O=[S@@]1CCc2nc(N3CC4CC3C[C@]4(O)c3ccc(Cl)cc3)nc(NC3(CO)CCC3)c21]

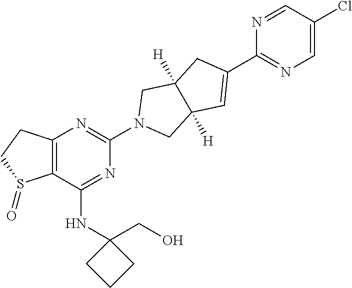 [SMILES: O=[S@@]1CCc2nc(N3C[C@H]4CC(c5ncc(Cl)cn5)=C[C@H]4C3)nc(NC3(CO)CCC3)c21]

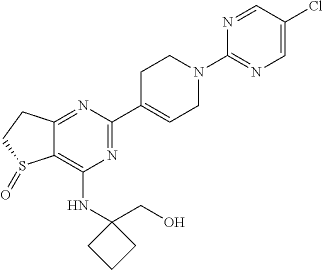 [SMILES: O=[S@@]1CCc2nc(C3=CCN(c4ncc(Cl)cn4)CC3)nc(NC3(CO)CCC3)c21]

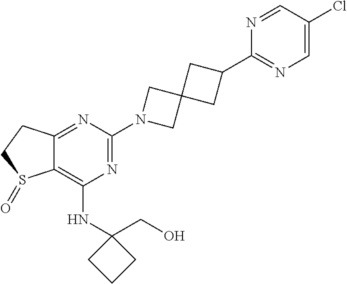 [SMILES: O=[S@]1CCc2nc(N3CC4(CC(c5ncc(Cl)cn5)C4)C3)nc(NC3(CO)CCC3)c21]

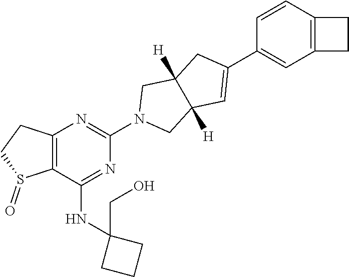 [SMILES: O=[S@@]1CCc2nc(N3C[C@@H]4CC(c5ccc6c(c5)CC6)=C[C@@H]4C3)nc(NC3(CO)CCC3)c21]

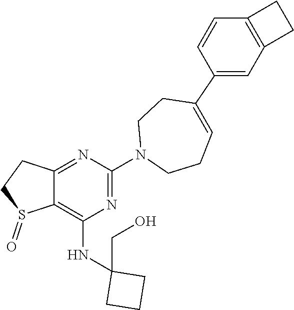 [SMILES: O=[S@]1CCc2nc(N3CCC=C(c4ccc5c(c4)CC5)CC3)nc(NC3(CO)CCC3)c21]

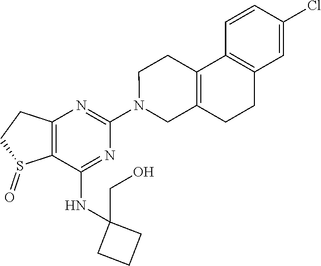 [SMILES: O=[S@@]1CCc2nc(N3CCC4=C(CCc5cc(Cl)ccc54)C3)nc(NC3(CO)CCC3)c21]

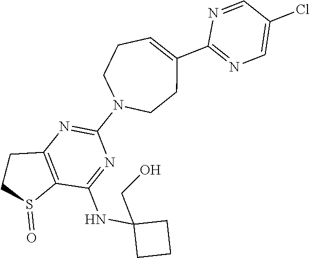 [SMILES: O=[S@]1CCc2nc(N3CCC=C(c4ncc(Cl)cn4)CC3)nc(NC3(CO)CCC3)c21]

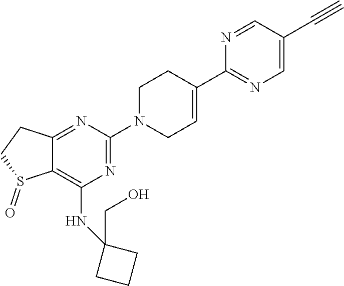 [SMILES: C#Cc1cnc(C2=CCN(c3nc4c(c(NC5(CO)CCC5)n3)[S@](=O)CC4)CC2)nc1]

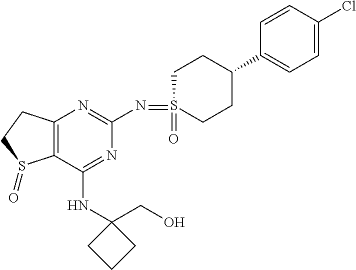 [SMILES: O=[S@]1CCc2nc(N=[S@]3(=O)CC[C@@H](c4ccc(Cl)cc4)CC3)nc(NC3(CO)CCC3)c21]

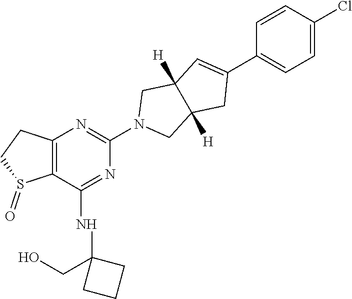 [SMILES: O=[S@@]1CCc2nc(N3C[C@H]4CC(c5ccc(Cl)cc5)=C[C@H]4C3)nc(NC3(CO)CCC3)c21]

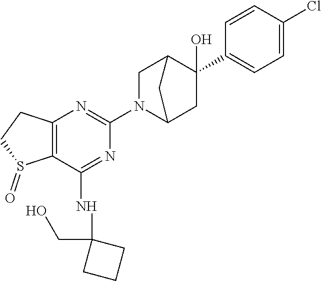 [SMILES: O=[S@@]1CCc2nc(N3CC4CC3C[C@@]4(O)c3ccc(Cl)cc3)nc(NC3(CO)CCC3)c21]

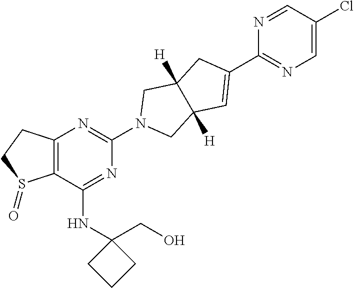 [SMILES: O=[S@]1CCc2nc(N3C[C@@H]4CC(c5ncc(Cl)cn5)=C[C@@H]4C3)nc(NC3(CO)CCC3)c21]

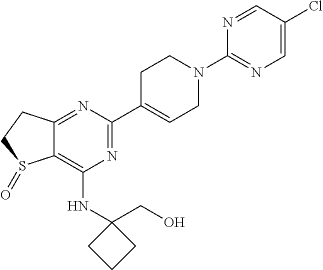 [SMILES: O=[S@]1CCc2nc(C3=CCN(c4ncc(Cl)cn4)CC3)nc(NC3(CO)CCC3)c21]

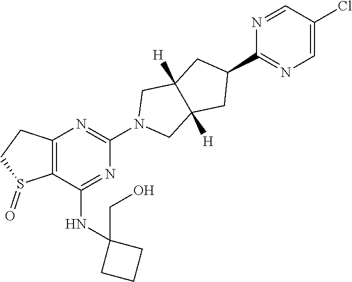 [SMILES: O=[S@@]1CCc2nc(N3C[C@H]4C[C@H](c5ncc(Cl)cn5)C[C@H]4C3)nc(NC3(CO)CCC3)c21]

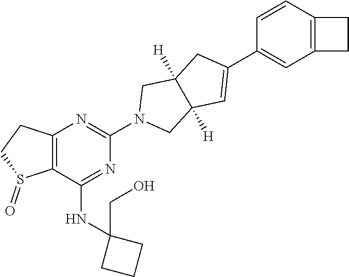 [SMILES: O=[S@@]1CCc2nc(N3C[C@H]4CC(c5ccc6c(c5)CC6)=C[C@H]4C3)nc(NC3(CO)CCC3)c21]

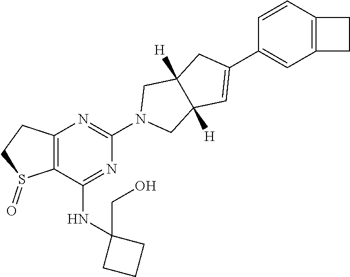 [SMILES: O=[S@]1CCc2nc(N3C[C@@H]4CC(c5ccc6c(c5)CC6)=C[C@@H]4C3)nc(NC3(CO)CCC3)c21]

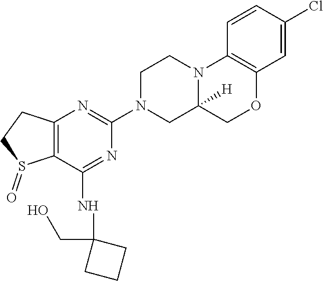

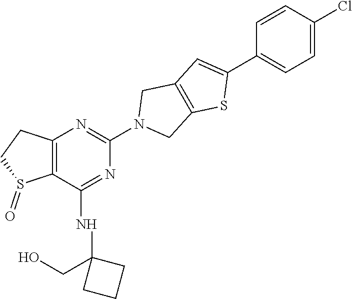 [SMILES: O=[S@@]1CCc2nc(N3Cc4cc(-c5ccc(Cl)cc5)sc4C3)nc(NC3(CO)CCC3)c21]

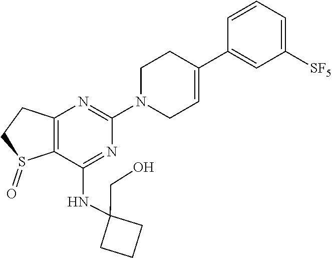

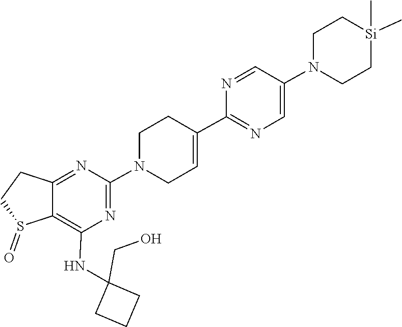

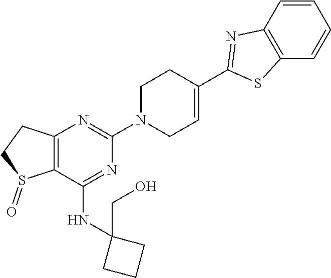 [SMILES: O=[S@]1CCc2nc(N3CC=C(c4nc5ccccc5s4)CC3)nc(NC3(CO)CCC3)c21]

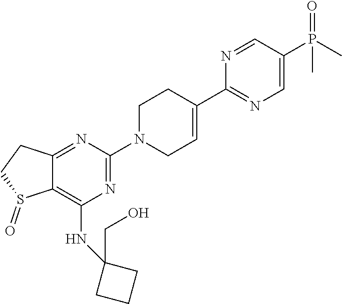 [SMILES: CP(C)(=O)c1cnc(C2=CCN(c3nc4c(c(NC5(CO)CCC5)n3)[S@](=O)CC4)CC2)nc1]

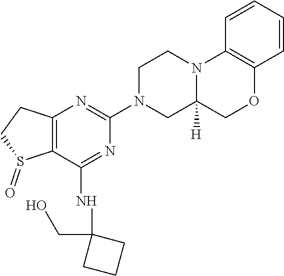 [SMILES: O=[S@@]1CCc2nc(N3CCN4c5ccccc5OC[C@H]4C3)nc(NC3(CO)CCC3)c21]

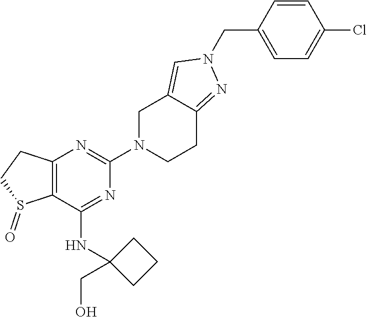 [SMILES: O=[S@@]1CCc2nc(N3CCc4nn(Cc5ccc(Cl)cc5)cc4C3)nc(NC3(CO)CCC3)c21]

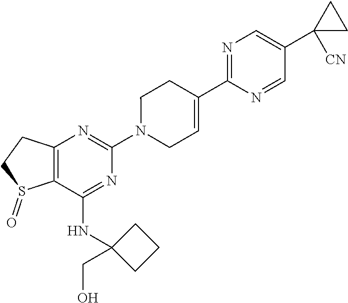 [SMILES: N#CC1(c2cnc(C3=CCN(c4nc5c(c(NC6(CO)CCC6)n4)[S@@](=O)CC5)CC3)nc2)CC1]

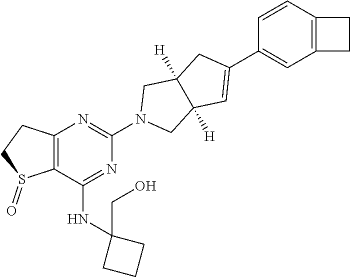 [SMILES: O=[S@]1CCc2nc(N3C[C@H]4CC(c5ccc6c(c5)CC6)=C[C@H]4C3)nc(NC3(CO)CCC3)c21]

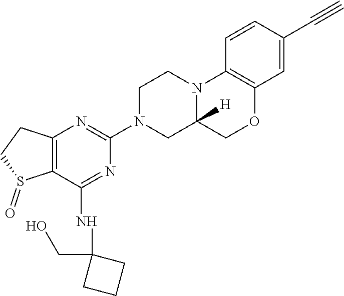

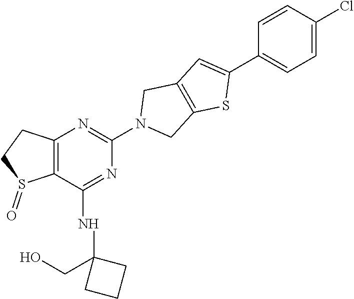 [SMILES: O=[S@]1CCc2nc(N3Cc4cc(-c5ccc(Cl)cc5)sc4C3)nc(NC3(CO)CCC3)c21]

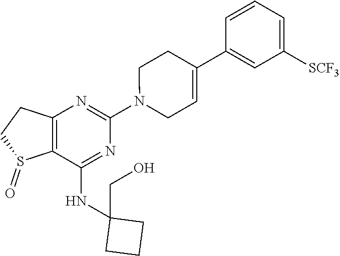

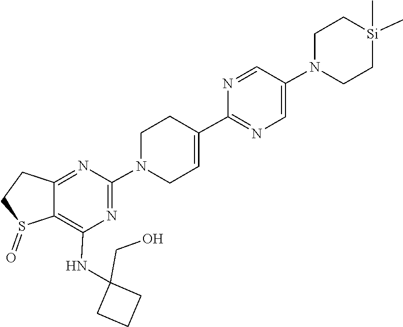

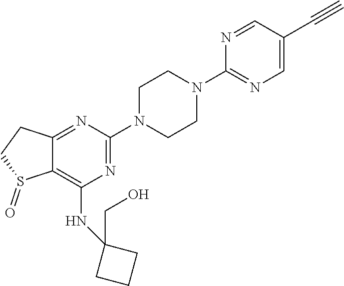 [SMILES: C#Cc1cnc(N2CCN(c3nc4c(c(NC5(CO)CCC5)n3)[S@](=O)CC4)CC2)nc1]

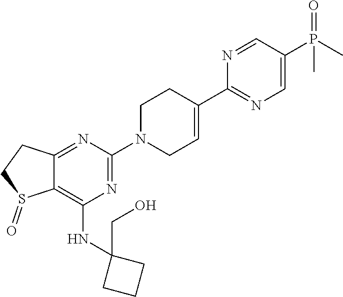 [SMILES: CP(C)(=O)c1cnc(C2=CCN(c3nc4c(c(NC5(CO)CCC5)n3)[S@@](=O)CC4)CC2)nc1]

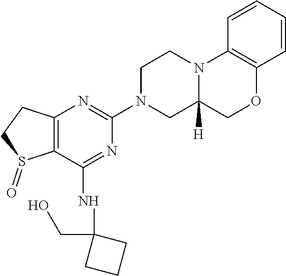 [SMILES: O=[S@]1CCc2nc(N3CCN4c5ccccc5OC[C@@H]4C3)nc(NC3(CO)CCC3)c21]

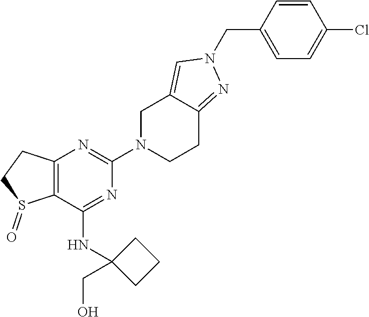 [SMILES: O=[S@]1CCc2nc(N3CCc4nn(Cc5ccc(Cl)cc5)cc4C3)nc(NC3(CO)CCC3)c21]

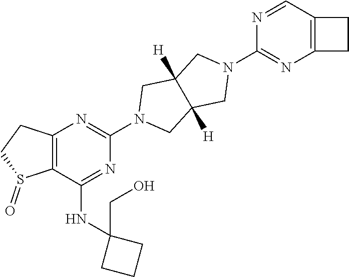 [SMILES: O=[S@@]1CCc2nc(N3C[C@H]4CN(c5ncc6c(n5)CC6)C[C@H]4C3)nc(NC3(CO)CCC3)c21]

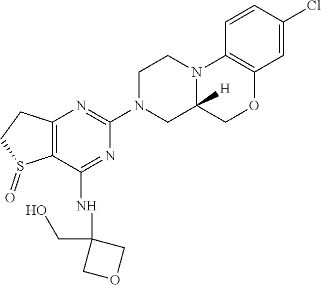

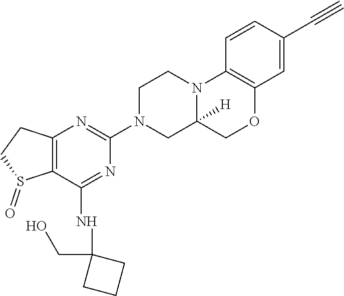

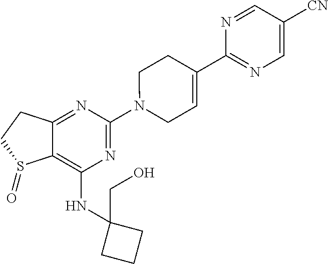 [SMILES: N#Cc1cnc(C2=CCN(c3nc4c(c(NC5(CO)CCC5)n3)[S@](=O)CC4)CC2)nc1]

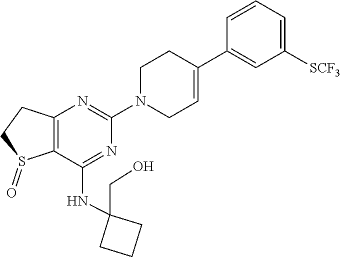

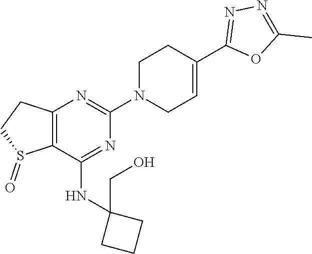 [SMILES: Cc1nnc(C2=CCN(c3nc4c(c(NC5(CO)CCC5)n3)[S@](=O)CC4)CC2)o1]

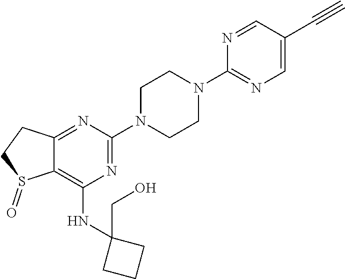 [SMILES: C#Cc1cnc(N2CCN(c3nc4c(c(NC5(CO)CCC5)n3)[S@@](=O)CC4)CC2)nc1]

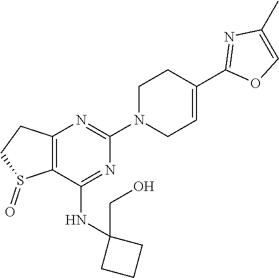 [SMILES: Cc1coc(C2=CCN(c3nc4c(c(NC5(CO)CCC5)n3)[S@](=O)CC4)CC2)n1]

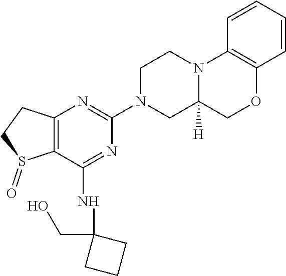 [SMILES: O=[S@]1CCc2nc(N3CCN4c5ccccc5OC[C@H]4C3)nc(NC3(CO)CCC3)c21]

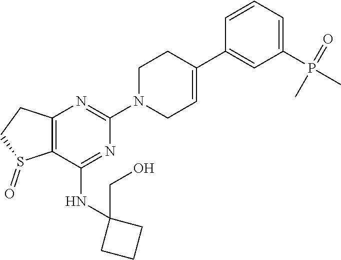 [SMILES: CP(C)(=O)c1cccc(C2=CCN(c3nc4c(c(NC5(CO)CCC5)n3)[S@](=O)CC4)CC2)c1]

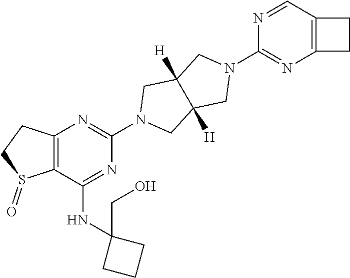 [SMILES: O=[S@]1CCc2nc(N3C[C@H]4CN(c5ncc6c(n5)CC6)C[C@H]4C3)nc(NC3(CO)CCC3)c21]

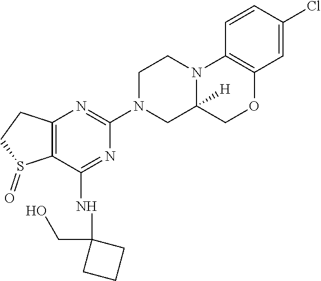

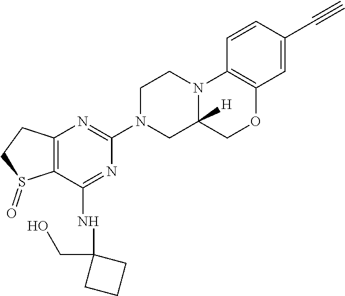

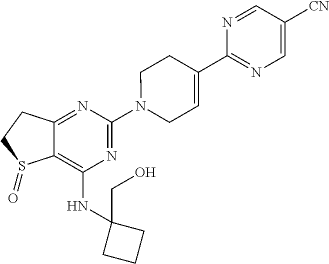 [SMILES: N#Cc1cnc(C2=CCN(c3nc4c(c(NC5(CO)CCC5)n3)[S@@](=O)CC4)CC2)nc1]

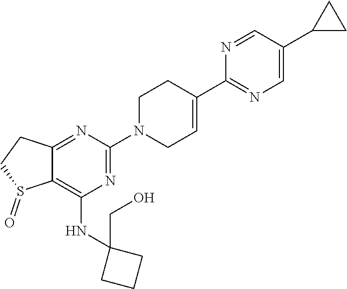 [SMILES: O=[S@@]1CCc2nc(N3CC=C(c4ncc(C5CC5)cn4)CC3)nc(NC3(CO)CCC3)c21]

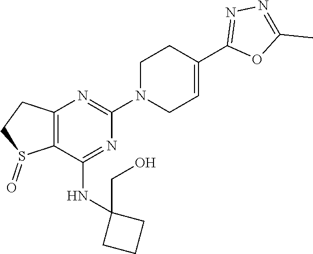 [SMILES: Cc1nnc(C2=CCN(c3nc4c(c(NC5(CO)CCC5)n3)[S@@](=O)CC4)CC2)o1]

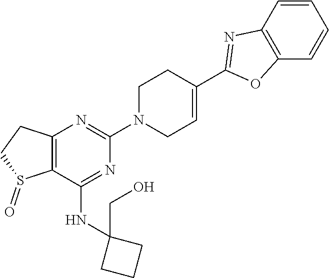 [SMILES: O=[S@@]1CCc2nc(N3CC=C(c4nc5ccccc5o4)CC3)nc(NC3(CO)CCC3)c21]

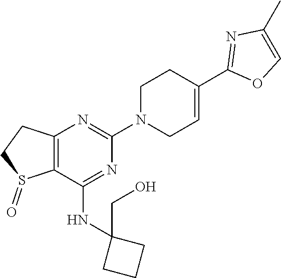 [SMILES: Cc1coc(C2=CCN(c3nc4c(c(NC5(CO)CCC5)n3)[S@@](=O)CC4)CC2)n1]

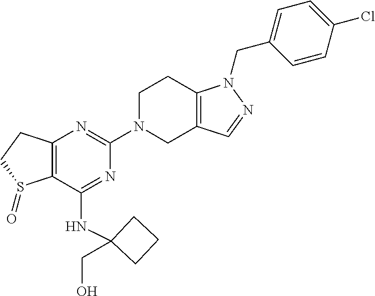 [SMILES: O=[S@@]1CCc2nc(N3CCc4c(cnn4Cc4ccc(Cl)cc4)C3)nc(NC3(CO)CCC3)c21]

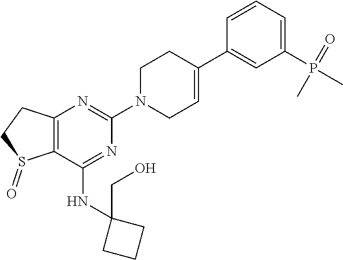 [SMILES: CP(C)(=O)c1cccc(C2=CCN(c3nc4c(c(NC5(CO)CCC5)n3)[S@@](=O)CC4)CC2)c1]

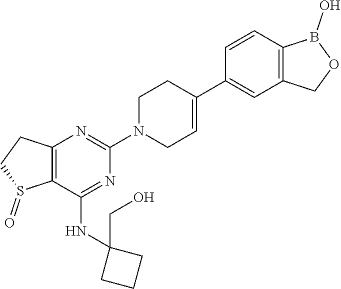 [SMILES: O=[S@@]1CCc2nc(N3CC=C(c4ccc5c(c4)COB5O)CC3)nc(NC3(CO)CCC3)c21]

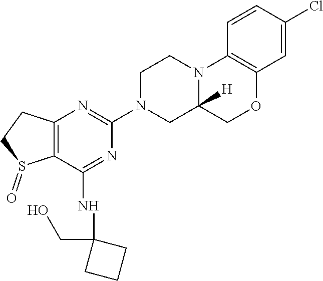

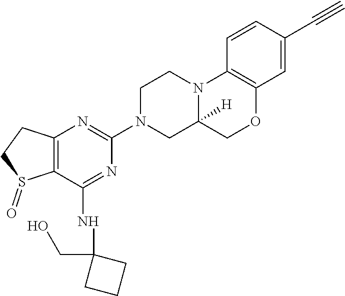

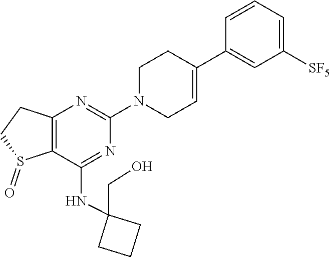

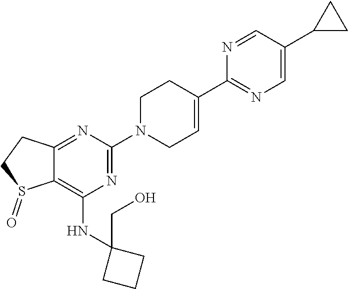 [SMILES: O=[S@]1CCc2nc(N3CC=C(c4ncc(C5CC5)cn4)CC3)nc(NC3(CO)CCC3)c21]

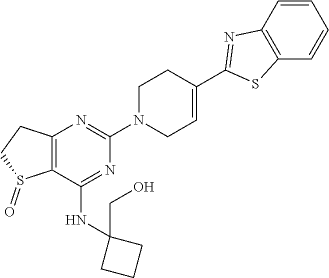 [SMILES: O=[S@@]1CCc2nc(N3CC=C(c4nc5ccccc5s4)CC3)nc(NC3(CO)CCC3)c21]

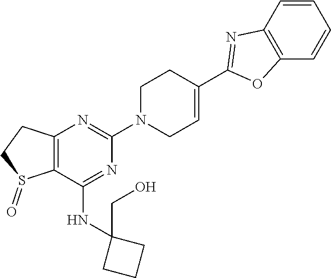 [SMILES: O=[S@]1CCc2nc(N3CC=C(c4nc5ccccc5o4)CC3)nc(NC3(CO)CCC3)c21]

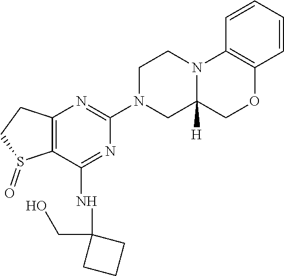 [SMILES: O=[S@@]1CCc2nc(N3CCN4c5ccccc5OC[C@@H]4C3)nc(NC3(CO)CCC3)c21]

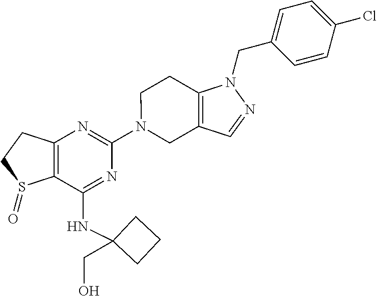 [SMILES: O=[S@]1CCc2nc(N3CCc4c(cnn4Cc4ccc(Cl)cc4)C3)nc(NC3(CO)CCC3)c21]

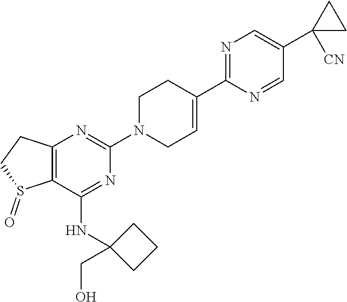 [SMILES: N#CC1(c2cnc(C3=CCN(c4nc5c(c(NC6(CO)CCC6)n4)[S@](=O)CC5)CC3)nc2)CC1]

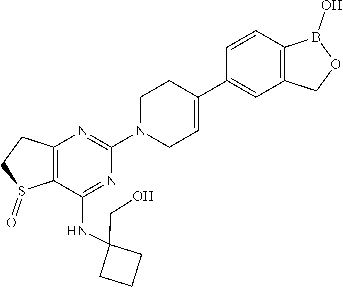 [SMILES: O=[S@]1CCc2nc(N3CC=C(c4ccc5c(c4)COB5O)CC3)nc(NC3(CO)CCC3)c21]

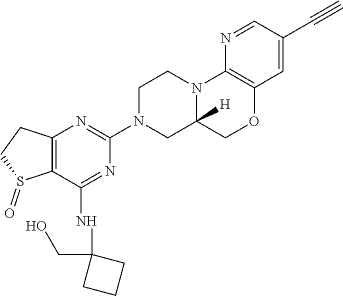

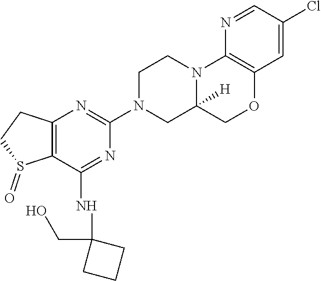

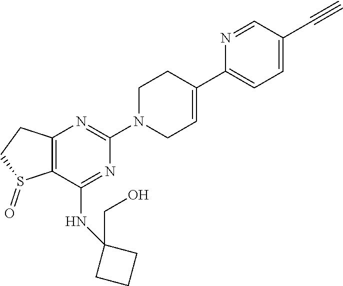 [SMILES: C#Cc1ccc(C2=CCN(c3nc4c(c(NC5(CO)CCC5)n3)[S@](=O)CC4)CC2)nc1]

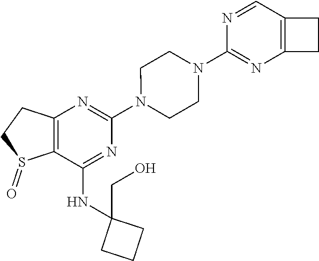 [SMILES: O=[S@]1CCc2nc(N3CCN(c4ncc5c(n4)CC5)CC3)nc(NC3(CO)CCC3)c21]

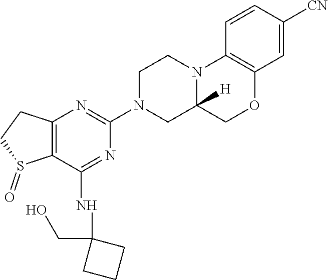

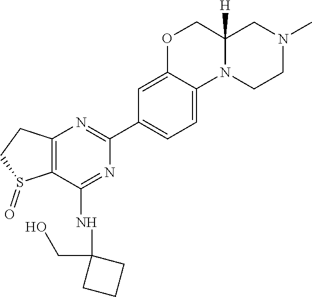 [SMILES: CN1CCN2c3ccc(-c4nc5c(c(NC6(CO)CCC6)n4)[S@](=O)CC5)cc3OC[C@@H]2C1]

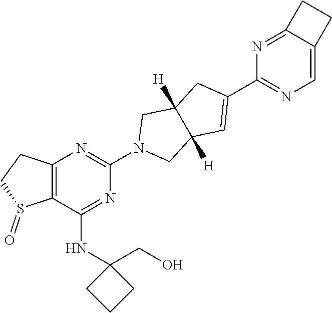 [SMILES: O=[S@@]1CCc2nc(N3C[C@@H]4CC(c5ncc6c(n5)CC6)=C[C@@H]4C3)nc(NC3(CO)CCC3)c21]

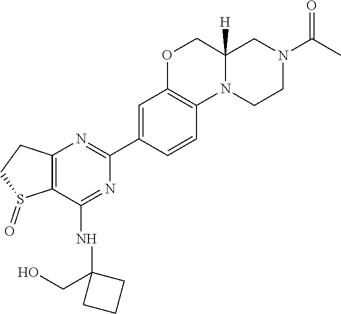 [SMILES: CC(=O)N1CCN2c3ccc(-c4nc5c(c(NC6(CO)CCC6)n4)[S@](=O)CC5)cc3OC[C@@H]2C1]

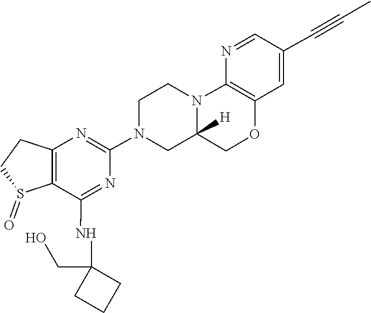

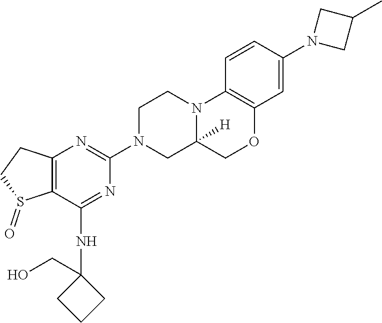

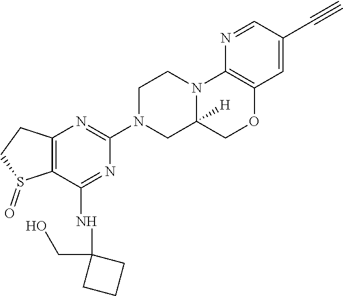

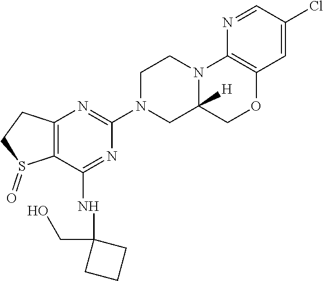

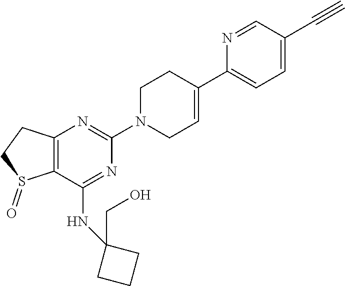 [SMILES: C#Cc1ccc(C2=CCN(c3nc4c(c(NC5(CO)CCC5)n3)[S@@](=O)CC4)CC2)nc1]

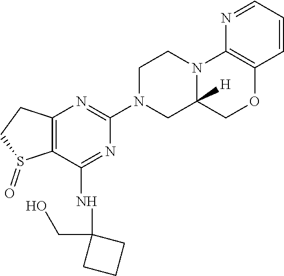

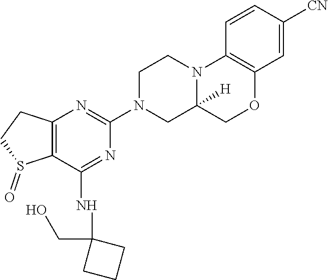

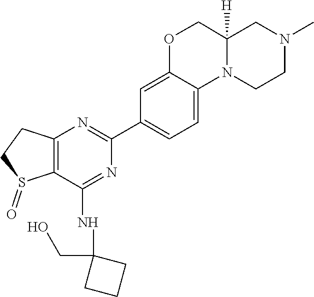 [SMILES: CN1CCN2c3ccc(-c4nc5c(c(NC6(CO)CCC6)n4)[S@@](=O)CC5)cc3OC[C@H]2C1]

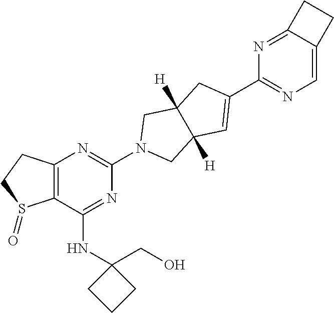 [SMILES: O=[S@]1CCc2nc(N3C[C@@H]4CC(c5ncc6c(n5)CC6)=C[C@@H]4C3)nc(NC3(CO)CCC3)c21]

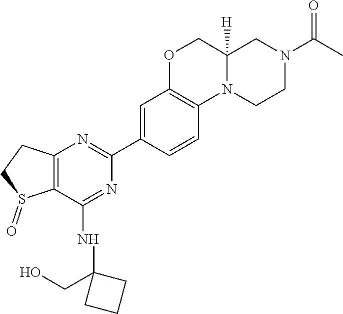 [SMILES: CC(=O)N1CCN2c3ccc(-c4nc5c(c(NC6(CO)CCC6)n4)[S@@](=O)CC5)cc3OC[C@H]2C1]

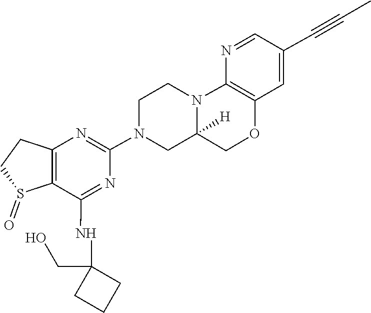

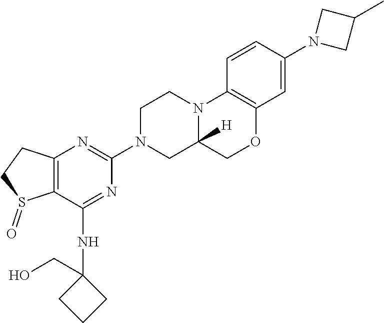

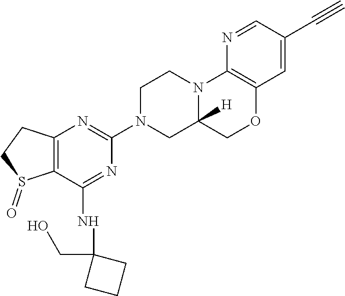

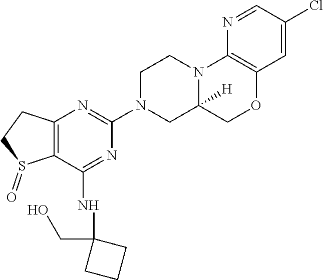

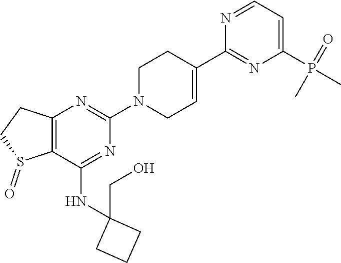 [SMILES: CP(C)(=O)c1ccnc(C2=CCN(c3nc4c(c(NC5(CO)CCC5)n3)[S@](=O)CC4)CC2)n1]

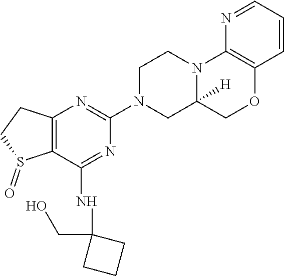

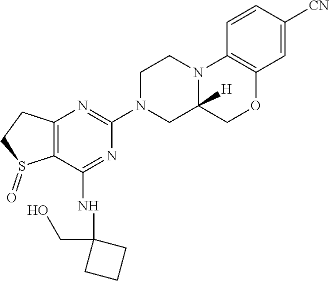

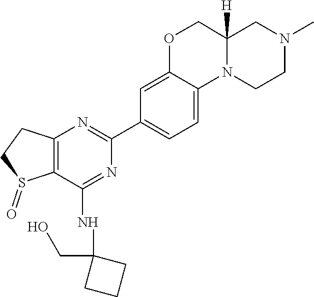 [SMILES: CN1CCN2c3ccc(-c4nc5c(c(NC6(CO)CCC6)n4)[S@@](=O)CC5)cc3OC[C@@H]2C1]

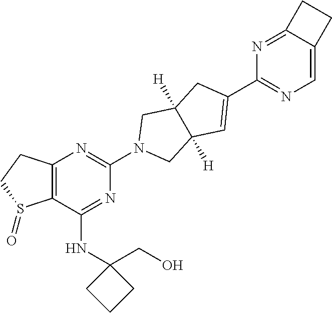 [SMILES: O=[S@@]1CCc2nc(N3C[C@H]4CC(c5ncc6c(n5)CC6)=C[C@H]4C3)nc(NC3(CO)CCC3)c21]

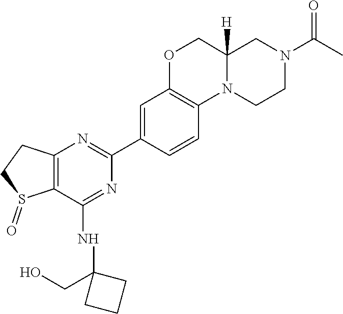 [SMILES: CC(=O)N1CCN2c3ccc(-c4nc5c(c(NC6(CO)CCC6)n4)[S@@](=O)CC5)cc3OC[C@@H]2C1]

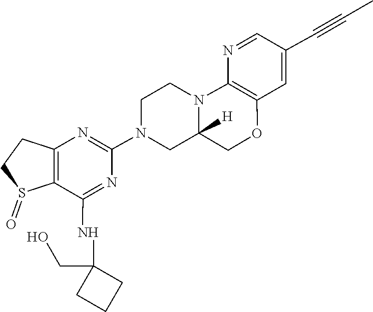

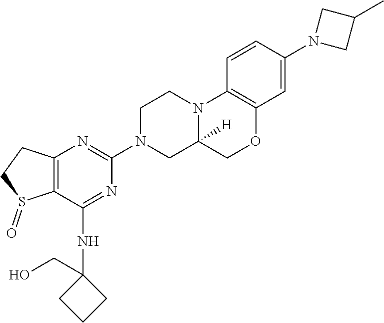

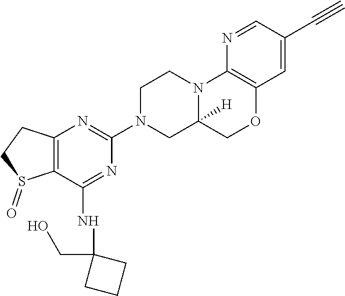

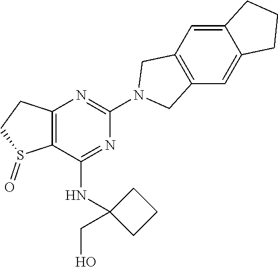 [SMILES: O=[S@@]1CCc2nc(N3Cc4cc5c(cc4C3)CCC5)nc(NC3(CO)CCC3)c21]

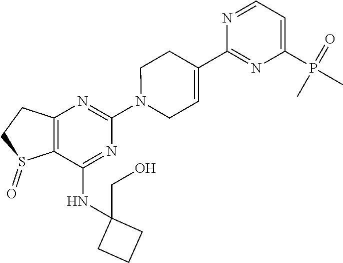 [SMILES: CP(C)(=O)c1ccnc(C2=CCN(c3nc4c(c(NC5(CO)CCC5)n3)[S@@](=O)CC4)CC2)n1]

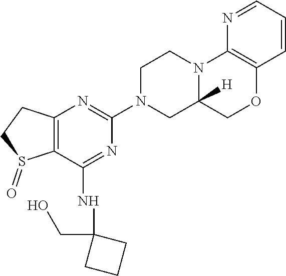

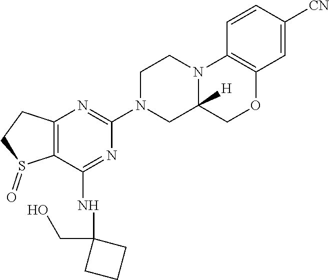

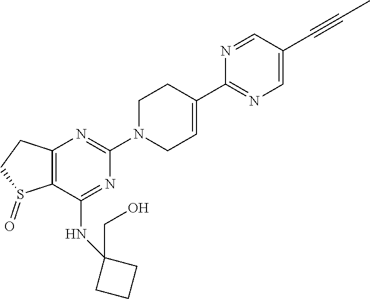 [SMILES: CC#Cc1cnc(C2=CCN(c3nc4c(c(NC5(CO)CCC5)n3)[S@](=O)CC4)CC2)nc1]

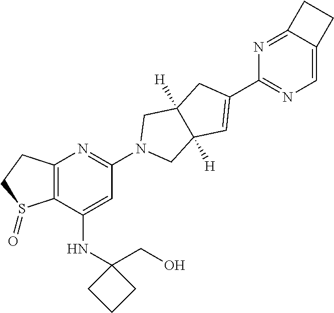 [SMILES: O=[S@]1CCc2nc(N3C[C@H]4CC(c5ncc6c(n5)CC6)=C[C@H]4C3)cc(NC3(CO)CCC3)c21]

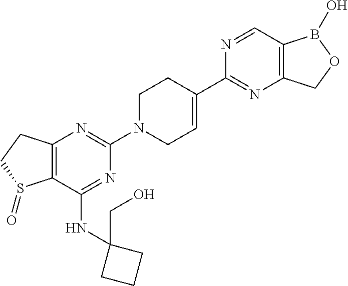 [SMILES: O=[S@@]1CCc2nc(N3CC=C(c4ncc5c(n4)COB5O)CC3)nc(NC3(CO)CCC3)c21]

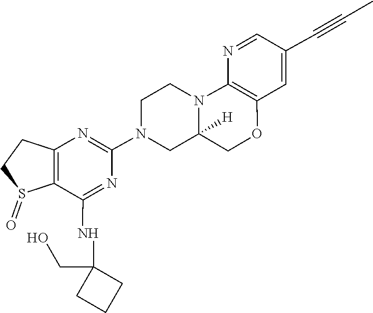

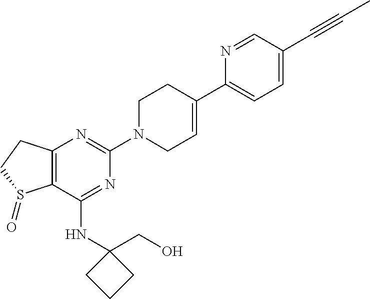 [SMILES: CC#Cc1ccc(C2=CCN(c3nc4c(c(NC5(CO)CCC5)n3)[S@](=O)CC4)CC2)nc1]

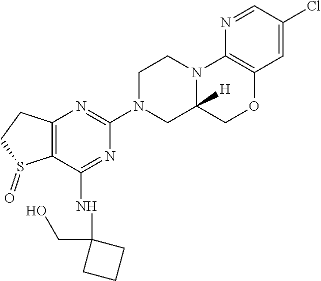

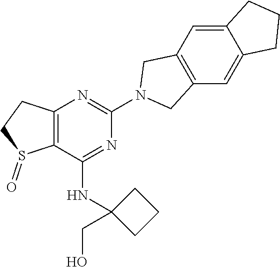 [SMILES: O=[S@]1CCc2nc(N3Cc4cc5c(cc4C3)CCC5)nc(NC3(CO)CCC3)c21]

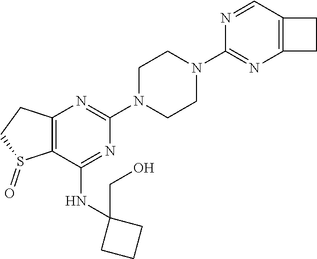 [SMILES: O=[S@@]1CCc2nc(N3CCN(c4ncc5c(n4)CC5)CC3)nc(NC3(CO)CCC3)c21]

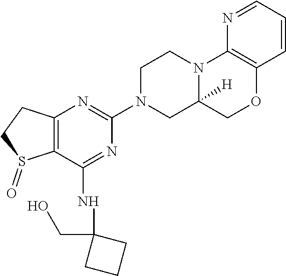

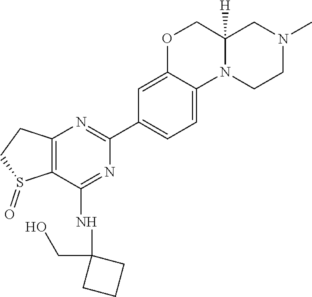 [SMILES: CN1CCN2c3ccc(-c4nc5c(c(NC6(CO)CCC6)n4)[S@](=O)CC5)cc3OC[C@H]2C1]

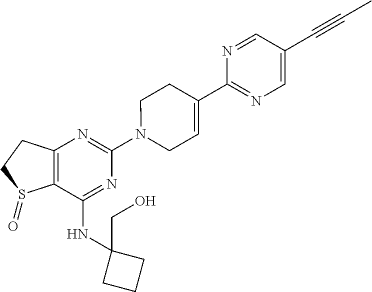 [SMILES: CC#Cc1cnc(C2=CCN(c3nc4c(c(NC5(CO)CCC5)n3)[S@@](=O)CC4)CC2)nc1]

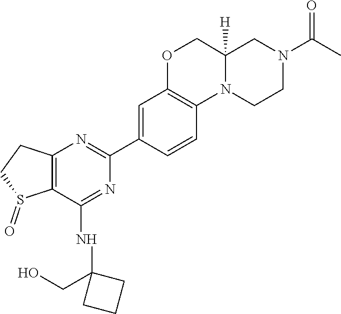 [SMILES: CC(=O)N1CCN2c3ccc(-c4nc5c(c(NC6(CO)CCC6)n4)[S@](=O)CC5)cc3OC[C@H]2C1]

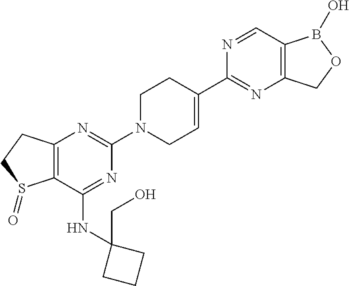 [SMILES: O=[S@]1CCc2nc(N3CC=C(c4ncc5c(n4)COB5O)CC3)nc(NC3(CO)CCC3)c21]

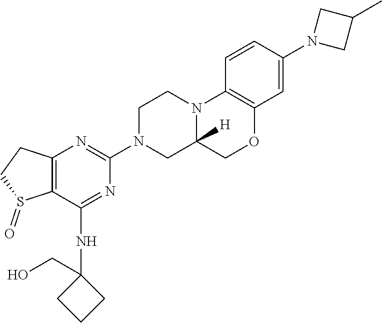

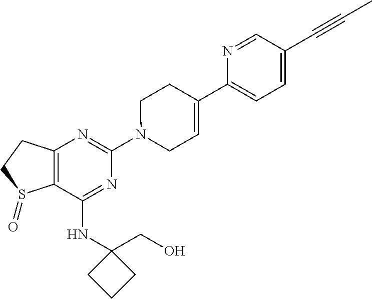 [SMILES: CC#Cc1ccc(C2=CCN(c3nc4c(c(NC5(CO)CCC5)n3)[S@@](=O)CC4)CC2)nc1]

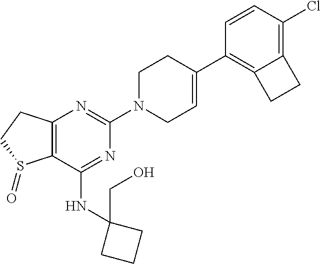 [SMILES: O=[S@@]1CCc2nc(N3CC=C(c4ccc(Cl)c5c4CC5)CC3)nc(NC3(CO)CCC3)c21]

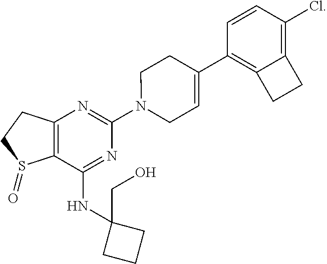 [SMILES: O=[S@]1CCc2nc(N3CC=C(c4ccc(Cl)c5c4CC5)CC3)nc(NC3(CO)CCC3)c21]

The present invention further provides a composition or pharmaceutical preparation comprising the compound or the stereoisomer or pharmaceutically acceptable salt thereof according to any one of the preceding solutions, and a pharmaceutically acceptable carrier and/or auxiliary material. The pharmaceutical composition can be in a unit preparation form (the unit preparation is also referred to as “preparation specification”).

Furthermore, the composition or pharmaceutical preparation of the present invention comprises 1-1500 mg of the compound or the stereoisomer or pharmaceutically acceptable salt thereof according to any one of the preceding solutions, and a pharmaceutically acceptable carrier and/or auxiliary material.

The present invention further provides the use of the compound or the stereoisomer or pharmaceutically acceptable salt thereof according to any one of the preceding solutions in the preparation of a medicament for treating/preventing a PDE4B-mediated disease. Furthermore, the PDE4B-mediated disease is cancer, COPD, idiopathic pulmonary fibrosis, or interstitial lung disease.

The present invention further provides a method for treating a disease in a mammal, which comprises administering to a subject a therapeutically effective amount of the compound or the stereoisomer or pharmaceutically acceptable salt thereof as shown in any one of the preceding solutions, wherein the disease is preferably cancer, COPD, idiopathic pulmonary fibrosis, or interstitial lung disease, and the therapeutically effective amount is preferably 1-1500 mg. In some embodiments, the mammal mentioned in the present invention includes human.

The “effective amount” or “therapeutically effective amount” as described in the present application refers to administration of a sufficient amount of the compound disclosed in the present application that will alleviate to some extent one or more symptoms of the diseases or conditions being treated. In some embodiments, the outcome is reduction and/or remission of signs, symptoms, or causes of the disease, or any other desired change in the biological system. For example, an “effective amount” with reference to therapeutic use is an amount of the composition comprising the compound disclosed in the present application that is required to provide clinically significant reduction of the symptoms of the disease. Examples of the therapeutically effective amount include, but are not limited to, 1-1500 mg, 1-1400 mg, 1-1300 mg, 1-1200 mg, 1-1000 mg, 1-900 mg, 1-800 mg, 1-700 mg, 1-600 mg, 1-500 mg, 1-400 mg, 1-300 mg, 1-250 mg, 1-200 mg, 1-150 mg, 1-125 mg, 1-100 mg, 1-80 mg, 1-60 mg, 1-50 mg, 1-40 mg, 1-25 mg, 1-20 mg, 5-1500 mg, 5-1000 mg, 5-900 mg, 5-800 mg, 5-700 mg, 5-600 mg, 5-500 mg, 5-400 mg, 5-300 mg, 5-250 mg, 5-200 mg, 5-150 mg, 5-125 mg, 5-100 mg, 5-90 mg, 5-70 mg, 5-80 mg, 5-60 mg, 5-50 mg, 5-40 mg, 5-30 mg, 5-25 mg, 5-20 mg, 10-1500 mg, 10-1000 mg, 10-900 mg, 10-800 mg, 10-700 mg, 10-600 mg, 10-500 mg, 10-450 mg, 10-400 mg, 10-300 mg, 10-250 mg, 10-200 mg, 10-150 mg, 10-125 mg, 10-100 mg, 10-90 mg, 10-80 mg, 10-70 mg, 10-60 mg, 10-50 mg, 10-40 mg, 10-30 mg, and 10-20 mg; 20-1500 mg, 20-1000 mg, 20-900 mg, 20-800 mg, 20-700 mg, 20-600 mg, 20-500 mg, 20-400 mg, 20-350 mg, 20-300 mg, 20-250 mg, 20-200 mg, 20-150 mg, 20-125 mg, 20-100 mg, 20-90 mg, 20-80 mg, 20-70 mg, 20-60 mg, 20-50 mg, 20-40 mg, and 20-30 mg; 50-1500 mg, 50-1000 mg, 50-900 mg, 50-800 mg, 50-700 mg, 50-600 mg, 50-500 mg, 50-400 mg, 50-300 mg, 50-250 mg, 50-200 mg, 50-150 mg, 50-125 mg, and 50-100 mg; and 100-1500 mg, 100-1000 mg, 100-900 mg, 100-800 mg, 100-700 mg, 100-600 mg, 100-500 mg, 100-400 mg, 100-300 mg, 100-250 mg, and 100-200 mg;
in some embodiments, the pharmaceutical composition or preparation of the present invention contains the above-mentioned therapeutically effective amount of the compound, or the stereoisomer, solvate, or pharmaceutically acceptable salt thereof according to the present invention; and
the present invention relates to a pharmaceutical composition or pharmaceutical preparation, comprising a therapeutically effective amount of the compound or the stereoisomer or pharmaceutically acceptable salt thereof according to the present invention, and a carrier and/or excipient. The pharmaceutical composition can be in a unit preparation form (the amount of the active drug in the unit preparation is also referred to as “preparation specification”). In some embodiments, the pharmaceutical composition comprises, without limitation, 1 mg, 1.25 mg, 2.5 mg, 5 mg, 10 mg, 12.5 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 850 mg, 900 mg, 950 mg, 1000 mg, 1100 mg, 1200 mg, 1300 mg, 1400 mg, or 1500 mg of the compound or the stereoisomer or pharmaceutically acceptable salt thereof according to the present invention.

A method for treating a disease in a mammal is provided, comprising administering to a subject a therapeutically effective amount of the compound or the stereoisomer or pharmaceutically acceptable salt thereof according to the present invention, and a pharmaceutically acceptable carrier and/or excipient, wherein the therapeutically effective amount is preferably 1-1500 mg, and the disease is preferably cancer, COPD, idiopathic pulmonary fibrosis, or interstitial lung disease.

A method for treating a disease in a mammal is provided, comprising administering to a subject a drug, i.e., the compound or the stereoisomer or pharmaceutically acceptable salt thereof according to the present invention, and a pharmaceutically acceptable carrier and/or excipient in a daily dose of 1-1500 mg/day, wherein the daily dose may be a single dose or divided doses; in some embodiments, the daily dose includes, but is not limited to, 10-1500 mg/day, 20-1500 mg/day, 25-1500 mg/day, 50-1500 mg/day, 75-1500 mg/day, 100-1500 mg/day, 200-1500 mg/day, 10-1000 mg/day, 20-1000 mg/day, 25-1000 mg/day, 50-1000 mg/day, 75-1000 mg/day, 100-1000 mg/day, 200-1000 mg/day, 25-800 mg/day, 50-800 mg/day, 100-800 mg/day, 200-800 mg/day, 25-400 mg/day, 50-400 mg/day, 100-400 mg/day, and 200-400 mg/day; in some embodiments, the daily dose includes, but is not limited to, 1 mg/day, 5 mg/day, 10 mg/day, 20 mg/day, 25 mg/day, 50 mg/day, 75 mg/day, 100 mg/day, 125 mg/day, 150 mg/day, 200 mg/day, 400 mg/day, 600 mg/day, 800 mg/day, 1000 mg/day, 1200 mg/day, 1400 mg/day, and 1500 mg/day.

The present invention relates to a kit, which may comprise a composition in a single-dose or multi-dose form, wherein the kit comprises the compound or the stereoisomer or pharmaceutically acceptable salt thereof according to the present invention, and the amount of the compound or the stereoisomer or pharmaceutically acceptable salt thereof according to the present invention is the same as that in the above-mentioned pharmaceutical composition.

In the present invention, the amount of the compound or the stereoisomer or pharmaceutically acceptable salt thereof according to the present invention is calculated in the form of a free base in each case.

The term “preparation specification” refers to the weight of the active drug contained in each vial, tablet, or other unit preparation.

### Synthesis Route

Patent documents such as WO 2013026797 A1 have introduced a method for preparing a PDE4B inhibitor, and those skilled in the art would have been able to prepare the compounds of the present invention in conjunction with this document and known organic synthesis techniques, wherein the starting materials used therein are commercially available chemicals and (or) compounds described in chemical documents. “Commercially available chemicals” are obtained from regular commercial sources, and suppliers include: Titan Technology Co., Ltd., Energy Chemical Co., Ltd., Shanghai Demo Co., Ltd., Chengdu Kelong Chemical Co., Ltd., Accela ChemBio Co., Ltd., PharmaBlock Sciences (Nanjing), Inc., WuXi Apptec Co., Ltd., J&K Scientific Co., Ltd., etc.

Specific and similar reactants can be selectively identified by the indexes of known chemicals prepared by the Chemical Abstracts Service of the American Chemical Society, wherein the indexes are available in most public libraries and university libraries and online. Chemicals that are known but not commercially available in the catalog are optionally prepared by custom chemical synthesis plants, wherein many of standard chemical supply plants (such as those listed above) provide custom synthesis services.

### Terminology

Unless otherwise specified, the terms of the present invention have the following meanings.

The term “halogen” herein refers to F, Cl, Br, I, or isotopes thereof.

The term “halo” or “substituted with halogen” means that a hydrogen atom is replaced with one or more groups selected from F, Cl, Br, I, or isotopes thereof, wherein the upper limit of the number of halogen substituents is equal to the sum of the number of hydrogens that can be replaced in the group to be substituted. Without particular limitation, the number of halogen substituents is any integer between 1 and the upper limit, and when the number of halogen substituents is greater than 1, the group to be substituted can be substituted with the same or different halogen.

The term “deuterated” or “deuterated product” refers to the case where a hydrogen atom on alkyl, cycloalkyl, alkylene, aryl, heteroaryl, mercapto, heterocycloalkyl, alkenyl, alkynyl and other groups is replaced with at least one isotope deuterium, wherein the upper limit of the number of deuterium substituents is equal to the sum of the number of hydrogens that can be replaced in the group to be substituted. Without particular limitation, the number of deuterium substituents is any integer between 1 and the upper limit, preferably 1-20 deuterium atoms, more preferably 1-10 deuterium atoms, more preferably 1-6 deuterium atoms, further preferably 1-3 deuterium atoms.

The term “alkyl” refers to a monovalent linear or branched saturated aliphatic hydrocarbon group.

Unless otherwise specified, the alkyl refers to an alkyl group comprising 1 to 20 carbon atoms, preferably an alkyl group comprising 1 to 8 carbon atoms, more preferably an alkyl group comprising 1 to 6 carbon atoms, further preferably an alkyl group comprising 1 to 4 carbon atoms, and further preferably an alkyl group comprising 1-2 carbon atoms. Non-limiting examples include methyl, ethyl, n-propyl, isopropyl, n-butyl, sec-butyl, neobutyl, tert-butyl, n-pentyl, isoamyl, neopentyl, n-hexyl, and various branched isomers thereof.

The term “alkylene” refers to a divalent linear or branched chain divalent saturated alkyl, and examples of alkylene include, but are not limited to, methylene, ethylene, propylene, butylene, etc.

The term “cycloalkyl” refers to a monovalent non-aromatic, partially unsaturated or fully saturated, substituted or unsubstituted carbocyclic hydrocarbon group. Unless otherwise specified, it generally has 3 to 12 carbon atoms, preferably 3-10 carbon atoms, more preferably 3-6 carbon atoms, and further preferably 3-4 carbon atoms. Non-limiting examples include cyclopropyl, cyclobutyl, cyclopentyl, cyclohexyl,

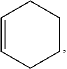

cycloheptyl, cyclopropenyl, cyclobutenyl, cyclopentenyl, cyclohexenyl, etc.

The term “cycloalkylene” refers to a divalent group of “cycloalkyl”, and non-limiting examples include cyclopropylene, cyclobutylene, etc.

The term “heterocyclic” or “heterocyclyl” refers to a substituted or unsubstituted aromatic ring or a substituted or unsubstituted, saturated or partially unsaturated non-aromatic ring, which comprises 1 to 3 heteroatoms selected from N, O or S unless otherwise defined, including monocyclic heterocyclic ring, bridged bicyclic heterocyclic ring, bicyclic fused heterocyclic ring, bicyclic spiro heterocyclic ring, etc. Unless otherwise specified, it is a 3- to 12-membered heterocyclic ring, more preferably 4- to 12-membered heterocyclic ring, more preferably 4- to 10-membered heterocyclic ring, further preferably 4- to 7-membered heterocyclic ring. The definition thereof comprises heterocycloalkyl and heteroaryl. The N and S in the heterocyclyl ring may be oxidized to various oxidation states. Heterocyclyl can be connected to a heteroatom or a carbon atom, and non-limiting examples of heterocyclyl include oxiranyl, aziridinyl, oxetanyl, azetidinyl, 1,3-dioxolanyl, 1,4-dioxolanyl, 1,3-dioxanyl, azepanyl, pyridinyl, furyl, thienyl, pyranyl, N-alkylpyrrolyl, pyrimidinyl, pyrazinyl, pyrazolyl, pyridazinyl, imidazolyl, piperidinyl, piperidyl, morpholinyl, thiomorpholinyl, 1,3-dithianyl, dihydrofuryl, dihydropyranyl, dithiolanyl, tetrahydrofuryl, tetrahydropyrrolyl, tetrahydroimidazolyl, oxazolyl, dihydrooxazolyl, tetrahydrooxazolyl, tetrahydrothiazolyl, tetrahydropyranyl, benzimidazolyl, benzopyridinyl, pyrrolopyridinyl, benzodihydrofuryl, azabicyclo[3.2.1]octyl, azabicyclo[5.2.0]nonanyl, oxatricyclo[5.3.1.1]dodecyl, azaadamantyl, oxaspiro[3.3]heptyl,

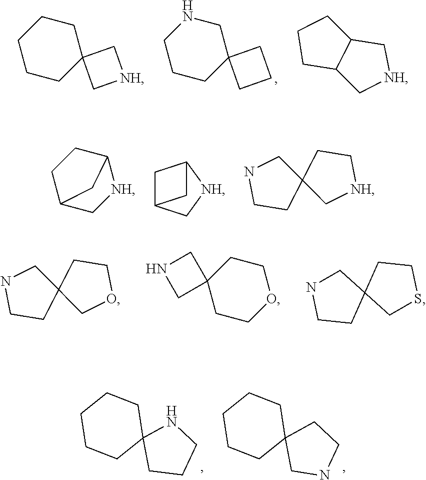

The term “heterocyclylene” is a divalent group corresponding to “heterocyclyl”, and non-limiting examples include imidazolylene, piperidinylene, aziridinylene, etc.

The term “carbocyclic” or “carbocyclyl” refers to a substituted or unsubstituted, saturated or unsaturated, aromatic or non-aromatic carbocyclic group, including a monocyclic carbocyclic ring, a bridged bicyclic ring, a fused bicyclic ring, a spiro bicyclic ring, etc. Unless otherwise specified, it contains 3 to 12 carbon atoms, preferably 3-10 carbon atoms, further preferably 3-6 carbon atoms. The definition thereof comprises cycloalkyl and aryl. In non-limiting embodiments, monocyclic carbocyclic rings include cyclopropyl, cyclobutyl, cyclopentyl, cyclohexyl, cycloheptyl or phenyl,

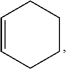

etc., bicyclic bridged rings include,

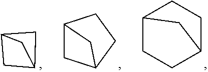

etc., bicyclic fused rings include

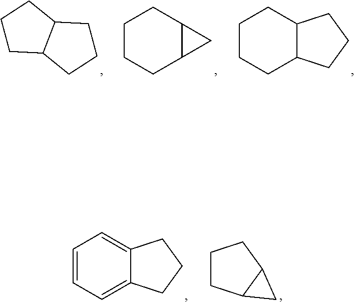

etc., and bicyclic spiro rings include

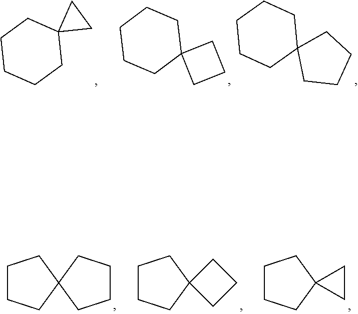

The term “aryl” refers to an aromatic carbocyclic ring. Non-limiting examples include phenyl, naphthyl, etc.

The term “alkynyl” refers to a linear or branched monovalent unsaturated hydrocarbon group containing one or more carbon-carbon triple bonds. Unless otherwise specified, the alkynyl contains 2-6 carbon atoms, preferably contains 2-4 carbon atoms, and non-limiting examples include ethynyl, propynyl, propargyl, etc.

The term “alkenyl” refers to a linear or branched monovalent unsaturated hydrocarbon group containing one or more carbon-carbon double bonds. Unless otherwise specified, the alkenyl contains 2-6 carbon atoms, preferably contains 2-4 carbon atoms, and non-limiting examples are ethenyl, propenyl, allyl, 2-butenyl, 1-butenyl, etc.

The term “alkoxy” or “alkyloxy” refers to —O-alkyl. Without particular limitation, alkoxy or alkyloxy is —O—C₁₋₈ alkyl, preferably —O—C₁₋₆ alkyl, more preferably —O—C₁₋₄ alkyl, and further preferably —O—C₁₋₂ alkyl. Non-limiting examples include methoxy, ethoxy, n-propoxy, isopropoxy, n-butoxy, secbutoxy, tert-butoxy, n-pentoxy, n-hexyloxy, cyclopropoxy, cyclobutoxy, etc.

The term “haloalkoxy” refers to —O-haloalkyl. Without particular limitation, the haloalkoxy is —O-halo C₁₋₈ alkyl, preferably —O-halo C₁₋₆ alkyl, more preferably —O-halo C₁₋₄ alkyl, and further preferably —O-halo C₁₋₂ alkyl. Non-limiting examples include monofluoromethoxy, difluoromethoxy, trifluoromethoxy, difluoroethyloxy, etc.

The term “C₁₋₄ alkylacyl” refers to C₁₋₄ alkyl-C(O)—. Non-limiting examples include formyl, acetyl, and propionyl.

The term “C₁₋₄ alkylsulfonyl” refers to C₁₋₄ alkyl-S(O)₂—. Non-limiting examples include methylsulfonyl, ethylsulfonyl, and propylsulfonyl.

The term “heteroaromatic ring” or “heteroaryl” refers to an aromatic heterocyclic ring. Non-limiting examples include pyrazolyl, pyrimidinyl, thiazolyl, pyridinyl, furyl, etc.

The term “heterocycloalkyl” refers to a non-aromatic, partially unsaturated or fully saturated heterocyclic ring, which generally has 4 to 12 ring members, preferably 4 to 10 ring members, more preferably 4 to 7 ring members, and further preferably 5 or 6 ring members. In addition to carbon atoms, heterocyclic alkyl groups also comprise 1-3 heteroatoms selected from N, S and O as ring members. Non-limiting examples include azetidinyl, morpholinyl, piperazinyl, piperidinyl, tetrahydropyranyl, oxetanyl, etc.

The term “alkylamino” or “alkamino” refers to amino substituted with one or two alkyl, and is also written as —N-(alkyl)₂ or —NH-alkyl, wherein the latter is also known as monoalkylamino. Non-limiting examples include dimethylamino, monomethylamino, diethylamino, monoethylamino, etc.

The term “optional” or “optionally” means that the events or circumstances described subsequent thereto may but not necessarily occur, and the description includes occasions where the events or circumstances occur or do not occur. For example, the term “alkyl optionally substituted with F” means that the alkyl may but not necessarily be substituted with F, and the description includes the case where the alkyl is substituted with F and the case where the alkyl is not substituted with F.

The term “pharmaceutically acceptable salt” refers to a salt of the compound of the present invention, which salt maintains the biological effectiveness and characteristics of a free acid or a free base and is obtained by reacting the free acid with a non-toxic inorganic base or organic base, or reacting the free base with a non-toxic inorganic acid or organic acid.

The term “pharmaceutical composition” represents a mixture of one or more compounds or stereoisomers, solvates, pharmaceutically acceptable salts or co-crystals thereof as described herein and other components including physiologically/pharmaceutically acceptable carriers and/or excipients.

The term “carrier” refers to: a system that does not cause significant irritation to the organism and does not eliminate the biological activity and characteristics of the administered compound and can change the way the drug enters the human body and the distribution of the drug in the body, control the release rate of the drug and delivery the drug to targeted organs. Non-limiting examples of the carrier include microcapsule, microsphere, nanoparticle, liposome, etc.

The term “excipient” refers to: a substance that is not a therapeutic agent per se, but used as a diluent, auxiliary material, adhesive and/or vehicle for addition to a pharmaceutical composition, thereby improving the disposal or storage properties thereof, or allowing to or promoting the formation of a compound or a pharmaceutical composition into a unit dosage form for administration. As is known to those skilled in the art, pharmaceutically acceptable excipients can provide various functions and can be described as a wetting agent, a buffer, a suspending agent, a lubricant, an emulsifier, a disintegrating agent, an absorbent, a preservative, a surfactant, a colorant, a flavoring agent, and a sweetening agent. Examples of pharmaceutically acceptable excipients include, but are not limited to: (1) sugars, such as lactose, glucose, and sucrose; (2) starch, such as corn starch and potato starch; (3) cellulose and derivatives thereof, such as sodium carboxymethyl cellulose, ethyl cellulose, cellulose acetate, hydroxypropyl methylcellulose, hydroxypropyl cellulose, microcrystalline cellulose, and croscarmellose (such as croscarmellose sodium); (4) tragacanth powder; (5) malt; (6) gelatin; (7) talc; (8) excipients, such as cocoa butter or suppository wax; (9) oils, such as peanut oil, cottonseed oil, safflower oil, sesame oil, olive oil, corn oil, and soybean oil; (10) diols, such as propylene glycol; (11) polyols, such as glycerol, sorbitol, mannitol, and polyethylene glycol; (12) esters, such as ethyl oleate and ethyl laurate; (13) agar; (14) buffers, such as magnesium hydroxide and aluminum hydroxide; (15) alginic acid; (16) pyrogen-free water; (17) isotonic saline; (18) Ringer's solution; (19) ethanol; (20) pH buffer solution; (21) polyester, polycarbonate and/or polyanhydride; and (22) other non-toxic compatible substances used in a pharmaceutical preparation.

The term “stereoisomer” refers to an isomer produced as a result of different spatial arrangement of atoms in molecules, including cis-trans isomers, enantiomers, and conformational isomers.

The term “solvate” refers to a substance formed by the compound or the salt thereof according to the present invention and a stoichiometric or non-stoichiometric amount of a solvent by bonding via an intermolecular non-covalent force. When the solvent is water, the solvate is a hydrate.

The term “co-crystal” refers to a crystal formed by the combination of an active pharmaceutical ingredient (API) and a co-crystal former (CCF) under the action of hydrogen bonding or other non-covalent bonding. The pure state of API and CCF are both solid at room temperature, and there is a fixed stoichiometric ratio between various components. The co-crystal is a multi-component crystal, which includes both a binary co-crystal formed between two neutral solids and a multi-element co-crystal formed between a neutral solid and a salt or solvate.

### DETAILED DESCRIPTION OF EMBODIMENTS

The content of the present invention is described in detail by means of the following examples. In examples in which no specific conditions are indicated, experimental methods are carried out under conventional conditions. The listed examples are intended to better illustrate the content of the present invention but should not be construed as limiting the content of the present invention. Non-essential improvements and adjustments made to the embodiments by those skilled in the art according to the above Summary of the Invention still fall within the scope of protection of the present invention.
Dess-Martin reagent; 1,1,1-Triacetoxy-1,1-dihydro-1,2-benziodoxol-3-(1H)-one
DIPEA: N,N-diisopropylethylamine
NMP; N-methylpyrrolidone
TBDMSCl: Tert-butyl dimethylsilyl chloride
DMAP: 4-Dimethylaminopyridine
HATU: 2-(7-azabenzotriazole)-N,N,N′,N′-tetramethyluronium hexafluorophosphate
T₃P: Propylphosphonic cyclic anhydride

### Example 1

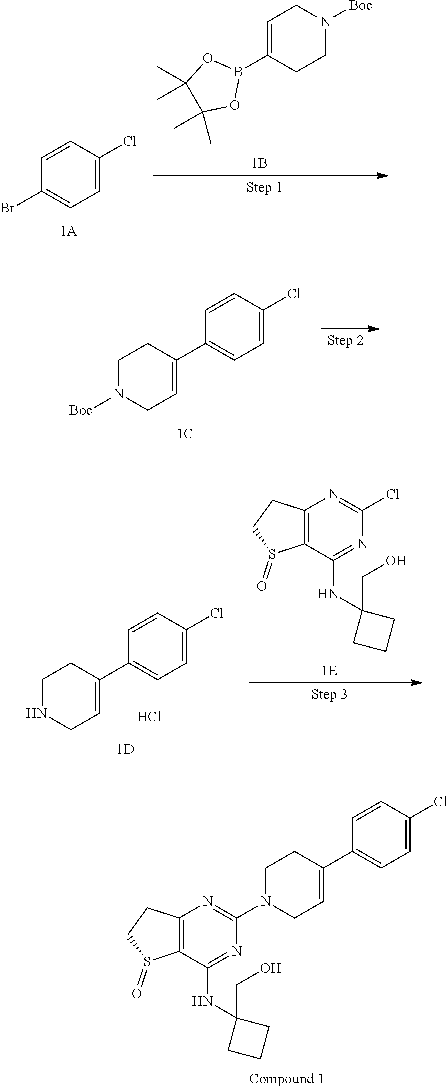

Step 1: Substrate 1A (5.0 g, 26.11 mmol) was added to a 250 mL single-mouth flask and dissolved with dioxane (40 mL), and a sodium carbonate solution (2 M, 40 mL), 1B (9.7 g, 31.33 mmol), and [1,1′-bis(diphenylphosphino)ferrocene]palladium dichloride (2.1 g, 2.61 mmol) were added and stirred at 100 degrees Celsius overnight under nitrogen protection. The reaction liquid was diluted by adding water (40 mL) and suction-filtered with diatomite, the filtrate was extracted with ethyl acetate (2×50 mL), and the organic phases were combined, dried over anhydrous sodium sulfate, concentrated, and subjected to column chromatography (petroleum ether:ethyl acetate (v/v)=10:1) to obtain the title compound 1C (7.2 g, 94%).

LC-MS (ESI): m/z=238.1 [M−56+H]⁺.

Step 2: Substrate 1C (0.6 g, 2.04 mmol) was added to a 50 mL single-mouth flask, dissolved with methanol (5 mL) and hydrogen chloride dioxane (4 M, 5 mL), and stirred at room temperature for 3 h, and the reaction liquid was concentrated to obtain the title compound 1D (0.47 g, 100%).

LC-MS (ESI): m/z=194.1 [M+H]⁺.

Step 3: Substrate 1E (0.15 g, 0.52 mmol) (compound 1E was synthesized according to the method of Patent WO 2013026797) was added to a 50 mL single-mouth flask and dissolved with dioxane (10 mL). 1C (0.14 g, 0.62 mmol) and diisopropylethylamine (0.2 g, 1.55 mmol) were added, and the mixture was reacted at 100 degrees Celsius overnight. The reaction liquid was concentrated, purified by HPLC, and freeze-dried to obtain the title compound 1 (120 mg, 52%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 30-80%; c. flow rate: 15 mL/min; d. elution time: 15 min; retention time: 7.0 min.

¹H NMR (400 MHz, DMSO-d6) δ 7.49-7.46 (m, 2H), 7.42-7.37 (m, 2H), 7.35 (s, 1H), 6.31 (t, 1H), 4.83 (t, 1H), 4.35 (s, 2H), 3.97 (t, 2H), 3.75 (d, 2H), 3.45-3.39 (m, 1H), 3.26-3.16 (m, 1H), 2.99-2.81 (m, 2H), 2.51-2.49 (m, 2H), 2.43-2.27 (m, 2H), 2.23-2.16 (m, 2H), 1.87-1.71 (m, 2H).

LC-MS (ESI): m/z=445.2 [M+H]⁺.

### Example 2

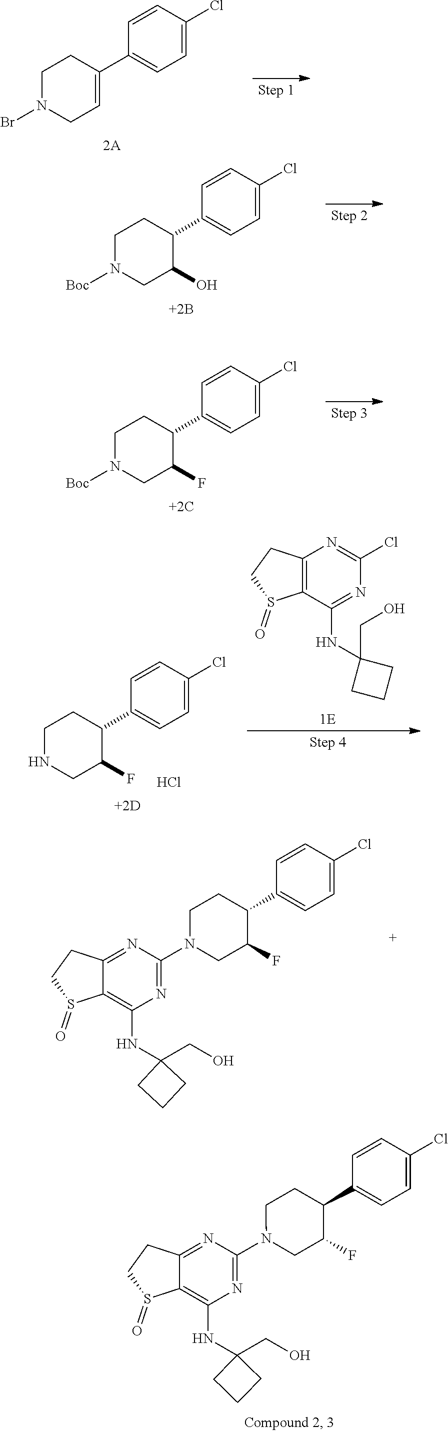

Step 1: Sodium borohydride (1.9 g, 51.05 mmol) and anhydrous tetrahydrofuran (50 mL) were added to a 500 mL single-mouth flask. Boron trifluoride diethyl etherate (7.25 g, 51.05 mmol) was added dropwise in an ice-water bath. After the dropwise addition was complete, the mixture was stirred in the ice bath for 0.5 h, and a solution of 2A (5.0 g, 17.02 mmol) in tetrahydrofuran (20 mL) was further added dropwise in the ice bath, naturally warmed to room temperature, and stirred overnight. Water (19 mL), ethanol (19 mL), and a sodium hydroxide solution (10%, 103 mL) were successively added to the reaction liquid. Finally, hydrogen peroxide (9.4 mL, 51.05 mmol) was added and stirred at 70 degrees Celsius overnight. The reaction liquid was adjusted to pH=5-6 with HCl (2 M) and extracted with dichloromethane (3×100 mL), the organic phases were combined, dried over anhydrous sodium sulfate, concentrated, and subjected to column chromatography (petroleum ether:ethyl acetate (v/v)=4:1) to obtain the title compound ±2B (±represented a racemate with the shown configuration) (4.0 g, 75% g).

LC-MS (ESI): m/z=256.1 [M−56+H]⁺.

Step 2: ±2B (1.9 g, 6.09 mmol) was added to a 100 mL single-mouth flask and dissolved with anhydrous dichloromethane (20 mL). Bis(2-methoxyethyl)aminosulfur trifluoride (1.48 g, 6.67 mmol) was added dropwise in an ice bath, and the mixture was slowly warmed to room temperature and stirred overnight. The reaction liquid was washed with a saturated sodium bicarbonate solution (20 mL), the aqueous phase was extracted with dichloromethane (2×20 mL), and the organic phases were combined, dried over anhydrous sodium sulfate, concentrated, and subjected to column chromatography (petroleum ether:ethyl acetate (v/v)=10:1) to obtain the title compound ±2C (1.56 g, 82%).

LC-MS (ESI): m/z=258.1 [M−56+H]⁺.

Step 3: ±2C (1.56 g, 4.97 mmol) was added to a 100 mL single-mouth flask and dissolved with anhydrous methanol (10 mL), hydrogen chloride dioxane (10 mL) was then added, and the mixture was stirred at room temperature for 2 h and concentrated to obtain the title compound ±2D (1.24 g, 100%).

LC-MS (ESI): m/z=214.1 [M+H]⁺.

Step 4: 1E (0.30 g, 1.04 mmol) was added to a 50 mL single-mouth flask and dissolved with dioxane (10 mL), and ±2D (0.32 g, 1.26 mmol) and diisopropylethylamine (0.4 g, 3.14 mmol) were added and reacted at 100 degrees Celsius overnight. The reaction liquid was concentrated and subjected to column chromatography (dichloromethane:methanol=20: 1-8:1) to obtain a mixture, which was subjected to chiral preparation to obtain the title compound 2 (0.11 g, 23%) and the title compound 3 (90 mg, 19%).

Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 100 mL/min; back pressure: 100 bar; column temperature: 25° C.; wavelength: 220 nm; elution time: 2.9 min.

Compound 2, retention time: 1.94 min, ¹H NMR (400 MHz, DMSO-d6) δ 7.43 (s, 1H), 7.40-7.33 (m, 4H), 5.04-5.01 (d, 1H), 4.82 (t, 1H), 4.71-4.63 (m, 1H), 4.57-4.51 (m, 1H), 3.77-3.66 (m, 2H), 3.47-3.39 (m, 1H), 3.27-3.19 (m, 1H), 3.09-2.81 (m, 5H), 2.46-2.25 (m, 2H), 2.20-2.14 (m, 2H), 1.91-1.55 (m, 4H).

Compound 3, retention time: 2.23 min, ¹H NMR (400 MHz, DMSO-d6) δ 7.43 (s, 1H), 7.39-7.34 (m, 4H), 5.05-5.02 (m, 1H), 4.82 (t, 1H), 4.69-4.52 (m, 2H), 3.71 (d, 2H), 3.48-3.39 (m, 1H), 3.27-3.19 (m, 1H), 3.08-2.81 (m, 5H), 2.41-2.29 (m, 2H), 2.21-2.10 (m, 2H), 1.89-1.55 (m, 4H).

LC-MS (ESI): m/z=465.2 [M+H]⁺.

### Example 3

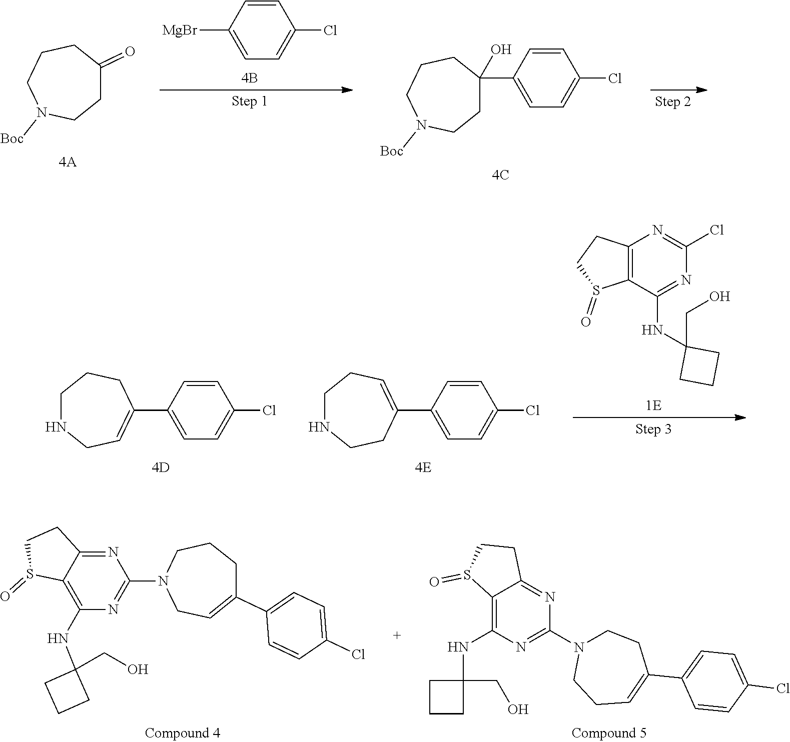

Step 1: 4A (2.0 g, 9.38 mmol) was added to a 100 mL single-mouth flask and dissolved with anhydrous tetrahydrofuran (10 mL), 4B (10.3 mL, 1 M, 10.32 mmol) was added dropwise in an ice bath, the mixture was stirred for 3 h at room temperature, quenched with a saturated ammonium chloride solution (30 mL), and extracted with ethyl acetate (2×20 mL), and the organic phases were combined, dried over anhydrous sodium sulfate, concentrated, and subjected to column chromatography (petroleum ether:ethyl acetate (v/v)=4:1) to obtain the title compound 4C (2.6 g, 85%).

LC-MS (ESI): m/z=252.1 [M−56-18+H]+.

Step 2: Substrate 4C (1.0 g, 3.07 mmol) was added to a 50 mL single-mouth flask and dissolved with dichloromethane (10 mL). Trifluoroacetic acid (5 mL) was added, the mixture was stirred overnight at room temperature, adjusted to pH 8-9 with an NaOH (2 M) solution, and extracted with dichloromethane (2×20 mL), and the organic phases were combined, dried over anhydrous sodium sulfate, and concentrated to obtain a mixture of the title compounds 4D and 4E (0.6 g, 94%).

LC-MS (ESI): m/z=208.1 [M+H]⁺.

Step 3: 1E (0.20 g, 0.69 mmol) was added to a 50 mL single-mouth flask and dissolved with dioxane (5 mL), and a mixture of 4D and 4E (0.21 g, 1.01 mmol) and diisopropylethylamine (0.26 g, 2.05 mmol) were added and reacted at 100 degrees Celsius overnight. The reaction liquid was concentrated and separated by column chromatography (dichloromethane:methanol=20: 1-10:1) to obtain 0.5 g of a crude product, which was purified by HPLC to obtain the title compound 4 (0.13 g, 42%) and the title compound 5 (50 mg, 16%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution: mobile phase A: 35-70%; c. flow rate: 15 mL/min; d. elution time: 15 min; retention time: compound 4, 4.0 min, and compound 5, 7.0 min.

Compound 4, ¹H NMR (400 MHz, DMSO-d6) δ 7.33 (brs, 4H), 7.24 (s, 1H), 6.17 (t, 1H), 4.82 (t, 1H), 4.30-4.32 (m, 2H), 4.09-3.85 (m, 2H), 3.71 (d, 2H), 3.44-3.36 (m, 1H), 3.24-3.16 (m, 1H), 2.98-2.78 (m, 2H), 2.66-2.56 (m, 2H), 2.44-2.27 (m, 2H), 2.17 (brs, 2H), 1.93-1.69 (m, 4H).

Compound 5, ¹H NMR (400 MHz, DMSO-d6) δ 7.34 (brs, 4H), 7.25 (s, 1H), 6.03 (t, 1H), 4.82 (t, 1H), 4.05-3.80 (m, 4H), 3.72 (d, 2H), 3.45-3.37 (m, 1H), 3.24-3.15 (m, 1H), 2.98-2.80 (m, 2H), 2.77-2.65 (m, 2H), 2.53-2.45 (m, 2H), 2.42-2.25 (m, 2H), 2.20-2.07 (m, 2H), 1.82-1.68 (m, 2H).

LC-MS (ESI): m/z=459.1 [M+H]⁺.

### Example 4

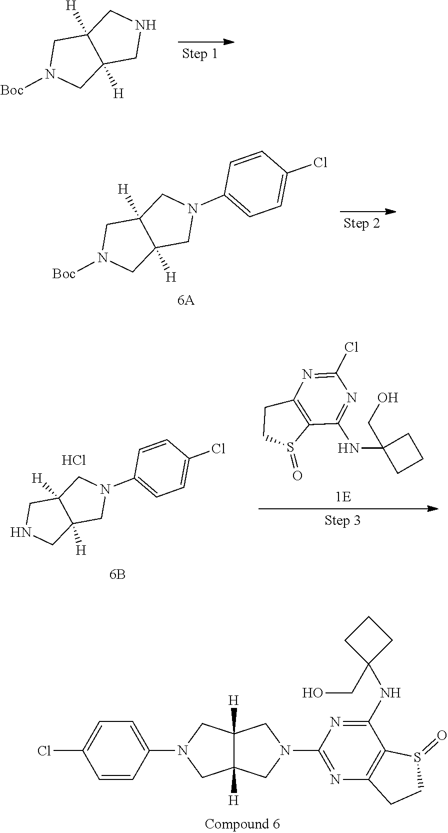 [SMILES: CC(C)(C)OC(=O)N1C[C@H]2CNC[C@H]2C1 ; CC(C)(C)OC(=O)N1C[C@@H]2CN(c3ccc(Cl)cc3)C[C@@H]2C1 ; Clc1ccc(N2C[C@H]3CNC[C@H]3C2)cc1 ; Cl ; O=[S@@]1CCc2nc(Cl)nc(NC3(CO)CCC3)c21 ; O=[S@@]1CCc2nc(N3C[C@H]4CN(c5ccc(Cl)cc5)C[C@H]4C3)nc(NC3(CO)CCC3)c21]

Step 1: Tert-butyl (3aR,6aS)-hexahydropyrrolo[3,4-c]pyrrole-2(1H)-carboxylate (2.12 g, 10 mmol), 1-chloro-4-iodobenzene (2.61 g, 11 mmol), Pd₂(dba)₃ (915 mg, 1 mmol), Xphos (952 mg, 2 mmol), and sodium tert-butoxide (1.15 g, 12 mmol) were weighed into a 250 mL three-mouth flask, and toluene (50 mL) and tert-butyl alcohol (10 mL) were added. After nitrogen displacement, a reaction was carried out at 100° C. for 16 h. After the completion of the reaction was monitored by LCMS, the system was cooled to room temperature and filtered, and the filter cake was washed with ethyl acetate. The filtrate was concentrated and then separated by silica gel column chromatography (PE:EA=20:1 to 5:1) to obtain the target compound 6A (2.76 g, 85.2%).

LC-MS (ESI): m/z=323.2 [M+H]⁺.

Step 2: 6A (2.76 g, 0.85 mmol) was dissolved in methanol (30 mL), and a solution of hydrogen chloride dioxane (5 mL, 4 M) was added. The mixture was reacted at room temperature for four hours and spin-dried to obtain title compound 6B (2.56 mg, crude).

LC-MS (ESI): m/z=223.2 [M+H]⁺.

Step 3: 1E (287 mg, 1 mmol) and 6B (300 mg) were dissolved in 1,4-dioxane (20 mL). DIPEA (1 mL) was added, and the mixture was left at 90° C. overnight. After the complete reaction of raw materials was detected by LCMS, the system was concentrated. Water (20 mL) was added, and the mixture was extracted with a mixed solution of DCM:MeOH=20:1 (20 mL×3). The organic phases were combined, dried over anhydrous sodium sulfate, concentrated, and then passed through a column (DCM:MeOH=1:0 to 0:1) to obtain compound 6 (289 mg, 60.9%). [00102]¹H NMR (400 MHz, DMSO-d₆) δ 7.33-7.07 (m, 3H), 6.66-6.38 (m, 2H), 4.85-4.76 (m, 1H), 3.82-3.74 (m, 2H), 3.74-3.67 (m, 2H), 3.52-3.34 (m, 5H), 3.26-3.13 (m, 3H), 3.13-3.04 (m, 2H) 2.96-2.79 (m, 2H), 2.43-2.27 (m, 2H), 2.19-2.09 (m, 2H), 1.86-1.65 (m, 2H).

LC-MS (ESI): m/z=474.2 [M+H]⁺.

### Example 5

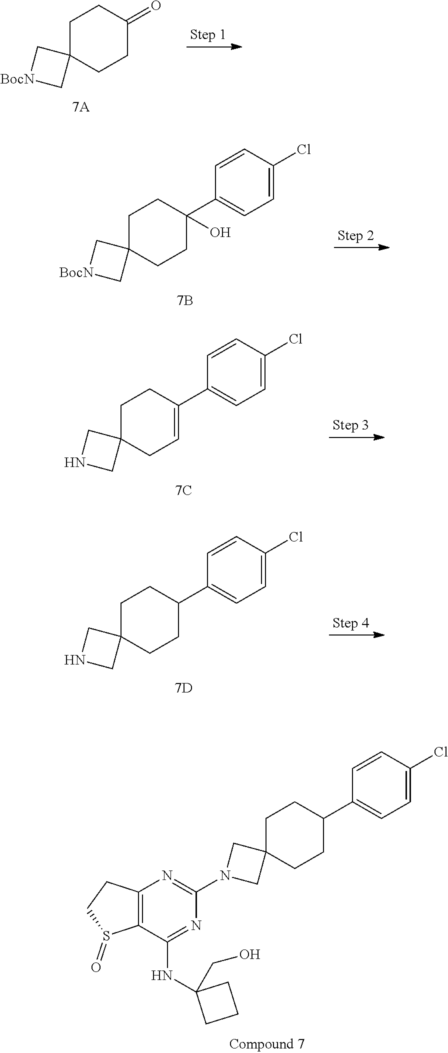 [SMILES: CC(C)(C)OC(=O)N1CC2(CCC(=O)CC2)C1 ; CC(C)(C)OC(=O)N1CC2(CCC(O)(c3ccc(Cl)cc3)CC2)C1 ; Clc1ccc(C2=CCC3(CC2)CNC3)cc1 ; Clc1ccc(C2CCC3(CC2)CNC3)cc1 ; O=[S@@]1CCc2nc(N3CC4(CCC(c5ccc(Cl)cc5)CC4)C3)nc(NC3(CO)CCC3)c21]

Step 1: Compound 7A (1 g, 4.2 mmol) was dissolved in 50 mL of tetrahydrofuran in a nitrogen atmosphere, and the system was cooled to 0° C. 4.6 mL of 4-chlorophenylmagnesium bromide (1 M in THF) was slowly added dropwise, and the mixture was warmed to room temperature and reacted for 3 h. The reaction was quenched by adding a saturated ammonium chloride aqueous solution. The reaction system was concentrated and extracted with 50 mL of ethyl acetate three times, and the resulting product was dried over anhydrous sodium sulfate. The organic phase was concentrated to obtain crude compound 7B, which was directly used in the next step.

LCMS m/z=296.1 [M−55]+.

Step 2: Crude compound 7B was dissolved in a mixed solvent (DCM:TFA=50 mL:4 mL) and reacted for 2 h at room temperature. After the reaction was complete, the system was concentrated to obtain crude compound 7C, which was directly used in the next step.

LCMS m/z=234.2 [M+1]⁺.

Step 3: Crude compound 7C was dissolved in 50 mL of methanol, and 300 mg palladium on carbon (10%) was added and reacted overnight in a hydrogen atmosphere. The solid residue in the system was removed by suction filtration, and the filtrate was concentrated to obtain crude compound 7D, which was directly used in the next reaction.

LCMS m/z=236.1 [M+1]⁺.

Step 4: In a nitrogen atmosphere, crude compound 7D, compound 1E (150 mg, 0.52 mmol), and DIPEA (0.86 mL, 5.2 mmol) were dissolved in 30 mL of 1,4-dioxane, and the mixture was warmed to 100° C. and reacted for 18 h. The reaction system was concentrated and then separated by thin-layer chromatography to obtain compound 7 (98 mg, 38%).

LCMS m/z=487.1 [M+1]⁺. [00112]¹H NMR (400 MHz, DMSO-d6) δ 7.34-7.26 (m, 5H), 4.87-4.84 (m, 1H), 3.85-3.59 (m, 6H), 3.44-3.34 (m, 1H), 3.26-3.15 (m, 1H), 2.95-2.81 (m, 2H), 2.54-2.46 (m, 1H), 2.41-2.27 (m, 2H), 2.15 (s, 2H), 1.99-1.93 (m, 2H), 1.86-1.68 (m, 4H), 1.64-1.57 (m, 2H), 1.50-1.41 (m, 2H).

### Example 6

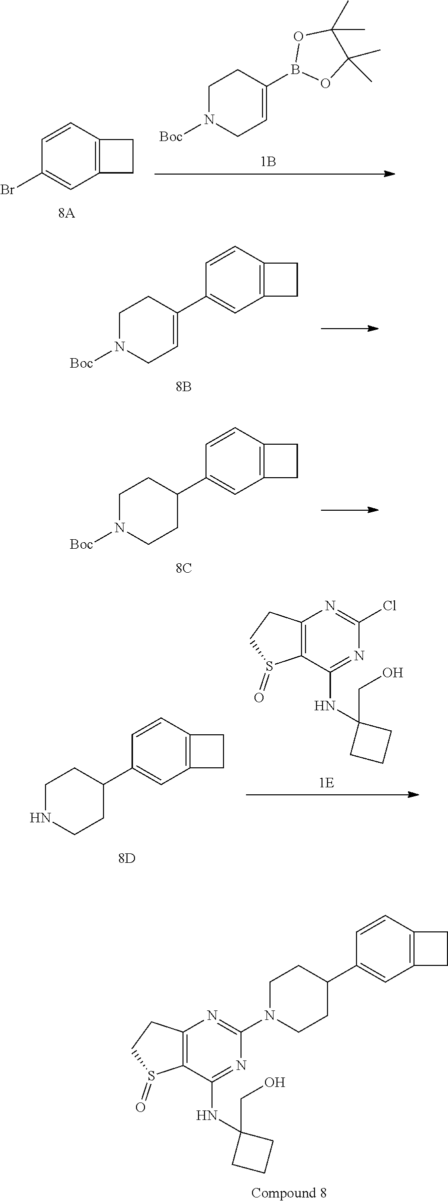

Step 1: Compound 8A (2.00 g, 10.91 mmol) and compound 1B (3.31 g, 10.91 mmol) were weighed into a 100 mL single-mouth flask and then dissolved by adding 1,4-dioxane (15 mL), and a sodium carbonate solution (2N, 15 mL) and Pd(dppf)Cl₂ (0.66 g, 1.1 mmol) were added to the reaction liquid. After the addition was complete, the mixture was stirred at 100° C. for 4 h. After the reaction was found complete by TLC plate spotting (petroleum ether:ethyl acetate=5:1), water (20 mL) was added to the reaction liquid and stirred for 5 min. Ethyl acetate (20 mL) was added for extraction. An organic phase was separated. The organic phase was dried over anhydrous sodium sulfate, and the crude product was concentrated under reduced pressure, and purified by column chromatography (eluents: petroleum ether:ethyl acetate=5:1) to obtain the target compound 8B (2.50 g, 80%).

LCMS m/z=286.1 [M+1]⁺.

Step 2: Compound 8B (1.00 g, 3.50 mmol) was weighed into a 100 mL single-mouth flask and then dissolved by adding ethyl acetate (20 mL), palladium on carbon (0.19 g, 1.74 mmol) was added to the reaction liquid, and after the addition was complete, the mixture was stirred for 1 h under hydrogen condition. After the reaction was complete, the reaction liquid was filtered and the filtrate was concentrated under reduced pressure to obtain the target compound 8C (1.00 g, 99%).

LCMS m/z=288.1 [M+1]⁺.

Step 3: Compound 8C (1.00 g, 3.48 mmol) was weighed into a 100 mL single-mouth flask and then dissolved by adding methanol (20 mL), a hydrogen chloride dioxane solution (4N, 20 mL) was added, and after the addition was complete, the mixture was stirred for 2 h. The organic phase was concentrated under reduced pressure to obtain the target compound 8D (0.60 g, yield: 92%).

LCMS m/z=188.2 [M+1]⁺.

Step 4: Compound 8D (0.20 g, 1.04 mmol) and compound 1E (0.30 g, 1.04 mmol) were added to a 100 mL single-mouth flask and dissolved by adding 1,4-dioxane (10 mL), and finally, N,N-diisopropylethylamine (0.40 g, 3.12 mmol) was added. After the addition was complete, the system was placed under nitrogen protection and stirred at 100° C. for 16 h, and the reaction liquid was concentrated under reduced pressure to obtain a crude product of the target compound, which was subjected to preparative HPLC to obtain the title compound 8 (190 mg, 62%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 40-80%; c. flow rate: 15 mL/min; d. elution time: 20 min; retention time: 10.38 min.

LCMS m/z=439.2 [M+1]⁺. [00122]¹H NMR (400 MHz, DMSO-d6) δ7.26 (s, 1H), 7.02-7.04 (m, 1H), 6.94-6.97 (m, 1H), 4.75-4.82 (m, 3H), 3.71 (d, 3H), 3.36-3.45 (m, 1H), 3.17-3.25 (m, 1H), 3.07 (s, 4H), 2.74-2.96 (m, 5H), 2.27-2.41 (m, 2H), 2.12-2.19 (m, 2H), 1.70-1.80 (m, 4H), 1.44-1.54 (m, 2H).

### Example 7

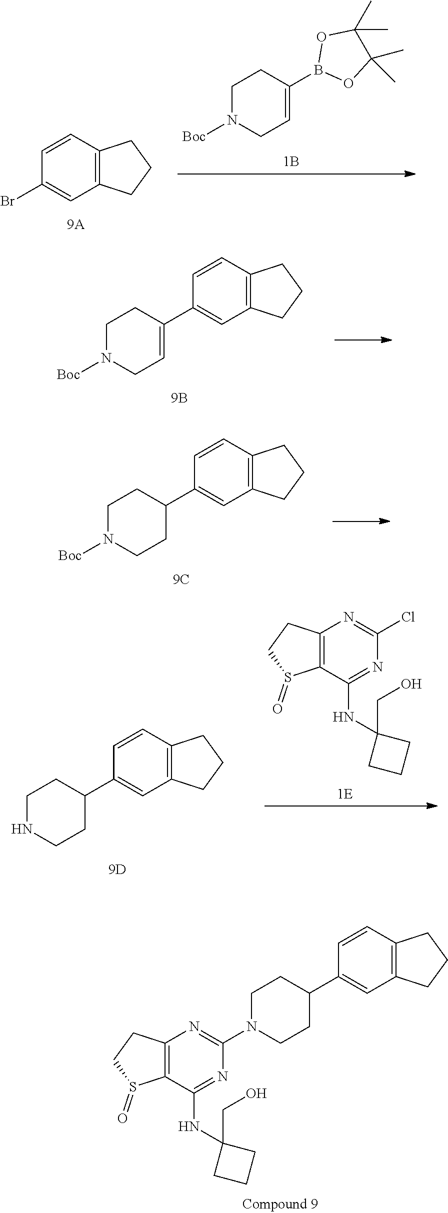

Step 1: Compound 9A (2.00 g, 10.05 mmol) and compound 1B (3.11 g, 10.05 mmol) were weighed into a 100 mL single-mouth flask and then dissolved by adding 1,4-dioxane (15 mL), and a sodium carbonate solution (2N, 15 mL) and [1,1′-bis(diphenylphosphino)ferrocene]palladium dichloride (0.66 g, 1.01 mmol) were added to the reaction liquid. After the addition was complete, the mixture was stirred at 100° C. for 4 h. After the completion of the reaction was monitored by TLC (petroleum ether:ethyl acetate=5:1), water (20 mL) was added to the reaction liquid and stirred for 5 min. Ethyl acetate (20 mL) was added for extraction, and an organic phase was separated. The organic phase was dried over anhydrous sodium sulfate, and the crude product was concentrated under reduced pressure and purified by column chromatography (eluents: petroleum ether:ethyl acetate=5:1) to obtain the target compound 9B (2.50 g, 80%).

LCMS m/z=300.1 [M+1]⁺.

Step 2: Compound 9B (3.00 g, 9.95 mmol) was weighed into a 100 mL single-mouth flask and then dissolved by adding ethyl acetate (20 mL), palladium on carbon (0.50 g, 4.97 mmol) was added to the reaction liquid, and after the addition was complete, the mixture was stirred for 1 h under hydrogen condition. After the reaction was complete, the reaction liquid was filtered and the filtrate was concentrated under reduced pressure until no liquid dripped out to obtain the target compound 9C (3.00 g, 99%).

LCMS m/z=302.1 [M+1]⁺.

Step 3: Compound 9C (3.00 g, 9.89 mmol) was weighed into a 100 mL single-mouth flask and dissolved by adding methanol (20 mL), a hydrogen chloride dioxane solution (4N, 20 mL) was added, and after the addition was complete, the mixture was stirred for 2 h. The organic phase was concentrated under reduced pressure to obtain the target compound 9D (2.00 g, 99%).

LCMS m/z=202.2 [M+1]⁺.

Step 4: Compound 9D (0.20 g, 1.03 mmol) and compound 1E (0.29 g, 1.03 mmol) were added to a 100 mL single-mouth flask and dissolved by adding 1,4-dioxane (10 mL), and finally, N,N-diisopropylethylamine (0.26 g, 2.05 mmol) was added. After the addition was complete, the system was placed under nitrogen protection and stirred at 100° C. for 16 h, and the reaction liquid was concentrated under reduced pressure to obtain a crude product of the target compound, which was subjected to preparative HPLC to obtain the title compound 9 (190 mg, 64%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 45-80%; c. flow rate: 20 mL/min; d. elution time: 20 min; retention time: 8.11 min.

LCMS m/z=453.2 [M+1]⁺. [00132]¹H NMR (400 MHz, DMSO-d6) δ7.26 (s, 1H), 7.07-7.12 (m, 2H), 6.96-6.97 (m, 1H), 4.75-4.82 (m, 3H), 3.72 (d, 2H), 3.36-3.45 (m, 1H), 3.18-3.25 (m, 1H), 2.75-2.97 (m, 9H), 2.28-2.41 (m, 2H), 2.11-2.20 (m, 2H), 1.93-2.01 (m, 2H), 1.72-1.80 (m, 4H), 1.44-1.54 (m, 2H).

### Example 8

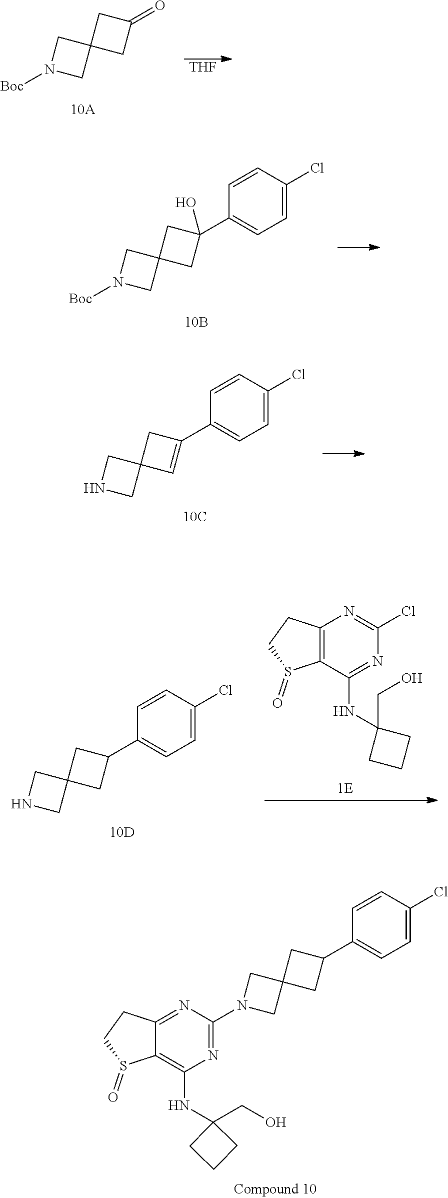

Step 1: Compound 10A (2.00 g, 9.47 mmol) was weighed into a 100 mL single-mouth flask and then dissolved by adding tetrahydrofuran (30 mL), 4-chlorophenylmagnesium chloride (18.94 mL, 18.94 mmol) was added to the reaction liquid. After the addition was complete, the mixture was stirred at room temperature for 4 h. After the reaction was found complete by TLC plate spotting (petroleum ether:ethyl acetate=3:1), water (20 mL) was added to the reaction liquid and stirred for 5 min, ethyl acetate (20 mL) was added for extraction, and an organic phase was separated. The organic phase was dried over anhydrous sodium sulfate, and the crude product was concentrated under reduced pressure and purified by column chromatography (eluents: petroleum ether:ethyl acetate=5:1) to obtain the target compound 10B (1.50 g, 48%).

LCMS m/z=324.1 [M+1]⁺.

Step 2: Compound 10B (0.50 g, 1.54 mmol) was weighed into a 100 mL single-mouth flask and then dissolved by adding dichloromethane (15 mL), trifluoroacetic acid (5 mL) was added to the reaction liquid, and after the addition was complete, the mixture was stirred at room temperature for 16 h. After the reaction was complete, the filtrate was concentrated under reduced pressure to obtain the target compound 10C (0.40 g, crude).

LCMS m/z=206.1 [M+1]⁺.

Step 3: Compound 10C (0.40 g, 1.94 mmol) was weighed into a 100 mL single-mouth flask and then dissolved by adding methanol (20 mL), palladium on carbon (0.10 g, 0.97 mmol) was added, and after the addition was complete, the mixture was stirred for 2 h. After the reaction was complete, the reaction liquid was filtered, and the organic phase was concentrated under reduced pressure to obtain the target compound 10D (0.40 g, crude).

LCMS m/z=208.2 [M+1]⁺.

Step 4: Compound 10D (0.40 g, 1.91 mmol) and compound 1E (0.55 g, 1.91 mmol) were added to a 100 mL single-mouth flask and dissolved by adding 1,4-dioxane (10 mL), and finally, N,N-diisopropylethylamine (0.74 g, 5.73 mmol) was added. After the addition was complete, the system was placed under nitrogen protection and stirred at 100° C. for 16 h, and the reaction liquid was concentrated under reduced pressure to obtain a crude product of the target compound, which was subjected to preparative HPLC to obtain the title compound 10 (25 mg, 4%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 50-90%; c. flow rate: 15 mL/min; d. elution time: 20 min; retention time: 6.45 min.

LCMS m/z=459.2 [M+1]⁺.

¹H NMR (400 MHz, DMSO-d6) δ7.34-7.36 (m, 2H), 7.25-7.30 (m, 3H), 4.86 (t, 1H), 4.14 (s, 2H), 3.92 (s, 2H), 3.70 (d, 2H), 3.35-3.44 (m, 2H), 3.17-3.24 (m, 1H), 2.82-2.95 (m, 2H), 2.54-2.61 (m, 2H), 2.24-2.34 (m, 4H), 2.10-2.16 (m, 2H), 1.69-1.85 (m, 2H).

### Example 9

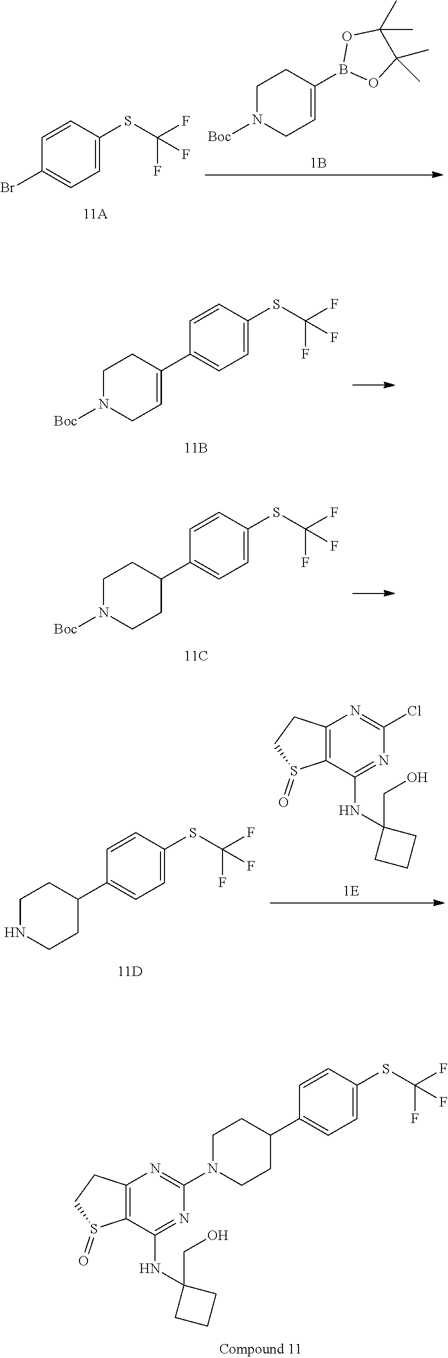

Step 1: Compound 11A (2.00 g, 7.78 mmol) and compound 1B (2.89 g, 9.34 mmol) were weighed into a 100 mL single-mouth flask and then dissolved by adding 1,4-dioxane (15 mL), and a sodium carbonate solution (2N, 15 mL) and [1,1′-bis(diphenylphosphino)ferrocene]palladium dichloride (0.57 g, 0.78 mmol) were added to the reaction liquid. After the addition was complete, the mixture was stirred at 100° C. for 4 h. After the completion of the reaction was monitored by TLC plate spotting (petroleum ether:ethyl acetate=5:1), water (20 mL) was added to the reaction liquid and stirred for 5 min, ethyl acetate (20 mL) was added for extraction, and an organic phase was separated. The organic phase was dried over anhydrous sodium sulfate, and the crude product was concentrated under reduced pressure and purified by column chromatography (eluents: petroleum ether:ethyl acetate=5:1) to obtain the target compound 11B (2.60 g, 93%).

LCMS m/z=360.1 [M+1]⁺.

Step 2: Compound 11B (0.50 g, 1.39 mmol) was weighed into a 100 mL single-mouth flask and then dissolved by adding ethanol (20 mL), palladium on carbon (0.15 g, 1.39 mmol) was added to the reaction liquid, and after the addition was complete, the mixture was stirred for 16 h under hydrogen condition. After the reaction was complete, the reaction liquid was filtered and the filtrate was concentrated under reduced pressure to obtain the target compound 11C (0.50 g, 99%).

LCMS m/z=362.1 [M+1]⁺.

Step 3: Compound 11C (0.50 g, 1.38 mmol) was weighed into a 100 mL single-mouth flask and then dissolved by adding methanol (20 mL), a hydrogen chloride dioxane solution (4N, 20 mL) was added, and after the addition was complete, the mixture was stirred for 2 h. The organic phase was concentrated under reduced pressure to obtain the target compound 11D (0.40 g, crude).

LCMS m/z=262.2 [M+1]⁺.

Step 4: Compound 11D (0.20 g, 0.76 mmol) and compound 1E (0.22 g, 0.76 mmol) were added to a 100 mL single-mouth flask and dissolved by adding 1,4-dioxane (10 mL), and finally, N,N-diisopropylethylamine (0.29 g, 2.28 mmol) was added. After the addition was complete, the system was placed under nitrogen protection and stirred at 100° C. for 16 h, and the reaction liquid was concentrated under reduced pressure to obtain a crude product of the target compound, which was subjected to preparative HPLC to obtain the title compound 11 (240 mg, 61%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution: mobile phase A: 30-70%; c. flow rate: 15 mL/min; d. elution time: 20 min; retention time: 12.08 min.

LCMS m/z=513.2 [M+1]⁺. [00152]¹H NMR (400 MHz, DMSO-d6) δ7.62 (d, 2H), 7.43 (d, 2H), 7.29 (s, 1H), 4.77-4.84 (m, 3H), 3.71 (d, 2H), 3.37-3.45 (m, 1H), 3.18-3.26 (m, 1H), 2.82-2.98 (m, 5H), 2.28-2.41 (m, 2H), 2.12-2.19 (m, 2H), 1.70-1.85 (m, 4H), 1.48-1.58 (m, 2H).

### Example 10

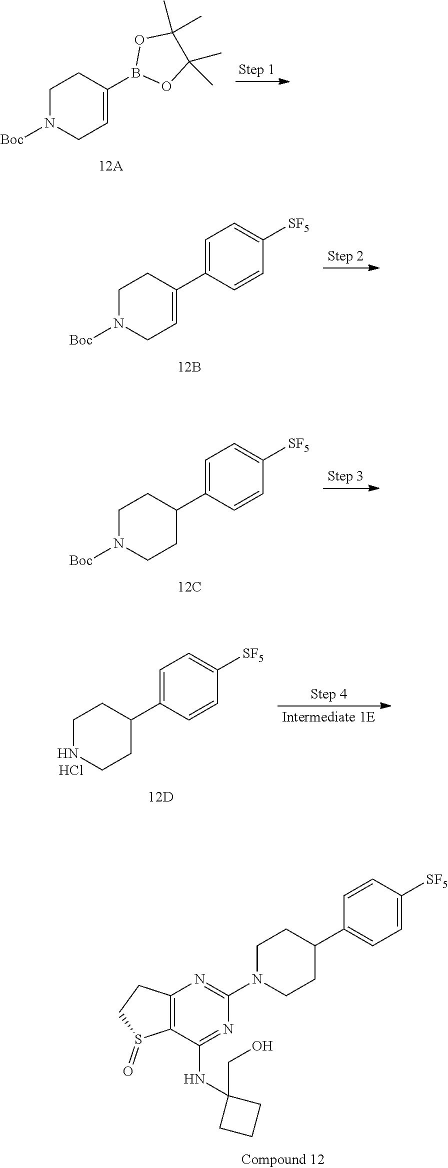

Step 1: (4-Bromophenyl)sulfur pentafluoride (1.0 g, 3.55 mmol) and N-Boc-1,2,5,6-tetrahydropyridine-4-boronic acid pinacol ester (12A, 1.65 g, 5.33 mmol) were dissolved in 1,4-dioxane (10 mL). A sodium carbonate aqueous solution (2 mol/L, 10 mL) and 1,1′-bis(diphenylphosphino)ferrocene palladium dichloride (200 mg) were added, the mixture was refluxed for 4 hours under nitrogen protection and extracted with ethyl acetate (30 mL×2), and the resulting product was dried over anhydrous sodium sulfate, concentrated, and then purified by silica gel column chromatography (petroleum ether:ethyl acetate (v/v)=100:10) to obtain product (12B) (700 mg, yield 51%).

LCMS m/z=330.1 [M+1-56]+.

Step 2: Compound 12B (700 mg, 1.8 mmol) was dissolved in methanol (10 mL), palladium on carbon (100 mg) was added, a hydrogenation reaction was carried out at room temperature for 3 hours, and after filtration, the filtrate was concentrated to obtain a crude product (12C) (700 mg, yield 100%).

LCMS m/z=332.1 [M+1-56]+.

Step 3: Hydrogen chloride-dioxane (10 mL) was added to compound 12C (700 mg, 1.8 mmol), and the mixture was reacted at room temperature for 1 hour and concentrated to obtain a crude product (12D) (400 mg, yield 72%).

LCMS m/z=288.1 [M+1]⁺.

Step 4: Compound 12D (0.1 g, 0.31 mmol) and intermediate 1E (89 mg, 0.31 mmol) were dissolved in 1,4-dioxane (10 mL), N,N-diisopropylethylamine (120 mg, 0.93 mmol) was added, the mixture was refluxed and stirred overnight for a reaction. After concentration, the reaction product was directly separated and purified by a liquid phase preparative column (liquid phase preparation conditions: C18 reverse-phase preparative column, mobile phase: deionized water containing 0.1% aqueous ammonia (A) and acetonitrile (B), gradient elution, mobile phase B=5-50%, elution time: 15 min, flow rate: 12 mL/min, and column temperature: 30° C.) to obtain the title compound 12 (50 mg, yield 29%, retention time: about 9.0 min).

¹H NMR (400 MHz, DMSO-d₆) δ7.79 (d, 2H), 7.50 (d, 2H), 7.30 (s, 1H), 4.91-4.72 (m, 3H), 3.72 (d, 2H), 3.47-3.36 (m, 1H), 3.27-3.17 (m, 1H), 3.02-2.80 (m, 5H), 2.44-2.26 (m, 2H), 2.22-2.09 (m, 2H), 1.87-1.69 (m, 4H), 1.60-1.47 (m, 2H).

MS M/Z (ESI): m/z=539.1 [M+1]⁺.

### Example 11

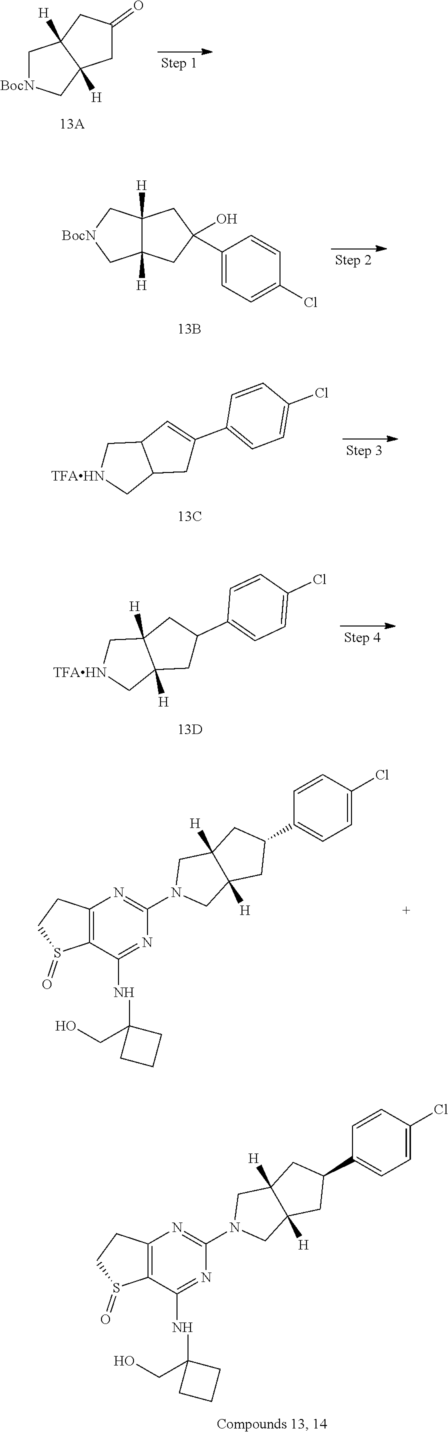

Step 1: p-Chloroiodobenzene (5.82 g, 24.41 mmol) and anhydrous tetrahydrofuran (50 mL) were added to a 250 mL three mouth flask, and after nitrogen displacement, n-butyl lithium (39 mmol, 11.5 mL) was dropwise added at −78° C. and reacted at −78° C. for 2 h. A solution of 12A (5 g, 22.19 mmol) in tetrahydrofuran (20 mL) was dropwise added, and the mixture was maintained at −78° C. and reacted for 1 h. Water (100 mL) was added. The mixture was extracted with ethyl acetate (50 mL×2), the organic phases were combined, washed with a saturated sodium chloride solution, dried over anhydrous sodium sulfate, and filtered, and the filtrate was concentrated under reduced pressure and then separated and purified by silica gel column chromatography (eluent EA/PE=0-35%) to obtain the target compound 13B (4 g, 53.36%).

LC-MS (ESI): m/z=338.1 [M+H]⁺.

Step 2: 13B (4 g, 11.84 mmol) was added to a mixed system of dichloromethane (45 mL) and trifluoroacetic acid (15 mL) and reacted at room temperature for 3 h. The reaction liquid was directly concentrated under reduced pressure and then brought to the next reaction.

LC-MS (ESI): m/z=220.1 [M+H]⁺.

Step 3: 13C (4 g, 11.99 mmol), methanol (50 mL), and palladium on carbon 10% (0.8 g) were added to a 100 mL single-mouth flask and reacted at room temperature for 1 h under hydrogen displacement. After filtration, the filtrate was concentrated under reduced pressure and then separated and purified by HPLC to obtain the target compound 13D (1.5 g, 37.26%).

LC-MS (ESI): m/z=222.1 [M+H]⁺.

Preparative HPLC separation method: 1. Instrument: waters 2767 preparative liquid chromatographic instrument; chromatographic column: SunFire@Prep C18 (19 mm×250 mm). 2. The sample was dissolved with DMF and filtered with a 0.45 m filter to prepare a sample liquid. 3. Preparative chromatography conditions: a. composition of mobile phases A and B: mobile phase A: acetonitrile; mobile phase B: water (containing 0.1% acetic acid); b. gradient elution, mobile phase A: 5-40%; c. flow rate: 12 mL/min. d. elution time: 20 min.

Step 4: Compound 1E (0.15 g, 0.52 mmol), 13D (0.21 g, 0.63 mmol), dioxane (5 mL), and DIPEA (0.27 g, 2.05 mmol) were successively added to a 50 mL single-mouth flask and reacted at 100° C. for 5 h. The reaction product was cooled to room temperature, and the reaction liquid was directly concentrated under reduced pressure and then separated and purified by silica gel column chromatography (eluent MeOH/DCM=0-10%) to obtain 220 mg of a mixture.

After chiral preparation, the title compound 13 (0.14 g, 56%, retention time: 0.787 min) and the title compound 14 (20 mg, 8%, retention time: 0.995 min) were obtained. Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 120 mL/min; back pressure: 100 bar; column temperature: 25° C.; wavelength: 220 nm; elution time: 5.5 min.

Compound 13 ¹H NMR (400 MHz, DMSO-d6) δ 7.34-7.25 (m, 4H), 7.22 (s, 1H), 4.83 (t, 1H), 3.73 (d, 2H), 3.66-3.62 (m, 2H), 3.52-3.50 (m, 2H), 3.45-3.33 (m, 1H), 3.25-3.08 (m, 2H), 2.97-2.74 (m, 4H), 2.44-2.23 (m, 4H), 2.17-2.13 (m, 2H), 1.84-1.67 (m, 2H), 1.45-1.38 (m, 2H).

LC-MS (ESI): m/z=473.2 [M+H]⁺.

Compound 14 ¹H NMR (400 MHz, DMSO-d6) δ 7.35-7.28 (m, 4H), 7.22 (s, 1H), 4.84 (t, 1H), 3.84-3.80 (m, 2H), 3.74 (d, 2H), 3.45-3.33 (m, 3H), 3.24-3.17 (m, 2H), 2.95-2.82 (m, 4H), 2.42-2.30 (m, 2H), 2.18-2.13 (m, 2H), 1.94-1.72 (m, 6H).

LC-MS (ESI): m/z=473.2 [M+H]⁺.

### Example 12

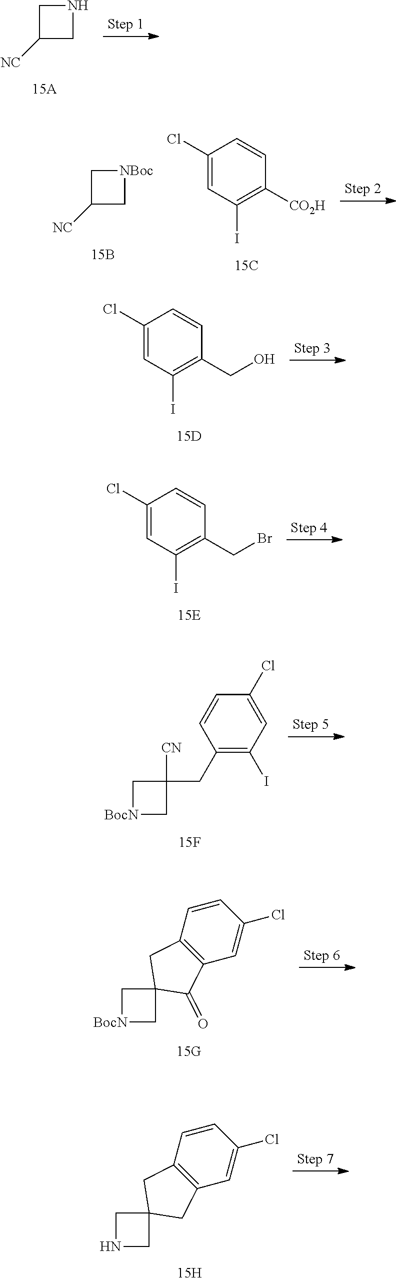

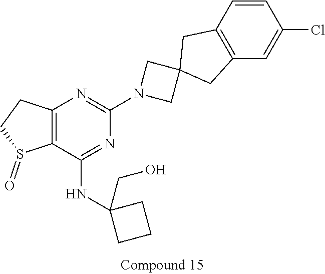 [SMILES: O=[S@@]1CCc2nc(N3CC4(Cc5ccc(Cl)cc5C4)C3)nc(NC3(CO)CCC3)c21]

Step 1: Compound 15A (5 g, 45 mmol) was dissolved in 200 mL of dichloromethane, triethylamine (17 mL, 122 mmol) and di-tert-butyl dicarbonate (11.5 mL, 50 mmol) were added and reacted at room temperature for 5 h. After the raw materials disappeared, a saturated ammonium chloride aqueous solution was added for quenching, followed by extraction with 200 mL of dichloromethane three times and then drying with anhydrous sodium sulfate, and the organic phase was concentrated to obtain compound 15B (8 g, 95%).

LCMS m/z=127.2 [M−55]+.

Step 2: In a nitrogen atmosphere, compound 15C (15 g, 53.2 mmol) was dissolved in 200 mL of tetrahydrofuran. The system was then cooled to 0° C., and a borane tetrahydrofuran solution (133 mL, 1 M in THF) was slowly added. The mixture was warmed to room temperature, heated to reflux for 3 h, and then cooled to 0° C., and the reaction was quenched by adding 1N hydrochloric acid. The system was concentrated, extracted three times by adding 200 mL of ethyl acetate and dried over anhydrous sodium sulfate, and then the organic phase was concentrated to obtain 15 g of crude compound 15D. The compound was directly used for the next reaction. [00178]¹H NMR (400 MHz, CDCl₃) δ 7.82-7.81 (m, 1H), 7.41-7.33 (m, 2H), 4.64 (s, 2H), 1.92 (s, 1H).

Step 3: Triphenylphosphine (22 g, 83.8 mmol) was dissolved in 250 mL of dichloromethane, and the system was cooled to 0° C. Bromine (4.5 mL, 83.5 mmol) was added slowly and stirred for 10 min, imidazole (7.6 g, 111 mmol) and 15 g of crude compound 15D were then added, and the mixture was warmed to room temperature and reacted for 3 h. The reaction was quenched by adding sodium thiosulfate, and the reaction product was then extracted with 200 mL of dichloromethane.

After drying with anhydrous sodium sulfate, the organic phase was concentrated and separated by column chromatography to obtain compound 15E (12 g, 68%).

¹H NMR (400 MHz, CDCl₃) δ 7.86-7.84 (m, 1H), 7.42-7.29 (m, 2H), 4.55 (s, 2H).

Step 4: In a nitrogen atmosphere, compound 15B (2 g, 11 mmol) was dissolved in 100 mL of tetrahydrofuran and cooled to −40° C., and sodium bis(trimethylsilyl)amide (11 mL, 2 M in THF) was then added and stirred for 30 min. Compound 15E (3.6 g, 10.8 mmol) was dissolved in a small amount of tetrahydrofuran and then added to the system. The system was maintained at −40° C. and reacted for 2 h. After the reaction was complete, the reaction was quenched by adding a saturated ammonium chloride aqueous solution, and the reaction product was warmed to room temperature and extracted with 100 mL of ethyl acetate three times. After drying with anhydrous sodium sulfate, the organic phase was concentrated and separated by column chromatography to obtain compound 15F (2 g, 41%).

LCMS m/z=376.9 [M−55]⁺.

Step 5: Under nitrogen, compound 15F (1.9 g, 4.4 mmol) was dissolved in 20 mL of tetrahydrofuran, and the system was cooled to −78° C. n-Butyl lithium (9.5 mL, 2.5 M) was slowly added dropwise, and the mixture was maintained at −78° C. and reacted for 15 min. After the raw materials disappeared, the reaction was quenched by adding a saturated ammonium chloride aqueous solution, and the reaction product was extracted with 50 mL of ethyl acetate three times. After drying with anhydrous sodium sulfate, the organic phase was concentrated and separated by column chromatography to obtain compound 15G (800 mg, 59%).

LCMS m/z=252.0 [M−55]+.

Step 6: In a nitrogen atmosphere, compound 15G (400 mg, 1.3 mmol) was dissolved in 20 mL of dichloromethane, boron trifluoride diethyl etherate (1.5 mL, 11.4 mmol) and triethylsilane (3 mL, 23 mmol) were added, and the mixture was warmed to 45° C. and reacted for 10 h. After the raw materials disappeared, the reaction system was cooled to room temperature, poured into a saturated sodium bicarbonate aqueous solution, and extracted with 50 mL of dichloromethane, and after drying with anhydrous sodium sulfate, the organic phase was concentrated to obtain 280 mg of crude compound 15H, which was directly brought to the next reaction.

LCMS m/z=194.1 [M+H]⁺.

Step 7: In a nitrogen atmosphere, 280 mg of crude compound 15H, compound 1E (150 mg, 0.52 mmol), and DIPEA (0.86 mL, 5.2 mmol) were dissolved in 30 mL of 1,4-dioxane, and the mixture was warmed to 100° C. and reacted for 18 h. The reaction system was concentrated and then separated by thin-layer chromatography to obtain compound 15 (71 mg, 31%).

LCMS m/z=445.1 [M+H]⁺. [00189]¹H NMR (400 MHz, DMSO-d6) δ 7.33-7.17 (m, 4H), 4.85-4.81 (m, 1H), 3.95 (s, 4H), 3.74-3.61 (m, 2H), 3.44-3.34 (m, 1H), 3.26-3.10 (m, 5H), 2.97-2.81 (m, 2H), 2.40-2.25 (m, 2H), 2.16-2.06 (m, 2H), 1.83-1.63 (m, 2H).

### Example 13

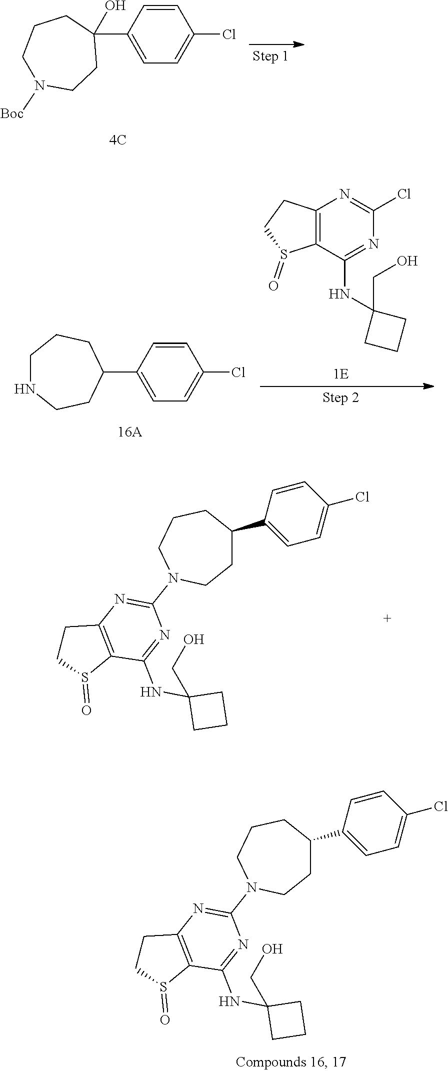 [SMILES: CC(C)(C)OC(=O)N1CCCC(O)(c2ccc(Cl)cc2)CC1 ; Clc1ccc(C2CCCNCC2)cc1 ; O=[S@@]1CCc2nc(Cl)nc(NC3(CO)CCC3)c21 ; O=[S@@]1CCc2nc(N3CCC[C@@H](c4ccc(Cl)cc4)CC3)nc(NC3(CO)CCC3)c21 ; O=[S@@]1CCc2nc(N3CCC[C@H](c4ccc(Cl)cc4)CC3)nc(NC3(CO)CCC3)c21]

Step 1: 4C (0.5 g, 1.53 mmol) was added to a 50 mL single-mouth flask and dissolved with dichloromethane (10 mL), triethylsilane (0.53 g, 4.59 mmol) was added, boron trifluoride diethyl etherate (0.43 g, 3.06 mmol) was dropwise added in an ice bath, and the mixture was stirred at room temperature overnight. The reaction liquid was washed with a saturated sodium bicarbonate solution (20 mL) and extracted with dichloromethane (2×20 mL), and the organic phases were combined, dried over anhydrous sodium sulfate, and concentrated to obtain the title compound 16A (0.35 g, crude).

LC-MS (ESI): m/z=210.1 [M+H]⁺.

Step 2: 1E (0.40 g, 1.39 mmol) was added to a 50 mL single-mouth flask and dissolved with dioxane (10 mL), and 16A (0.35 g, 1.67 mmol) and diisopropylethylamine (0.54 g, 4.18 mmol) were added and reacted at 100 degrees Celsius overnight. The reaction liquid was concentrated and separated by column chromatography (dichloromethane:methanol=20: 1-10:1) to obtain 0.2 g of a crude product, which was subjected to chiral preparation to obtain the title compound 16 (40 mg, 6%) and the title compound 17 (35 mg, 5%).

Chiral preparation method: instrument: Waters 150 MGM; chromatographic column: Chiralpak Column; mobile phase: A: carbon dioxide, and B: isopropanol (0.1% aqueous ammonia); isocratic elution: 25% mobile phase B; flow rate: 100 mL/min; back pressure: 100 bar; column temperature: 25° C.; wavelength: 220 nm; elution time: 2.8 min.

Compound 16, retention time: 2.16 min, ¹H NMR (400 MHz, DMSO-d6) δ 7.29 (d, 2H), 7.22 (d, 2H), 7.16 (s, 1H), 4.85-4.74 (m, 1H), 4.09-3.87 (m, 2H), 3.73-3.61 (m, 3H), 3.45-3.36 (m, 2H), 3.26-3.19 (m, 1H), 3.00-2.77 (m, 2H), 2.71-2.62 (m, 1H), 2.42-2.34 (m, 2H), 2.15-2.10 (m, 2H), 1.98-1.91 (m, 2H), 1.75-1.60 (m, 6H).

Compound 17, retention time: 2.47 min, ¹H NMR (400 MHz, DMSO-d6) δ 7.32-7.27 (m, 2H), 7.25-7.13 (m, 3H), 4.86-4.76 (m, 1H), 4.06-3.96 (m, 1H), 3.83-3.64 (m, 4H), 3.58-3.55 (m, 1H), 3.50-3.36 (m, 1H), 3.26-3.18 (m, 1H), 2.98-2.80 (m, 2H), 2.69-2.66 (m, 1H), 2.42-2.32 (m, 2H), 2.21-2.06 (m, 2H), 1.98-1.95 (m, 2H), 1.89-1.53 (m, 6H).

LC-MS (ESI): m/z=461.2 [M+H]⁺.

### Example 14

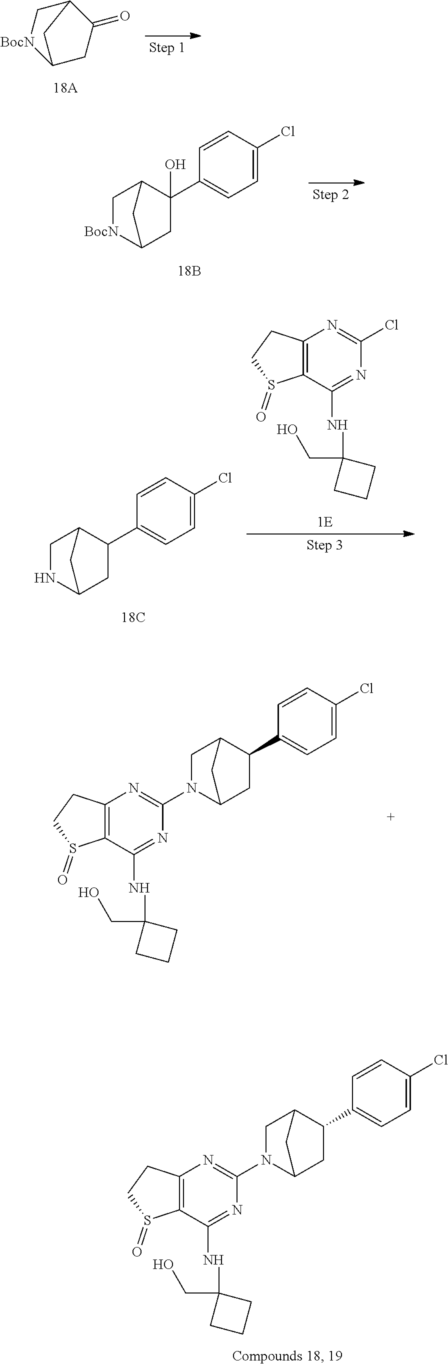 [SMILES: CC(C)(C)OC(=O)N1CC2CC1CC2=O ; CC(C)(C)OC(=O)N1CC2CC1CC2(O)c1ccc(Cl)cc1 ; Clc1ccc(C2CC3CC2CN3)cc1 ; O=[S@@]1CCc2nc(Cl)nc(NC3(CO)CCC3)c21 ; O=[S@@]1CCc2nc(N3CC4CC3C[C@@H]4c3ccc(Cl)cc3)nc(NC3(CO)CCC3)c21 ; O=[S@@]1CCc2nc(N3CC4CC3C[C@H]4c3ccc(Cl)cc3)nc(NC3(CO)CCC3)c21]

Step 1: p-Chloroiodobenzene (2.5 g, 10.42 mmol) and anhydrous tetrahydrofuran (20 mL) were added to a 100 mL three mouth flask, and after nitrogen displacement, n-butyl lithium (12 mmol, 4.8 mL) was dropwise added at −78° C. and reacted at −78° C. for 2 h. A solution of 18A (2 g, 9.5 mmol) in tetrahydrofuran (10 mL) was then added dropwise, and the mixture was maintained at −78° C. and reacted for 1 h. Water (100 mL) was added. The mixture was extracted with ethyl acetate (50 mL×2), the organic phases were combined, washed with a saturated sodium chloride solution, dried over anhydrous sodium sulfate, and filtered, and the filtrate was concentrated under reduced pressure and then separated and purified by silica gel column chromatography (eluent EA/PE=0-35%) to obtain the target compound 18B (2.4 g, 78%).

LC-MS (ESI): m/z=324.1 [M+H]⁺.

Step 2: 18B (2 g, 6.18 mmol), dichloroethane (30 mL), triethylsilane (3.59 g, 30.9 mmol), and boron trifluoride diethyl etherate (4.4 g, 3.81 mmol) were successively added to a 100 ml single-mouth flask. The mixture was reacted at 80° C. for 3 h. The reaction product was cooled to room temperature, and the reaction liquid was directly concentrated under reduced pressure and then purified by C18 reverse-phase column, wherein the composition of mobile phases A and B were: mobile phase A: acetonitrile; mobile phase B: water (containing 0.1% TFA), (A/B=30%/70%).

After separation and purification, the title compound 18C (450 mg, 22%) was obtained.

LC-MS (ESI): m/z=208.1 [M+H]⁺.

Step 3: 1E (0.15 g, 0.52 mmol), 18C (0.14 g, 0.68 mmol), dioxane (5 mL), and DIPEA (0.27 g, 2.05 mmol) were successively added to a 50 mL single-mouth flask and reacted at 100° C. for 5 h.

The reaction product was cooled to room temperature, and the reaction liquid was directly concentrated under reduced pressure and then separated and purified by silica gel column chromatography (eluent MeOH/DCM=0-10%) to obtain 220 mg of a mixture.

After chiral preparation, the title compound 18 (80 mg, 33%, retention time: 1.253 min) and the title compound 19 (78 mg, 32%, retention time: 1.392 min) were obtained. Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 100 mL/min; back pressure: 100 bar; column temperature: 25° C.; wavelength: 220 nm; elution time: 2.7 min.

Compound 18: [00204]¹H NMR (400 MHz, DMSO-d6) δ 7.37-7.31 (m, 2H), 7.21 (d, 1H), 7.15 (d, 1H), 7.09 (d, 1H), 4.89-4.78 (m, 1H), 4.65 (d, 1H), 3.79-3.69 (m, 1H), 3.65-3.57 (m, 1H), 3.52-3.44 (m, 1H), 3.43-3.33 (m, 1H), 3.27-3.03 (m, 3H), 3.01-2.68 (m, 3H), 2.46-2.22 (m, 2H), 2.21-2.09 (m, 2H), 2.04-1.93 (m, 1H), 1.91-1.58 (m, 5H).

LC-MS (ESI): m/z=459.2 [M+H]⁺.

Compound 19: [00207]¹H NMR (400 MHz, DMSO-d6) δ 7.35-7.25 (m, 3H), 7.15-7.11 (m, 2H), 4.84 (d, 1H), 4.66 (d, 1H), 3.78-3.71 (m, 1H), 3.66-3.57 (m, 1H), 3.48-3.42 (m, 2H), 3.24-3.19 (m, 1H), 3.18-3.04 (m, 2H), 3.00-2.69 (m, 3H), 2.45-2.32 (m, 1H), 2.30-2.11 (m, 3H), 2.05-1.93 (m, 1H), 1.92-1.83 (m, 2H), 1.81-1.59 (m, 3H).

LC-MS (ESI): m/z=459.2 [M+H]⁺.

### Example 15

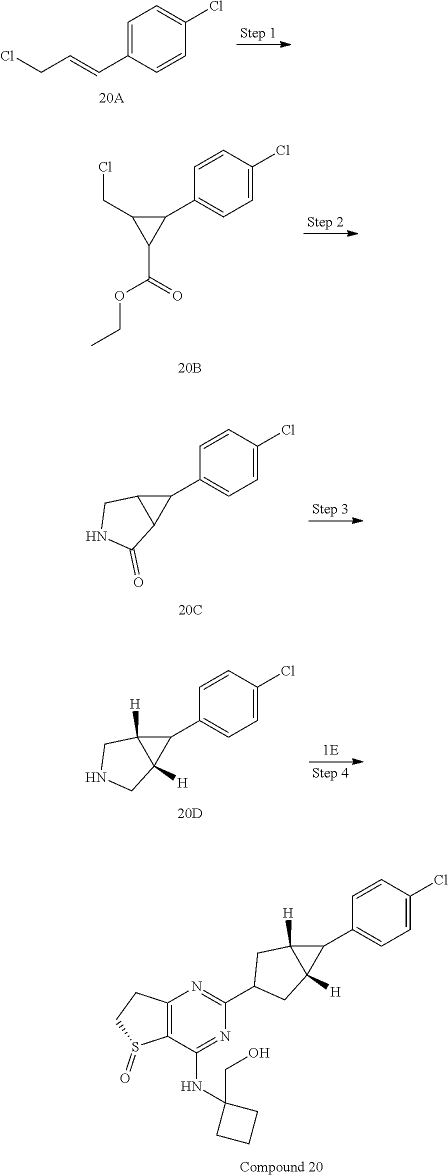 [SMILES: ClC/C=C/c1ccc(Cl)cc1 ; CCOC(=O)C1C(CCl)C1c1ccc(Cl)cc1 ; O=C1NCC2C1C2c1ccc(Cl)cc1 ; Clc1ccc(C2[C@H]3CNC[C@@H]23)cc1 ; O=[S@@]1CCc2nc(C3C[C@@H]4C(c5ccc(Cl)cc5)[C@@H]4C3)nc(NC3(CO)CCC3)c21]

Step 1: Under nitrogen protection at room temperature, to a solution charged with 20A (2.00 g, 10.70 mmol, synthesis reference: Chemistry-A European Journal, 2019, 25(14), 3521-3524) and rhodium acetate (190 mg, 0.4 mmol) in dichloromethane (200 mL), ethyl diazoacetate (2.50 g, 21.40 mmol) was slowly added dropwise, and after the addition was complete, the mixture was stirred at room temperature overnight. The reaction liquid was filtered by diatomite to obtain a filtrate, which was concentrated in vacuum to obtain a crude product. The crude product was purified by column chromatography (eluents: PE:EA=100:1 to 20:1) to obtain the target compound 20B (1.10 g, yield: 37.67%).

¹H NMR (400 MHz, CDCl₃) δ 7.19 (d, 2H), 6.97 (d, 2H), 4.20-4.13 (m, 2H), 3.96-3.91 (m, 1H), 3.81-3.76 (m, 1H), 2.60-2.57 (m, 1H), 2.14-2.10 (m, 1H), 2.02-1.96 (m, 1H), 1.27-1.22 (m, 3H).

Step 2: Compound 20B (1.00 g, 3.66 mmol) was dissolved in a 7N ammonia methanol solution (40 mL), then heated to 80° C., and stirred for 16 h. The reaction liquid was cooled to room temperature and then concentrated under reduced pressure to obtain a crude product, which was purified by column chromatography (eluents: PE:EA=100:1 to 1:1) to obtain the target compound 20C (400 mg, yield: 52.62%).

LC-MS (ESI): m/z=208.0 [M+H]⁺. [00213]¹H NMR (400 MHz, CDCl₃) δ 7.27-7.23 (m, 2H), 6.99-6.96 (m, 2H), 5.58 (br s, 1H), 3.67-3.63 (m, 1H), 3.55-3.49 (m, 1H), 2.23-2.20 (m, 1H), 2.13-2.09 (m, 2H).

Step 3: Compound 20C (400 mg, 1.93 mmol) was dissolved in 20 mL of tetrahydrofuran, lithium tetrahydroaluminate (220 mg, 5.79 mmol) was added in portions at 0° C., and the mixture was then warmed to room temperature and stirred for 16 h. 0.3 mL of water, 0.3 mL of 15% sodium hydroxide aqueous solution, and 0.9 mL of water were added, stirred for 1 h, and then suction-filtered to remove solids. The obtained filtrate was spin-dried to obtain compound 20D (310 mg, 83.10%), which was directly used in the next reaction.

¹H NMR (400 MHz, CDCl₃) δ 7.22-7.19 (m, 2H), 6.99-6.95 (m, 2H), 3.12 (d, 2H), 2.99 (d, 2H), 1.69-1.64 (m, 3H).

Step 4: Compound 1E (200 mg, 0.70 mmol) and compound 20D (150 mg, 0.78 mmol) were dissolved in 1,4-dioxane (8 mL), and DIPEA (270 mg, 2.09 mmol) was then added. After the addition was complete, the mixture was warmed to 100° C. under nitrogen protection and stirred for 16 h. After the reaction was complete, the reaction liquid was concentrated under reduced pressure to obtain a crude product, which was purified by column chromatography (eluents: DCM:MeOH=100:1 to 9:1) and then further purified by prep.HPLC (preparation method: instrument: waters 2767 preparative liquid chromatographic instrument; chromatographic column: SunFire@Prep C18 (19 mm×250 mm; the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 20-85%; c. flow rate: 15 mL/min; d. elution time: 15 min; retention time: 6.0 min) to obtain the target compound 20 (120 mg, 38.80%).

LCMS m/z=445.1 [M+1]⁺.

¹H NMR (400 MHz, CDCl₃) δ 7.22 (d, 2H), 6.95 (d, 2H), 5.86 (s, 1H), 4.88 (s, 1H), 4.11-4.04 (m, 1H), 3.97-3.91 (m, 1H), 3.86 (s, 2H), 3.68-3.59 (m, 3H), 3.45-3.38 (m, 1H), 3.07-2.98 (m, 2H), 2.37-2.25 (m, 4H), 1.99-1.85 (m, 4H), 1.69-1.62 (m, 1H).

### Example 16

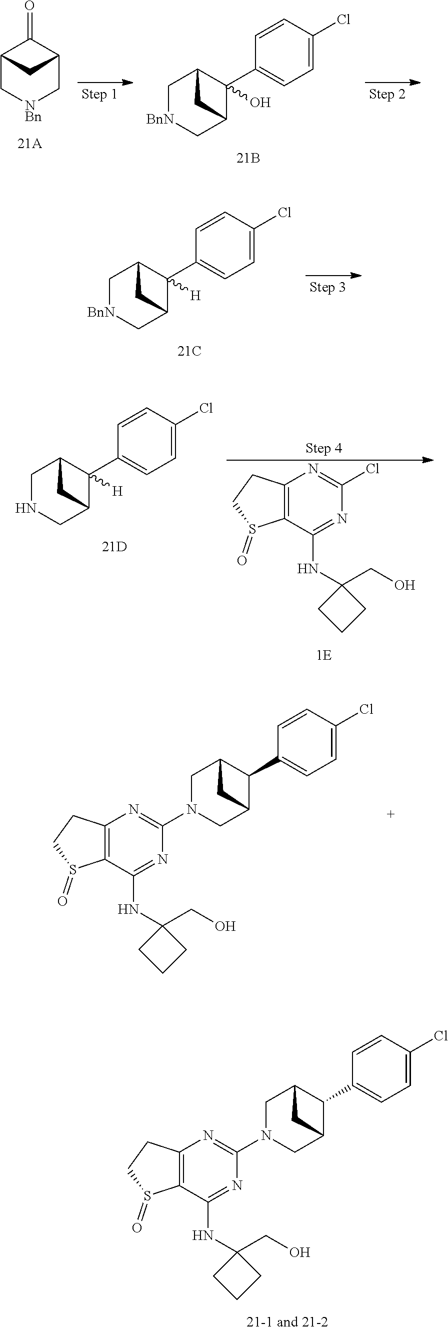

Step 1: Under nitrogen protection, (1R,5S)-3-benzyl-3-azabicyclo[3.1.1]heptan-6-one (3.00 g, 14.9 mmol) was dissolved in dry tetrahydrofuran (80 mL), and the mixture was cooled to 0° C. A solution of 4-chlorophenylmagnesium bromide in tetrahydrofuran (22.4 mL, 22.4 mmol) was dropwise added, and after the addition was complete, the mixture was reacted at 0° C. for about 1 hour. After the completion of the reaction was monitored by TLC, a saturated ammonium chloride aqueous solution (80 mL) was added. After the mixture was warmed to room temperature, the aqueous phase was separated from the organic phases. The aqueous phase was extracted with ethyl acetate (50 mL×6). The organic phases were combined, dried over anhydrous sodium sulfate, filtered, and concentrated, and the obtained residue was then separated and purified by silica gel column chromatography (petroleum ether:ethyl acetate (v/v)=5:1) to obtain compound 21B (3.69 g, 78.8%).

LC-MS (ESI): m/z=314.2 [M+H]⁺.

Step 2: Compound 21B (3.00 g, 9.58 mmol) was dissolved in 1,2-dichloroethane (80 mL), and boron trifluoride diethyl ether complex (4.08 g, 28.8 mmol) and triethylsilane (5.57 g, 47.9 mmol) were added at room temperature. After the addition was complete, the mixture was warmed to 80° C. and reacted overnight. After the completion of the reaction was monitored by TLC, the reaction product was cooled to room temperature, a saturated sodium bicarbonate aqueous solution (80 mL) and water (80 mL) were added, the aqueous phase was separated from the organic phase, and the aqueous phase was extracted with ethyl acetate (50 mL×4). The organic phases were combined, dried over anhydrous sodium sulfate, filtered, and concentrated, and the obtained residue was then separated and purified by silica gel column chromatography (petroleum ether:ethyl acetate (v/v)=10:1) to obtain compound 21C (1.38 g, 48.5%).

LC-MS (ESI): m/z=298.1 [M+H]⁺.

Step 3: Compound 21C (700 mg, 2.35 mmol) was added to a microwave reaction tube and dissolved with dry 1,2-dichloroethane (12 mL), and 1-chloroethyl chloroformate (1.68 g, 11.7 mmol) was then added. After the addition was complete, the mixture was placed in a microwave reactor and warmed to 140° C. for a reaction for 100 minutes. After the reaction was complete, the reaction liquid was transferred to a round-bottomed flask, suction-dried, re-dissolved by adding methanol (30 mL), and reacted at 70° C. for about 3 hours. The reaction liquid was concentrated again and separated and purified by silica gel column chromatography (dichloromethane:methanol (v/v)=10:1 to 5:1) to obtain compound 21D (280 mg, 57.4%).

LC-MS (ESI): m/z=208.0 [M+H]⁺.

Step 4: 1E (300 mg, 1.04 mmol), compound 21D (238 mg, 1.15 mmol), and N,N-diisopropylethylamine (673 mg, 5.21 mmol) were dissolved in 1,4-dioxane (15 mL), and the mixture was warmed to 100° C. and reacted for about 3 hours. After the completion of the reaction was monitored by TLC, the reaction product was cooled to room temperature, directly concentrated, and separated and purified by silica gel column chromatography (dichloromethane:methanol (v/v)=10:1) to obtain compound 21.

LC-MS (ESI): m/z=459.2 [M+H]⁺.

After further chiral preparation, compound 21-1 (80.5 mg, 16.8%) and compound 21-2 (87.4 mg, 18.3%) were obtained. Chiral preparation method: instrument: Waters 150 MGM; chromatographic column: Chiralpak Column; mobile phase: A: carbon dioxide, and B: methanol (0.1% aqueous ammonia); isocratic elution: 35% mobile phase B; flow rate: 100 mL/min; back pressure: 100 bar; column temperature: 25° C.; wavelength: 220 nm; elution time: 2.9 min.

Compound 21-1, retention time: 1.78 min, ¹H NMR (400 MHz, Methanol-d4) δ 7.26 (d, 2H), 7.06 (d, 2H), 3.93 (d, 2H), 3.81 (q, 1H), 3.70-3.49 (m, 3H), 3.42-3.29 (m, 2H), 3.22-3.10 (m, 2H), 3.09-2.96 (m, 2H), 2.58 (q, 1H), 2.44-2.16 (m, 3H), 2.13-1.99 (m, 2H), 1.94-1.67 (m, 3H);

Compound 21-2, retention time: 1.94 min, ¹H NMR (400 MHz, Methanol-d4) δ 7.26 (d, 2H), 7.06 (d, 2H), 4.02-3.87 (m, 2H), 3.81 (q, 1H), 3.75-3.47 (m, 3H), 3.42-3.29 (m, 2H), 3.21-2.94 (m, 4H), 2.58 (q, 1H), 2.44-2.16 (m, 3H), 2.13-1.98 (m, 2H), 1.95-1.68 (m, 3H).

### Example 17

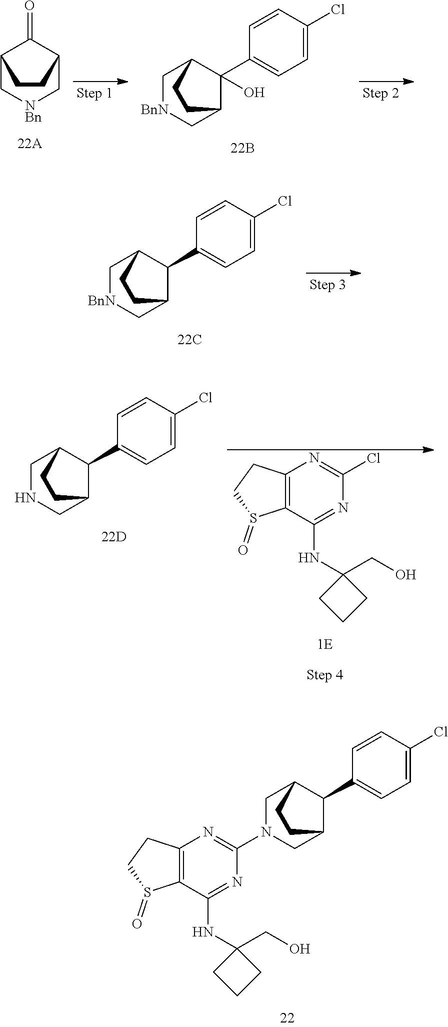

Step 1: Under nitrogen protection, 22A (2.0 g, 9.29 mmol) was dissolved in dry tetrahydrofuran (80 mL), and the mixture was cooled to 0° C. A solution of 4-chlorophenylmagnesium bromide in tetrahydrofuran (18.6 mL, 18.6 mmol) was dropwise added, and after the addition was complete, the mixture was reacted at 0° C. for about 1 hour. After the completion of the reaction was monitored by TLC, a saturated ammonium chloride aqueous solution (80 mL) was added, and after the mixture was warmed to room temperature, the aqueous phase was separated from the organic phase. The aqueous phase was extracted with ethyl acetate (50 mL×6). The organic phases were combined, dried over anhydrous sodium sulfate, filtered, and concentrated, and the obtained residue was then separated and purified by silica gel column chromatography (petroleum ether:ethyl acetate (v/v)=5:1) to obtain compound 22B (2.89 g, 94.8%).

LC-MS (ESI): m/z=328.1 [M+H]⁺.

Step 2: Compound 22B (710 mg, 2.16 mmol) was dissolved in 1,2-dichloroethane (30 mL), and boron trifluoride diethyl ether complex (920 mg, 6.48 mmol) and triethylsilane (1.26 g, 10.8 mmol) were added at room temperature. After the addition was complete, the mixture was warmed to 80° C. and reacted for 3 hours. After the completion of the reaction was monitored by TLC, the reaction product was cooled to room temperature, and a saturated sodium bicarbonate aqueous solution (30 mL) and water (30 mL) were added. The aqueous phase was separated from the organic phase, and the aqueous phase was extracted with ethyl acetate (30 mL×4). The organic phases were combined, dried over anhydrous sodium sulfate, filtered, and concentrated, and the obtained residue was then separated and purified by silica gel column chromatography (petroleum ether:ethyl acetate (v/v)=10:1) to obtain compound 22C (560 mg, 75.6%).

LC-MS (ESI): m/z=312.1 [M+H]⁺.

Step 3: Compound 22C (560 mg, 1.80 mmol) was added to a microwave reaction tube and dissolved with dry 1,2-dichloroethane (12 mL). 1-Chloroethyl chloroformate (1.28 g, 8.98 mmol) was then added, and after the addition was complete, the mixture was placed in a microwave reactor and warmed to 140° C. for a reaction for 100 minutes. After the reaction was complete, the reaction liquid was transferred to a round-bottomed flask, suction-dried, re-dissolved by adding methanol (30 mL), and reacted at 70° C. for about 3 hours. The reaction liquid was concentrated again and separated and purified by silica gel column chromatography (dichloromethane:methanol (v/v)=10:1 to 5:1) to obtain 22D (340 mg, 85.4%).

LC-MS (ESI): m/z=222.1 [M+H]⁺.

Step 4: 1E (300 mg, 1.04 mmol), compound 22D (255 mg, 1.15 mmol), and N,N-diisopropylethylamine (673 mg, 5.21 mmol) were dissolved in 1,4-dioxane (15 mL), and the mixture was warmed to 100° C. and reacted for about 3 hours. After the completion of the reaction was monitored by TLC, the reaction liquid was cooled to room temperature, directly concentrated, and preliminarily separated and purified by silica gel column chromatography (dichloromethane: methanol (v/v)=10:1) to obtain 22, and after further HPLC preparation and purification, compound 22 (450 mg, 91.1%) was obtained.

HPLC preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 100 mL/min; back pressure: 100 bar; column temperature: 25° C.; wavelength: 220 nm; elution time: 2.9 min. Retention time: 1.81 min. [00238]¹H NMR (400 MHz, Methanol-d4) δ 7.44-7.20 (m, 4H), 4.26 (d, 2H), 3.91 (dd, 2H), 3.52 (dt, 1H), 3.38-3.27 (m, 1H, overlapped), 3.20-3.07 (m, 3H), 3.07-2.97 (m, 2H), 2.85-2.76 (m, 2H), 2.38-2.21 (m, 4H), 2.03-1.83 (m, 4H), 1.74-1.61 (m, 2H).

LC-MS (ESI): m/z=473.5 [M+H]⁺.

### Example 18

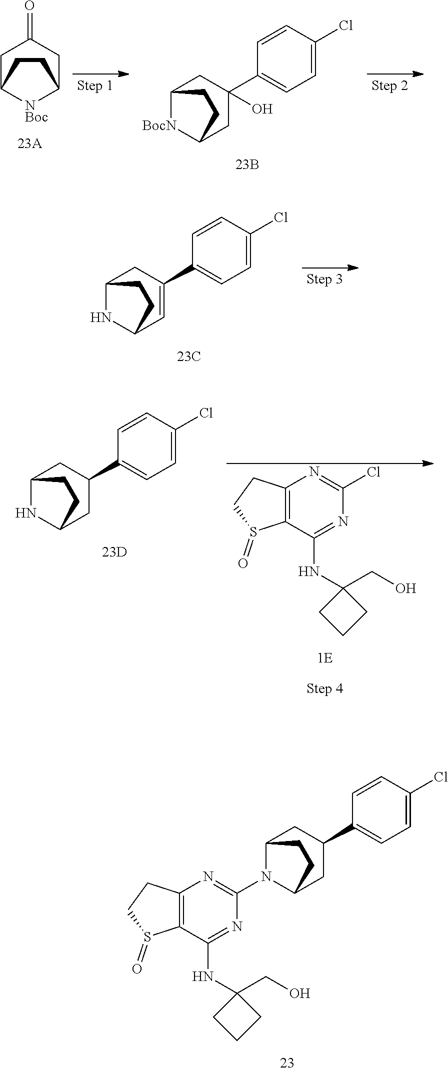

Step 1: Under nitrogen protection, p-chloroiodobenzene (6.19 g, 26.0 mmol) was dissolved in dry tetrahydrofuran (150 mL), the mixture was cooled to −78° C., and n-butyl lithium in n-hexane solution (10.4 mL, 26.0 mmol) was then dropwise added. After the addition was complete, the reaction continued for about 1 hour. Furthermore, 23A (4.5 g, 20.0 mmol) in dry tetrahydrofuran (80 mL) was dropwise added, and after the addition was complete, the mixture was warmed to −10° C. and stirred for 2 hours. After the completion of the reaction was monitored by TLC, a saturated ammonium chloride aqueous solution (200 mL) was added, and the mixture was warmed to room temperature, after which the aqueous phase was separated from the organic phase. The aqueous phase was extracted with ethyl acetate (50 mL×6). The organic phases were combined, dried over anhydrous sodium sulfate, filtered, and concentrated, and the obtained residue was then separated and purified by silica gel column chromatography (petroleum ether:ethyl acetate (v/v)=5:1) to obtain compound 23B (5.70 g, 84.4%).

LC-MS (ESI): m/z=338.2 [M+H]⁺.

Step 2: Compound 23B (3.00 g, 15.1 mmol) was dissolved in dichloromethane (50 mL), and trifluoroacetic acid (10 mL) was added at room temperature. After the addition was complete, the mixture was reacted for another 1 hour. After the completion of the reaction was monitored by TLC, the reaction product was directly concentrated to obtain 23C trifluoroacetate, and the crude product was directly brought to the next reaction without further purification.

LC-MS (ESI): m/z=220.1 [M+H]⁺.

Step 3: The crude compound 23C trifluoroacetate (4.10 g) was added to a round-bottomed flask and dissolved by adding methanol (80 mL), and a palladium on carbon catalyst (615 mg, 0.580 mmol) was then added. A hydrogen balloon was inserted, and the mixture was maintained at room temperature and reacted for 20 minutes. After the disappearance of raw materials was monitored by TLC, the reaction liquid was directly filtered and washed with methanol. The obtained filtrate was suction-dried under reduced pressure and then directly sent to preparative liquid phase purification to obtain compound 23D trifluoroacetate (312 mg, two-step yield 10.5%).

Preparative HPLC separation purification method: 1. Instrument: waters 2767 preparative liquid chromatographic instrument; chromatographic column: SunFire@Prep C18 (19 mm×250 mm). 2.

The sample was filtered with a 0.45 m filter to prepare a sample liquid. 3. Preparative chromatography conditions: a. composition of mobile phases A and B: mobile phase A: acetonitrile; mobile phase B: water (containing 0.5% trifluoroacetic acid). b. gradient elution, mobile phase A: 5-50%; c. flow rate: 12 mL/min; d. elution time: 10 min,

LC-MS (ESI): m/z=222.1 [M+H]⁺.

Step 4: 1E (266 mg, 0.923 mmol), compound 23D trifluoroacetate (310 mg, 0.923 mmol), and N,N-diisopropylethylamine (595 mg, 4.62 mmol) were dissolved in 1,4-dioxane (15 mL), and the mixture was warmed to 100° C. and reacted for about 3 hours. After the completion of the reaction was monitored by TLC, the reaction liquid was cooled to room temperature, directly concentrated, and preliminarily separated and purified by silica gel column chromatography (dichloromethane: methanol (v/v)=10:1), and after concentration followed by HPLC preparation and purification, compound 23 (252 mg, 57.7%) was obtained.

HPLC preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 100 mL/min; back pressure: 100 bar; column temperature: 25° C.; wavelength: 220 nm; elution time: 2.9 min. Retention time: 1.62 min. [00249]¹H NMR (400 MHz, Methanol-d4) δ 7.23 (q, 4H), 4.81-4.76 (m, 1H), 4.75-4.68 (m, 1H), 4.01-3.87 (m, 2H), 3.66-3.53 (m, 1H), 3.44-3.33 (m, 1H), 3.16-3.02 (m, 2H), 2.57-2.43 (m, 3H), 2.43-2.23 (m, 4H), 2.13-2.00 (m, 2H), 2.01-1.84 (m, 2H), 1.79 (d, 2H), 1.75-1.61 (m, 2H).

LC-MS (ESI): m/z=473.3 [M+H]⁺.

### Example 19

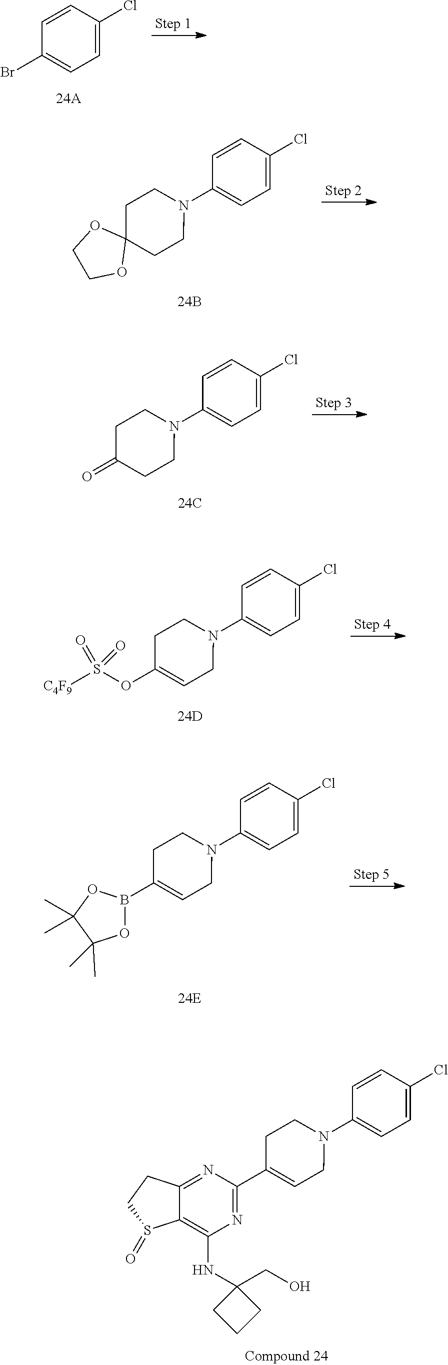

Step 1: 1-Bromo-4-chlorobenzene (15.0 g, 78.3 mmol), 1,4-dioxa-8-azaspiro[4.5]decane (11.8 g, 82.2 mmol), 1,1′-binaphthyl-2,2′-bisphenylphosphine (2.9 g, 4.7 mmol), and tris[dibenzylideneacetone]dipalladium (2.2 g, 2.4 mmol) were dissolved in 150 mL of dioxane, the mixture was then stirred at room temperature for 15 min, and sodium tert-butoxide (0.75 g, 7.8 mmol) was added. The system was warmed to 80° C. and reacted for 2 h. The reaction system was poured into ice water and extracted twice with ethyl acetate, the organic phases were combined and dried, and the solvent was removed by rotary evaporation. A mobile phase of PE:EA=20:1 was used for purification by silica gel column chromatography to obtain the target compound 24B (3.5 g, yield 19%).

LCMS (ESI): m/z=254.2 [M+H]⁺.

Step 2: The raw material 24B (3.5 g, 13.8 mmol) was dissolved in 30 mL of tetrahydrofuran, and 120 mL of 10% sulfuric acid was added, heated to 90° C., and reacted under reflux for 12 h. After the reaction was confirmed to be complete by TLC plate spotting, the mixture was cooled to room temperature and then adjusted to neutral pH with saturated sodium bicarbonate. After washing with 100 mL×3 of ethyl acetate, the organic phases were washed with a saturated sodium chloride aqueous solution and dried over anhydrous sodium sulfate. The solvent was removed by rotary evaporation to obtain 24C (2.2 g, yield 76%), which was directly used in the next reaction.

LCMS (ESI): m/z=210.1 [M+H]⁺.

Step 3: The raw material 24C (2.2 g, 10.5 mmol) and 1,1,2,2,3,3,4,4,4-nonafluorobutane-1-sulfonyl fluoride (3.82 g, 12.6 mmol) were dissolved in 25 mL of tetrahydrofuran, and DBU (1.92 g, 12.6 mmol) was added and stirred for 4 h at room temperature. After the completion of the reaction was monitored by TLC, the reaction product was concentrated and then purified by silica gel column chromatography with the mobile phase system PE:EA=50:1 to obtain 24D (3.9 g, yield 75%).

LCMS (ESI): m/z=492.1 [M+H]⁺.

Step 4: The raw material 24D (3.9 g, 8.0 mmol), pinacol diboronate (2.42 g, 9.6 mmol), [1,1′-bis(diphenylphosphino)ferrocene]palladium dichloride (173 mg, 0.24 mmol), and 1,1′-bis(diphenylphosphino)ferrocene (132 mg, 0.24 mmol) were suspended in dioxane (100 mL) and reacted at room temperature under stirring for 15 min. Potassium acetate (2.4 g, 24.5 mmol) was added and reacted at 90° C. for 16 h. TLC, LCMS (ESI): After the completion of the reaction was monitored, sample mixing was directly carried out for silica gel column chromatography with PE:EA =20:1 as the mobile phase to obtain the title compound 24E (0.9 g, yield 38%).

LCMS (ESI): m/z=320.1 [M+H]⁺.

Step 5: Compound 24E (900 mg, 2.8 mmol), XphosPd G2 (443 mg, 0.56 mmol), Xphos (538 mg, 1.12 mmol), and potassium phosphate (1.50 g, 7.0 mmol) were suspended in 10 mL of a mixed solvent of dioxane:water=4:1 and reacted at 90° C. for 4 h. After the reaction was complete, the reaction liquid was dropwise added to ice water and filtered. The filtrate was extracted with dichloromethane three times, the organic phases were combined with the filter residue, concentrated, and subjected to silica gel column chromatography with DCM:MeOH=15:1 as the mobile phase to obtain the target compound 24 (280 mg, 22%).

¹H NMR (400 MHz, DMSO-d6) δ 7.84-7.82 (s, 1H), 7.28-7.19 (m, 3H), 7.03-6.94 (m, 2H), 4.87-4.83 (m, 1H), 3.94-3.90 (m, 2H), 3.77-3.73 (m, 2H), 3.56 (m, 1H), 3.46-3.40 (m, 2H), 3.35-3.30 (m, 1H), 3.20-3.07 (m, 1H), 3.03-2.96 (m 1H), 2.68-2.64 (s, 2H), 2.44-2.18 (m, 4H), 1.88-1.72 (m, 2H).

LCMS (ESI): =445.1 [M+H]⁺.

### Example 20

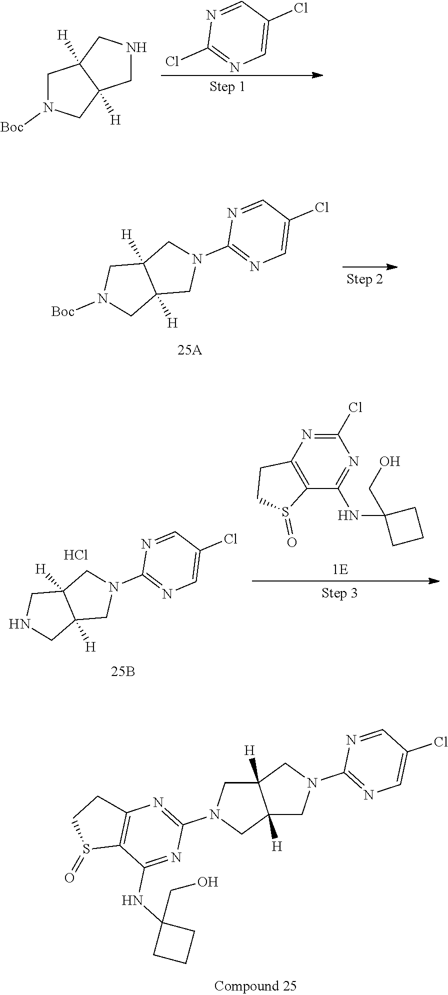

Step 1: Tert-butyl (3aR,6aS)-hexahydropyrrolo[3,4-c]pyrrole-2(1H)-carboxylate (2.12 g, 10 mmol), 2,5-dichloropyrimidine (1.64 g, 11 mmol), tris[dibenzylideneacetone]dipalladium (915 mg, 1 mmol), 2-dicyclohexylphosphine-2′,4′,6′-triisopropyl biphenyl (952 mg, 2 mmol), and sodium tert-butoxide (1.15 g, 12 mmol) were weighed into a 250 mL three-mouth flask, and toluene (50 mL) and tert-butyl alcohol (10 mL) were added. After nitrogen displacement, the reaction was carried out at 100° C. for 2 h. After the completion of the reaction was monitored by LCMS, the system was cooled to room temperature and filtered, and the filter cake was washed with ethyl acetate (50 mL). The filtrate was concentrated and then separated by silica gel column chromatography (PE:EA=10:1 to 5:1) to obtain the target compound 25A (2.86 g, 88.2%).

LC-MS (ESI): m/z=325.1 [M+H]⁺.

Step 2: 25A (2.86 g, 0.85 mmol) was dissolved in methanol (30 mL), and a solution of hydrogen chloride dioxane (5 mL, 4 M) was added. The mixture was reacted at room temperature for four hours and spin-dried to obtain title compound 25B (2.01 g, crude).

LC-MS (ESI): m/z=225.1 [M+H]⁺.

Step 3: 1E (287 mg, 1 mmol) and 25B (300 mg) were dissolved in 1,4-dioxane (20 mL), DIPEA (1 mL) was added, and the mixture was left at 90° C. overnight. After the complete reaction of raw materials was detected by LCMS, the system was concentrated, water (20 mL) was added, and the mixture was extracted with a mixed solution of DCM:MeOH=20:1 (20 mL×3). The organic phases were combined, dried over anhydrous sodium sulfate, concentrated, and then passed through a column (DCM:MeOH=1:0 to 0:1) to obtain compound 25 (262 mg, 55.1%).

¹H NMR (400 MHz, DMSO-d₆) δ 8.39 (s, 2H), 7.22 (s, 1H), 4.84-4.71 (m, 1H), 3.82-3.69 (m, 6H), 3.47-3.34 (m, 5H), 3.25-3.15 (m, 1H), 3.12-3.03 (m, 2H), 2.96-2.79 (m, 2H), 2.42-2.27 (m, 2H), 2.19-2.10 (m, 2H), 1.85-1.67 (m, 2H).

LC-MS (ESI): m/z=476.5 [M+H]⁺.

### Example 21

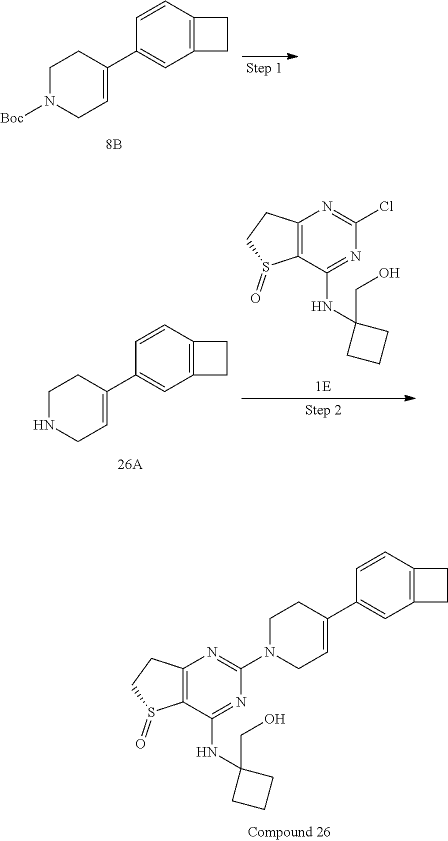 [SMILES: CC(C)(C)OC(=O)N1CC=C(c2ccc3c(c2)CC3)CC1 ; C1=C(c2ccc3c(c2)CC3)CCNC1 ; O=[S@@]1CCc2nc(Cl)nc(NC3(CO)CCC3)c21 ; O=[S@@]1CCc2nc(N3CC=C(c4ccc5c(c4)CC5)CC3)nc(NC3(CO)CCC3)c21]

Step 1: Compound 8B (0.50 g, 1.75 mmol) was weighed into a 100 mL single-mouth flask and then dissolved by adding methanol (10 mL), and a hydrogen chloride dioxane solution (4N, 10 mL) was added. After the addition was complete, the mixture was stirred for 2 h. The organic phase was concentrated under reduced pressure until no liquid dripped out to obtain the target compound 26A (0.40 g, yield: 100%).

LCMS m/z=186.1 [M+1]⁺.

Step 2: Compound 1E (0.20 g, 0.69 mmol), compound 26A (0.15 g, 0.83 mmol), and N,N-dimethylethylamine (0.27 g, 2.07 mmol) were added to a 100 mL single-mouth flask. After the addition was complete, the system was placed under nitrogen protection and stirred at 90° C. for 16 h, and the reaction liquid was concentrated under reduced pressure to obtain a crude product of the target compound, which was subjected to preparative HPLC to obtain the title compound 26 (170 mg, 56%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 40-80%; c. flow rate: 15 mL/min; d. elution time: 20 min; retention time: 11.25 min.

LCMS m/z=437.2 [M+1]⁺.

¹H NMR (400 MHz, DMSO-d6) δ7.31 (s, 1H), 7.25 (d, 1H), 7.16 (s, 1H), 7.04 (s, 1H), 6.13 (s, 1H), 4.82 (t, 1H), 4.33 (s, 2H), 3.96 (t, 2H), 3.74 (d, 2H), 3.46-3.38 (m, 1H), 3.30 (s, 2H), 3.18-3.25 (m, 1H), 3.12 (s, 4H), 2.83-2.98 (m, 2H), 2.30-2.42 (m, 2H), 2.15-2.23 (m, 2H), 1.75-1.85 (m, 2H).

### Example 22

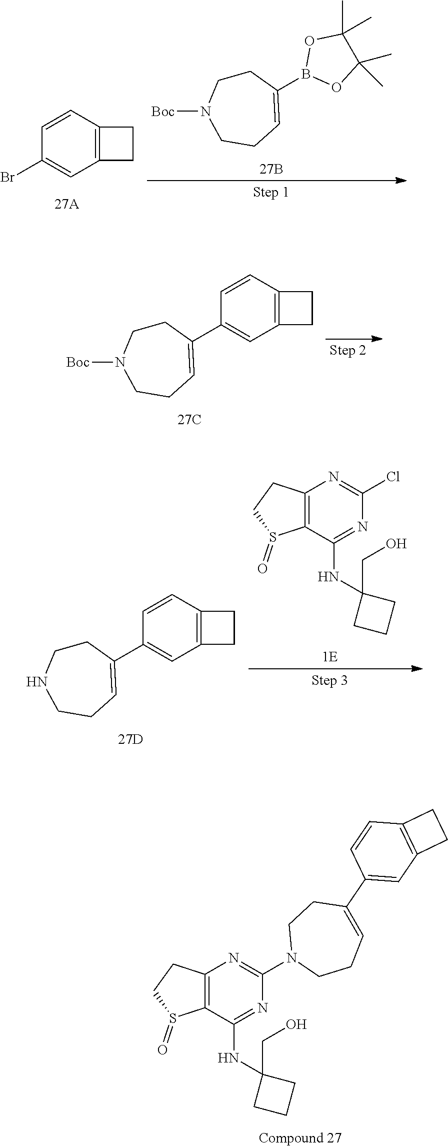

Step 1: Compound 27A (2.00 g, 10.91 mmol) and compound 27B (3.52 g, 10.91 mmol, referring to Patent WO 2021158829 for the synthesis method) were weighed into a 100 mL single-mouth flask and then dissolved by adding 1,4-dioxane (15 mL), and a sodium carbonate solution (2N, 15 mL) and [1,1′-bis(diphenylphosphino)ferrocene]palladium dichloride (0.71 g, 1.09 mmol) were added to the reaction liquid. After the addition was complete, the mixture was stirred at 100° C. for 4 h. After the completion of the reaction was monitored by TLC plate spotting (petroleum ether:ethyl acetate=5:1), water (20 mL) was added to the reaction liquid and stirred for 5 min, ethyl acetate (20 mL) was added for extraction, and an organic phase was separated. The organic phase was dried over anhydrous sodium sulfate, and the crude product was concentrated under reduced pressure and purified by column chromatography (eluents: petroleum ether:ethyl acetate=5:1) to obtain the target compound 27C (0.50 g, yield 15%).

LCMS m/z=300.1 [M+1]⁺.

Step 2: Compound 27C (0.50 g, 1.67 mmol) was weighed into a 100 mL single-mouth flask and then dissolved by adding methanol (10 mL), and a hydrogen chloride dioxane solution (4N, 10 mL) was added. After the addition was complete, the mixture was stirred for 2 h. The organic phase was concentrated under reduced pressure until no liquid dripped out to obtain the target compound 27D (0.40 g, yield: 100%).

LCMS m/z=200.2 [M+1]⁺.

Step 3: Compound 1E (0.20 g, 0.69 mmol), compound 27D (0.17 g, 0.83 mmol), and N,N-dimethylethylamine (0.27 g, 2.07 mmol) were added to a 100 mL single-mouth flask. After the addition was complete, the system was placed under nitrogen protection and stirred at 100° C. for 4 h, and the reaction liquid was concentrated under reduced pressure to obtain a crude product of the target compound, which was subjected to preparative HPLC to obtain the title compound 27 (12 mg, 3.9%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 40-80%; c. flow rate: 15 mL/min; d. elution time: 15 min; retention time: 9.0 min.

LCMS m/z=451.6 [M+1]⁺.

¹H NMR (400 MHz, DMSO-d6) δ7.24 (s, 1H), 7.11 (d, 1H), 6.97-7.00 (m, 2H), 5.84 (t, 1H), 5.82 (t, 1H), 3.77-4.03 (m, 4H), 3.71 (d, 2H), 3.37-3.45 (m, 1H), 3.17-3.25 (m, 1H), 3.09 (s, 4H), 2.83-2.96 (m, 2H), 3.72 (m, 2H), 2.29-2.47 (m, 4H), 2.12-2.18 (m, 2H), 1.69-1.81 (m, 2H).

### Example 23

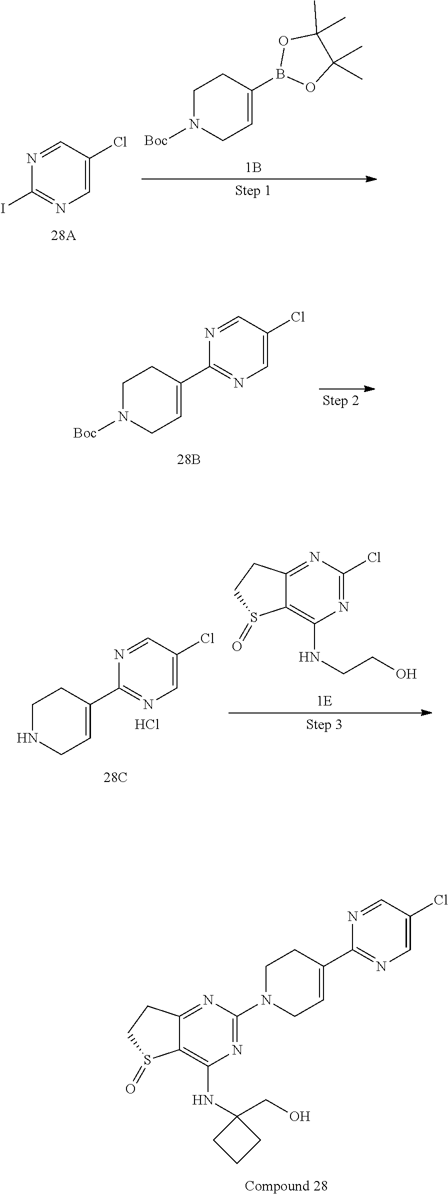

Step 1: Substrate 28A (5.0 g, 20.77 mmol) was added to a 250 mL single-mouth flask and dissolved with dioxane (100 mL) and water (20 mL), and sodium carbonate (6.6 g, 62.31 mmol), 1B (7.1 g, 22.85 mmol), and [1,1′-bis(diphenylphosphino)ferrocene]palladium dichloride (1.5 g, 2.08 mmol) were added and stirred at 100 degrees Celsius overnight under nitrogen protection. The reaction liquid was diluted by adding water (50 mL) and suction-filtered with diatomite, the filtrate was extracted with ethyl acetate (2×100 mL), and the organic phases were combined, dried over anhydrous sodium sulfate, concentrated, and subjected to column chromatography (petroleum ether: ethyl acetate (v/v)=10:1) to obtain the title compound 28B (5.0 g, 81%).

LC-MS (ESI): m/z=240.1 [M−56+H]⁺.

Step 2: Substrate 28B (1.0 g, 3.38 mmol) was added to a 50 mL single-mouth flask, dissolved with methanol (5 mL) and hydrogen chloride dioxane (4 M, 5 mL), and stirred at room temperature for 3 h, and the reaction liquid was concentrated to obtain the title compound 28C (0.79 g, 100%).

LC-MS (ESI): m/z=196.2 [M+H]⁺.

Step 3: Substrate 1E (0.20 g, 0.69 mmol) was added to a 50 mL single-mouth flask and dissolved with dioxane (10 mL), 28C (0.21 g, 0.92 mmol) and diisopropylethylamine (0.26 g, 2.05 mmol) were added and reacted at 90 degrees Celsius overnight, and the reaction liquid was concentrated, purified by HPLC, and freeze-dried to obtain the title compound 28 (130 mg, 42%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution: mobile phase A: 30-70%; c. flow rate: 15 mL/min; d. elution time: 15 min; retention time: 8.0 min.

¹H NMR (400 MHz, DMSO-d6) δ 8.89 (s, 2H), 7.34 (s, 1H), 7.31-7.26 (m, 1H), 4.83 (t, 1H), 4.46 (s, 2H), 3.97 (t, 2H), 3.75 (d, 2H), 3.45-3.41 (m, 1H), 3.24-3.21 (m, 1H), 2.98-2.83 (m, 2H), 2.65 (s, 2H), 2.43-2.28 (m, 2H), 2.25-2.14 (m, 2H), 1.90-1.73 (m, 2H).

LC-MS (ESI): m/z=447.1 [M+H]⁺.

### Example 24

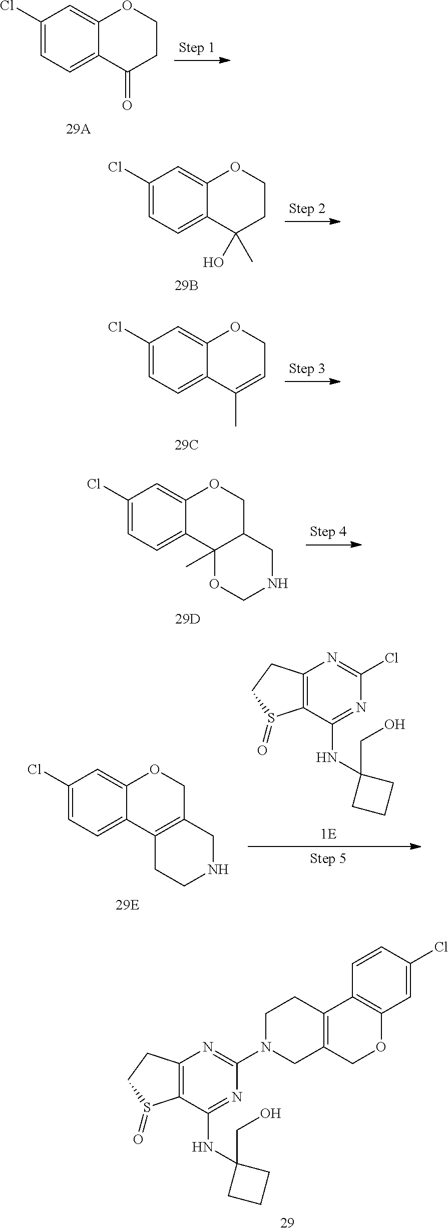

Step 1: Compound 29A (7.3 g, 40.0 mmol) was dissolved in tetrahydrofuran (100 mL), and a methyl magnesium chloride solution (20.0 mL, 3.0 M in THF, 60.0 mmol) was slowly added in ice bath. After the addition was complete, the mixture was slowly warmed to room temperature, reacted for 2 h, and quenched by adding a saturated ammonium chloride solution, and the reaction product was extracted with ethyl acetate (50.0 mL×3), dried, filtered, and concentrated to obtain compound 29B (8.0 g, 100%), which was directly used for the next reaction.

LC-MS (ESI): m/z=181.0 [M−OH]⁺.

Step 2: Compound 29B (8.0 g, 40.0 mmol) was dissolved in methanol (100 mL), p-toluenesulfonic acid (350.0 mg, 2.0 mmol) was then added, the mixture was heated to 70° C. and reacted for 2 h, and the reaction product was then cooled to room temperature, concentrated to remove the solvent, and subjected to column chromatography (petroleum ether/ethyl acetate=20/1) to obtain compound 29C (2.6 g, 36%).

LC-MS (ESI): m/z=181.0 [M+H]⁺.

Step 3: Compound 29C (1.8 g, 10.0 mmol) was dissolved in glacial acetic acid (3.5 mL), a formaldehyde aqueous solution (37% in H₂O, 43.0 mmol) and ammonium chloride (1.1 g, 20.0 mmol) were then added, and the mixture was heated to 100° C. and reacted for 18 h, then adjusted to alkalinity by adding a sodium hydroxide solution, and extracted with dichloromethane (50 mL×3). After drying and concentration, compound 29D (1.9 g, 80%) was obtained, which was used for the next step.

Step 4: Compound 29D (1.9 g, 8.0 mmol) was dissolved in concentrated hydrochloric acid (30.0 mL), and the mixture was heated to reflux and reacted for 3 h, then cooled to room temperature, adjusted to alkalinity by adding a sodium hydroxide solution, and extracted with ethyl acetate (50 mL×3). After drying, concentration, and column chromatography (dichloromethane/methanol=15/1), compound 29E (0.6 g, 27%) was obtained.

LC-MS (ESI): m/z=222.0 [M+H]⁺.

Step 5: Compound 29E (110.0 mg, 0.5 mmol) was dissolved in 1,4-dioxane (10.0 mL), compound 1E (140.0 mg, 0.5 mmol) was then added, and the mixture was heated back to 90° C. and reacted for 12 h, after which the reaction product was cooled to room temperature, concentrated, and subjected to column chromatography (dichloromethane/methanol=15/1) to obtain compound 29 (0.05 g, 21%).

LC-MS (ESI): m/z=473.1 [M+H]⁺.

¹H NMR (400 MHz, DMSO-d6) δ 7.39 (s, 1H), 7.14 (d, 1H), 6.98-6.95 (m, 1H), 6.88 (d, 1H), 4.85-4.82 (m, 1H), 4.74 (s, 2H), 4.20 (s, 2H), 4.01-3.98 (m, 2H), 3.74 (d, 2H), 3.50-3.35 (m, 1H), 3.26-3.18 (m, 1H), 2.97-2.84 (m, 2H), 2.46-2.26 (m, 4H), 2.19 (s, 2H), 1.91-1.70 (m, 2H).

### Example 25

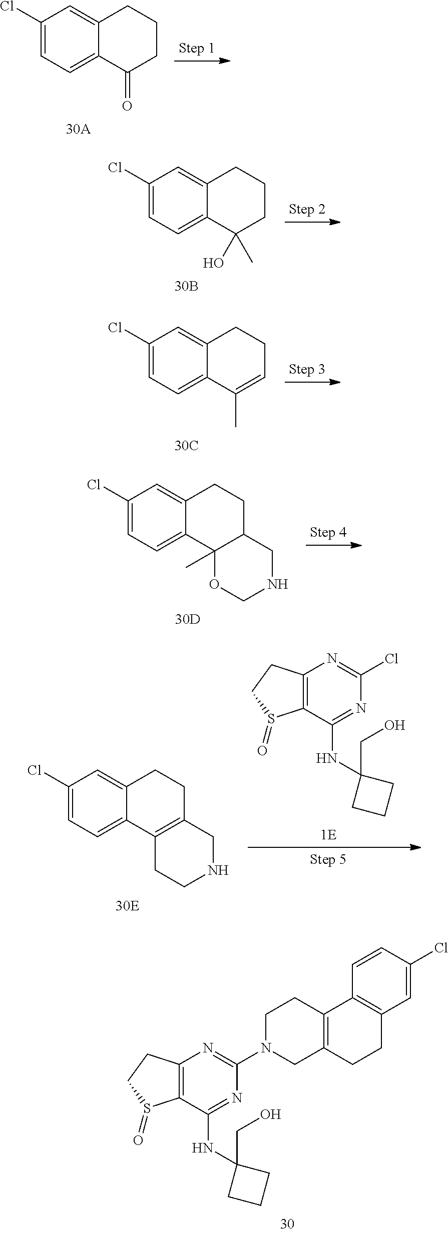

Step 1: Compound 30A (7.2 g, 40.0 mmol) was dissolved in tetrahydrofuran (100 mL), a methyl magnesium chloride solution (20.0 mL, 3.0 M in THF, 60.0 mmol) was slowly added in ice bath. After the addition was complete, the mixture was slowly warmed to room temperature, reacted for 2 h, and quenched by adding a saturated ammonium chloride solution, and the reaction product was extracted with ethyl acetate (50.0 mL×3), dried, filtered, and concentrated to obtain compound 30B (8.0 g, 99%), which was directly used for the next reaction.

LC-MS (ESI): m/z=179.0 [M−OH]⁺.

Step 2: Compound 30B (8.0 g, 40.0 mmol) was dissolved in methanol (100 mL), p-toluenesulfonic acid (350.0 mg, 2.0 mmol) was then added, and the mixture was heated to 70° C. and reacted for 2 h, after which the reaction product was then cooled to room temperature, concentrated to remove the solvent, and subjected to column chromatography (petroleum ether/ethyl acetate=20/1) to obtain compound 30C (5.6 g, 80%).

LC-MS (ESI): m/z=179.0 [M+H]⁺.

Step 3: Compound 30C (3.6 g, 20.0 mmol) was dissolved in glacial acetic acid (7.0 mL), a formaldehyde aqueous solution (37% in H₂O, 86.0 mmol) and ammonium chloride (2.2 g, 40.0 mmol) were then added, and the mixture was heated to 100° C. and reacted for 18 h, then adjusted to alkalinity by adding a sodium hydroxide solution, and extracted with dichloromethane (50 mL×3).

After drying and concentration, compound 30D (5.0 g, 89%) was obtained, which was used for the next step.

Step 4: Compound 30D (5.0 g, 21.0 mmol) was dissolved in concentrated hydrochloric acid (50.0 mL), and the mixture was heated to reflux and reacted for 3 h, then cooled to room temperature, adjusted to alkalinity by adding a sodium hydroxide solution, and extracted with ethyl acetate (50 mL×3). After drying, concentration, and column chromatography (dichloromethane/methanol=15/1), compound 30E (1.0 g, 22%) was obtained.

LC-MS (ESI): m/z=220.0 [M+H]⁺.

Step 5: Compound 30E (110.0 mg, 0.5 mmol) was dissolved in 1,4-dioxane (10.0 mL), compound 1E (140.0 mg, 0.5 mmol) was then added, and the mixture was heated back to 90° C. and reacted for 12 h, after which the reaction product was cooled to room temperature, concentrated, and subjected to column chromatography (dichloromethane/methanol=15/1) to obtain compound 30 (0.04 g, 19%).

LC-MS (ESI): m/z=471.1 [M+H]⁺.

¹H NMR (400 MHz, DMSO-d6) δ 7.33 (s, 1H), 7.22 (s, 2H), 7.20-7.13 (m, 1H), 4.85-4.83 (m, 1H), 4.27 (s, 2H), 3.99 (s, 2H), 3.75 (s, 2H), 3.45-3.38 (m, 1H), 3.27-3.16 (m, 1H), 2.97-2.83 (m, 2H), 2.79-2.75 (t, 2H), 2.45-2.34 (m, 4H), 2.20 (d, 4H), 1.79 (d, 2H).

### Example 26

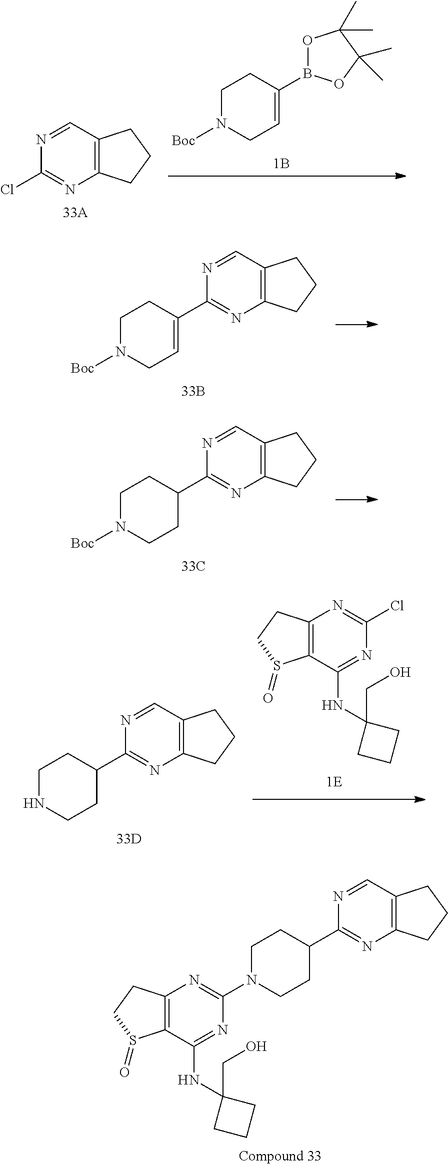

Step 1: Compound 33A (3.00 g, 19.40 mmol) and compound 1B (7.20 g, 23.28 mmol) were weighed into a 100 mL single-mouth flask and then dissolved by adding 1,4-dioxane (15 mL). A sodium carbonate solution (2N, 15 mL) and [1,1′-bis(diphenylphosphino)ferrocene]palladium dichloride (1.42 g, 1.94 mmol) were added to the reaction liquid. After the addition was complete, the mixture was stirred at 100° C. for 4 h. After the completion of the reaction was monitored by TLC plate spotting (petroleum ether:ethyl acetate=5:1), water (20 mL) was added to the reaction liquid and stirred for 5 min, ethyl acetate (20 mL) was added for extraction, and an organic phase was separated. The organic phase was dried over anhydrous sodium sulfate, and the crude product was concentrated under reduced pressure and purified by column chromatography (eluents: petroleum ether:ethyl acetate=5:1) to obtain the target compound 33B (4.0 g, yield 68%).

LCMS m/z=302.1 [M+1]⁺.

¹H NMR (400 MHz, CDCl₃) δ 8.46 (s, 1H), 7.10 (s, 1H), 4.16 (s, 2H), 3.62 (t, 2H), 2.92-3.00 (m, 4H), 2.73 (s, 2H), 2.11-2.19 (m, 2H), 1.49 (s, 9H).

Step 2: Compound 33B (1.00 g, 3.32 mmol) was weighed into a 100 mL single-mouth flask and then dissolved by adding ethyl acetate (20 mL), and palladium on carbon (0.18 g, 1.66 mmol) was added to the reaction liquid. After the addition was complete, the mixture was stirred for 2 h under hydrogen and atmospheric pressure conditions. After the reaction was complete, the reaction liquid was filtered with diatomite and the filtrate was concentrated under reduced pressure to obtain the target compound 33C (0.90 g, yield: 89%).

LCMS m/z=304.2 [M+1]⁺.

Step 3: Compound 33C (0.90 g, 2.97 mmol) was weighed into a 100 mL single-mouth flask and then dissolved by adding methanol (10 mL), and a hydrogen chloride dioxane solution (4N, 10 mL) was added. After the addition was complete, the mixture was stirred for 2 h. The organic phase was concentrated under reduced pressure to obtain the target compound 33D (0.70 g, crude).

LCMS m/z=204.1 [M+1]⁺.

Step 4: Compound 1E (0.20 g, 0.69 mmol), compound 33D (0.17 g, 0.83 mmol), and N,N-diisopropylethylamine (0.26 g, 2.05 mmol) were added to a 100 mL single-mouth flask. After the addition was complete, the system was placed under nitrogen protection and stirred at 90° C. for 16 h, and the reaction liquid was concentrated under reduced pressure to obtain a crude product of the target compound, which was subjected to preparative HPLC to obtain the title compound 33 (120 mg, 38%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 40-80%; c. flow rate: 15 mL/min; d. elution time: 20 min; retention time: 10.38 min.

LCMS m/z=455.2 [M+1]⁺.

¹H NMR (400 MHz, DMSO-d6) δ 8.50 (s, 1H), 7.29 (s, 1H), 4.84 (s, 1H), 4.71 (d, 2H), 3.72 (t, 2H), 3.37-3.45 (m, 1H), 3.18-3.25 (m, 1H), 2.94-3.15 (m, 4H), 2.82-2.89 (m, 5H), 2.27-2.38 (m, 2H), 2.12-2.19 (m, 2H), 2.00-2.11 (m, 2H), 1.86-1.93 (m, 2H), 1.61-1.80 (m, 4H).

### Example 27

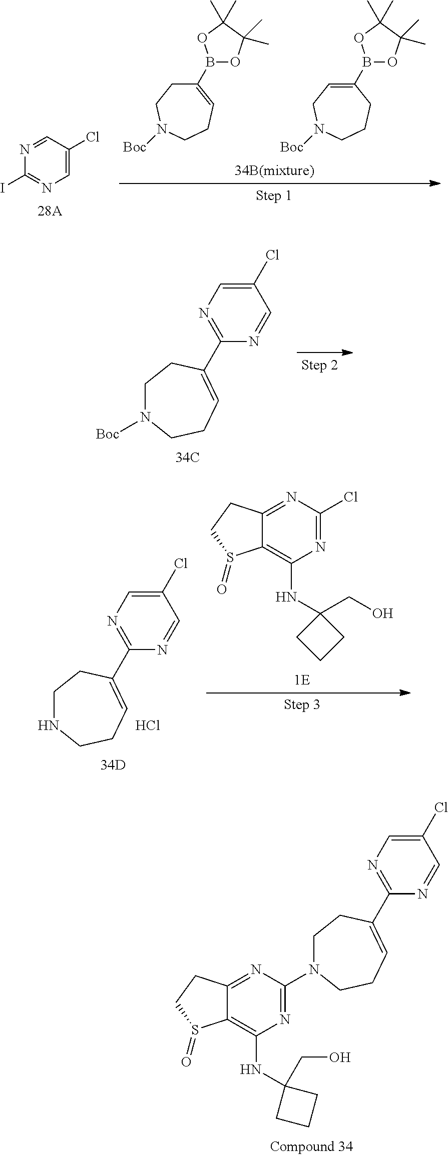

Step 1: Substrate 28A (1.1 g, 4.55 mmol) was added to a 100 mL single-mouth flask and dissolved with dioxane (20 mL) and water (5 mL). Sodium carbonate (1.5 g, 13.70 mmol), 34B (2.1 g, 4.55 mmol, containing 50% regioisomer, the synthesis method was according to Patent WO 2021158829), and [1,1′-bis(diphenylphosphino)ferrocene]palladium dichloride (0.33 g, 0.45 mmol) were added and stirred at 100 degrees Celsius overnight under nitrogen protection. The reaction liquid was diluted by adding water (50 mL) and suction-filtered with diatomite, and the filtrate was extracted with ethyl acetate (2×50 mL). The organic phases were combined, dried over anhydrous sodium sulfate, concentrated, and subjected to column chromatography (petroleum ether:ethyl acetate (v/v)=10:1) to obtain the title compound 34C (1.0 g, 71%, containing 50% regioisomer).

LC-MS (ESI): m/z=254.3 [M−56+H]⁺.

Step 2: Substrate 34C (1.0 g, 3.23 mmol) was added to a 50 mL single-mouth flask, dissolved with methanol (10 mL) and hydrogen chloride dioxane (4 M, 10 mL), and stirred at room temperature for 3 h, and the reaction liquid was concentrated to obtain the title compound 34D (0.80 g, 100%, containing 50% regioisomer).

LC-MS (ESI): m/z=210.2 [M+H]⁺.

Step 3: Substrate 1E (0.10 g, 0.35 mmol) was added to a 50 mL single-mouth flask and dissolved with dioxane (10 mL). 34D (0.11 g, 0.52 mmol) and diisopropylethylamine (0.14 g, 1.05 mmol) were added and reacted at 90 degrees Celsius overnight, and the reaction liquid was concentrated, purified by HPLC, and freeze-dried to obtain the title compound 34 (16 mg, 10%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 30-80%; c. flow rate: 15 mL/min; d. elution time: 15 min; retention time: 7.5 min.

¹H NMR (400 MHz, DMSO-d6) δ 8.84 (s, 2H), 7.39 (s, 1H), 7.24 (s, 1H), 4.83 (t, 1H), 3.93-3.88 (m, 4H), 3.73-3.72 (m, 2H), 3.23-3.20 (m, 1H), 3.06-2.79 (m, 5H), 2.60 (s, 2H), 2.42-2.25 (m, 2H), 2.17 (s, 2H), 1.79 (s, 2H).

LC-MS (ESI): m/z=461.2 [M+H]⁺.

### Example 28

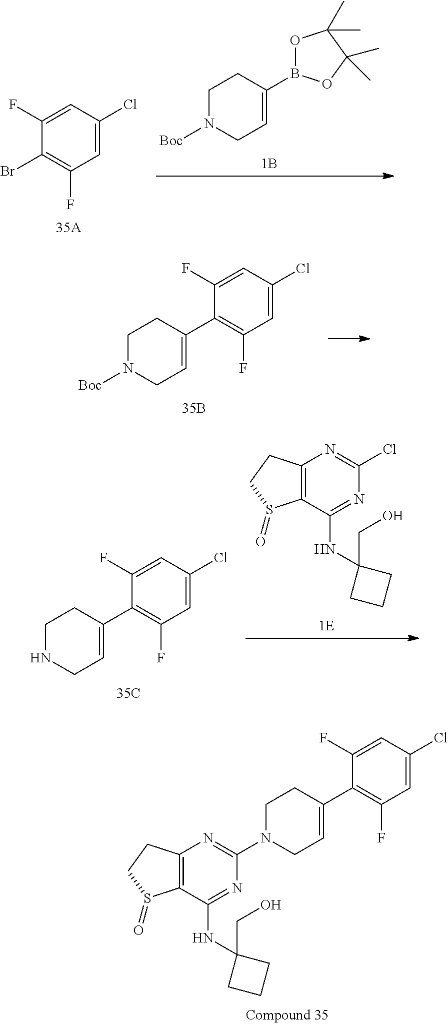

Step 1: Compound 35A (2.00 g, 8.79 mmol) and compound 1B (3.26 g, 10.55 mmol) were weighed into a 100 mL single-mouth flask and then dissolved by adding 1,4-dioxane (20 mL), and a sodium carbonate solution (2N, 20 mL) and [1,1′-bis(diphenylphosphino)ferrocene]palladium dichloride (0.64 g, 0.88 mmol) were added to the reaction liquid. After the addition was complete, the mixture was stirred at 100° C. for 4 h. After the completion of the reaction was monitored by TLC plate spotting (petroleum ether:ethyl acetate=5:1), water (20 mL) was added to the reaction liquid and stirred for 5 min, ethyl acetate (20 mL) was added for extraction, and an organic phase was separated. The organic phase was dried over anhydrous sodium sulfate, and the crude product was concentrated under reduced pressure and purified by column chromatography (eluents: petroleum ether:ethyl acetate=5:1) to obtain the target compound 35B (2.0 g, yield 69%).

LCMS m/z=330.1 [M+1]⁺.

¹H NMR (400 MHz, CDCl₃) δ 6.93 (s, 1H), 6.91 (s, 1H), 5.81 (s, 1H), 4.06 (d, 2H), 3.62 (t, 2H), 2.38 (s, 2H), 1.49 (s, 9H).

Step 2: Compound 35B (0.30 g, 1.02 mmol) was weighed into a 100 mL single-mouth flask and then dissolved by adding methanol (10 mL), and a hydrogen chloride dioxane solution (4N, 10 mL) was added. After the addition was complete, the mixture was stirred for 2 hours. The organic phase was concentrated under reduced pressure to obtain the target compound 35C (0.20 g, crude).

LCMS m/z=230.1 [M+1]⁺.

Step 3: Compound 1E (0.20 g, 0.69 mmol), compound 35C (0.19 g, 0.83 mmol), and N,N-diisopropylethylamine (0.26 g, 2.05 mmol) were added to a 100 mL single-mouth flask. After the addition was complete, the system was placed under nitrogen protection and stirred at 90° C. for 16 h, and the reaction liquid was concentrated under reduced pressure to obtain a crude product of the target compound, which was subjected to preparative HPLC to obtain the title compound 35 (220 mg, 66%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 40-80%; c. flow rate: 15 mL/min; d. elution time: 20 min; retention time: 11.20 min.

LCMS m/z=481.1 [M+1]⁺.

¹H NMR (400 MHz, DMSO-d6) δ 7.35-7.40 (m, 3H), 6.00 (s, 1H), 4.83 (t, 1H), 4.34 (s, 2H), 3.96 (t, 2H), 3.73 (d, 2H), 3.39-3.47 (m, 1H), 3.19-3.26 (m, 1H), 2.84-2.98 (m, 2H), 2.27-2.40 (m, 4H), 2.15-2.21 (m, 2H), 1.69-1.82 (m, 2H).

### Example 29

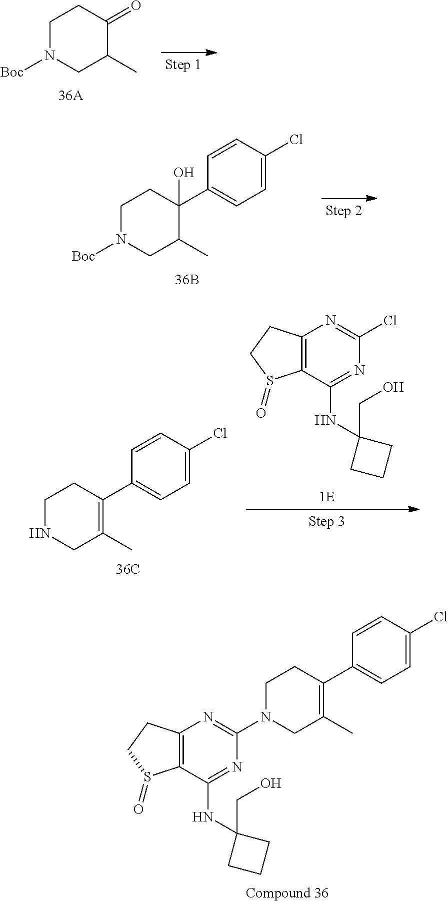

Step 1: Compound 36A (2.00 g, 9.39 mmol) was weighed into a 100 mL single-mouth flask and then dissolved by adding tetrahydrofuran (40 mL). 4-Chlorophenylmagnesium chloride (11.27 mL, 11.27 mmol) was added to the reaction liquid, and after the addition was complete, the mixture was stirred at room temperature for 4 h. After the reaction was found complete by TLC plate spotting (petroleum ether:ethyl acetate=3:1), water (40 mL) was added to the reaction liquid, and the mixture was stirred for 5 min and extracted by adding ethyl acetate (20 mL). The organic phase was separated and dried over anhydrous sodium sulfate, and the crude product was concentrated under reduced pressure and purified by column chromatography (eluents: petroleum ether:ethyl acetate=5:1) to obtain the target compound 36B (2.0 g, yield 65%).

LCMS m/z=326.1 [M+1]⁺.

Step 2: Compound 36B (0.30 g, 0.97 mmol) was weighed into a 100 mL single-mouth flask and then dissolved by adding methanol (10 mL). A hydrogen chloride dioxane solution (4N, 10 mL) was added, and after the addition was complete, the mixture was stirred for 2 h. The organic phase was concentrated under reduced pressure until no liquid dripped out to obtain the target compound 36C (0.20 g, crude).

LCMS m/z=208.1 [M+1]⁺.

Step 3: Compound 1E (0.20 g, 0.69 mmol), compound 36C (0.17 g, 0.83 mmol), and N,N-diisopropylethylamine (0.26 g, 2.05 mmol) were added to a 100 mL single-mouth flask. After the addition was complete, the system was placed under nitrogen protection and stirred at 90° C. for 16 h, and the reaction liquid was concentrated under reduced pressure and purified by preparative HPLC to obtain the title compound 36 (88 mg, 27%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM ammonium acetate); b. gradient elution, mobile phase A: 30-90%; c. flow rate: 15 mL/min; d. elution time: 20 min; retention time: 12.82 min.

LCMS m/z=459.2 [M+1]⁺.

¹H NMR (400 MHz, DMSO-d6) δ7.40 (d, 2H), 7.32 (s, 1H), 7.24 (d, 2H), 4.84 (t, 1H), 4.20 (s, 2H), 3.94 (t, 2H), 3.73 (d, 2H), 3.39-3.47 (m, 1H), 3.19-3.27 (m, 1H), 2.84-2.99 (m, 2H), 2.29-2.42 (m, 4H), 2.14-2.19 (m, 2H), 1.73-1.84 (m, 2H), 1.63 (s, 3H).

### Example 30

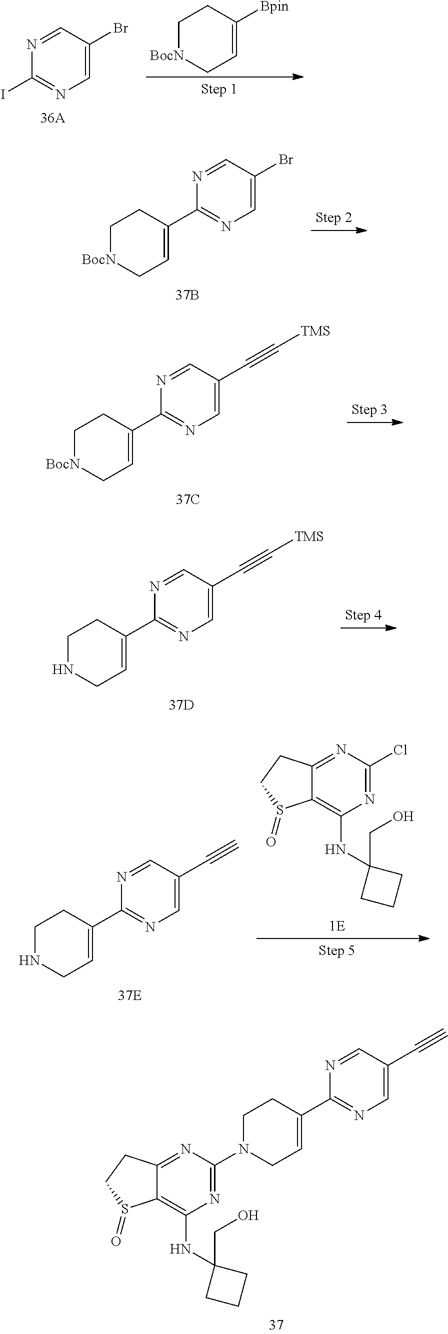

Step 1: Compound 37A (1.4 g, 5.0 mmol) was dissolved in 1,4-dioxane (30 mL), 1,1-bis(diphenylphosphino)ferrocene palladium dichloride (360.0 mg, 0.5 mmol), potassium carbonate (1.4 g, 10.0 mmol), N-Boc-1,2,5,6-tetrahydropyridine-4-boronic acid pinacol ester (1.8 g, 6.0 mmol), and water (6.0 mL) were then added. After nitrogen displacement three times, the mixture was heated to 45° C. and reacted for 14 h, after which the reaction product was passed through a short silica gel column, rinsed with ethyl acetate, concentrated, and subjected to column chromatography (dichloromethane/methanol=20/1) to obtain compound 37B (1.7 g, 99%).

LC-MS (ESI): m/z=284.1 [M−56]⁺.

Step 2: Compound 37B (340.0 mg, 1.0 mmol) was dissolved in tetrahydrofuran (10 mL). Bis(triphenylphosphino)palladium dichloride (70.0 mg, 0.1 mmol), triethylamine (202.0 mg, 2.0 mmol), trimethylsilylacetylene (130.0 mg, 1.3 mmol), and cuprous iodide (8.0 mg, 0.05 mmol) were then added. After nitrogen displacement three times, the mixture was heated to 50° C. and reacted for 14 h, after which the reaction product was passed through a short silica gel column, rinsed with ethyl acetate, concentrated, and subjected to column chromatography (dichloromethane/methanol=20/1) to obtain compound 37C (240.0 mg, 72%).

LC-MS (ESI): m/z=302.1 [M−56]⁺.

Step 3: Compound 37C (240.0 mg, 0.67 mmol) was dissolved in dichloromethane (10 mL), trifluoroacetic acid (1.0 mL) was then added, and the mixture was reacted at room temperature for half an hour and then concentrated to obtain compound 37D (150.0 mg, 87%), which was directly used in the next step.

LC-MS (ESI): m/z=258.1 [M+H]⁺.

Step 4: Compound 37D (150.0 mg, 0.58 mmol) was dissolved in methanol (10.0 mL), potassium carbonate (150.0 mg, 1.1 mmol) was then added, and the mixture was then reacted at room temperature for 2 h, concentrated, and then subjected to column chromatography (dichloromethane/methanol=15/1) to obtain compound 37E (70.0 mg, 65%).

LC-MS (ESI): m/z=186.1 [M+H]⁺.

Step 5: Compound 37E (70.0 mg, 0.38 mmol) was dissolved in 1,4-dioxane (10.0 mL), compound 1E (115.0 mg, 0.4 mmol) was then added, and the mixture was heated back to 90° C. and reacted for 12 h, after which the reaction product was cooled to room temperature, concentrated, and subjected to column chromatography (dichloromethane/methanol=10/1) to obtain compound 37 (20.0 mg, 12%).

LC-MS (ESI): m/z=437.1 [M+H]⁺.

¹H NMR (400 MHz, DMSO-d6) δ 8.90 (s, 2H), 7.36 (s, 1H), 7.34 (s, 1H), 4.84 (t, 1H), 4.68 (s, 1H), 4.48 (s, 2H), 3.97 (t, 2H), 3.75 (d, 2H), 3.51-3.36 (m, 1H), 3.28-3.18 (m, 1H), 2.98-2.84 (m, 2H), 2.66 (s, 2H), 2.43-2.30 (m, 2H), 2.24-2.17 (m, 2H), 1.86-1.71 (m, 2H).

### Example 31

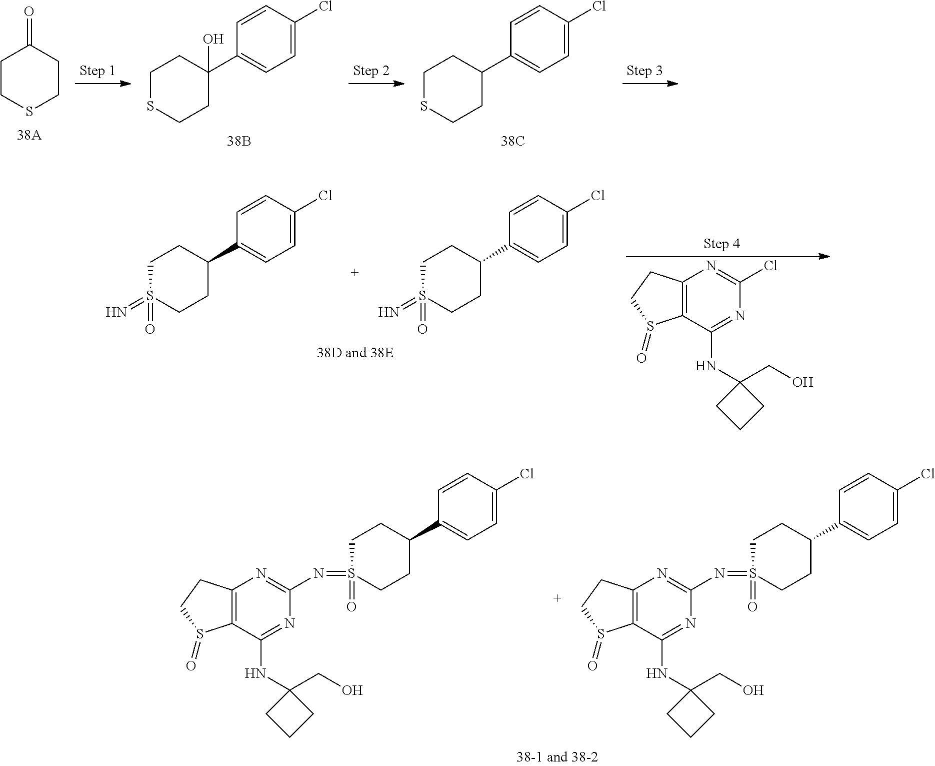

Step 1: Under nitrogen protection, 38 A (2.50 g, 21.5 mmol) was dissolved in dry tetrahydrofuran (80 mL), and the mixture was cooled to 0° C. A solution of 4-chlorophenylmagnesium bromide in tetrahydrofuran (32.3 mL, 32.3 mmol) was dropwise added, and after the addition was complete, the mixture was reacted at 0° C. for about 1 hour. After the completion of the reaction was monitored by TLC, a saturated ammonium chloride aqueous solution (100 mL) was added. After the mixture was warmed to room temperature, the aqueous phase was separated from the organic phase. The aqueous phase was extracted with ethyl acetate (50 mL×5). The organic phases were combined, dried over anhydrous sodium sulfate, filtered, and concentrated, and the obtained residue was then separated and purified by silica gel column chromatography (petroleum ether:ethyl acetate (v/v)=5:1) to obtain compound 38B (4.51 g, 91.7%).

LC-MS (ESI): m/z=229.1 [M+H]⁺.

Step 2: Compound 38B (4.00 g, 17.5 mmol) was dissolved in 1,2-dichloroethane (200 mL), and triethylsilane (10.2 g, 87.4 mmol) and trifluoroacetic acid (5.98 g, 52.5 mmol) were successively added at room temperature. After the addition was complete, the reaction continued overnight at room temperature. After the completion of the reaction was monitored by TLC, a saturated sodium bicarbonate aqueous solution (80 mL) and water (80 mL) were added, and the aqueous phase was separated from the organic phase. The aqueous phase was extracted with ethyl acetate (50 mL×4). The organic phases were combined, dried over anhydrous sodium sulfate, filtered, and concentrated, and the obtained residue was then preliminarily separated and purified by silica gel column chromatography (petroleum ether:ethyl acetate (v/v)=10:1) to obtain crude compound 38C. After further purification by preparative HPLC, compound 38C (950 mg, 25.5%) was obtained.

Preparative HPLC separation purification method: 1. Instrument: waters 2767 preparative liquid chromatographic instrument; chromatographic column: SunFire@Prep C18 (19 mm×250 mm). 2. The sample was filtered with a 0.45 m filter to prepare a sample liquid. 3. Preparative chromatography conditions: a. composition of mobile phases A and B: mobile phase A: acetonitrile; mobile phase B: water (containing 0.1% ammonium acetate); b. gradient elution, mobile phase A: 5-50%; c. flow rate: 12 mL/min; d. elution time: 30 min. Compound 38C, retention time: 17.2 min.

¹H NMR (400 MHz, Chloroform-d) δ 7.27 (d, 2H), 7.12 (d, 2H), 2.83 (ddd, 2H), 2.75-2.65 (m, 2H), 2.50 (tt, 1H), 2.11 (dq, 2H), 1.82 (qd, 2H).

Step 3: Compound 38C (600 mg, 2.82 mmol) and ammonium carbamate (418 mg, 5.36 mmol) were added to methanol (15 mL) and stirred until uniform, and iodobenzene diacetic acid (2.27 g, 7.05 mmol) was then added. After the addition was complete, the mixture was reacted overnight at room temperature in an air environment. After the reaction was complete, a saturated ammonium chloride aqueous solution (50 mL) was added, and the aqueous phase was extracted with ethyl acetate (30 mL×6). The organic phases were combined, dried over anhydrous sodium sulfate, filtered, and concentrated, and the obtained residue was then separated and purified by silica gel column chromatography (petroleum ether:ethyl acetate (v/v)=1:2) to obtain compound 38D (250 mg, 36.4%) and 38E (240 mg, 34.9%).

LC-MS (ESI): m/z=244.1 [M+H]⁺.

Step 4: Under nitrogen protection, compound 38D (150 mg, 0.615 mmol), (R)-2-chloro-4-(1-(hydroxymethyl)cyclobutyl)amino)-6,7-dihydrothieno[3,2-d]pyrimidine 5-oxide (230 mg, 0.800 mmol), palladium acetate (27.6 mg, 0.123 mmol), 2,2′-bis(diphenylphosphino)-1,1′-binaphthyl (153 mg, 0.246 mmol), and potassium phosphate (327 mg, 1.54 mmol) were dissolved in toluene (10 mL), warmed to 110° C., and reacted for about 5 hours. After the completion of the reaction was monitored by TLC, the reaction product was cooled to room temperature, directly concentrated, and preliminarily separated and purified by silica gel column chromatography (dichloromethane: methanol (v/v)=10:1), and after preparative HPLC purification, compound 38-1 (95 mg, 31.2%) was obtained.

Preparative HPLC separation purification method: 1. Instrument: waters 2767 preparative liquid chromatographic instrument; chromatographic column: SunFire@Prep C18 (19 mm×250 mm). 2.

The sample was filtered with a 0.45 m filter to prepare a sample liquid. 3. Preparative chromatography conditions: a. composition of mobile phases A and B: mobile phase A: methanol; mobile phase B: water (containing 0.1% ammonium acetate); b. gradient elution, mobile phase A: 5-50%; c. flow rate: 12 mL/min; d. elution time: 30 min.

Compound 38-1, retention time: 20.2 min, ¹H NMR (400 MHz, DMSO-d6) δ 7.42-7.32 (m, 3H), 7.29-7.21 (m, 2H), 4.88 (t, 1H), 4.00-3.87 (m, 2H), 3.77-3.63 (m, 2H), 3.58-3.39 (m, 3H), 3.30-3.22 (m, 1H), 3.12-2.83 (m, 3H), 2.40-2.17 (m, 4H), 2.16-2.00 (m, 4H), 1.87-1.67 (m, 2H).

LC-MS (ESI): m/z=495.1 [M+H]⁺;
referring to the above synthesis method, compound 38-2 (107 mg, 35.1%) was synthesized from compound 38E (150 mg, 0.615 mmol) as a raw material.

Compound 38-2, retention time: 16.3 min, ¹H NMR (400 MHz, DMSO-d6) δ 7.45-7.36 (m, 2H), 7.36-7.26 (m, 3H), 4.90 (t, 1H), 3.89-3.75 (m, 2H), 3.75-3.63 (m, 2H), 3.58-3.37 (m, 3H), 3.29-3.22 (m, 1H), 3.10-2.83 (m, 3H), 2.39-2.24 (m, 2H), 2.24-2.09 (m, 6H), 1.86-1.65 (m, 2H).

LC-MS (ESI): m/z=495.1 [M+H]⁺.

### Example 32

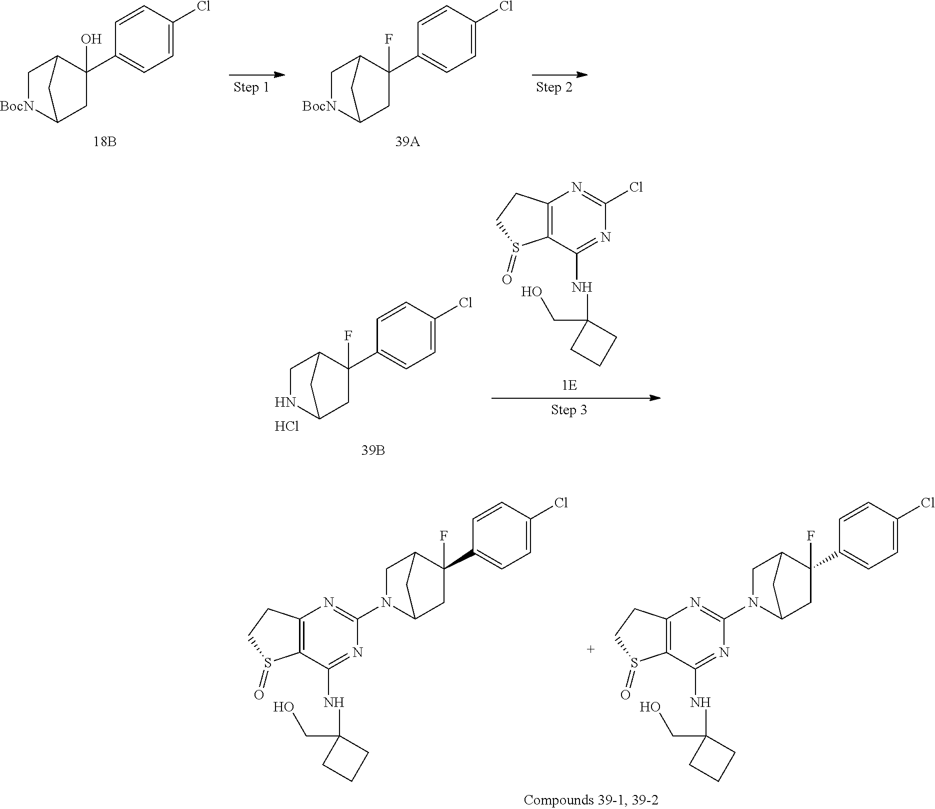

Step 1: 18B (500 mg, 1.55 mmol) and dichloromethane (20 mL) were added to a 100 mL three mouth flask. After nitrogen displacement, bis(2-methoxyethyl)aminosulfur trifluoride (685 mg, 3.1 mmoL) was dropwise added at −78° C., and the mixture was naturally warmed to room temperature and reacted for 1 h. A saturated sodium bicarbonate aqueous solution (50 mL) and dichloromethane (50 mL×2) were added. The organic phases were combined, washed with a saturated sodium chloride solution, dried over anhydrous sodium sulfate, and filtered, and the filtrate was concentrated under reduced pressure and then separated and purified by silica gel column chromatography (eluent EA/PE=0-20%) to obtain the target compound (0.5 g, 99%).

LC-MS (ESI): m/z=270.1 [M−56+H]⁺.

Step 2: 39A (0.5 g, 1.54 mmol) and hydrogen chloride-dioxane (4 M, 20 mL) were successively added to a 50 mL single-mouth flask. The mixture was reacted at room temperature for 1 h. The reaction liquid was directly concentrated under reduced pressure to obtain the title compound (400 mg, 100%).

LC-MS (ESI): m/z=226.1 [M+H]⁺.

Step 3: 1E (287 mg, 1 mmol), 39B (261 mg, 1 mmol), dioxane (5 mL), and DIPEA (645 mg, 5 mmol) were successively added to a 50 mL single-mouth flask and reacted at 100° C. for 8 h. The reaction product was cooled to room temperature, and the reaction liquid was directly concentrated under reduced pressure and then separated and purified by silica gel column chromatography (eluent MeOH/DCM=0-10%) to obtain 400 mg of a mixture.

After chiral preparation, the title compound 39-1 (160 mg, 33%, retention time: 1.311 min) and the title compound 39-2 (101 mg, 21%, retention time: 1.501 min) were obtained. Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 100 mL/min; back pressure: 100 bar; column temperature: 25° C.; wavelength: 220 nm; elution time: 2.0 min.

### Compound 39-1:

¹H NMR (400 MHz, DMSO-d6) δ7.49-7.41 (m, 2H), 7.38-7.25 (m, 2H), 4.91-4.79 (m, 2H), 3.80-3.69 (m, 1H), 3.66-3.53 (m, 1H), 3.47-3.31 (m, 2H), 3.30-3.05 (m, 3H), 3.02-2.93 (m, 1H), 2.88-2.77 (m, 2H), 2.49-2.14 (m, 6H), 2.08-1.89 (m, 2H), 1.86-1.52 (m, 2H).

LC-MS (ESI): m/z=477.1 [M+H]⁺.

### Compound 39-2:

¹H NMR (400 MHz, DMSO-d6) δ 7.48-7.46 (m, 1H), 7.38-7.32 (m, 2H), 7.20-7.17 (m, 1H), 7.07-7.04 (m, 1H), 4.84-4.71 (m, 1H), 3.81-3.71 (m, 1H), 3.61-3.09 (m, 5H), 3.08-2.65 (m, 4H), 2.49-2.11 (m, 6H), 2.05-1.90 (m, 2H), 1.88-1.70 (m, 1H), 1.71-1.45 (m, 1H).

LC-MS (ESI): m/z=477.1 [M+H]⁺.

### Example 33

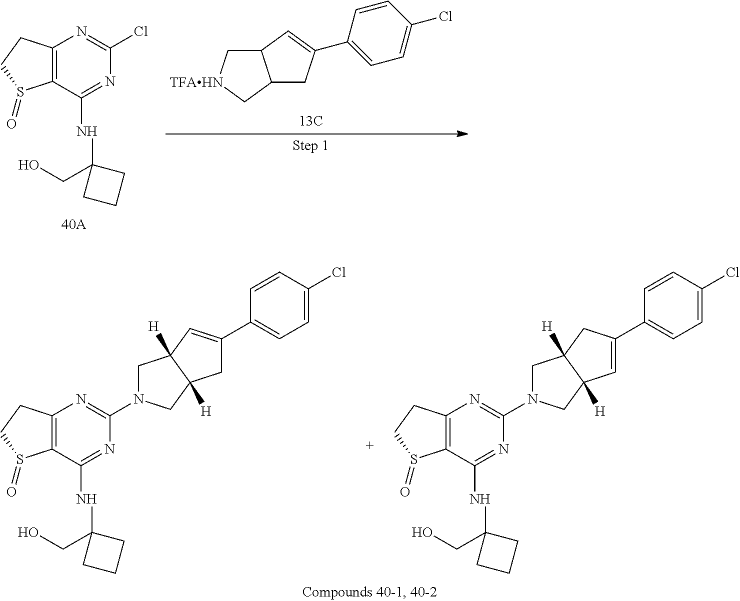

Step 1: 40A (0.15 g, 0.52 mmol), 13C (0.21 g, 0.62 mmol), dioxane (5 mL), and DIPEA (0.34 g, 2.59 mmol) were successively added to a 50 mL single-mouth flask and reacted at 100° C. for 5 h. The reaction product was cooled to room temperature, and the reaction liquid was directly concentrated under reduced pressure and then separated and purified by silica gel column chromatography (eluent MeOH/DCM=0-10%) to obtain 220 mg of a mixture.

After chiral preparation, the title compound 40-1 (42 mg, 17%, retention time: 0.786 min) and the title compound 40-2 (82 mg, 33%, retention time: 0.993 min) were obtained. Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 120 mL/min; back pressure: 100 bar; column temperature: 25° C.; wavelength: 220 nm; elution time: 5.5 min.

### Compound 40-1:

¹H NMR (400 MHz, DMSO-d6) δ 7.50-7.48 (m, 2H), 7.39-7.37 (m, 2H), 7.18 (s, 1H), 6.26 (s, 1H), 4.82 (s, 1H), 3.87 (s, 1H), 3.79-3.54 (m, 5H), 3.44-3.34 (m, 1H), 3.22-3.12 (m, 2H), 3.12-3.05 (m, 1H), 2.98-2.77 (m, 3H), 2.62 (d, 1H), 2.40-2.29 (s, 2H), 2.15 (s, 2H), 1.85-1.70 (m, 2H).

LC-MS (ESI): m/z=471.2 [M+H]⁺.

### Compound 40-2:

¹H NMR (400 MHz, DMSO-d6) δ 7.50-7.48 (m, 2H), 7.39-7.37 (m, 2H), 7.18 (s, 1H), 6.26 (s, 1H), 4.82 (s, 1H), 3.90-3.83 (m, 1H), 3.79-3.54 (m, 5H), 3.46-3.32 (m, 1H), 3.24-3.13 (m, 2H), 3.12-3.05 (m, 1H), 2.97-2.78 (m, 3H), 2.65-2.58 (m, 1H), 2.43-2.28 (m, 2H), 2.14 (s, 2H), 1.85-1.68 (m, 2H).

LC-MS (ESI): m/z=471.2 [M+H]⁺.

### Example 34

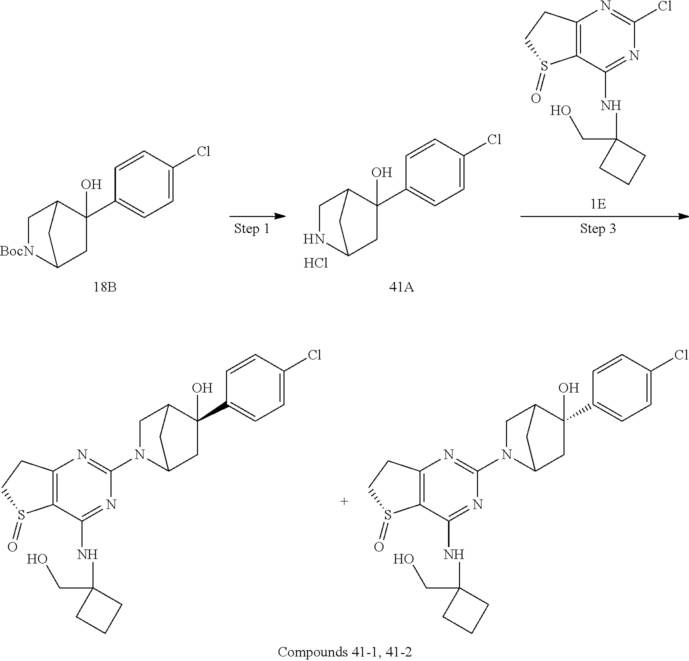 [SMILES: CC(C)(C)OC(=O)N1CC2CC1CC2(O)c1ccc(Cl)cc1 ; Cl ; OC1(c2ccc(Cl)cc2)CC2CC1CN2 ; O=[S@@]1CCc2nc(Cl)nc(NC3(CO)CCC3)c21 ; O=[S@@]1CCc2nc(N3CC4CC3C[C@]4(O)c3ccc(Cl)cc3)nc(NC3(CO)CCC3)c21 ; O=[S@@]1CCc2nc(N3CC4CC3C[C@@]4(O)c3ccc(Cl)cc3)nc(NC3(CO)CCC3)c21]

Step 1: 18B (0.5 g, 1.55 mmol) and hydrogen chloride-dioxane (4 M, 20 mL) were successively added to a 50 mL single-mouth flask. The mixture was reacted at room temperature for 1 h. The reaction liquid was directly concentrated under reduced pressure to obtain the title compound (400 mg, 100%).

LC-MS (ESI): m/z=224.1 [M+H]⁺.

Step 2: 1E (287 mg, 1.0 mmol), 41A (233 mg, 1.0 mmol), dioxane (5 mL), and DIPEA (645 mg, 5.0 mmol) were successively added to a 50 mL single-mouth flask and reacted at 100° C. for 8 h. The reaction product was cooled to room temperature, and the reaction liquid was directly concentrated under reduced pressure and then separated and purified by silica gel column chromatography (eluent MeOH/DCM=0-10%) to obtain 410 mg of a mixture.

After chiral preparation, the title compound 41-1 (150 mg, 32%, retention time: 1.302 min) and the title compound 41-2 (100 mg, 21%, retention time: 1.488 min) were obtained. Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 100 mL/min; back pressure: 100 bar; column temperature: 25° C.; wavelength: 220 nm; elution time: 2.0 min.

### Compound 41-1:

¹H NMR (400 MHz, DMSO-d6) δ 7.53 (d, 2H), 7.42-7.33 (m, 2H), 7.20 (s, 1H), 5.43 (d, 1H), 4.87 (s, 1H), 4.55 (d, 1H), 4.12-3.97 (m, 1H), 3.90-3.62 (m, 2H), 3.46-3.38 (m, 1H), 3.30-3.16 (m, 2H), 3.01-2.81 (m, 2H), 2.79-2.67 (m, 1H), 2.46-2.07 (m, 5H), 1.87-1.54 (m, 5H).

LC-MS (ESI): m/z=475.2 [M+H]⁺.

### Compound 41-2:

¹H NMR (400 MHz, DMSO-d6) δ 7.55 (d, 2H), 7.43-7.38 (m, 2H), 5.60 (d, 2H), 4.71-4.48 (m, 1H), 4.05 (d, 1H), 3.99-2.93 (m, 8H), 2.84 (d, 1H), 2.44-2.13 (m, 5H), 1.91-1.67 (m, 5H).

LC-MS (ESI): m/z=475.2 [M+H]⁺.

### Example 35

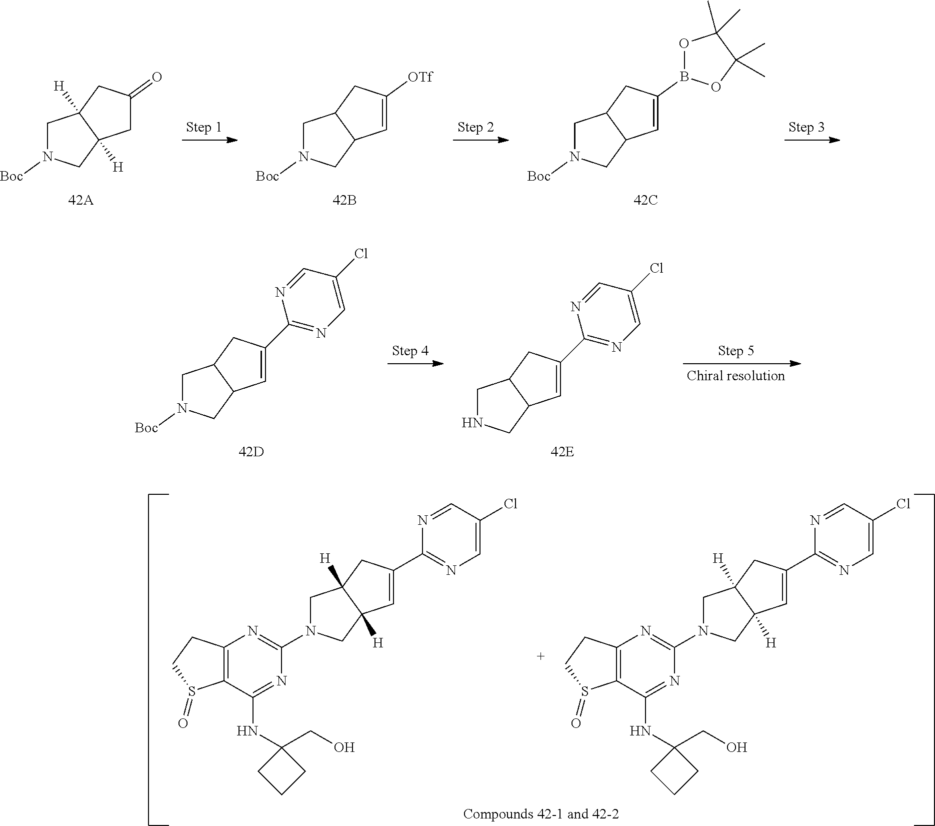

Step 1: Compound 42A (1 g, 4.44 mmol) was dissolved in anhydrous tetrahydrofuran (10 ml). After nitrogen displacement three times, the reaction system was cooled to −78° C. Lithium bis(trimethylsilyl)amide (8.88 mmol) was dropwise added, and the mixture was maintained at −78° C. for 30 min. N-phenyl bis(trifluoromethyl)sulfonimide (2.59 g, 6.66 mmol) dissolved in anhydrous tetrahydrofuran (5 ml) was added dropwise. The mixture was maintained at −78° C. for another 30 min, and then reacted at room temperature for 2 h. The reaction was complete and then quenched by adding a saturated ammonium chloride solution (30 ml). The organic phase was extracted with ethyl acetate (3×40 ml). The organic phases were combined, dried over anhydrous sodium sulfate, filtered and concentrated, and the residue was quickly separated and purified by column chromatography (eluent ratio: EA/PE=0-10%) to obtain compound 42B (1.3 g, 82%).

LCMS (ESI): m/z=302.2 [M+H−56].

Step 2: Under nitrogen, bis(triphenylphosphino)palladium dichloride (260 mg, 0.37 mmol), pinacol diboronate (1.11 g, 4.37 mmol), and potassium acetate (1.07 g, 10.92 mmol) were added to compound 42B (1.3 g, 3.64 mmol) dissolved in 1,4-dioxane (20 ml), and the mixture was reacted for 18 h in an oil bath at 90° C. After the reaction was complete, the reaction product was cooled to room temperature, filtered with diatomite, and washed with ethyl acetate, and the filtrate was concentrated and directly quickly separated and purified by direct column chromatography (eluent ratio: EA/PE=0-10%) to obtain the target compound 42C (0.89 g, 73%).

LCMS (ESI): m/z=280.2 [M+H−56].

Step 3: Under nitrogen, 5-chloro-2-iodopyrimidine (578 mg, 2.41 mmol), 1,1-bis(diphenylphosphino)ferrocene palladium dichloride (176 mg, 0.24 mmol), and sodium carbonate (766 mg, 7.32 mmol) were added to compound 42C (0.89 g, 2.65 mmol) dissolved in 1,4-dioxane/water (10 ml/2 ml), and the mixture was reacted for 18 h in an oil bath at 90° C. After the reaction was complete, the reaction product was cooled to room temperature, filtered with diatomite, and washed with ethyl acetate, and the filtrate was concentrated and directly quickly separated and purified by direct column chromatography (eluent ratio: EA/PE=0-10%) to obtain the target compound 42D (0.44 g, 52%).

LCMS (ESI): m/z=266.1 [M+H−56].

Step 4: Compound 42D (220 mg, 0.69 mmol) was dissolved in hydrogen chloride 1,4-dioxane (2 ml), stirred at room temperature for 1 h, and directly spin-dried to obtain compound 42E hydrochloride (170 mg).

LCMS (ESI): m/z=222.1 [M+H]⁺.

Step 5: Intermediate 1E (198 mg, 0.69 mmol) was dissolved in 1,4-dioxane (5 ml), 42E (170 mg, 0.69 mmol) and diisopropylethylamine (267 mg, 2.07 mmol) were added, and the reaction system was placed in an oil bath at 100° C. and reacted for 15 h. After the reaction was complete, the reaction product was cooled to room temperature. The reaction was monitored by LCMS. The reaction product was spin-dried and then subjected to preparation. Preparative HPLC separation method: 1.

Instrument: waters 2767 preparative liquid chromatographic instrument; chromatographic column: SunFire@Prep C18 (19 mm×250 mm). 2. The sample was filtered with a 0.45 m filter to prepare a sample liquid. 3. Preparative chromatography conditions: a. composition of mobile phases A and B: mobile phase A: acetonitrile; mobile phase B: water (containing 0.1% ammonium acetate); b. gradient elution, mobile phase A: 10-55%; c. flow rate: 12 mL/min. Retention time: 7.0 min. Thus, compound 42 (240 mg, 74%) was obtained.

LCMS (ESI): m/z=473.2 [M+H]⁺.

### Resolution of Compound 42:

Compound 42 (240 mg) was taken for resolution, and after separation, compound 42-1 (retention time: 1.257 min, 74 mg, ee %=100%) and compound 42-2 (retention time: 1.553 min, 77 mg, ee %=100%) were obtained.

Resolution conditions: instrument: Waters 150 MGM; column: DAICEL CHIRALPAK AD; mobile phases: A for CO2 and B for EtOH (0.1% NH3·H2O)); gradient: B 40%; flow rate: 120 mL/min; back pressure: 100 bar; column temperature: 35° C.; wavelength: 220 nm; period: 13 min;

### Compound 42-1:

¹H NMR (400 MHz, CDCl₃) δ=8.61 (s, 2H), 6.85 (s, 1H), 5.91 (s, 1H), 5.11 (s, 1H), 3.99 (d, 1H), 3.84 (s, 2H), 3.78 (s, 1H), 3.70 (d, 1H), 3.64-3.51 (m, 1H), 3.39 (d, 1H), 3.30 (d, 1H), 3.24-3.12 (m, 1H), 3.11-2.91 (m, 3H), 2.85 (d, 1H), 2.45-2.12 (m, 4H), 1.91 (d, 2H), 1.67 (s, 1H).

### Compound 42-2:

¹H NMR (400 MHz, CDCl₃) δ=8.62 (s, 2H), 6.85 (s, 1H), 5.88 (s, 1H), 5.10 (d, 1H), 4.10-3.92 (m, 1H), 3.81 (d, 3H), 3.68 (s, 1H), 3.58 (d, 1H), 3.43-3.28 (m, 2H), 3.17 (s, 1H), 3.12-2.92 (m, 3H), 2.85 (d, 1H), 2.29 (t, 4H), 1.91 (d, 2H), 1.69 (d, 1H).

### Example 36

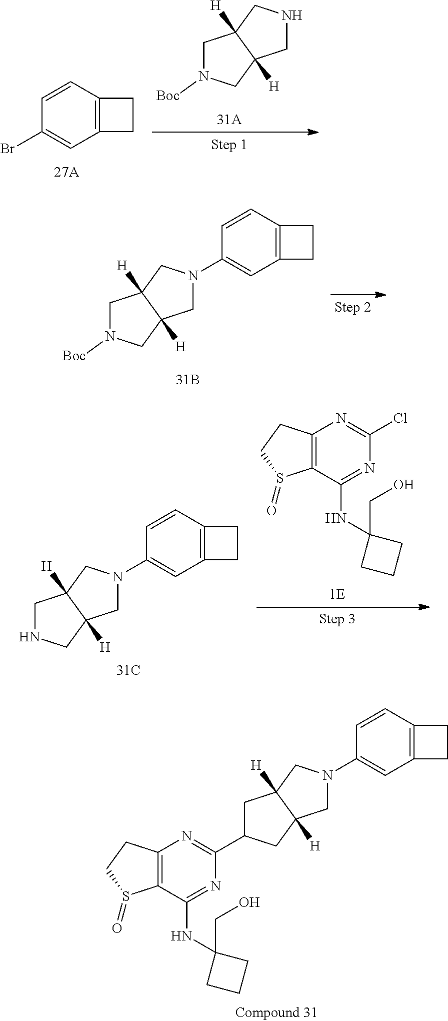

Step 1: Compound 27A (1.00 g, 5.45 mmol) and compound 31A (1.16 g, 5.45 mmol) were weighed into a 100 mL single-mouth flask and then dissolved by adding 1,4-dioxane (30 mL). Sodium tert-butoxide (1.57 g, 16.34 mmol), tris(dibenzylideneacetone)dipalladium (0.15 g, 0.27 mmol), and 2,2′-bis(diphenylphosphino)-1,1′-binaphthyl (0.34 g, 0.55 mmol) were added to the reaction liquid. After the addition was complete, the mixture was stirred at 90° C. for 16 h. After the completion of the reaction was monitored by TLC plate spotting (petroleum ether:ethyl acetate=5:1), water (20 mL) was added to the reaction liquid and stirred for 5 min, ethyl acetate (20 mL) was added for extraction, and an organic phase was separated. The organic phase was dried over anhydrous sodium sulfate, and the crude product was concentrated under reduced pressure and purified by column chromatography (eluents: petroleum ether:ethyl acetate=5:1) to obtain the target compound 31B (1.0 g, yield 58%).

LCMS m/z=315.1 [M+1]⁺.

Step 2: Compound 31B (0.30 g, 0.95 mmol) was weighed into a 100 mL single-mouth flask and then dissolved by adding methanol (10 mL), and a hydrogen chloride dioxane solution (4N, 10 mL) was added. After the addition was complete, the mixture was stirred for 2 h. The organic phase was concentrated under reduced pressure until no liquid dripped out to obtain the target compound 31C (0.20 g, crude).

LCMS m/z=215.1 [M+1]⁺.

Step 3: Compound 1E (0.20 g, 0.69 mmol), compound 31C (0.15 g, 0.69 mmol), and N,N-diisopropylethylamine (0.26 g, 2.05 mmol) were added to a 100 mL single-mouth flask. After the addition was complete, the system was placed under nitrogen protection and stirred at 90° C. for 16 h, and the reaction liquid was concentrated under reduced pressure to obtain a crude product of the target compound, which was subjected to preparative HPLC to obtain the title compound 31 (120 mg, 37%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 40-80%; c. flow rate: 15 mL/min; d. elution time: 20 min; retention time: 10.20 min.

LCMS m/z=466.2 [M+1]⁺.

¹H NMR (400 MHz, DMSO-d6) δ 7.21 (s, 1H), 6.85 (d, 1H), 6.34-6.37 (m, 2H), 4.81 (t, 1H), 3.75-3.80 (m, 2H), 3.72 (d, 2H), 3.35-3.45 (m, 5H), 2.97-3.24 (m, 9H), 2.81-2.94 (m, 2H), 2.29-2.42 (m, 2H), 2.11-2.17 (m, 2H), 1.69-1.81 (m, 2H).

### Example 37

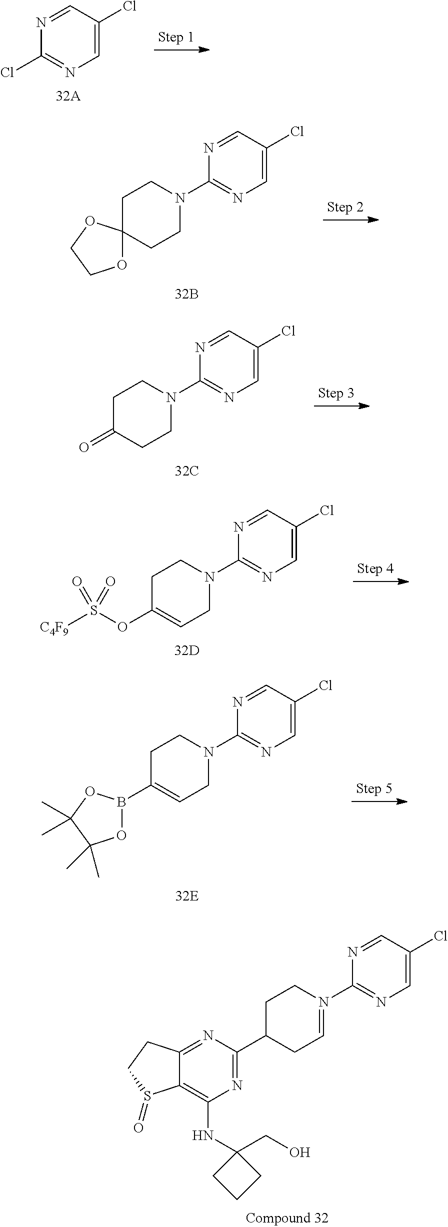

Step 1: 2,5-Dichloropyrimidine (5.0 g, 33.6 mmol), 1,4-dioxa-8-azaspiro[4.5]decane (5.3 g, 36.9 mmol), and DIPEA(13.0 g, 100.8 mmol) were dissolved in 100 mL of dioxane, and the system was warmed to 80° C. and reacted for 2 h. The reaction system was poured into ice water and extracted twice with ethyl acetate, the organic phases were combined and dried, and the solvent was removed by rotary evaporation. A mobile phase of PE:EA=20:1 was used for silica gel column chromatography purification to obtain the target compound 32B. (8.5 g, yield 99%).

LCMS (ESI): m/z=256.2 [M+H]⁺.

Step 2: The raw material 32B (8.5 g, 33.2 mmol) was dissolved in 30 mL of tetrahydrofuran, and 120 mL of 10% sulfuric acid was added, heated to 90° C., and reacted under reflux for 12 h. After the reaction was confirmed to be complete by TLC plate spotting, the mixture was cooled to room temperature and then adjusted to neutral pH with saturated sodium bicarbonate. After washing with ethyl acetate (100 mL×3), the organic phases were washed with a saturated sodium chloride aqueous solution and dried over anhydrous sodium sulfate, and the solvent was removed by rotary evaporation to obtain 32C as a solid (6.2 g, yield 88%), which was directly used in the next reaction.

LCMS (ESI): m/z=212.3 [M+H]⁺.

Step 3: The raw material 32C (6.2 g, 29.2 mmol) and 1,1,2,2,3,3,4,4,4-nonafluorobutane-1-sulfonyl fluoride (10.6 g, 35.0 mmol) were dissolved in 62 mL of tetrahydrofuran, and DBU (5.3 g, 35.0 mmol) was added and stirred for 4 h at room temperature. After the completion of the reaction was monitored by TLC, the reaction product was concentrated and then purified by silica gel column chromatography with the mobile phase system PE:EA=50:1 to obtain 32D as an oil (10.6 g, yield 73%).

LCMS (ESI): m/z=494.0 [M+H]⁺.

Step 4: The raw material 32D (6.50 g, 13.2 mmol), pinacol diboronate (4.02 g, 15.8 mmol), [1,1′-bis(diphenylphosphine)ferrocene]palladium dichloride (190 mg, 0.26 mmol), and 1,1′-bis(diphenylphosphine)ferrocene (144 mg, 0.26 mmol) were suspended in dioxane (65 mL) and reacted at room temperature under stirring for 15 min. Potassium acetate (2.58 g, 26.4 mmol) was added and reacted at 90° C. for 16 h. After the completion of the reaction was monitored by TLC and LCMS, silica gel column chromatography was carried out with PE:EA=20:1 as the mobile phase to obtain a white title compound 32E (3.37 g, yield 79%).

LCMS (ESI): m/z=322.4 [M+H]⁺.

Step 5: Compounds 32E (500 mg, 1.55 mmol) and 1E (405 mg, 1.40 mmol), [1,1′-bis(diphenylphosphino)ferrocene]palladium dichloride (22 mg, 0.03 mmol), 1,1′-bis(diphenylphosphino)ferrocene (17 mg, 0.03 mmol), and sodium carbonate (493 mg, 4.65 mmol) were suspended in 10 mL of a mixed solvent of dioxane:water=4:1 and reacted at 90° C. for 4 h.

After the reaction was complete, the reaction liquid was dropwise added to ice water and extracted with 20 mL of dichloromethane three times, and the organic phases were combined, concentrated, and subjected to silica gel column chromatography with DCM:MeOH=15:1 as the mobile phase to obtain the target compound 32. (142 mg, 23%).

¹H NMR (400 MHz, DMSO-d6) δ 8.45 (s, 2H), 7.84 (s, 1H), 7.32-7.16 (m, 1H), 4.87 (t, 1H), 4.39 (q, 2H), 3.94 (t, 2H), 3.80-3.70 (m, 2H), 3.61-3.55 (m, 1H), 3.35-3.29 (m, 1H), 3.18-3.09 (m, 1H), 3.03-2.95 (m, 1H), 2.65-2.57 (m, 2H), 2.49-2.12 (m, 4H), 1.86-1.72 (m, 2H).

LCMS (ESI): m/z=447.6 [M+H]⁻.

### Example 38

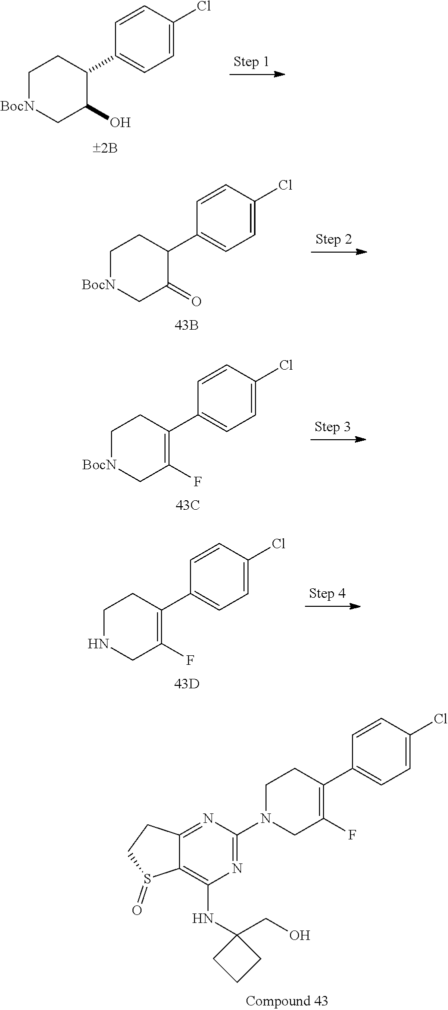 [SMILES: CC(C)(C)OC(=O)N1CC[C@H](c2ccc(Cl)cc2)[C@@H](O)C1 ; CC(C)(C)OC(=O)N1CCC(c2ccc(Cl)cc2)C(=O)C1 ; CC(C)(C)OC(=O)N1CCC(c2ccc(Cl)cc2)=C(F)C1 ; FC1=C(c2ccc(Cl)cc2)CCNC1 ; O=[S@@]1CCc2nc(N3CCC(c4ccc(Cl)cc4)=C(F)C3)nc(NC3(CO)CCC3)c21]

Step 1: The raw material ±2B (1.5 g, 4.81 mmol) and Dess-Martin periodinane (5.1 g, 12.0 mmol) were dissolved in 15 mL of dichloromethane and reacted for 4 h. After the completion of the reaction was confirmed by TLC monitoring, the reaction system was filtered, the filter cake was soaked in 15 mL of dichloromethane for ultrasonic treatment. After filtration, the filtrates were combined, the solvent was removed by rotary evaporator, and after purification by silica gel column chromatography (PE:EA=5:1), the target compound 43B (970 mg, yield 66%) was obtained.

LCMS (ESI): m/z=254.2 [M+H−tBu]⁺.

Step 2: The raw material 43B (970 mg, 3.13 mmol) was dissolved in 10 mL of tetrahydrofuran, and diethylaminosulfur trifluoride (2.53 g, 15.69 mmol) was slowly dropwise added at −20 degrees Celsius and reacted for 4 h. After the completion of the reaction was confirmed by TLC monitoring, the reaction was quenched by adding 20 ml of saturated sodium bicarbonate in an ice bath. After extraction with dichloromethane (20 mL×2), the organic phase was washed with a saturated sodium chloride aqueous solution and dried over anhydrous sodium sulfate. The solvent was removed by rotary evaporator, and after silica gel column chromatography (PE:EA=40:1), the target compound 43C (126 mg, yield 13%) was obtained.

LCMS (ESI): m/z=256.3 [M+H-tBu]+.

Step 3: The raw material 43C (126 mg, 0.41 mmol) was dissolved in a mixed solution of 0.5 mL 4N hydrogen chloride dioxane and 0.5 mL methanol and stirred at room temperature for 4 h. After the completion of the reaction was monitored by LCMS, the reaction product was concentrated to obtain the target compound 43D (84 mg, yield 98%).

LCMS (ESI): m/z=212.1 [M+H]⁺.

Step 4: Compounds 43D (84 mg, 0.40 mmol) and 1E (104 mg, 0.37 mmol) and DIPEA (258 mg, 2.00 mmol) were dissolved in 2 mL of dioxane and reacted at 80° C. for 4 h. After the reaction was complete, the reaction liquid was dropwise added to 10 mL of ice water and extracted with dichloromethane (10 mL×3) three times, and the organic phases were combined, dried over anhydrous sodium sulfate, concentrated, and subjected to HPLC preparation to obtain the target compound 43 (39 mg, 23%). [00445]¹H NMR (400 MHz, DMSO-d6) δ 7.50 (s, 1H), 7.49-7.40 (m, 4H), 4.86 (s, 1H), 4.42 (s, 2H), 3.99-3.93 (m, 2H), 3.76-3.70 (m, 2H), 3.51-3.40 (m, 1H), 3.27-3.19 (m, 1H), 3.02-2.85 (m, 2H), 2.61-2.51 (m, 2H), 2.43-2.27 (m, 2H), 2.24-2.15 (m, 2H), 1.78-1.73 (m, 2H).

LCMS (ESI): =463.1 [M+H]⁺.

¹⁹F NMR (376 MHz, DMSO) δ −116.81.

### Example 39

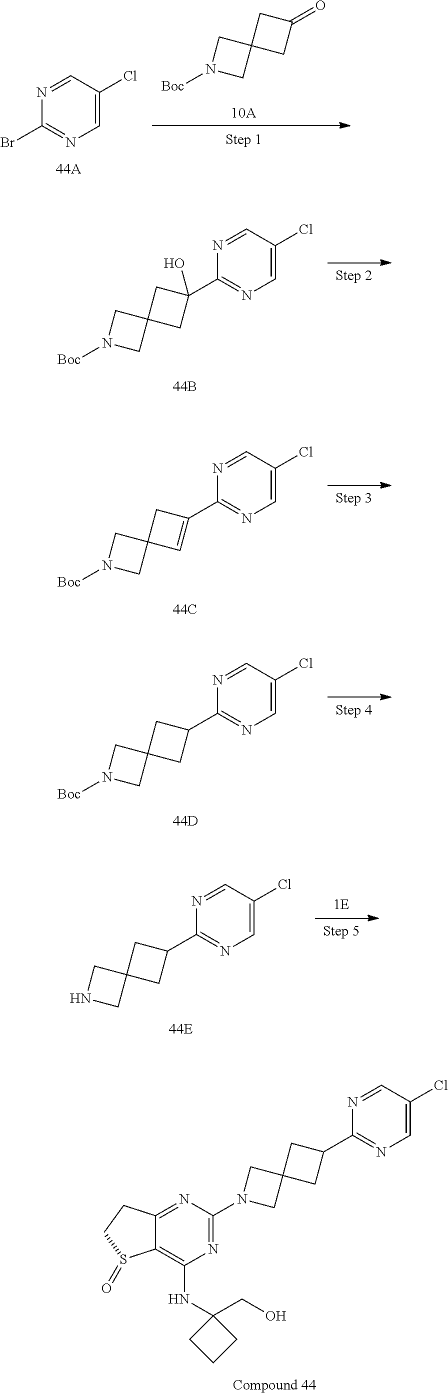

Step 1: Compound 44A (4.00 g, 20.67 mmol) was weighed into a 100 mL single-mouth flask and then dissolved by adding anhydrous toluene (50 mL). At −78° C., n-butyl lithium (1.32 g, 20.67 mmol) was added to the reaction liquid. After the addition was complete, the mixture was stirred for 0.5 h, compound 10A (5.24 g, 24.80 mmol) was then added. After the addition was complete, the mixture was warmed to 0° C. and further stirred for 2 hours. After the reaction was found complete by TLC plate spotting (petroleum ether:ethyl acetate=3:1), a saturated ammonium chloride solution (50 mL) was added to the reaction liquid and stirred for 5 min, ethyl acetate (30 mL) was added for extraction, and an organic phase was separated. The organic phase was dried over anhydrous sodium sulfate, and the crude product was concentrated under reduced pressure and purified by column chromatography (eluents: petroleum ether:ethyl acetate=5:1) to obtain the target compound 44B (1.0 g, 15%).

LCMS m/z=326.1 [M+1]⁺.

Step 2: Compound 44B (1.00 g, 3.01 mmol) was weighed into a 100 mL single-mouth flask and then dissolved by adding pyridine (15 mL). Thionyl chloride (0.22 mL) was added to the reaction liquid, and after the addition was complete, the mixture was stirred at room temperature for 16 h. After the reaction was complete, the filtrate was concentrated under reduced pressure until no liquid dripped out, and the crude product was purified by column chromatography (eluents: petroleum ether: ethyl acetate=10:1) to obtain the target compound 44C (0.26 g, 27%).

LCMS m/z=308.1 [M+1]⁺.

Step 3: Compound 44C (0.10 g, 0.32 mmol) was weighed into a 100 mL single-mouth flask and then dissolved by adding ethyl acetate (5 mL), palladium on carbon (0.10 g, 0.97 mmol) was added, and after the addition was complete, the mixture was stirred for 2 h. After the reaction was complete, the reaction liquid was filtered, and the organic phase was concentrated under reduced pressure until no liquid dripped out to obtain the target compound 44D (0.09 g, crude).

LCMS m/z=310.1 [M+1]⁺.

Step 4: Compound 44D (0.09 g, 0.29 mmol) was weighed into a 50 mL single-mouth flask and then dissolved by adding methanol (5 mL). A hydrogen chloride dioxane solution (4N, 5 mL) was added, and after the addition was complete, the mixture was stirred for 2 h. The organic phase was concentrated under reduced pressure until no liquid dripped out to obtain the target compound 44E (0.07 g, crude).

LCMS m/z=210.2 [M+1]⁺.

Step 5: Compound 1E (0.10 g, 0.35 mmol) and compound 44E (0.07 g, 0.35 mmol) were added to a 100 mL single-mouth flask and dissolved by adding 1,4-dioxane (10 mL). Finally, N,N-diisopropylethylamine (0.13 g, 1.03 mmol) was added. After the addition was complete, the system was placed under nitrogen protection and stirred at 100° C. for 16 h, and the reaction liquid was concentrated under reduced pressure to obtain a crude product of the target compound, which was subjected to preparative HPLC to obtain the title compound 44 (3.3 mg, 2%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 30-80%; c. flow rate: 15 mL/min; d. elution time: 20 min; retention time: 10.20 min.

LCMS m/z=461.2 [M+1]⁺.

¹H NMR (400 MHz, DMSO-d6) δ 8.86 (s, 2H), 7.29 (s, 1H), 4.87 (t, 1H), 4.13 (s, 2H), 3.95 (s, 2H), 3.62-3.72 (m, 3H), 3.36-3.43 (m, 2H), 3.14-3.25 (m, 1H), 2.82-2.95 (m, 2H), 2.59-2.65 (m, 2H), 2.51-2.54 (m, 1H), 2.23-2.41 (m, 2H), 2.13 (t, 2H), 1.69-1.85 (m, 2H).

### Example 40

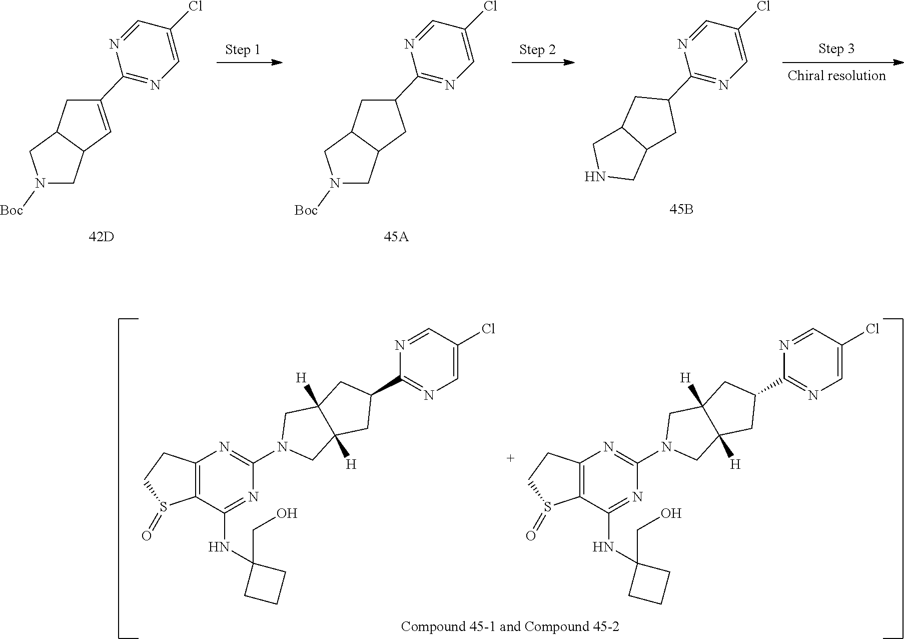

Step 1: Compound 42D (1 g, 3.11 mmol) was dissolved in 150 ml of ethyl acetate, palladium on carbon (100 mg) was added. After hydrogen displacement three times, the mixture was stirred overnight in a hydrogen atmosphere at room temperature. After the reaction was complete, the reaction product was filtered, spin-dried, and subjected to preparation. Preparative HPLC separation method: 1. Instrument: waters 2767 preparative liquid chromatographic instrument; chromatographic column: SunFire@Prep C18 (19 mm×250 mm). 2. The sample was filtered with a 0.45 m filter to prepare a sample liquid. 3. Preparative chromatography conditions: a. composition of mobile phases A and B: mobile phase A: acetonitrile; mobile phase B: water (containing 0.1% ammonium acetate). b. gradient elution, mobile phase A: 10-55%. c. flow rate: 12 mL/min. Retention time: 10.0 min. Thus, compound 45 (300 mg, 30%) was obtained.

LCMS (ESI): m/z=268.3 [M+H−56]+.

Step 2: Compound 45A (300 mg, 0.93 mmol) was dissolved in hydrogen chloride 1,4-dioxane (2 ml), stirred at room temperature for 1 h, and directly spin-dried to obtain compound 45B hydrochloride (242 mg).

LCMS (ESI): m/z=224.1 [M+H]⁺.

Step 3: Intermediate 1E (267 mg, 0.93 mmol) was dissolved in 1,4-dioxane (5 ml). Compound 45B hydrochloride (242 mg, 0.93 mmol) and diisopropylethylamine (360 mg, 2.79 mmol) were added, and the reaction system was placed in an oil bath at 100° C. and reacted for 15 h. After the reaction was complete, the reaction product was cooled to room temperature. The reaction was monitored by LCMS. The reaction product was spin-dried and then subjected to preparation. Preparative HPLC separation method: 1. Instrument: waters 2767 preparative liquid chromatographic instrument; chromatographic column: SunFire@Prep C18 (19 mm×250 mm) 2. The sample was filtered with a 0.45 m filter to prepare a sample liquid. 3. Preparative chromatography conditions: a. composition of mobile phases A and B: mobile phase A: acetonitrile; mobile phase B: water (containing 0.1% ammonium acetate). b. gradient elution, mobile phase A: 10-55%. c. flow rate: 12 mL/min. Retention time: 7.0 min. Thus, compound 45 (300 mg, 68%) was obtained.

LCMS (ESI): m/z=475.2 [M+H]⁺.

### Resolution of Compound 45:

Compound 45 (130 mg) was taken for resolution, and after separation, compound 45-1 (retention time: 1.344 min, 254 mg, ee %=100%) and compound 45-2 (retention time: 1.621 min, 20 mg, ee %=100%) were obtained.

### Resolution Conditions:

instrument: Waters 150 MGM; column: DAICEL CHIRALPAK AD;
mobile phase: A for CO₂ and B for EtOH (0.1% NH₃·H₂O)); gradient: B 40%; flow rate: 120 mL/min; back pressure: 100 bar;
column temperature: 35° C.; wavelength: 220 nm; period: 13 min;

### Compound 45-1:

¹H NMR (400 MHz, CDCl₃) δ=8.58 (s, 2H), 5.94 (s, 1H), 5.17 (s, 1H), 3.85 (s, 2H), 3.75-3.70 (m, 2H), 3.68-3.48 (m, 4H), 3.43-3.36 (m, 1H), 3.09-2.94 (m, 2H), 2.88 (s, 2H), 2.44-2.37 (m, 2H), 2.36-2.17 (m, 4H), 2.02-1.81 (m, 4H).

### Compound 45-2:

¹H NMR (400 MHz, CDCl₃) δ=8.60 (s, 2H), 5.93 (s, 1H), 3.92-3.85 (m, 4H), 3.64-3.58 (m, 2H), 3.53-3.39 (m, 3H), 3.15-2.96 (m, 4H), 2.33 (s, 2H), 2.33-2.23 (m, 5H), 2.09-2.01 (m, 2H), 2.00-1.94 (m, 1H), 1.94-1.87 (m, 1H).

### Example 41

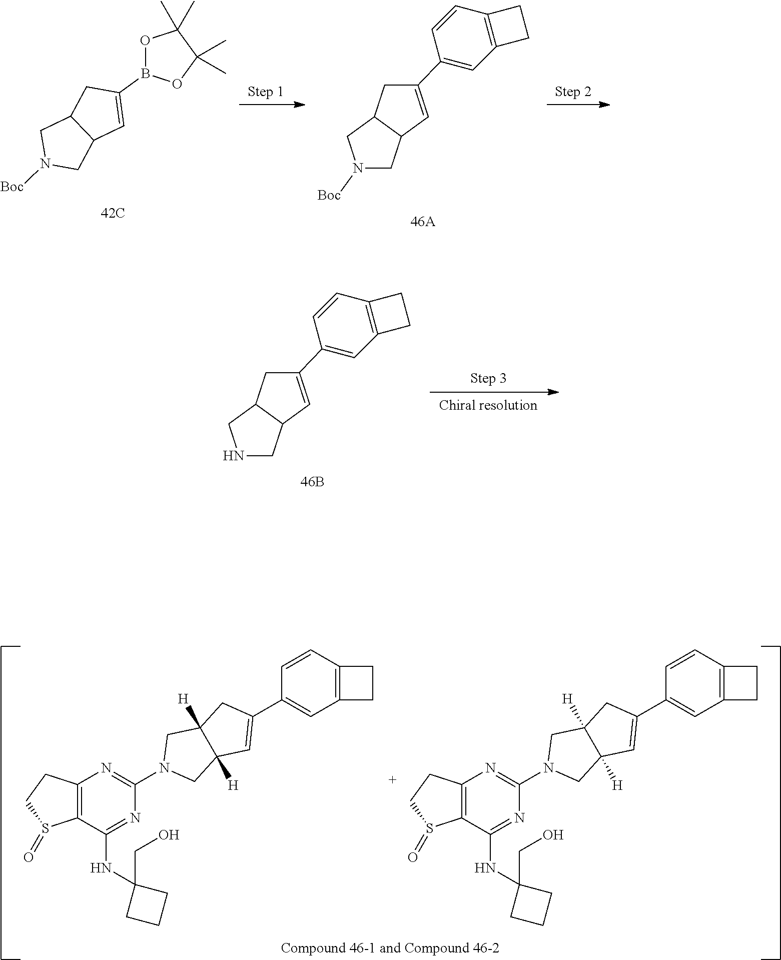

Step 1: Under nitrogen, 4-bromobenzocyclobutane (497 mg, 2.71 mmol), 1,1-bis(diphenylphosphino)ferrocene palladium dichloride (220 mg, 0.3 mmol), and sodium carbonate (951 mg, 8.97 mmol) were added to compound 42C (1 g, 2.99 mmol) dissolved in 1,4-dioxane/water (10 ml/2 ml), and the mixture was reacted for 18 h in an oil bath at 90° C. After the reaction was complete, the reaction product was cooled to room temperature, filtered with diatomite, and washed with ethyl acetate, and the filtrate was concentrated and directly quickly separated and purified by direct column chromatography (eluent ratio: EA/PE=0-10%) to obtain the target compound 46A (0.44 g, 52%).

LCMS (ESI): m/z=256.2 [M+H−56]+.

Step 2: Compound 46A (220 mg, 0.69 mmol) was dissolved in hydrogen chloride 1,4-dioxane (2 ml), stirred at room temperature for 1 h, and directly spin-dried to obtain compound 46B hydrochloride (170 mg).

LCMS (ESI): m/z=212.1 [M+H]⁺.

Step 3: Intermediate 1E (200 mg, 0.69 mmol) was dissolved in 1,4-dioxane (5 ml). Compound 46B hydrochloride (170 mg) and diisopropylethylamine (267 mg, 2.07 mmol) were added, and the reaction system was placed in an oil bath at 100° C. and reacted for 15 h. After the reaction was complete, the reaction product was cooled to room temperature. The reaction was monitored by LCMS. The reaction product was spin-dried and then subjected to preparation. Preparative HPLC separation method: 1. Instrument: waters 2767 preparative liquid chromatographic instrument; chromatographic column: SunFire@Prep C18 (19 mm×250 mm). 2. The sample was filtered with a 0.45 m filter to prepare a sample liquid. 3. Preparative chromatography conditions: a. composition of mobile phases A and B: mobile phase A: acetonitrile; mobile phase B: water (containing 0.1% ammonium acetate). b. gradient elution, mobile phase A: 10-55%. c. flow rate: 12 mL/min. Retention time: 7.0 min. Thus, compound 46 (200 mg, 63%) was obtained.

LCMS (ESI): m/z=463.3 [M+H]⁺.

### Resolution of Compound 46:

Compound 46 (200 mg) was taken for resolution, and after separation, compound 46-1 (retention time: 1.214 min, 73 mg, ee %=100%) and compound 46-2 (retention time: 1.541 min, 68 mg, ee %=100%) were obtained.

### Resolution Conditions:

instrument: Waters 150 MGM; column: DAICEL CHIRALPAK AD;
mobile phases: A for CO2 and B for EtOH (0.1% NH3·H2O)); gradient: B 40%; flow rate: 120 mL/min; back pressure: 100 bar;
column temperature: 35° C.; wavelength: 220 nm; period: 13 min;

### Compound 46-1:

¹H NMR (400 MHz, CDCl₃) δ=7.24 (s, 1H), 7.11 (s, 1H), 6.99 (d, 1H), 5.95 (s, 1H), 5.89 (s, 1H), 4.08-3.87 (m, 1H), 3.88-3.78 (m, 3H), 3.80-3.65 (m, 2H), 3.64-3.52 (m, 2H), 3.44-3.33 (m, 1H), 3.32-3.19 (m, 1H), 3.15 (s, 4H), 3.13-3.05 (m, 1H), 3.06-2.92 (m, 3H), 2.72-2.58 (m, 1H), 2.36-2.21 (m, 4H), 1.98-1.85 (m, 2H).

### Compound 46-2:

¹H NMR (400 MHz, CDCl₃) δ=7.24 (s, 1H), 7.12 (s, 1H), 6.99 (d, 1H), 5.95 (s, 1H), 5.88 (s, 1H), 4.04-3.91 (m, 1H), 3.84 (d, J=14.1, 2H), 3.80-3.66 (m, 2H), 3.60 (s, 2H), 3.44-3.20 (m, 2H), 3.15 (s, 4H), 3.13-3.06 (m, 1H), 3.00 (t, 3H), 2.64 (d, 1H), 2.43-2.14 (m, 4H), 1.98-1.80 (m, 2H).

### Example 42

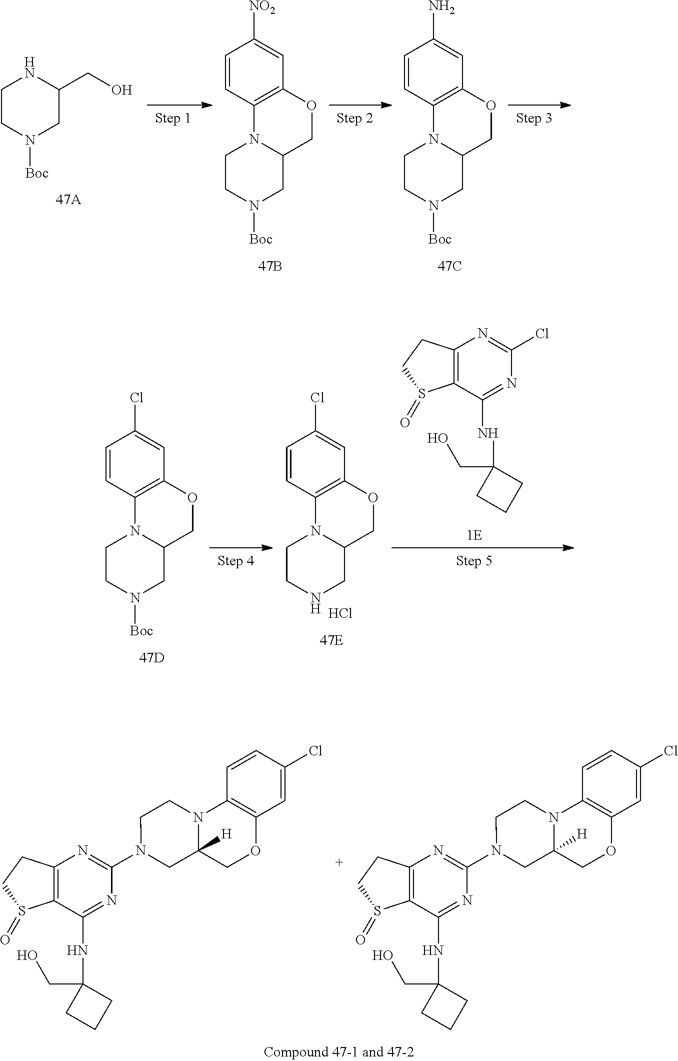

Step 1: 47A (10.0 g, 46.3 mmol), 1,2-difluoro-4-nitrobenzene (7.3 g, 46.3 mmol), potassium hydroxide (7.8 g, 138.9 mmol), and N,N-dimethylformamide (100 mL) were successively added to a 250 mL single-mouth flask. After reaction at room temperature for 6 hours, The reaction product was further warmed to 60° C. and reacted for 24 hours. After filtration, ethyl acetate (500 mL) was added to the filtrate, which was washed with water (100 mL×3). The organic layer was dried over anhydrous sodium sulfate and filtered, and the filtrate was concentrated under reduced pressure and then separated and purified by silica gel column chromatography (eluents: ethyl acetate/petroleum ether (v/v)=20/100) to obtain 47B (10.0 g, yield 64.5%).

LC-MS (ESI): m/z=336.2 [M+H]⁺.

Step 2: 47B (10.0 g, 29.9 mmol), ethyl acetate (100 mL), and palladium on carbon (2 g) were successively added to a 250 mL single-mouth flask, and the mixture was subjected to a hydrogenation reaction at room temperature for 12 hours. After filtration, the filtrate was concentrated under reduced pressure to obtain 47C (7.0 g, yield 76.9%).

LC-MS (ESI): m/z=306.2 [M+H]⁺.

Step 3: Acetonitrile (30 mL), tert-butyl nitrite (1.5 g, 14.7 mmol), and cuprous chloride (1.2 g, 11.8 mmol) were successively added to a 100 mL single-mouth flask and heated to 65° C., and a solution of 47C (3.0 g, 9.8 mmol) in acetonitrile (10 mL) was dropwise added. After the dropwise addition was complete, the mixture was reacted at 65° C. for 4 hours. After heating was stopped, the reaction was continued for 12 hours. The reaction product was concentrated under reduced pressure and then separated and purified by silica gel column chromatography (eluents: ethyl acetate/petroleum ether (v/v)=20/100) to obtain 47D (0.4 g, yield 12.6%).

LC-MS (ESI): m/z=269.2 [M−56+H]⁺.

Step 4: 47D (0.4 g, 1.2 mmol) and a hydrogen chloride-dioxane solution (4N, 10 mL) were successively added to a 50 mL single-mouth flask and reacted at room temperature for 1 hour, and the reaction product was then concentrated under reduced pressure to obtain 47E (0.4 g, yield 100.0%).

LC-MS (ESI): m/z=225.1 [M+H]⁺.

Step 5: 1E (344 mg, 1.2 mmol), 47E (400 mg, 1.2 mmol), dioxane (5 mL), and DIPEA (774 mg, 6.0 mmol) were successively added to a 50 mL single-mouth flask and reacted at 100° C. for 12 hours. The reaction product was cooled to room temperature, and the reaction liquid was directly concentrated under reduced pressure and then separated and purified by silica gel column chromatography (eluent dichloromethane/methanol (v/v)=100/8) to obtain 400 mg of a mixture.

After chiral preparation, the title compound 47-1 (90 mg, 31.6%, retention time: 1.897 min) and the title compound 47-2 (80 mg, 28.0%, retention time: 2.355 min) were obtained. Chiral preparation method: instrument: Waters 150 MGM; chromatographic column: Chiralpak Column; mobile phase: A: carbon dioxide, and B: isopropanol (0.1% aqueous ammonia); isocratic elution: 30% mobile phase B; flow rate: 100 mL/min; back pressure: 100 bar; column temperature: 25° C.; wavelength: 220 nm; elution time: 20 min.

### (Compound 47-1)

¹H NMR (400 MHz, DMSO-d₆) δ 7.45 (s, 1H), 6.92 (d, 1H), 6.86-6.81 (m, 1H), 6.77 (d, 1H), 4.93-4.80 (m, 1H), 4.77-4.58 (m, 2H), 4.45-4.30 (m, 1H), 4.05-3.92 (m, 1H), 3.88-3.78 (m, 1H), 3.77-3.65 (m, 2H), 3.52-3.39 (m, 1H), 3.26-3.17 (m, 1H), 3.14-2.84 (m, 4H), 2.77-2.57 (m, 2H), 2.40-2.25 (m, 2H), 2.23-2.14 (m, 2H), 1.89-1.66 (m, 2H).

LC-MS (ESI): m/z=476.2 [M+H]⁺.

### (Compound 47-2)

¹H NMR (400 MHz, DMSO-d₆) δ 7.45 (s, 1H), 6.92 (d, 1H), 6.86-6.80 (m, 1H), 6.77 (d, 1H), 4.90-4.78 (m, 1H), 4.80-4.58 (m, 2H), 4.43-4.31 (m, 1H), 4.07-3.93 (m, 1H), 3.89-3.80 (m, 1H), 3.76-3.68 (m, 2H), 3.52-3.38 (m, 1H), 3.27-3.17 (m, 1H), 3.13-2.83 (m, 4H), 2.76-2.57 (m, 2H), 2.44-2.25 (m, 2H), 2.24-2.12 (m, 2H), 1.86-1.66 (m, 2H).

LC-MS (ESI): m/z=476.2 [M+H]⁺.

### Example 43

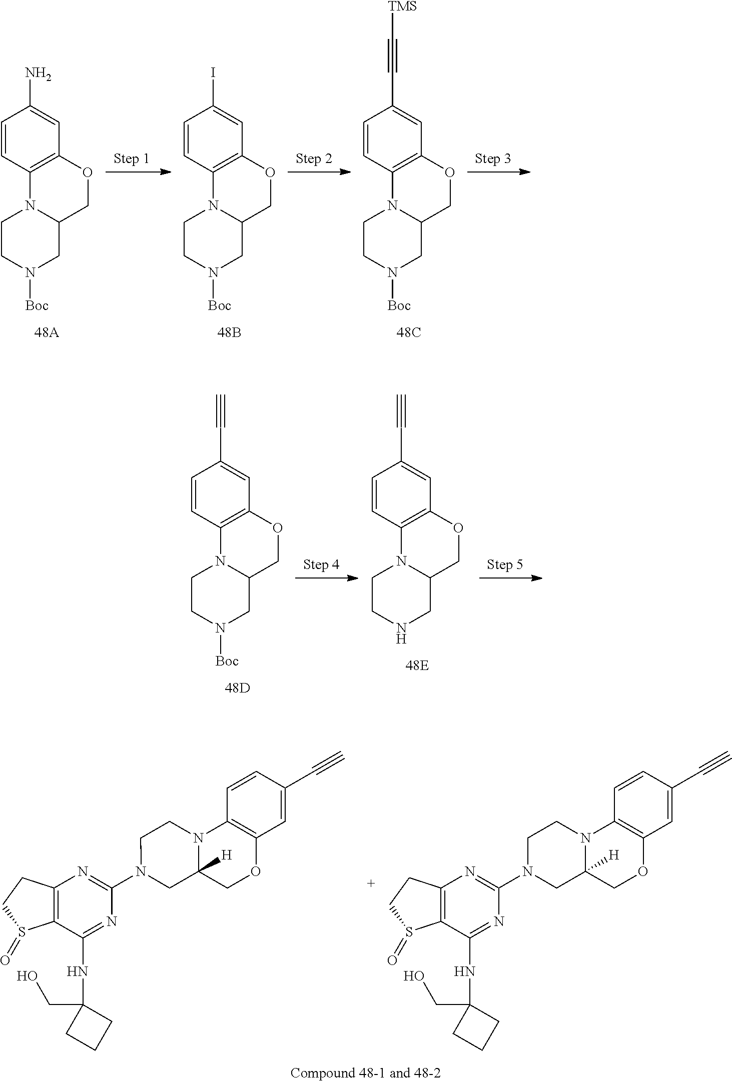

Step 1: Tert-butyl nitrite (3.55 g, 34.38 mmol) and cuprous iodide (5.24 g, 27.50 mmol) were added to acetonitrile (70 mL), the mixture was warmed to 65° C., and 48A (7 g, 22.92 mmol) in acetonitrile solution (20 ml) was then dropwise added. The system was maintained at 65° C. and reacted for 4 h. After cooling to room temperature, the reaction liquid was concentrated under reduced pressure. Water (50 ml) was then added, the liquid was washed with ethyl acetate, dried over anhydrous sodium sulfate, and filtered, and the filtrate was concentrated under reduced pressure and then separated and purified by silica gel column chromatography (eluent EA/PE=0-25%) to obtain crude product 48B (5 g). The compound was directly used for the next reaction.

LC-MS (ESI): m/z=361.3 [M+H−56]⁺.

Step 2: 48B (5 g), trimethylethynylsilane (1.42 g, 14.41 mmol), bis(triphenylphosphino)palladium dichloride (0.84 g, 1.20 mmol), cuprous iodide (0.46 g, 2.40 mmol), and triethylamine (3.65 g, 36.03 mmol) were added to tetrahydrofuran (40 mL). After nitrogen displacement, the mixture was reacted overnight at room temperature, and the reaction liquid was directly concentrated under reduced pressure and then separated and purified by silica gel column chromatography (eluent EA/PE=0-25%) to obtain 3.5 g of a crude product. Purification was further carried out with a C18 reverse-phase column, wherein the composition of mobile phases was: mobile phase A: acetonitrile; mobile phase B: water (containing 0.1% TFA), so that 48C (2 g, 43%) was obtained by separation and purification (A/B=25%/75%).

LC-MS (ESI): m/z=387.2 [M+H]⁺.

Step 3: 48C (1.5 g, 3.88 mmol) and potassium carbonate (1.07 g, 7.76 mmol) were added to methanol (15 mL) and reacted at room temperature for 3 h. The completion of the reaction was monitored by LC-MS. The reaction product was concentrated under reduced pressure to remove most methanol. Water (20 ml) was added and the mixture was extracted with ethyl acetate (20 ml×2). The organic phases were combined, washed with a saturated sodium chloride solution, dried over anhydrous sodium sulfate, and filtered. The filtrate was concentrated under reduced pressure to obtain 48D (1.1 g, 90.18%).

LC-MS (ESI): m/z=315.5 [M+H]⁺.

Step 4: 48D (0.35 g, 1.11 mmol) and triethylamine (1.12 g, 11.11 mmol) were added to dichloromethane (15 mL), and iodotrimethylsilane (1.34 g, 6.69 mmol) was then added dropwise and reacted at room temperature for 2 h. After the reaction was complete, the reaction liquid was directly concentrated under reduced pressure to obtain crude product 48E (1.7 g). The product was directly brought to the next reaction.

LC-MS (ESI): m/z=215.5 [M+H]⁺.

Step 5: Crude product 48E (1.7 g), 1E (0.32 g, 1.1 mmol), and N,N-diisopropylethylamine (0.57 g, 4.41 mmol) were added to 1,4-dioxane (15 mL). The mixture was warmed to 100° C. and reacted for 5 h. The reaction product was cooled to room temperature, and the reaction liquid was directly concentrated under reduced pressure and then separated and purified by silica gel column chromatography (eluent MeOH/DCM=0-10%) to obtain 0.4 g of a mixture. After chiral preparation, 48-1 (120 mg, 23.4%, retention time: 1.249 min) and 48-2 (130 mg, 25.4%, retention time: 1.586 min) were obtained. Chiral preparation method: instrument: Waters 150 preparative SFC (SFC-26); chromatographic column: ChiralPak AD, 250×30 mm I.D., 10 μm; mobile phase: A: carbon dioxide, and B: isopropanol (0.1% aqueous ammonia); isocratic elution: 40% mobile phase B; flow rate: 100 mL/min; back pressure: 100 bar; column temperature: 38° C.; wavelength: 220 nm; elution time: 3.4 min. [00517](Compound 48-1) [00518]¹H NMR (400 MHz, CDCl₃) δ 7.02-7.00 (m, 1H), 6.92 (d, 1H), 6.72 (d, 1H), 6.13 (s, 1H), 4.86-4.65 (m, 2H), 4.31-4.25 (m, 1H), 4.14-3.93 (m, 2H), 3.90-3.80 (m, 2H), 3.79-3.72 (m, 1H), 3.65-3.53 (m, 1H), 3.45-3.36 (m, 1H), 3.24-3.12 (m, 2H), 3.10-2.97 (m, 2H), 2.96 (s, 1H), 2.85-2.72 (m, 2H), 2.39-2.29 (m, 2H), 2.28-2.19 (m, 2H), 2.01-1.83 (m, 2H).

LC-MS (ESI): m/z=466.6 [M+H]⁺.

### (Compound 48-2)

¹H NMR (400 MHz, CDCl₃) δ 7.04-6.99 (m, 1H), 6.92 (d, 1H), 6.72 (d, 1H), 6.12 (s, 1H), 4.88-4.62 (m, 2H), 4.33-4.25 (m, 1H), 4.15-3.95 (m, 2H), 3.90-3.79 (m, 2H), 3.78-3.72 (m, 1H), 3.65-3.54 (m, 1H), 3.45-3.35 (m, 1H), 3.23-3.13 (m, 2H), 3.10-2.96 (m, 2H), 2.96 (s, 1H), 2.85-2.73 (m, 2H), 2.39-3.00 (m, 2H), 2.29-2.18 (m, 2H), 2.01-1.84 (m, 2H).

LC-MS (ESI): m/z=466.6 [M+H]⁺.

### Example 44

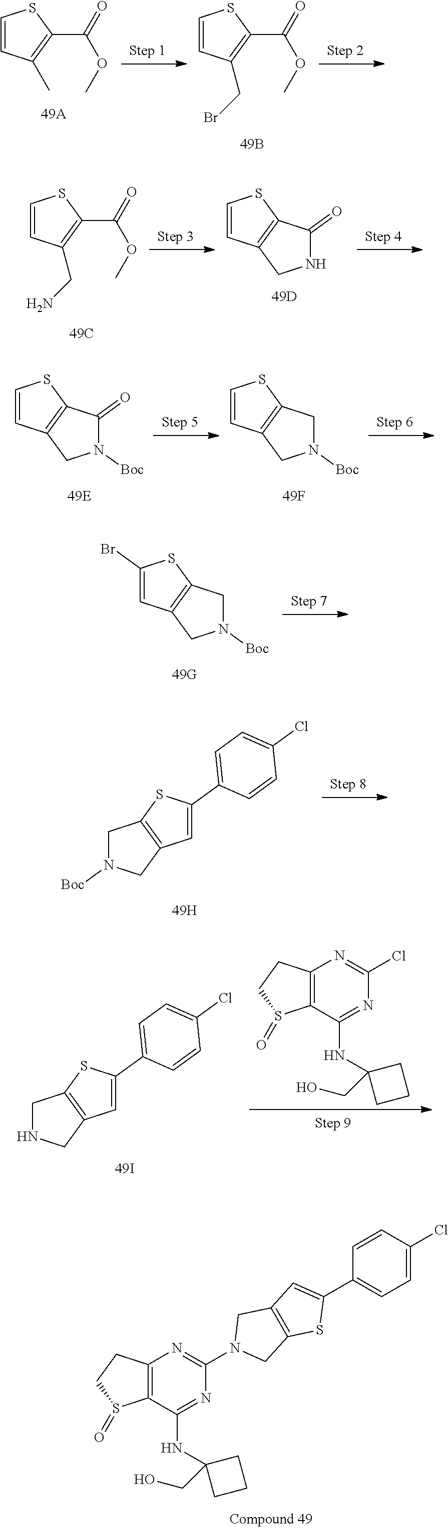

Step 1: Compound 49A (15 g, 96.03 mmol) was dissolved in carbon tetrachloride (600 mL). N-bromosuccinimide (17.09 g, 96.03 mmol) and azodiisobutyronitrile (1.58 g, 9.60 mmol) were added thereto. Under nitrogen protection, the mixture was heated to 70° C. and reacted under stirring overnight. The reaction product was cooled to room temperature, washed with water and a saturated sodium chloride aqueous solution, and dried by anhydrous sodium sulfate, and the filtrate was then spin-dried to obtain compound 49B (24 g, crude), which was directly used in the next reaction without separation and purification.

LCMS (ESI): =235.1, 237.1 [M+H]⁺.

Step 2: The crude product 49B (17 g, 63.8 mmol) was dissolved in an ammonia methanol solution (7.0 M, 20 mL) and methanol (20 mL), stirred at room temperature for 2 hours, evaporated under reduced pressure to remove the solvent, and then separated by a silica gel chromatographic column (DCM:MeOH, v/v=10:1) to obtain compound 49C (8 g, two-step yield 48.7%).

LCMS (ESI): =172.1 [M+H]⁺.

Step 3: Compound 49C (8 g, 46.73 mmol) was dissolved in a mixed solvent of methanol/ethanol (120 mL, v/v=1:1). Potassium carbonate (12.92 g, 93.45 mmol) was added thereto, and the mixture was stirred, heated to 90° C., and reacted for 3 hours. The reaction product was cooled to room temperature, evaporated under reduced pressure to remove the solvent, and 150 ml of ethyl acetate was then added. After dissolution, the solid was filtered off, and after the solvent was removed by spin drying, compound 49D (3.25 g, 36%) was obtained.

LCMS (ESI): =140.0 [M+H]⁺.

Step 4: 49D (3.25 g, 23.35 mmol) was dissolved in dichloromethane (50 mL), triethylamine (7.09 g, 70.06 mmol) and a catalytic amount of 4-dimethylaminopyridine (285 mg, 2.34 mmol) were added thereto. The mixture was cooled in ice bath, di-tert-butyl dicarbonate (10.2 g, 46.71 mmol) was added dropwise and stirred at room temperature for 3 hours, and the mixture was directly spin-dried and separated by a silica gel chromatographic column (EA:PE=1:3) to obtain compound 49E (4.45 g, 79.6%).

LCMS (ESI): =184.1 [M+H]⁺.

Step 5: 49E (4.45 g, 18.60 mmol) was dissolved in tetrahydrofuran (50 mL). Under nitrogen protection, the mixture was cooled by ice water, and a borane tetrahydrofuran complex (55.8 mL, 1.0 mol/L) was added dropwise thereto. After the dropwise addition was complete, the mixture was reacted at room temperature for 3 hours. After cooling in ice water, the reaction was quenched with a small amount of methanol, and the reaction product was evaporated under reduced pressure to remove the solvent to obtain a crude product, which was separated by a silica gel chromatographic column (EA:PE=1:3) to obtain 49F (2.26 g, 53.9%).

LCMS (ESI): =170.1 [M+H]⁺.

Step 6: 49F (2.26 g, 10.03 mmol) was dissolved in chloroform (25 mL). A catalytic amount of glacial acetic acid was added. Liquid bromine (1.6 g, 10.03 mmol) was added dropwise under ice water cooling. The mixture was stirred at room temperature overnight, and triethylamine (3.04 g, 30.09 mmol) and a catalytic amount of 4-dimethylaminopyridine (123 mg, 1.00 mmol) were added thereto. The mixture was cooled in ice bath, di-tert-butyl dicarbonate (4.38 g, 20.06 mmol) was added dropwise and stirred at room temperature for 3 hours, and the mixture was directly spin-dried and separated by a silica gel chromatographic column (EA:PE=1:3) to obtain compound 49G (2.26 g, 74.1%).

LCMS (ESI): =248.1, 250.1 [M+H]⁺.

Step 7: 49G (1 g, 3.29 mmol), 4-chlorophenylboronic acid (616.8 mg, 3.94 mmol), sodium carbonate (697 mg, 6.57 mmol), and tetrakis(triphenylphosphino)palladium (380 mg, 0.33 mmol) were added to a mixed solution of 1,4-dioxane and water (27.5 mL, v/v=10:1). After nitrogen displacement, the mixture was reacted at 80° C. for 3 hours, and the reaction was quenched by adding water. The reaction product was extracted with ethyl acetate, washed with a saturated sodium chloride aqueous solution, dried over anhydrous sodium sulfate, and separated by a silica gel chromatographic column to obtain 49H (640 mg, 58.0%).

LCMS (ESI): =280.1, 282.1 [M+H]⁺.

Step 8: 49H (300 mg) was added to hydrogen chloride dioxane (5 mL, 4N) and reacted at room temperature for 2 hours. Then, the system was spin-dried to obtain compound 491 (300 mg, crude), which was directly used in the next reaction.

LCMS (ESI): =236.1, 238.1 [M+H]⁺.

Step 9: Compounds 491 (0.28 g, 1.18 mmol) and 1E (0.34 g, 1.18 mmol) were dissolved in 1,4-dioxane (10 ml). N,N-diisopropylethylamine (0.46 g, 3.54 mmol) was added. After the addition was complete, the mixture was warmed to 100° C. and reacted for 5 hours. After the reaction was complete, the reaction product was cooled to room temperature and then filtered. The filter cake was warmed to 70° C. and slurried for 1 hour with acetonitrile/water (v/v=9/1, 5 mL), followed by hot filtration. The filter cake was rinsed with a small amount of acetonitrile and then dried to obtain the title compound 49 (350.0 mg, 60.9%).

¹H NMR (400 MHz, DMSO-d₆) δ 7.69-7.64 (m, 2H), 7.51-7.47 (m, 3H), 4.92-4.70 (m, 4H), 3.81 (s, 2H), 3.67-3.62 (m, 1H), 3.39-3.32 (m, 2H), 3.07-3.03 (m, 1H), 2.39-2.30 (m, 4H), 1.87-1.84 (m, 2H).

MS M/Z (ESI): m/z=487.1 [M+1]⁺.

### Example 45

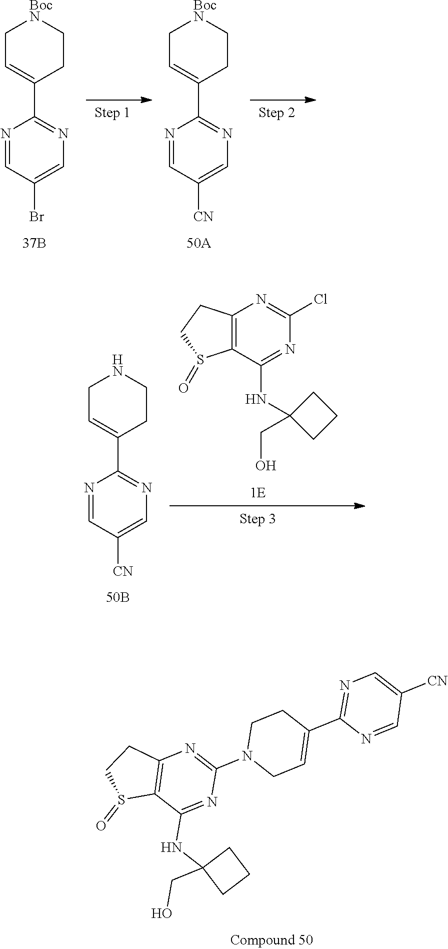

Step 1: Compound 37B (2.0 g, 5.9 mmol) was dissolved in N,N-dimethylformamide (50 ml). Zinc cyanide (0.7 g, 5.9 mmol) and tetrakis(triphenylphosphino)palladium (0.7 g, 5.9 mmol) were then added. After nitrogen displacement three times, the mixture was heated to 90° C. and reacted for 3 h, after which the reaction product was passed through a short silica gel column, rinsed with ethyl acetate, concentrated, and subjected to column chromatography (ethyl acetate/petroleum ether=1/9) to obtain compound 50A (1.5 g, 89%).

LC-MS (ESI): m/z=287.4 [M+H]⁺.

Step 2: Compound 50A (1.5 g, 5.2 mmol) was dissolved in dichloromethane (50 mL).

Trifluoroacetic acid (6.0 mL) was then added, and the mixture was reacted at room temperature for half an hour and then concentrated to obtain compound 50B (0.9 g, 92%), which was directly used in the next step.

LC-MS (ESI): m/z=187.2 [M+H]⁺.

Step 3: Compound 50B (0.9 mg, 4.8 mmol) was dissolved in 1,4-dioxane (50.0 mL). Compound 1E (1.4 g, 4.8 mmol) and N,N-diisopropylethylamine (0.7 g, 5.3 mmol) were then added, and the mixture was heated back to 90° C. and reacted for 12 h, after which the reaction product was cooled to room temperature, concentrated, and subjected to column chromatography (dichloromethane/methanol=10/1) to obtain compound 50 (0.3 g, 14%).

LC-MS (ESI): m/z=438.5 [M+H]⁺.

¹H NMR (400 MHz, CDCl₃) δ 8.92 (s, 1H), 7.52 (s, 1H), 6.21 (s, 1H), 4.55 (s, 1H), 4.05 (t, 2H), 3.93-3.82 (m, 2H), 3.65-3.57 (m, 1H), 3.43-3.35 (m, 1H), 3.08-2.99 (m, 2H), 2.78 (s, 2H), 2.39-2.31 (m, 2H), 2.31-2.25 (m, 2H), 1.97-1.84 (m, 2H).

### Example 46

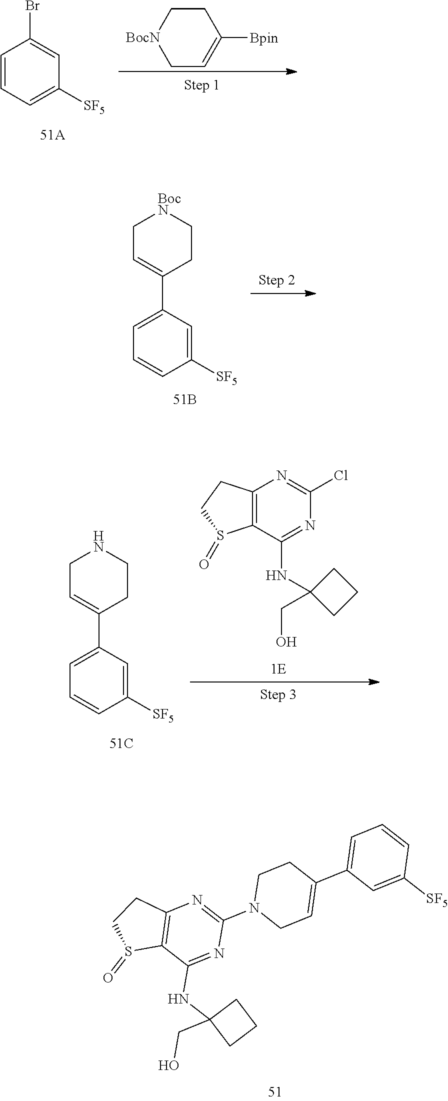

Step 1: Compound 51A (0.9 g, 3.2 mmol) was dissolved in 1,4-dioxane (40 mL). 1,1-Bis(diphenylphosphino)ferrocene palladium dichloride (0.2 mg, 0.3 mmol), cesium carbonate (2.1 g, 6.3 mmol), N-Boc-1,2,5,6-tetrahydropyridine-4-boronic acid pinacol ester (1.1 g, 3.5 mmol), and water (10 mL) were then added. After nitrogen displacement three times, the mixture was heated to 90° C. and reacted for 4 h, after which the reaction product was passed through a short silica gel column, rinsed with ethyl acetate, concentrated, and subjected to column chromatography (petroleum ether/ethyl acetate=9/1) to obtain compound 51B (0.7 g, 57%).

LC-MS (ESI): m/z=386.4 [M+H]⁺.

Step 2: Compound 51B (0.7 g, 1.8 mmol) was dissolved in dichloromethane (20 mL). Trifluoroacetic acid (2.8 mL) was then added, the mixture was reacted at room temperature for half an hour and then concentrated to obtain compound 51C (0.5 g, 96%), which was directly used in the next step.

Step 3: Compound 50C (0.5 mg, 1.8 mmol) was dissolved in 1,4-dioxane (30.0 mL). Compound 1E (0.5 g, 1.8 mmol) and N,N-diisopropylethylamine (1.8 g, 14.5 mmol) were then added, and the mixture was heated back to 90° C. and reacted for 12 h, after which the reaction product was cooled to room temperature, concentrated, and subjected to column chromatography (dichloromethane/methanol=10/1) to obtain compound 51 (0.5 g, 53%).

LC-MS (ESI): m/z=537.1 [M+H]⁺.

¹H NMR (400 MHz, CDCl₃) δ 7.74 (s, 1H), 7.64 (d, 1H), 7.51 (d, 1H), 7.43 (t, 1H), 6.34 (s, 1H), 6.18 (s, 1H), 4.43 (s, 2H), 4.07 (s, 2H), 3.90-3.81 (m, 2H), 3.67-3.59 (m, 1H), 3.42-3.34 (m, 1H), 3.08-3.04 (m, 2H), 2.61 (s, 2H), 2.40-2.33 (m, 2H), 2.27-2.22 (m, 2H), 1.96-1.83 (m, 2H).

¹⁹F NMR (377 MHz, CDCl₃) δ 64.91 (s), 64.62 (s).

### Example 47

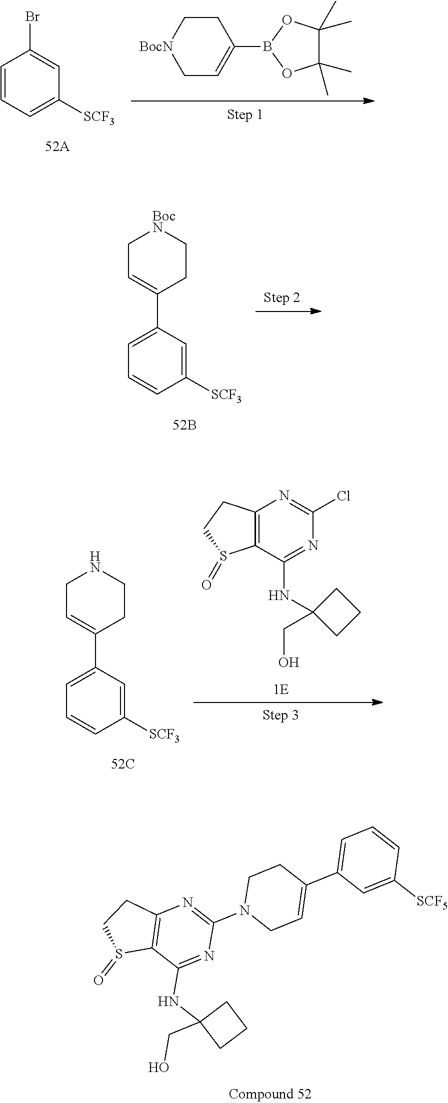

Step 1: Compound 52A (1.0 g, 3.8 mmol) was dissolved in 1,4-dioxane (40 mL). 1,1-Bis(diphenylphosphino)ferrocene palladium dichloride (0.3 mg, 0.4 mmol), cesium carbonate (2.5 g, 7.7 mmol), N-Boc-1,2,5,6-tetrahydropyridine-4-boronic acid pinacol ester (1.3 g, 4.3 mmol), and water (10 mL) were then added. After nitrogen displacement three times, the mixture was heated to 90° C. and reacted for 3 h, after which the reaction product was passed through a short silica gel column, rinsed with ethyl acetate, concentrated, and subjected to column chromatography (petroleum ether/ethyl acetate=9/1) to obtain compound 52B (1.3 g, 94%).

LC-MS (ESI): m/z=360.4 [M+H]⁺.

Step 2: Compound 52B (1.3 g, 3.6 mmol) was dissolved in dichloromethane (20 mL). Trifluoroacetic acid (2.8 mL) was then added, and the mixture was reacted at room temperature for half an hour and then concentrated to obtain compound 52C (0.8 g, 85%), which was directly used in the next step.

Step 3: Compound 52C (0.7 mg, 2.7 mmol) was dissolved in 1,4-dioxane (30.0 mL). Compound 1E (0.78 g, 2.7 mmol) and N,N-diisopropylethylamine (1.1 g, 8.1 mmol) were then added, and the mixture was heated back to 90° C. and reacted for 12 h, after which the reaction product was cooled to room temperature, concentrated, and subjected to column chromatography (dichloromethane/methanol=10/1) to obtain compound 52 (0.55 g, 40%).

LC-MS (ESI): m/z=511.1 [M+H]⁺.

¹H NMR (400 MHz, CDCl₃) δ 7.66 (s, 1H), 7.55 (d, 1H), 7.50 (d, 1H), 7.39 (t, 1H), 6.18 (s, 1H), 6.13 (s, 1H), 4.41 (s, 2H), 4.35 (s, 1H), 4.05 (s, 2H), 3.87 (s, 2H), 3.65-3.57 (m, 1H), 3.43-3.35 (m, 1H), 3.08-2.96 (m, 2H), 2.60 (s, 2H), 3.35-3.25 (m, 4H), 2.00-1.85 (m, 2H).

¹⁹F NMR (377 MHz, CDCl₃) δ −40.50 (s).

### Example 48

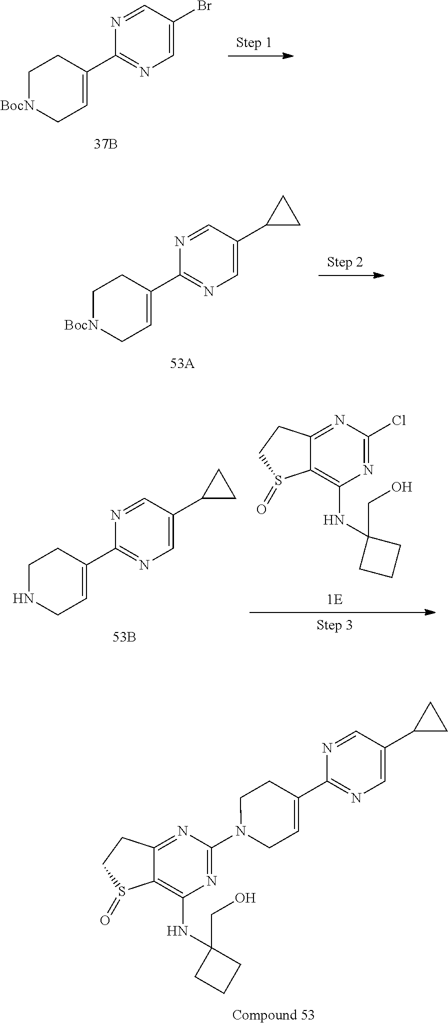

Step 1: Compound 37B (1.0 g, 3.0 mmol), cyclopropylboronic acid (356.0 mg, 4.0 mmol), palladium acetate (25.0 mg, 0.15 mmol), tricyclohexylphosphine (84.2 mg, 0.3 mmol), potassium phosphate (2.3 g, 10.5 mmol), and finally, toluene (14 mL) and water (0.7 mL) were added. After nitrogen displacement three times, the mixture was heated to 100° C. and reacted for 3 h, the reaction product was then passed through a short silica gel column, rinsed with ethyl acetate, concentrated, and subjected to column chromatography (petroleum ether/ethyl acetate=10/1) to obtain compound 53A (820.0 mg, 91%).

LC-MS (ESI): m/z=302.1 [M+H]⁺.

Step 2: Compound 53A (820.0 mg, 2.7 mmol) was dissolved in dichloromethane (20 mL). Trifluoroacetic acid (5.0 mL) was then added, the mixture was reacted at room temperature for half an hour and then concentrated to obtain compound 53B (1.0 g, 99%), which was directly used in the next step.

LC-MS (ESI): m/z=202.1 [M+H]⁺.

Step 3: Compound 53B (400.0 mg, 2.0 mmol) was dissolved in 1,4-dioxane (10.0 mL). Compound 1E (540.0 mg, 2.0 mmol) and diisopropylethylamine (1.0 g, 8.0 mmol) were then added, and the mixture was heated back to 90° C. and reacted for 12 h, after which the reaction product was cooled to room temperature, concentrated, and subjected to column chromatography (dichloromethane/methanol=10/1) to obtain compound 53 (180.0 mg, 20%).

LC-MS (ESI): m/z=453.1 [M+H]⁺.

¹H NMR (400 MHz, DMSO-d₆) δ 8.54 (s, 2H), 7.35 (s, 1H), 7.18 (s, 1H), 4.86-4.83 (m, 1H), 4.43 (s, 2H), 3.97-3.94 (m, 2H), 3.75 (d, 2H), 3.45-3.38 (m, 1H), 3.23-3.18 (m, 1H), 3.00-2.78 (m, 2H), 2.64 (s, 2H), 2.43-2.29 (m, 2H), 2.22-2.18 (m, 2H), 2.02-1.89 (m, 1H), 1.89-1.69 (m, 2H), 1.13-0.96 (m, 2H), 0.89-0.77 (m, 2H).

### Example 49

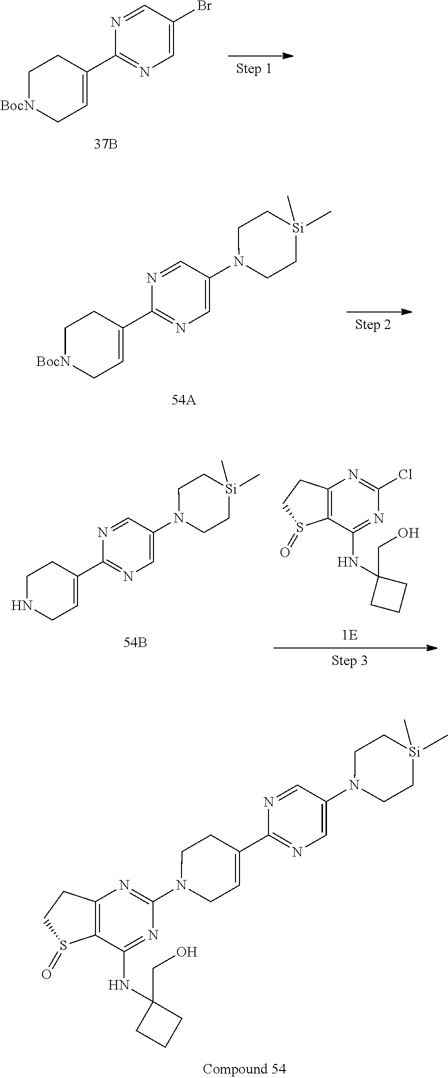

Step 1: Compound 37B (1.0 g, 2.9 mmol) was dissolved in toluene (50 mL), and tris(dibenzylideneacetone)dipalladium (266.1 mg, 0.29 mmol), 2-(di-tert-butylphosphino)biphenyl (179.0 mg, 0.6 mmol) and sodium tert-butoxide (576.0 mg, 6.0 mmol) were then added. After nitrogen displacement three times, the mixture was heated to 100° C. and reacted for 3 h. The reaction product was passed through a short silica gel column, rinsed with ethyl acetate, concentrated, and subjected to column chromatography (ethyl acetate/petroleum ether=1/9) to obtain compound 54A (258.0 mg, 23%).

LC-MS (ESI): m/z=389.2 [M+H]⁺.

Step 2: Compound 54A (258.0 mg, 0.66 mmol) was dissolved in dichloromethane (50 mL), and trifluoroacetic acid (3.0 mL) was then added. The mixture was reacted at room temperature for half an hour and then concentrated to obtain compound 54B (218.0 mg, 99%), which was directly used in the next step.

LC-MS (ESI): m/z=289.2 [M+H]⁺.

Step 3: Compound 54B (218.0 mg, 0.66 mmol) was dissolved in 1,4-dioxane (30.0 mL), compound 1E (190.0 mg, 0.66 mmol) and N,N-diisopropylethylamine (0.35 g, 2.6 mmol) were then added, and the mixture was heated back to 90° C. and reacted for 3 h, after which the reaction product was cooled to room temperature, concentrated, and subjected to column chromatography (dichloromethane/methanol=10/1) to obtain compound 54 (0.06 g, 17%).

LC-MS (ESI): m/z=540.2 [M+H]⁺.

¹H NMR (400 MHz, DMSO-d₆) δ 8.30 (s, 2H), 7.30 (s, 1H), 6.86 (s, 1H), 4.80-4.78 (m, 1H), 4.31 (s, 2H), 3.87 (s, 2H), 3.76-3.47 (m, 6H), 3.37-3.31 (m, 1H), 3.17-3.09 (m, 1H), 2.90-2.76 (m, 2H), 2.55 (s, 2H), 2.34-2.21 (m, 2H), 2.15-2.10 (m, 2H), 1.83-1.60 (m, 2H), 0.76-0.53 (m, 4H), 0.00 (s, 6H).

### Example 50

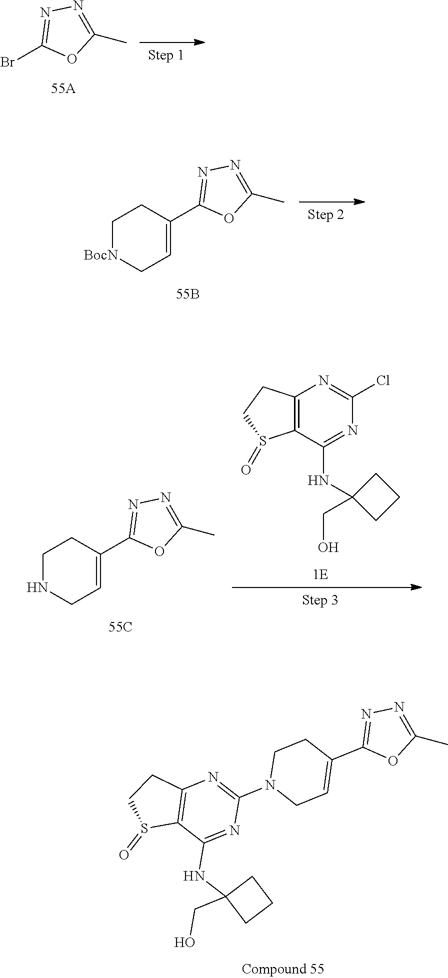

Step 1: Compound 55A (750 mg, 4.6 mmol) was dissolved in 1,4-dioxane (12 mL), 1,1-bis(diphenylphosphino)ferrocene palladium dichloride (340 mg, 0.46 mmol), cesium carbonate (4.5 g, 13.8 mmol), N-Boc-1,2,5,6-tetrahydropyridine-4-boronic acid pinacol ester (2.1 g, 6.89 mmol), and water (3 mL) were then added. After nitrogen displacement three times, the mixture was heated to 90° C. and reacted for 16 h, after which the reaction product was passed through a short silica gel column, rinsed with ethyl acetate, concentrated, and subjected to column chromatography (petroleum ether/ethyl acetate=1/1) to obtain compound 55B (0.8 g, 66%).

LC-MS (ESI): m/z=266.1 [M+H]⁺.

Step 2: Compound 55B (400 mg, 1.51 mmol) was dissolved in dichloromethane (10 mL), trifluoroacetic acid (5.0 mL) was then added, and the mixture was reacted at room temperature for half an hour and then concentrated. The residue was dissolved with dichloromethane and washed with a saturated sodium bicarbonate aqueous solution, and the organic phase was dried and concentrated to obtain compound 55C (240 mg, 96%), which was directly used in the next step.

LC-MS (ESI): m/z=166.1 [M+H]⁺.

Step 3: Compound 55C (390 mg, 1.36 mmol) was dissolved in 1,4-dioxane (10.0 mL), compound 1E (220 mg, 1.33 mmol) and N,N-diisopropylethylamine (530 mg, 4.08 mmol) were then added, and the mixture was heated back to 90° C. and reacted for 12 h, after which the reaction product was cooled to room temperature, concentrated, and subjected to column chromatography (dichloromethane/methanol=10/1) to obtain compound 55 (440 mg, 78%).

LC-MS (ESI): m/z=417.2 [M+H]⁺.

¹H NMR (400 MHz, CDCl₃) δ 6.75-6.70 (m, 1H), 6.24 (s, 1H), 4.46 (s, 2H), 4.03 (s, 2H), 3.95-3.79 (m, 2H), 3.67-3.53 (m, 1H), 3.44-3.34 (m, 1H), 3.10-2.96 (m, 2H), 2.76-2.68 (m, 2H), 2.54 (s, 3H), 2.42-2.20 (m, 4H), 2.00-1.78 (m, 2H).

### Example 51

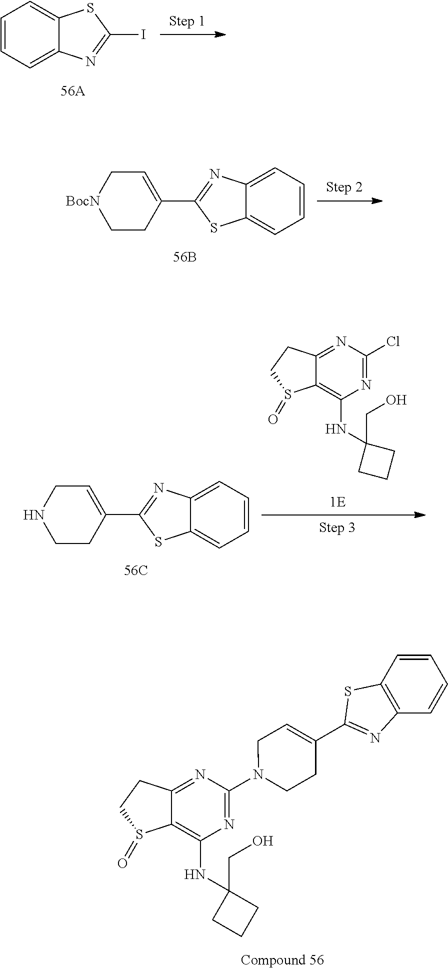 [SMILES: Ic1nc2ccccc2s1 ; CC(C)(C)OC(=O)N1CC=C(c2nc3ccccc3s2)CC1 ; O=[S@@]1CCc2nc(Cl)nc(NC3(CO)CCC3)c21 ; C1=C(c2nc3ccccc3s2)CCNC1 ; O=[S@@]1CCc2nc(N3CC=C(c4nc5ccccc5s4)CC3)nc(NC3(CO)CCC3)c21]

Step 1: Compound 56A (2.00 g, 7.67 mmol), N-Boc-1,2,5,6-tetrahydropyridine-4-boronic acid pinacol ester (2.84 mg, 9.19 mmol), tetrakis(triphenylphosphino)palladium (886 mg, 0.767 mmol), and sodium carbonate (2.43 mg, 23.0 mmol) were suspended in 50 mL of a mixed solvent of dioxane:water=4:1 and reacted at 80° C. for 16 h. After the reaction was complete, the reaction liquid was dropwise added to 100 mL of ice water and filtered. The filtrate was extracted with dichloromethane (100 mL×3), and the organic phases were combined, concentrated, and purified by silica gel column chromatography (PE:EA=40:1) to obtain the target compound 56B (420 mg, 17%).

LCMS (ESI): m/z=317.1 [M+H]⁺.

Step 2: Compound 56B (420 mg, 1.33 mmol) was dissolved in 2 mL of methanol, 2 mL of hydrogen chloride/dioxane was added and stirred at room temperature for 1.5 h, and the mixture was filtered. The filter cake was namely the target compound 56C (260 mg, 77%), which was directly used in the next reaction without purification.

LCMS (ESI): m/z=217.1 [M+H]⁺.

Step 3: Compound 56C (200 mg, 1.02 mmol), intermediate 1E (355 mg, 1.23 mmol), and DIPEA (33 mg, 0.03 mmol) were suspended in 2 mL of dioxane and reacted at 80° C. for 16 h. After the reaction was complete, the reaction product was filtered. The filter cake was slurried with 5 mL of methanol, stirred at room temperature for 0.5 h, and filtered. The filter cake was namely the target compound 56 (190 mg, 51%).

LCMS (ESI): m/z=468.1 [M+H]⁺.

¹H NMR (400 MHz, DMSO-d₆) δ 8.07 (d, 1H), 7.97 (d, 1H), 7.53-7.47 (m, 1H), 7.46-7.37 (m, 2H), 6.92-6.89 (m, 1H), 4.84 (t, 1H), 4.47 (s, 2H), 4.01 (t, 2H), 3.76 (d, 2H), 3.50-3.39 (m, 1H), 3.29-3.17 (m, 1H), 3.02-2.83 (m, 2H), 2.76 (s, 2H), 2.44-2.29 (m, 2H), 2.29-2.17 (m, 2H), 1.91-1.61 (m, 2H).

### Example 52

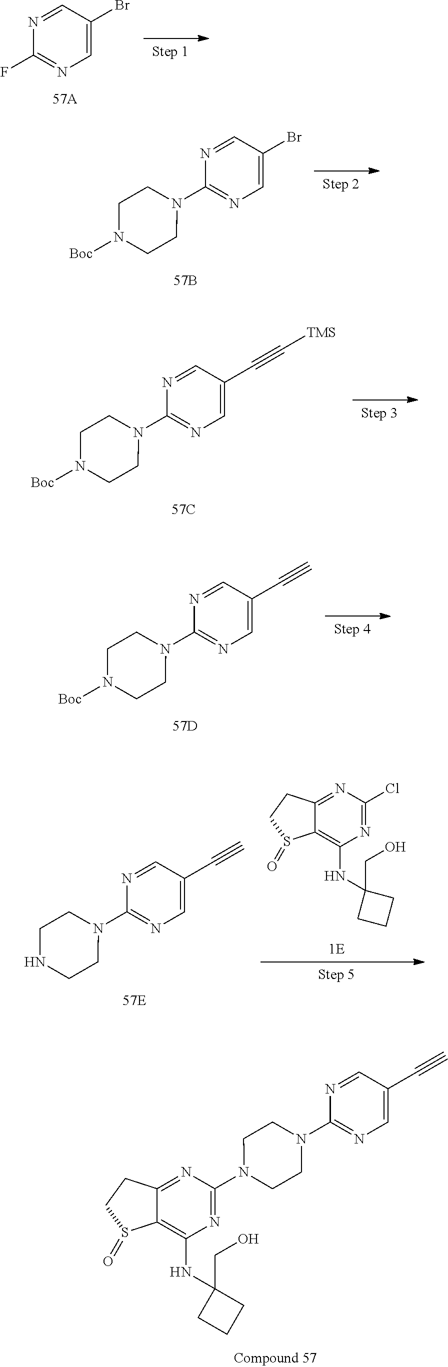

Step 1: Compound 57A (3.00 g, 16.92 mmol) and the compound tert-butyl piperazine-1-carboxylate (3.78 g, 20.30 mmol) were weighed into a 100 mL single-mouth flask and dissolved by adding 1,4-dioxane (30 mL), and N,N-diisopropylethylamine (6.56 g, 50.76 mmol) was added. After the addition was complete, the mixture was stirred at 80° C. for 16 h. After the completion of the reaction was detected by TLC plate spotting (petroleum ether:ethyl acetate=5:1), water (20 mL) was added and stirred for 5 min, and ethyl acetate (20 mL) was added for extraction. The organic phase was separated, which was dried over anhydrous sodium sulfate, concentrated under reduced pressure, and purified by column chromatography (eluents: petroleum ether:ethyl acetate=5:1) to obtain the target compound 57B (2.8 g, yield: 48%).

LCMS m/z=343.1 [M+1]⁺.

Step 2: Compound 57B (1.0 g, 2.92 mmol) and trimethylethynylsilane (1.43 g, 14.61 mmol) were weighed into a 100 mL stewing pot and dissolved with tetrahydrofuran (20 mL), and triethylamine (1.21 mL), [1,1′-bis(diphenylphosphino)ferrocene]palladium dichloride (0.24 g, 0.29 mmol), and cuprous iodide (56 mg, 0.29 mmol) were added. After the addition was complete, the mixture was stirred at 80° C. for 12 h. After the completion of the reaction was monitored by TLC plate spotting (petroleum ether:ethyl acetate=5:1), water (20 mL) was added and stirred for 5 min, ethyl acetate (20 mL) was added for extraction, and an organic phase was separated, which was dried over anhydrous sodium sulfate, concentrated under reduced pressure, and purified by column chromatography (eluents: petroleum ether:ethyl acetate=5:1) to obtain the target compound 57C (0.50 g, 47%).

LCMS m/z=361.1 [M+1]⁺.

Step 3: Compound 57C (0.5 g, 1.39 mmol) was weighed into a 100 mL single-mouth flask and dissolved with methanol (10 mL), and potassium carbonate (0.58 g, 4.2 mmol) was added. After the addition was complete, the mixture was stirred at room temperature for 2 h. After the reaction was found complete by TLC plate spotting (petroleum ether:ethyl acetate=3:1), the reaction liquid was filtered, and the filtrate was concentrated under reduced pressure and purified by column chromatography (eluents: petroleum ether:ethyl acetate=3:1) to obtain the target compound 57D (0.40 g, 100%).

LCMS m/z=289.2 [M+1]⁺.

Step 4: Compound 57D (0.2 g, 0.69 mmol) was weighed into a 100 mL single-mouth flask and dissolved with methanol (5 mL), and a hydrogen chloride dioxane solution (4N, 5 mL) was added. After the addition was complete, the mixture was stirred at room temperature for 2 h and concentrated under reduced pressure to obtain the target compound 57E (0.15 g, crude).

LCMS m/z=189.1 [M+1]⁺.

Step 5: Compound 1E (200 mg, 0.66 mmol) was added to a 100 mL single-mouth flask and dissolved in 1,4-dioxane (8 mL), and compound 57E (131 mg, 0.66 mmol) and N,N-diisopropylethylamine (0.26 g, 1.98 mmol) were added. After the addition was complete, the system was placed under nitrogen protection and stirred at 90° C. for 16 h, and the reaction liquid was concentrated under reduced pressure to obtain a crude product of the target compound, which was separated by preparative HPLC to obtain the title compound 57 (100 mg, 33%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 40-80%; c. flow rate: 15 mL/min; d. elution time: 20 min; retention time: 10.50 min.

LCMS m/z=440.2 [M+1]⁺.

¹H NMR (400 MHz, DMSO-d₆) δ 8.51 (s, 2H), 7.46 (s, 1H), 4.85 (t, 1H), 4.31 (s, 1H), 3.83 (s, 8H), 3.72 (d, 2H), 3.33-3.45 (m, 1H), 3.15-3.25 (m, 1H), 2.84-2.98 (m, 2H), 2.26-2.40 (m, 2H), 2.13-2.17 (m, 2H), 1.71-1.82 (m, 2H).

### Example 53

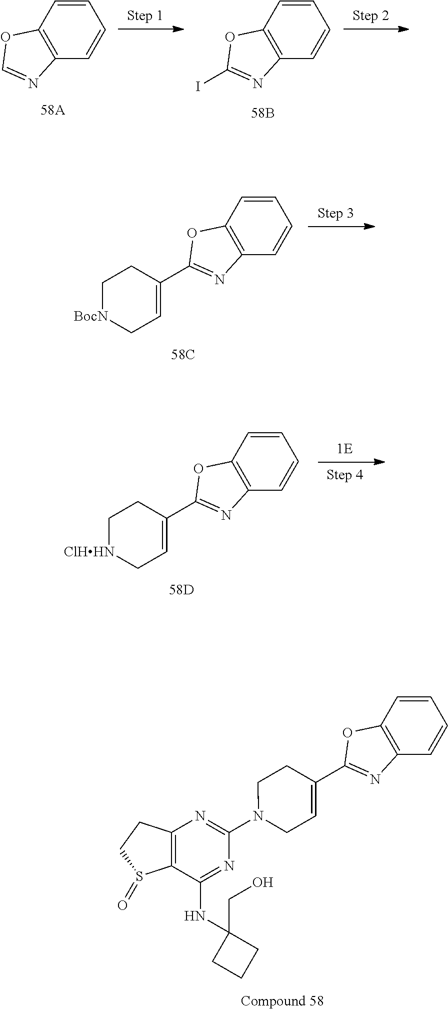

Step 1: Under nitrogen protection, 58A (1.00 g, 8.39 mmol) was dissolved in tetrahydrofuran (30 mL). After the solution was cooled to −78° C., n-butyl lithium (810 mg, 12.59 mmol) was slowly added. After the addition was complete, the reaction liquid was warmed to −40° C., stirred for 1 h, and then cooled to −78° C. A solution of iodine (2.56 g, 10.07 mmol) in tetrahydrofuran (10 mL) was added. The system naturally returned to room temperature and was stirred for 1 h. The reaction was quenched by adding water (100 mL) and extracted with ethyl acetate (100 mL) three times. The organic phases were combined, dried over anhydrous sodium sulfate, and then concentrated in vacuum to obtain a crude product. The crude product was purified by column chromatography (eluents: PE:EA=100:1 to 20:1) to obtain the target compound 58B (700 mg, yield: 34.03%).

¹H NMR (400 MHz, DMSO-d6) δ 7.76-7.73 (m, 2H), 7.38-7.36 (m, 2H).

Step 2: Compound 58B (700 mg, 2.86 mmol), [1-(tert-butoxycarbonyl)-1,2,3,6-tetrahydropyridin-4-yl]boronic acid (910 mg, 4.00 mmol), potassium carbonate (990 mg, 7.14 mmol), and [1,1′-bis(diphenylphosphino)ferrocene]palladium dichloride (110 mg, 0.14 mmol) were added to a reaction flask. 20 mL of tetrahydrofuran and water (5 mL) were added, and after nitrogen displacement, the mixture was warmed to 85° C. and reacted for 16 h. After the disappearance of raw materials was detected by plate spotting, the reaction product was diluted by adding water (100 mL) and extracted with ethyl acetate (100 mL) three times. The organic phases were combined, dried over anhydrous sodium sulfate, and then concentrated in vacuum to obtain a crude product. The crude product was purified by column chromatography (eluents: PE:EA=100:1 to 20:1) to obtain the target compound 58C (850 mg, yield: 99.06%).

LC-MS (ESI): m/z=301.2 [M+H]⁺.

¹H NMR (400 MHz, CDCl₃) δ 7.72-7.70 (m, 1H), 7.51-7.48 (m, 1H), 7.33-7.31 (m, 2H), 6.97 (s, 1H), 4.21-4.20 (m, 2H), 3.68-3.65 (m, 2H), 2.76 (s, 2H), 1.50 (s, 9H).

Step 3: Compound 58C (850 mg, 2.83 mmol) was dissolved in 20 mL of dichloromethane, and hydrogen chloride dioxane (4 mL) was added and then stirred at 0° C. for 30 min. After the disappearance of raw materials was detected by plate spotting, the reaction liquid was concentrated at room temperature to obtain crude 58D (850 mg), which was directly used in the next reaction.

Step 4: Compound 1E (300 mg, 1.04 mmol) and compound 58D (320 mg, 1.36 mmol) were dissolved in 1,4-dioxane (8 mL), and DIPEA (405 mg, 3.13 mmol) was then added. After the addition was complete, the mixture was warmed to 85° C. under nitrogen protection and stirred for 16 h. After the reaction was complete, the reaction liquid was concentrated under reduced pressure to obtain a crude product, which was purified by column chromatography (eluents: DCM:MeOH=100:1 to 9:1) and then further purified by preparative HPLC (preparation method: instrument: waters 2767 preparative liquid chromatographic instrument; chromatographic column: SunFire@PrepC18 (19 mm×250 mm; the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 25-90%; c. flow rate: 15 mL/min; d. elution time: 15 min; retention time: 6.0 min) to obtain the target compound 58 (105 mg, 22.30%).

LCMS m/z=452.2 [M+H]⁺.

¹H NMR (400 MHz, CDCl₃) δ 7.73-7.70 (m, 1H), 7.52-7.50 (m, 1H), 7.34-7.31 (m, 2H), 7.05 (s, 1H), 6.24 (s, 1H), 4.52 (s, 2H), 4.08 (s, 2H), 3.92-3.84 (m, 2H), 3.67-3.58 (m, 1H), 3.43-3.35 (m, 1H), 3.09-2.99 (m, 2H), 2.83 (s, 2H), 2.42-2.23 (m, 5H), 1.99-1.84 (m, 2H).

### Example 54

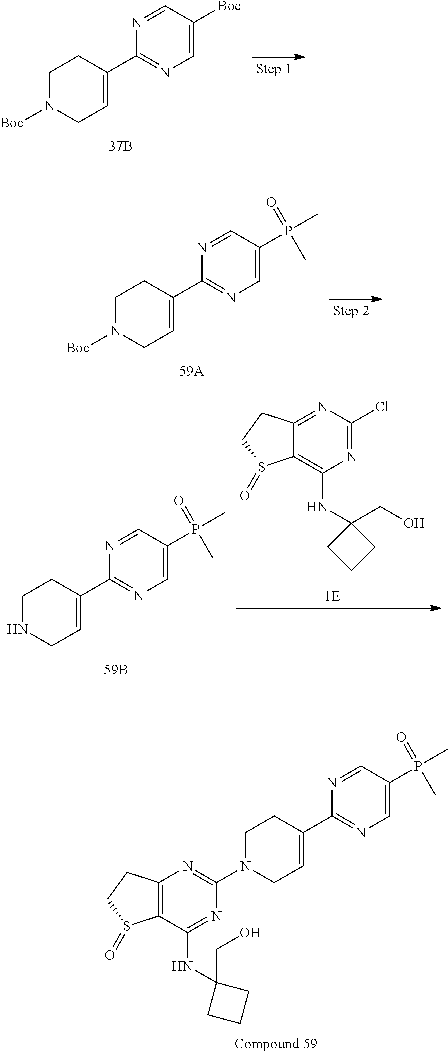

Step 1: 37B (1.1 g, 3.23 mmol) was added to a 100 mL single-mouth flask and dissolved with dioxane (20 mL). Dimethylphosphine oxide (0.38 g, 4.88 mmol), anhydrous potassium phosphate (0.75 g, 3.53 mmol), 4,5-bis(diphenylphosphino)-9,9-dimethylxanthene (0.37 g, 0.65 mmol), and tris(dibenzylideneacetone)dipalladium (0.30 g, 0.32 mmol) were successively added. Under nitrogen protection, the mixture was reacted at 100 degrees Celsius for 7 h, and the product was concentrated and purified by column chromatography (DCM:MeOH=10:1) to obtain the title compound 59A (0.8 g, 73%).

LC-MS (ESI): m/z=282.4 [M−56+H]⁺.

Step 2: 59A (0.5 g, 1.48 mmol) was added to a 50 mL single-mouth flask, dissolved with methanol (10 mL) and hydrogen chloride dioxane (4 M, 10 mL), and stirred at room temperature for 3 h, and the reaction liquid was concentrated to obtain the title compound 59B (0.40 g, 100%).

LC-MS (ESI): m/z=238.1 [M+H]⁺.

Step 3: 1E (0.15 g, 0.52 mmol) was added to a 50 mL single-mouth flask and dissolved with dioxane (10 mL), 59B (0.25 g, 1.06 mmol) and diisopropylethylamine (0.27 g, 2.05 mmol) were added and reacted at 90 degrees Celsius overnight, and the reaction liquid was concentrated, purified by HPLC, and freeze-dried to obtain the title compound 59 (23 mg, 7%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 0.1% TFA); b. gradient elution, mobile phase A: 5-60%; c. flow rate: 15 mL/min; d. elution time: 15 min; retention time: 8.0 min.

¹H NMR (400 MHz, DMSO-d6) δ 9.08 (d, 2H), 8.29-8.14 (m, 1H), 7.46-7.38 (m, 1H), 4.50 (s, 2H), 3.99 (t, 2H), 3.80-3.74 (m, 2H), 3.61-3.49 (m, 1H), 3.34-3.24 (m, 1H), 3.15-3.09 (m, 1H), 3.04-2.92 (m, 1H), 2.74 (s, 2H), 2.42-2.19 (m, 4H), 1.85-1.76 (m, 8H).

LC-MS (ESI): m/z=489.5 [M+H]⁺.

### Example 55

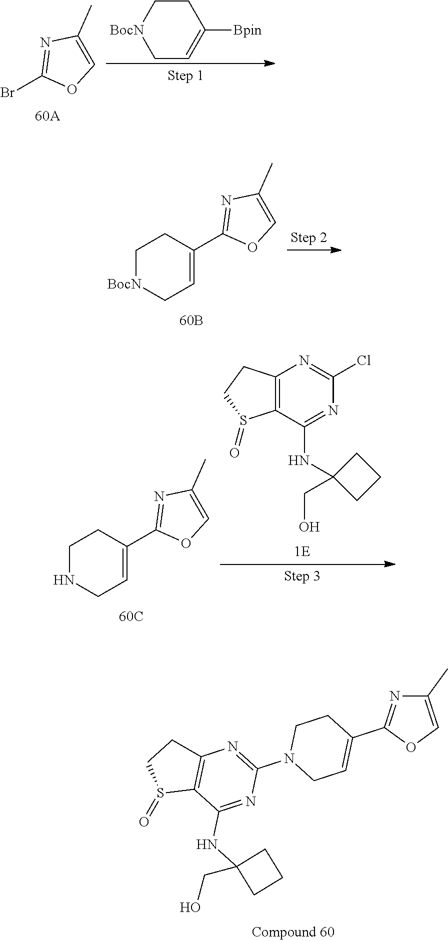

Step 1: Compound 60A (800 mg, 4.94 mmol) was dissolved in 1,4-dioxane (20 mL), and 1,1-bis(diphenylphosphino)ferrocene palladium dichloride (360 mg, 0.49 mmol), cesium carbonate (4.83 g, 14.82 mmol), N-Boc-1,2,5,6-tetrahydropyridine-4-boronic acid pinacol ester (2.29 g, 7.41 mmol), and water (4 mL) were then added. After nitrogen displacement three times, the mixture was heated to 90° C. and reacted for 16 h, after which the reaction product was passed through a short silica gel column, rinsed with ethyl acetate, concentrated, and subjected to column chromatography (petroleum ether/ethyl acetate=1/1) to obtain compound 60B (0.9 g, 69%).

LC-MS (ESI): m/z=265.1 [M+H]⁺.

Step 2: Compound 60B (5800 mg, 1.89 mmol) was dissolved in dichloromethane (10 mL), trifluoroacetic acid (5.0 mL) was then added, and the mixture was reacted at room temperature for half an hour and then concentrated. The residue was dissolved with dichloromethane and washed with a saturated sodium bicarbonate aqueous solution, and the organic phase was dried and concentrated to obtain compound 60C (300 mg, 97%), which was directly used in the next step.

LC-MS (ESI): m/z=165.1 [M+H]⁺.

Step 3: Compound 60C (300 mg, 1.04 mmol) was dissolved in 1,4-dioxane (10.0 mL), and compound 1E (170 mg, 1.04 mmol) and N,N-diisopropylethylamine (400 mg, 3.12 mmol) were then added. The mixture was heated back to 90° C. and reacted for 12 h, after which the reaction product was cooled to room temperature, concentrated, and subjected to column chromatography (dichloromethane/methanol=10/1) to obtain compound 60 (340 mg, 79%).

LC-MS (ESI): m/z=416.2 [M+H]⁺.

¹H NMR (400 MHz, CDCl₃) δ 7.30 (d, 1H), 6.71-6.67 (m, 1H), 6.30 (s, 1H), 4.42 (s, 2H), 4.01 (s, 2H), 3.93-3.77 (m, 2H), 3.67-3.54 (m, 1H), 3.42-3.32 (m, 1H), 3.09-2.96 m, 2H), 2.72-2.64 (m, 2H), 2.45-2.20 (m, 4H), 2.18 (d, 3H), 1.97-1.79 (m, 2H).

### Example 56

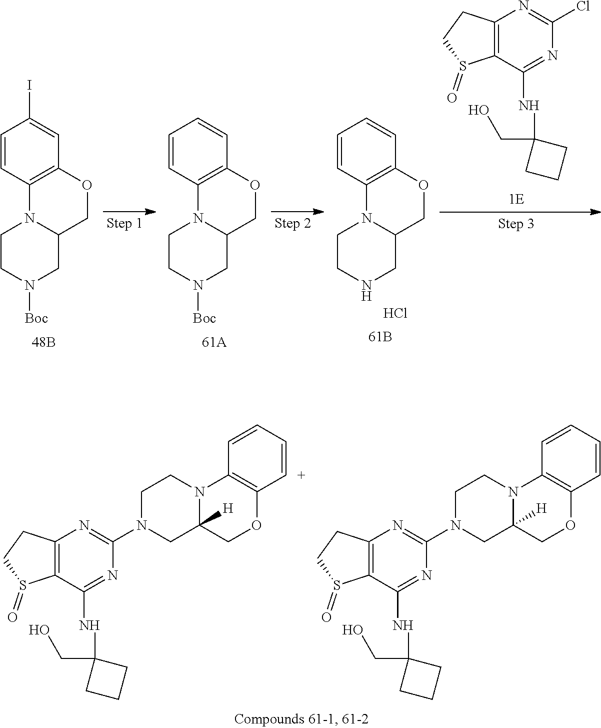

Step 1: Methanol (10 mL), 48B (0.5 g, 1.20 mmol), and palladium on carbon (100 mg) were successively added to a 50 mL single-mouth flask, the mixture was subjected to a hydrogenation reaction at room temperature for 16 hours, followed by filtration, and the filtrate was concentrated under reduced pressure to obtain 61A (0.3 g, yield 86.1%).

LC-MS (ESI): m/z=235.2 [M−56+H]⁺.

Step 2: 61A (0.3 g, 1.03 mmol) and a hydrogen chloride-dioxane solution (4N, 15 mL) were successively added to a 50 mL single-mouth flask and reacted at room temperature for 1 hour, and the reaction product was then concentrated under reduced pressure to obtain 61B (0.27 g, yield 99.6%).

LC-MS (ESI): m/z=191.2 [M+H]⁺.

Step 3: 1E (287 mg, 1.0 mmol), 61B (270 mg, 1.03 mmol), dioxane (10 mL), and DIPEA (774 mg, 6.0 mmol) were successively added to a 50 mL single-mouth flask and reacted at 100° C. for 12 hours. The reaction product was cooled to room temperature, and the reaction liquid was directly concentrated under reduced pressure and then separated and purified by silica gel column chromatography (eluent dichloromethane/methanol (v/v)=100/8) to obtain 400 mg of a mixture.

After chiral preparation, the title compound 61-1 (62 mg, 14.1%, retention time: 1.733 min) and the title compound 61-2 (51 mg, 11.5%, retention time: 1.884 min) were obtained. Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 100 mL/min; back pressure: 100 bar; column temperature: 25° C.; wavelength: 220 nm; elution time: 3.0 min.

### (Compound 61-1)

¹H NMR (400 MHz, DMSO-d₆) δ 7.43 (s, 1H), 6.98-6.86 (m, 1H), 6.86-6.75 (m, 1H), 6.76-6.59 (m, 2H), 4.90-4.79 (m, 1H), 4.78-4.59 (m, 2H), 4.37-4.28 (m, 1H), 4.00-3.92 (m, 1H), 3.90-3.78 (m, 1H), 3.78-3.68 (m, 2H), 3.49-3.36 (m, 1H), 3.27-2.81 (m, 5H), 2.76-2.55 (m, 2H), 2.41-2.12 (m, 4H), 1.87-1.70 (m, 2H).

LC-MS (ESI): m/z=442.2 [M+H]⁺.

### (Compound 61-2)

¹H NMR (400 MHz, DMSO-d₆) δ 7.44 (s, 1H), 6.95-6.89 (m, 1H), 6.84-6.77 (m, 1H), 6.75-6.62 (m, 2H), 4.89-4.78 (m, 1H), 4.79-4.59 (m, 2H), 4.39-4.28 (m, 1H), 4.02-3.81 (m, 2H), 3.79-3.68 (m, 2H), 3.51-3.39 (m, 1H), 3.26-3.16 (m, 1H), 3.14-2.83 (m, 4H), 2.76-2.55 (m, 2H), 2.43-2.25 (m, 2H), 2.26-2.13 (m, 2H), 1.89-1.69 (m, 2H).

LC-MS (ESI): m/z=442.2 [M+H]⁺.

### Example 57

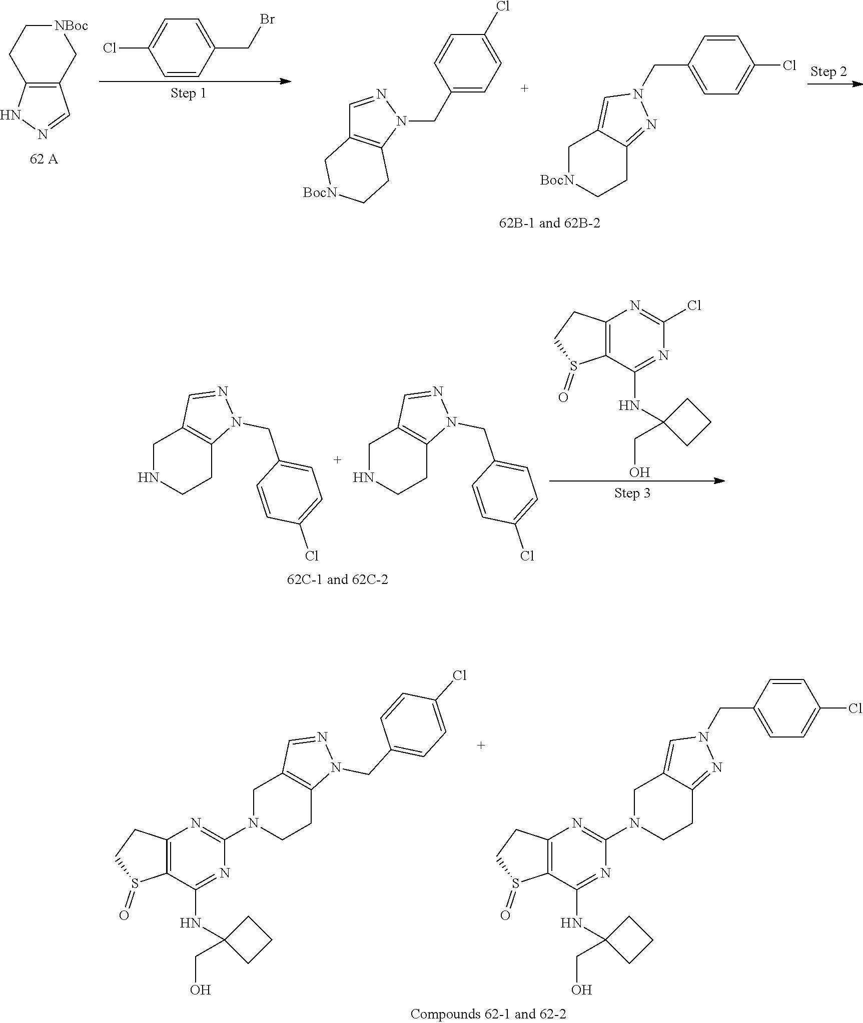

Step 1: Compound 62A (1.0 g, 4.5 mmol) was dissolved in DMF (30 mL), and cesium carbonate (2.2 g, 6.7 mmol) and 4-chlorobenzyl bromide (1.4 g, 6.7 mmol) were then added. After nitrogen displacement three times, the mixture was heated to 50° C. and reacted for 5 h. The reaction product was passed through a short silica gel column, rinsed with ethyl acetate, concentrated, and subjected to column chromatography (dichloromethane/methanol=9/1) to obtain a mixture of 62 B-1 and 62 B-2 (0.5 g, 32%).

LC-MS (ESI): m/z=348.8 [M+H]⁺.

Step 2: The mixture of compounds 62 B-1 and 62 B-2 (0.5 g, 1.4 mmol) was dissolved in dichloromethane (20 mL), and trifluoroacetic acid (2.5 mL) was then added. After reacting at room temperature for half an hour, the reaction product was concentrated to obtain a mixture of compounds 62 C-1 and 62 C-2 (0.3 g, 85%), which was directly used in the next step.

Step 3: The mixture of compounds 62 C-1 and 62 C-2 (0.3 g, 1.2 mmol) was dissolved in 1,4-dioxane (30.0 mL), and compound 1E (0.35 g, 1.2 mmol) and N,N-diisopropylethylamine (0.47 g, 3.7 mmol) were then added. The mixture was heated back to 90° C. and reacted for 12 h, after which the reaction product was cooled to room temperature, concentrated, and subjected to column chromatography (dichloromethane/methanol=10/1) to obtain a crude mixture of compounds 62-1 and 62-2.

The crude mixture of compounds 62-1 and 62-2 (380 mg) was subjected to chiral resolution to obtain compound 62-1 (175 mg) and compound 62-2 (45 mg).

Preparation method: Instrument: Waters 150 SFC, column: Chiralpak AS, mobile phase: (A for CO₂ and B for MeOH (0.1% NH₃H₂O)); gradient: 35% phase B elution; flow rate: 100 mL/min, back pressure: 100 bar; column temperature: 25° C.; wavelength: 220 nm; cycle time: 2.2 min; sample preparation: sample concentration 10 mg/mL, acetonitrile solution; injection: 3.5 mL/injection. After separation, the fractions were dried on a rotary evaporator at a bath temperature of 35° C. to obtain P1 (retention time: 1.905 minutes, assigned to compound 62-1) and P2 (retention time: 2.072 minutes, assigned to compound 62-2).

LC-MS (ESI): m/z=499.6 [M+H]⁺. [00646]¹H NMR (400 MHz, DMSO-d₆) δ 7.64 (s, 1H), 7.4-7.38 (m, 2H), 7.34 (s, 1H), 7.25-7.23 (m, 2H), 5.22 (s, 1H), 4.82 (t, 2H), 4.76 (s, 2H), 4.03-3.98 (m, 2H), 3.73 (d, 2H), 3.45-3.36 (m, 1H), 3.22-2.18 (m, 1H), 2.95-2.82 (m, 2H), 2.67 (t, 2H), 2.37-2.27 (m, 2H), 2.22-2.14 (m, 2H), 1.82-1.73 (m, 2H).

LC-MS (ESI): m/z=499.7 [M+H]⁺.

¹H NMR (400 MHz, CDCl₃) δ 7.38 (s, 1H), 7.29-7.27 (m, 2H), 7.07 (s, 1H), 7.05 (s, 1H), 5.98 (s, 1H), 5.19 (s, 2H), 4.79 (s, 2H), 4.07 (s, 2H), 3.91-3.83 (m, 2H), 3.62-3.54 (m, 2H), 3.41-3.32 (m, 2H), 3.07-2.94 (m, 2H), 2.62 (t, 2H), 2.34-2.23 (m, 2H), 1.97-1.86 (m, 2H).

### Example 58

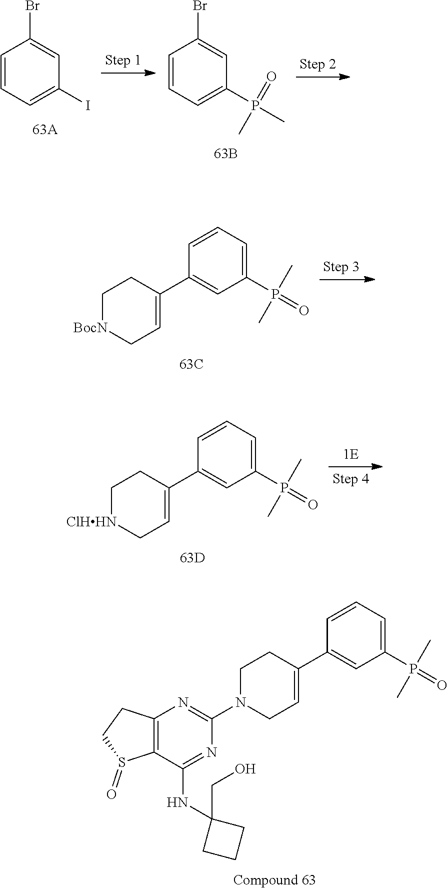

Step 1: Under nitrogen protection, 63A (5.00 g, 17.67 mmol) was dissolved in N,N-dimethylformamide (60 mL). Dimethylphosphine oxide (1.70 g, 21.20 mmol), palladium acetate (0.43 g, 1.77 mmol), 4,5-bis(diphenylphosphino)-9,9-dimethylxanthene (2.00 g, 3.53 mmol), and potassium phosphate (10.00 g, 47.11 mmol) were added successively and separately with stirring at room temperature. After the addition was complete, the reaction liquid was warmed to 100° C., stirred for 16 h, and then cooled to room temperature. The liquid was concentrated under reduced pressure using an oil pump, diluted by adding water (100 mL), and extracted with ethyl acetate (100 mL) three times. The organic phases were combined, dried over anhydrous sodium sulfate, and then concentrated in vacuum to obtain a crude product. The crude product was purified by column chromatography (eluents: DCM:MeOH=100:1 to 20:1) to obtain the target compound 63B (2.00 g, yield: 48.56%). [00650]¹H NMR (400 MHz, CDCl₃) δ 7.89-7.85 (m, 1H), 7.69-7.64 (m, 2H), 7.40-7.29 (m, 1H), 1.76 (s, 3H), 1.73 (s, 3H).

Step 2: Under nitrogen protection, compound 63B (2.00 g, 8.58 mmol) was added to a mixed solution of 1,4-dioxane (40 mL) and water (8 mL). [1-(tert-butoxycarbonyl)-1,2,3,6-tetrahydropyridin-4-yl]boronic acid (2.34 g, 10.30 mmol), [1,1′-bis(diphenylphosphino)ferrocene]palladium dichloride (630 mg, 0.86 mmol), and potassium carbonate (3.56 g, 25.75 mmol) were added successively and separately with stirring at room temperature. After the addition was complete, the reaction liquid was warmed to 85° C. and stirred under nitrogen protection for 16 h. After the disappearance of raw materials was detected by plate spotting, the reaction product was cooled to room temperature, concentrated under reduced pressure, then diluted by adding water (100 mL), and extracted with ethyl acetate (50 mL) three times. The organic phases were combined, dried over anhydrous sodium sulfate, and then concentrated in vacuum to obtain a crude product. The crude product was purified by column chromatography (eluents: DCM:MeOH=100:1 to 20:1) to obtain the target compound 63C (2.50 g, yield: 86.86%).

LC-MS (ESI): m/z=280.60 [M+H−56]+. [00653]¹H NMR (400 MHz, CDCl₃) δ 7.80-7.77 (m, 1H), 7.53-7.51 (m, 1H), 7.48-7.44 (m, 2H), 6.11 (s, 1H), 4.09-4.08 (m, 2H), 3.66-3.60 (m, 2H), 2.55 (s, 2H), 1.76 (s, 3H), 1.73 (s, 3H), 1.48 (s, 9H).

Step 3: Compound 63C (1.10 g, 3.28 mmol) was dissolved in 20 mL of dichloromethane, and hydrogen chloride dioxane (4 mL) was added and then stirred at 0° C. for 30 min. After the disappearance of raw materials was detected by TLC, the reaction liquid was concentrated at room temperature to obtain crude 63D (800 mg), which was directly used in the next reaction.

Step 4: Compound 1E (500 mg, 1.74 mmol) and compound 63D (800 mg, 2.94 mmol) were dissolved in 1,4-dioxane (8 mL), and DIPEA (675 mg, 5.21 mmol) was then added. After the addition was complete, the mixture was warmed to 85° C. under nitrogen protection and stirred for 16 h. After the reaction was complete, the reaction liquid was concentrated under reduced pressure to obtain a crude product, which was purified by column chromatography (eluents: DCM:MeOH=100:1 to 9:1) and then further purified by a reverse-phase column (eluents: water:acetonitrile=100:1 to 1:9) to obtain the target compound 63 (320 mg, 37.85%).

LCMS m/z=487.3 [M+H]⁺. [00657]¹H NMR (400 MHz, DMSO-d6) δ 7.82-7.79 (m, 1H), 7.68-7.61 (m, 2H), 7.51-7.47 (m, 1H), 7.39 (s, 1H), 6.35 (s, 1H), 4.87-4.85 (m, 1H), 4.38 (s, 2H), 4.00-3.98 (m, 2H), 3.76-3.75 (m, 2H), 3.48-2.39 (m, 1H), 3.26-3.18 (m, 1H), 2.98-2.84 (m, 2H), 2.57 (s, 2H), 2.43-2.30 (m, 2H), 2.23-2.17 (m, 2H), 1.86-1.73 (m, 2H), 1.67 (s, 3H), 1.64 (s, 3H).

### Example 59

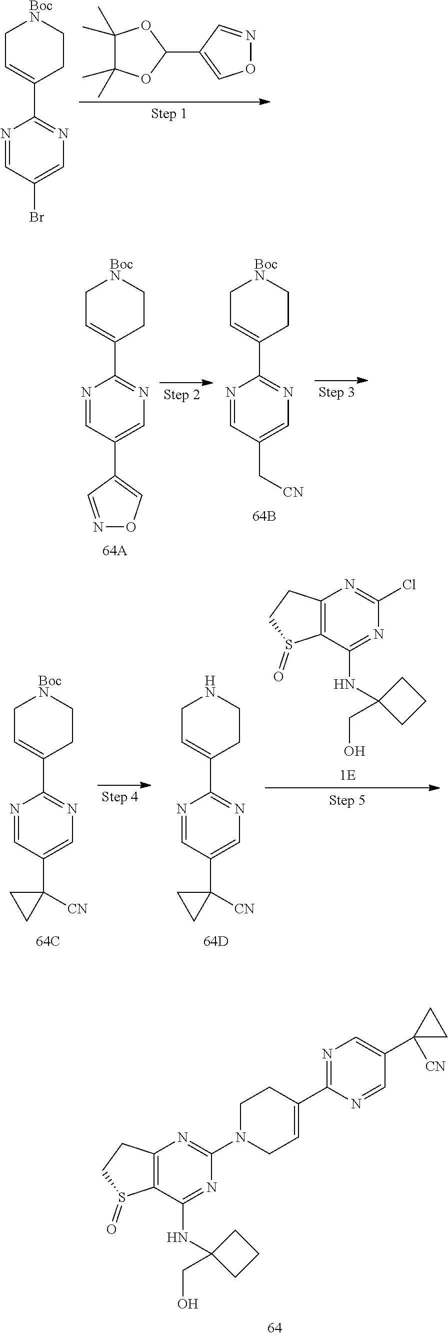

Step 1: Compound 37B (3.4 g, 10 mmol) was dissolved in N,N-dimethylformamide (100 mL), and 1,1-bis(diphenylphosphino)ferrocene palladium dichloride (725 mg, 1.0 mmol), potassium fluoride (1.7 g, 30 mmol), 4-isoxazoleboronic acid pinacol ester (2.4 g, 12 mmol), and water (30 mL) were then added. After nitrogen displacement three times, the mixture was heated to 90° C. and reacted for 4 h, after which the reaction product was passed through a short silica gel column, rinsed with ethyl acetate, concentrated, and subjected to column chromatography (petroleum ether/ethyl acetate=5/1) to obtain compound 64A (1.3 g, 39%).

LC-MS (ESI): m/z=329.1 [M+H]⁺.

Step 2: Compound 64A (1.3 g, 4.0 mmol) was dissolved in methanol (40 mL), and potassium fluoride (0.7 g, 12 mmol) and water (12 mL) were then added. The mixture was heated to 90° C. and reacted for 4 h, after which the reaction product was passed through a short silica gel column, rinsed with ethyl acetate, concentrated, and subjected to column chromatography (petroleum ether/ethyl acetate=4/1) to obtain compound 64B (1.0 g, 84%).

LC-MS (ESI): m/z=301.2 [M+H]⁺.

Step 3: Compound 64B (1.0 g, 3.3 mmol) was dissolved in water (5 mL), and tetrabutylammonium bromide (96 mg, 0.3 mmol), 1,2-dibromoethane (751 mg, 4 mmol), and potassium hydroxide (560 mg, 10 mmol) were then added. The mixture was heated to 50° C. and reacted for 4 h, after which the mixture was passed through a short silica gel column, rinsed with ethyl acetate, concentrated, and subjected to column chromatography (petroleum ether/ethyl acetate=9/1) to obtain compound 64C (0.7 g, 65%).

LC-MS (ESI): m/z=327.2 [M+H]⁺.

Step 4: Compound 64C (0.7 g, 2.1 mmol) was dissolved in dichloromethane (20 mL), trifluoroacetic acid (2.8 mL) was then added, and the mixture was reacted at room temperature for half an hour and then concentrated to obtain compound 64D (0.4 g, 90%), which was directly used in the next step.

Step 5: Compound 64D (0.4 mg, 1.7 mmol) was dissolved in 1,4-dioxane (30.0 mL), compound 1E (0.5 g, 1.8 mmol) and N,N-diisopropylethylamine (1.8 g, 14.5 mmol) were then added, and the mixture was heated back to 90° C. and reacted for 12 h, after which the reaction product was cooled to room temperature, concentrated, and subjected to column chromatography (dichloromethane/methanol=10/1) to obtain compound 64 (0.4 g, 49%).

LC-MS (ESI): m/z=478.2 [M+H]⁺. [00667]¹H NMR (400 MHz, CDCl₃) δ 8.77 (s, 2H), 7.36 (s, 1H), 7.29 (s, 1H), 4.84 (t, 1H), 4.46 (s, 2H), 3.97 (t, 2H), 3.75 (d, 2H), 3.47-3.39 (m, 1H), 3.26-3.18 (m, 1H), 2.98-2.92 (m, 1H), 2.89-2.84 (m, 1H), 2.66 (s, 2H), 2.43-2.30 (m, 2H), 2.23-2.18 (m, 2H), 1.84-1.75 (m, 4H), 1.71-1.67 (m, 2H).

### Example 60

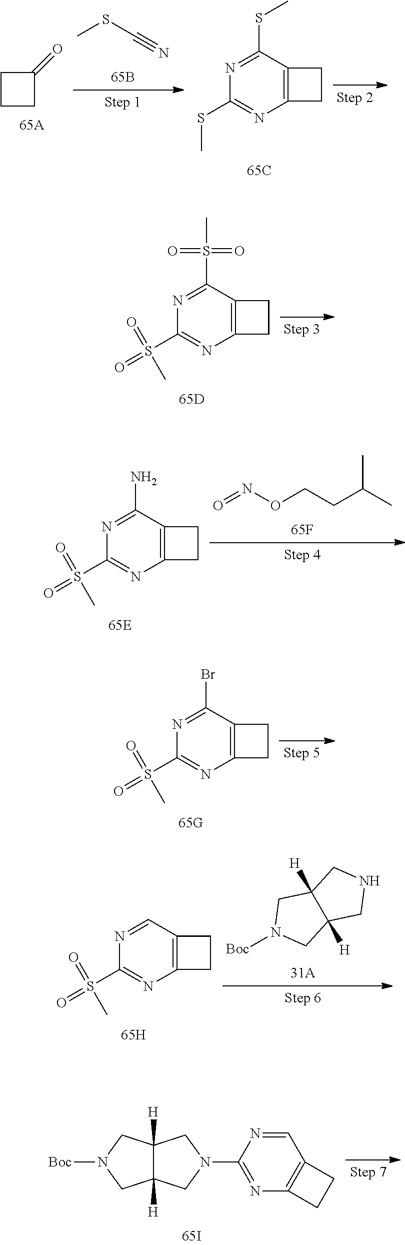

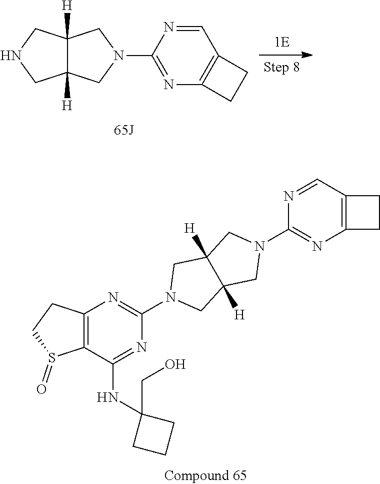 [SMILES: c1nc(N2C[C@H]3CNC[C@H]3C2)nc2c1CC2 ; O=[S@@]1CCc2nc(N3C[C@H]4CN(c5ncc6c(n5)CC6)C[C@H]4C3)nc(NC3(CO)CCC3)c21]

Step 1: Methyl thiocyanate (131.45 g, 1.80 mol) was added to a 3 μL three-mouth flask and dissolved by adding 1,2-dichloroethane (1.2 L), and trifluoromethanesulfonic anhydride (265.7 g, 0.94 mol) was then added. Cyclobutanone (60.0 g, 0.86 mol) was slowly dropwise added at room temperature, and after the addition was complete, the mixture was stirred overnight at room temperature. The reaction liquid was washed by adding a saturated sodium bicarbonate solution and adjusted to pH 8-9. After leaving to stand for layering, the organic phase was dried over anhydrous sodium sulfate, concentrated, and purified by silica gel column chromatography (DCM:EA=100: 1-20:1) and recrystallized (EA:PE=1:2) to obtain the target compound 65C (15.0 g, yield: 9%).

LCMS m/z=199.1 [M+1]⁺.

Step 2: 65C (15.0 g, 75.6 mmol) was added to a 500 mL single-mouth flask and dissolved with dichloromethane (300 mL), m-chloroperoxybenzoic acid (78.3 g, 453.8 mmol) was added, and the mixture was stirred at room temperature overnight. The reaction liquid was washed with 10% sodium hydroxide (100 mL), the organic phase was dried over anhydrous sodium sulfate, concentrated, and purified by column chromatography (PE:EA=1:1) to obtain the target compound 65D (3.8 g, yield: 19%).

LCMS m/z=263.1 [M+1]⁺.

Step 3: 65D (3.8 g, 14.5 mmol) was added to a 100 mL single-mouth flask and dissolved with dichloromethane (40 mL), and aqueous ammonia (20 mL) was then added and stirred at room temperature for 4 h. The reaction liquid was directly separated into phases, the aqueous phase was extracted once with dichloromethane (20 mL), and the organic phases were combined, dried over anhydrous sodium sulfate, concentrated and purified by column chromatography (DCM:EA=2:1) to obtain the target compound 65E (2.2 g, yield: 76%).

LCMS m/z=200.1 [M+1]⁺.

Step 4: 65E (2.2 g, 11.04 mmol) was added to a 100 mL single-mouth flask and dissolved with acetonitrile (40 ml), and isoamyl nitrite (3.9 g, 33.12 mmol) and copper bromide (4.9 g, 22.07 mmol) were then added and stirred for 6 h at 75 degrees Celsius under nitrogen protection. The reaction liquid was concentrated. Water (100 mL) was added, and the mixture was extracted with dichloromethane (2×100 mL). The organic phases were combined, dried over anhydrous sodium sulfate, concentrated, and purified by column chromatography (PE:EA=3: 1-1:1) to obtain the target compound 65G (2.0 g, yield: 69%).

LCMS m/z=263.0 [M+1]⁺.

Step 5: 65G (2.0 g, 7.6 mmol) was added to a 100 mL single-mouth flask and dissolved with ethyl acetate (40 mL), and palladium on carbon (10%, 6.0 g) was added and stirred at 60 degrees Celsius for 72 h. The reaction liquid was filtered by diatomite, and the filtrate was concentrated and purified by column chromatography (DCM:EA=2:1) to obtain the target compound 65H (0.48 g, yield: 34%).

LCMS m/z=185.1 [M+1]⁺.

Step 6: 65H (0.18 g, 0.98 mmol) was added to a 50 mL single-mouth flask and dissolved with dioxane (8 mL), and 31A (0.42 g, 1.96 mmol) and N,N-diisopropylethylamine (0.38 g, 2.94 mmol) were added and stirred at 95 degrees Celsius overnight. The reaction liquid was concentrated and purified by column chromatography (DCM:EA=5: 1-1:1) to obtain the target compound 651 (60 mg, yield: 19%).

LCMS m/z=317.2 [M+1]⁺.

Step 7: 651 (60 mg, 0.19 mmol) was added to a 50 mL single-mouth flask and dissolved with dioxane (3 mL), and hydrogen chloride dioxane (4 M, 3 mL) was added and stirred at room temperature for 4 h. The reaction liquid was concentrated to obtain the target compound 65J (48 mg, yield: 100%), which was directly used in the next reaction.

LCMS m/z=217.2 [M+1]⁺.

Step 8: 1E (42 mg, 0.15 mmol) was added to a 50 mL single-mouth flask and dissolved with dioxane (6 mL), and 65J (48 mg, 0.18 mmol) and N,N-diisopropylethylamine (59 mg, 0.45 mmol) were successively added and stirred at 95 degrees Celsius overnight. The reaction liquid was concentrated and purified by HPLC to obtain the target compound 65 (30 mg, yield: 43%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM ammonium acetate); b. gradient elution: mobile phase A: 20-70%; c. flow rate: 15 mL/min; d. elution time: 20 min; retention time: 9.5 min.

LCMS m/z=468.1 [M+1]⁺. [00685]¹H NMR (400 MHz, DMSO-d6) δ 7.90 (s, 1H), 7.23 (s, 1H), 4.81 (s, 1H), 3.83-3.66 (m, 6H), 3.43-3.35 (m, 6H), 3.23-3.18 (m, 2H), 3.07-3.00 (m, 4H), 2.94-2.76 (m, 2H), 2.42-2.26 (m, 2H), 2.17-2.12 (m, 2H), 1.84-1.68 (m, 2H).

### Example 61

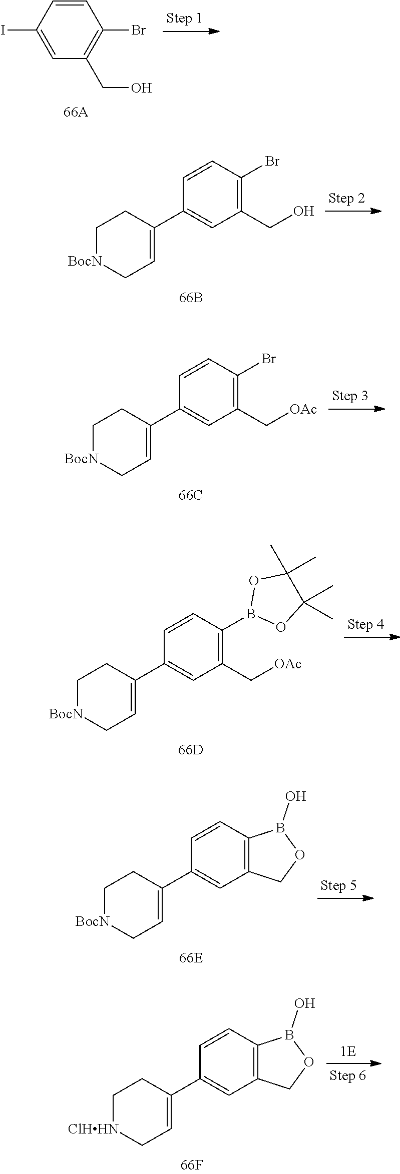

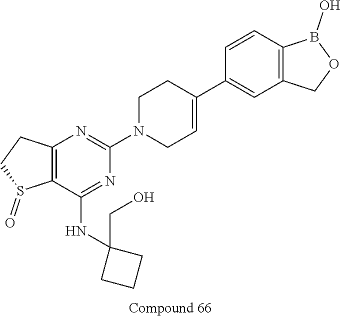 [SMILES: O=[S@@]1CCc2nc(N3CC=C(c4ccc5c(c4)COB5O)CC3)nc(NC3(CO)CCC3)c21]

Step 1: Under nitrogen protection, compound 66A (10.00 g, 31.96 mmol) was added to a mixed solution of 1,4-dioxane (100 mL) and water (10 mL). [1-(tert-butoxycarbonyl)-1,2,3,6-tetrahydropyridin-4-yl]boronic acid (7.98 g, 35.15 mmol), [1,1′-bis(diphenylphosphino)ferrocene]palladium dichloride (2.30 g, 3.20 mmol), and potassium carbonate (11.04 g, 79.89 mmol) were added successively and separately with stirring at room temperature.

After the addition was complete, the reaction liquid was warmed to 85° C. and stirred for 16 h. After the disappearance of raw materials was detected by TLC, the reaction product was cooled to room temperature, concentrated under reduced pressure into a small volume, then diluted by adding water (150 mL), and extracted with ethyl acetate (100 mL) three times. The organic phases were combined, dried over anhydrous sodium sulfate, and then concentrated in vacuum to obtain a crude product. The crude product was purified by column chromatography (eluents: DCM:MeOH=100:1 to 20:1) to obtain the target compound 66B (8.00 g, yield: 67.98%).

LC-MS (ESI): m/z=312.20 & 314.20 [M+H−56]+. [00688]¹H NMR (400 MHz, CDCl₃) δ 7.51-7.46 (m, 2H), 7.15-7.12 (m, 1H), 6.04 (s, 1H), 4.73 (s, 2H), 4.05-4.03 (m, 2H), 3.62-3.60 (m, 2H), 2.49 (s, 2H), 1.48 (s, 3H).

Step 2: Under nitrogen protection, compound 66B (2.00 g, 5.43 mmol) was dissolved in dichloromethane (40 mL), triethylamine (1.37 g, 13.58 mmol) was added with stirring in ice bath, and acetyl chloride (0.49 g, 6.25 mmol) was then slowly added dropwise. After the addition was complete, the reaction liquid was stirred at 0° C. for 1 h. After the disappearance of raw materials was detected by TLC, the reaction was quenched by adding a saturated ammonium chloride aqueous solution (100 mL), and the reaction product was extracted with dichloromethane (50 mL) three times. The organic phases were combined, dried over anhydrous sodium sulfate, and then concentrated in vacuum to obtain a crude product. The crude product was purified by column chromatography (eluents: PE:EA=100:1 to 4:1) to obtain the target compound 66C (2.00 g, yield: 95.96%). [00690]¹H NMR (400 MHz, CDCl₃) δ 7.54-7.52 (m, 1H), 7.40-7.39 (m, 1H), 7.20-7.17 (m, 1H), 6.05 (s, 1H), 5.19 (s, 2H), 4.07-4.06 (m, 2H), 3.65-3.62 (m, 2H), 2.50 (s, 2H), 2.14 (s, 3H), 1.49 (s, 9H).

Step 3: Under nitrogen protection, compound 66C (2.00 g, 4.87 mmol) was dissolved in 1,4-dioxane (80 mL). Pinacol diboronate (1.49 g, 5.85 mmol), [1,1′-bis(diphenylphosphino)ferrocene]palladium dichloride (178 mg, 0.24 mmol), and potassium acetate (1.44 g, 14.62 mmol) were added successively and separately with stirring at room temperature.

After the addition was complete, the reaction liquid was warmed to 80° C. and stirred for 5 h. After the disappearance of raw materials was detected by TLC, the reaction product was cooled to room temperature, concentrated under reduced pressure into a small volume, then diluted by adding water (100 mL), and extracted with ethyl acetate (100 mL) three times. The organic phases were combined, dried by anhydrous sodium sulfate, and then concentrated in vacuum to obtain crude product 66D (2.5 g), which was directly used in the next reaction.

Step 4: Crude compound 66D (2.5 g, 5.47 mmol) was dissolved in methanol (60 mL), and a sodium hydroxide aqueous solution (655 mg, 16.40 mmol, 1 M) was then added. After the addition was complete, the mixture was stirred at room temperature for 16 h under nitrogen protection. The reaction liquid was concentrated under reduced pressure into a small volume, diluted by adding water (50 mL), and extracted twice with ethyl acetate (100 mL). The aqueous phase was further concentrated and then purified by reverse-phase column (eluents: water:acetonitrile=100:1 to 1:9) to obtain compound 66E (1.20 g, 69.66%).

LCMS m/z=260.30 [M−56+H]⁺.

¹H NMR (400 MHz, D₂O) δ 7.34-7.32 (m, 1H), 7.18-7.16 (m, 1H), 7.11 (s, 1H), 5.96 (s, 1H), 4.63 (s, 2H), 3.93 (s, 2H), 3.50 (s, 2H), 2.41 (s, 2H), 1.34 (s, 9H).

Step 5: Compound 66E (380 mg, 1.21 mmol) was dissolved in dichloromethane (6 mL), a hydrogen chloride dioxane solution (176 mg, 4.82 mmol, 4 M) was then added in ice bath, and after the addition was complete, the mixture was stirred for 1.5 h under nitrogen protection. The reaction liquid was concentrated under reduced pressure to obtain crude compound 66F (310 mg), which was directly used in the next reaction.

LCMS m/z=216.30 [M+H]⁺.

Step 6: Compound 1E (300 mg, 1.04 mmol) and compound 66F (310 mg, 1.23 mmol) were dissolved in 1,4-dioxane (8 mL), and DIPEA (405 mg, 3.13 mmol) was then added. After the addition was complete, the mixture was warmed to 85° C. under nitrogen protection and stirred for 16 h. After the reaction was complete, the reaction liquid was concentrated under reduced pressure to obtain a crude product, which was purified by column chromatography (eluents: DCM:MeOH=100:1 to 9:1) and then further purified by a reverse-phase column (eluents: water:acetonitrile=100:1 to 1:9) to obtain the target compound 66 (150 mg, 30.85%).

LCMS m/z=467.20 [M+H]⁺. [00699]¹H NMR (400 MHz, DMSO-d6) δ 9.13 (s, 1H), 7.70-7.68 (m, 1H), 7.46-7.44 (m, 2H), 7.38 (s, 1H), 6.34 (s, 1H), 4.98 (s, 2H), 4.87-4.84 (m, 1H), 4.37 (s, 2H), 4.00-3.97 (m, 2H), 3.76-3.75 (m, 2H), 3.47-3.39 (m, 1H), 3.26-3.18 (m, 1H), 2.98-2.84 (m, 2H), 2.56 (s, 2H), 2.43-2.30 (m, 2H), 2.22-2.18 (m, 2H), 1.82-1.76 (m, 2H).

### Example 62

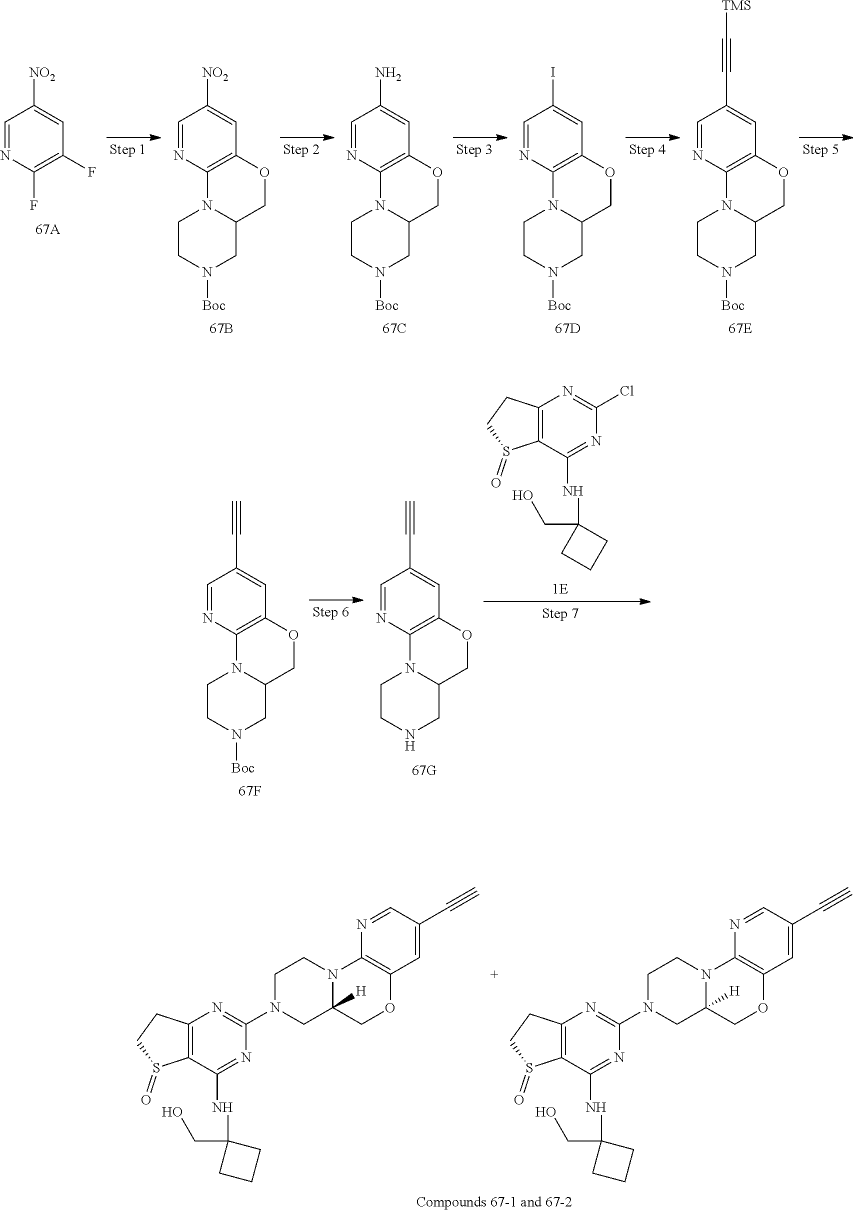

Step 1: 67A (5.0 g, 31.3 mmol), tert-butyl 3-(hydroxymethyl)piperazine-1-carboxylate (6.8 g, 31.3 mmol), potassium hydroxide (5.3 g, 93.9 mmol), and N,N-dimethylformamide (100 mL) were successively added to a 250 mL single-mouth flask. After reaction at room temperature for 6 hours, The reaction product was further warmed to 80° C. and reacted for 24 hours. After filtration, ethyl acetate (500 mL) was added to the filtrate, which was washed with water (100 mL×3) and with a saturated sodium chloride aqueous solution (100 mL×1), the organic phase was dried over anhydrous sodium sulfate and filtered, and the filtrate was concentrated under reduced pressure to obtain 67B (10.0 g, yield 95.2%).

LC-MS (ESI): m/z=281.1 [M−56+H]⁺.

Step 2: 67B (10.0 g, 29.8 mmol), ethyl acetate (300 mL), and palladium on carbon (2 g) were successively added to a 1000 mL single-mouth flask, and the mixture was subjected to a hydrogenation reaction at room temperature for 12 hours. After filtration, the filtrate was concentrated under reduced pressure to obtain 67C (8.0 g, yield 87.9%).

LC-MS (ESI): m/z=307.1 [M+H]⁺.

Step 3: Acetonitrile (30 mL), tert-butyl nitrite (3.5 g, 33.83 mmol), and cuprous iodide (5.1 g, 27.06 mmol) were successively added to a 100 mL single-mouth flask and heated to 65° C., and a solution of 67C (6.9 g, 22.55 mmol) in acetonitrile (40 mL) was dropwise added. After the dropwise addition was complete, the mixture was reacted at 65° C. for 4 hours. After heating was turned off, the reaction was continued for 12 hours. The reaction product was concentrated under reduced pressure and then separated and purified by silica gel column chromatography (eluents: ethyl acetate/petroleum ether (v/v)=20/100) to obtain 67D (1.5 g, yield 15.6%).

LC-MS (ESI): m/z=418.4 [M+H]⁺.

Step 4: 67D (1.4 g, 3.36 mmol), tetrahydrofuran (30 mL), cuprous iodide (127 mg, 0.67 mmol), triethylamine (1.0 g, 10.08 mmol), bis(triphenylphosphino)palladium dichloride (238 mg, 0.34 mmol), and trimethylsilylacetylene (1.01 g, 5.05 mmol) were successively added to a 100 mL single-mouth flask and reacted for 6 hours at room temperature under nitrogen protection, and the reaction product was concentrated under reduced pressure and then separated and purified by silica gel column chromatography (eluents: ethyl acetate/petroleum ether (v/v)=20/100) to obtain 67E (1.2 g, yield 92.3%).

LC-MS (ESI): m/z=388.2 [M+H]⁺.

Step 5: 67E (1.2 g, 3.1 mmol), methanol (30 mL), and potassium carbonate (1.3 g, 9.3 mmol) were successively added to a 50 mL single-mouth flask and reacted at room temperature for 2 hours.

The reaction product was filtered, and the filtrate was concentrated under reduced pressure and then separated and purified by silica gel column chromatography (eluents: ethyl acetate/petroleum ether (v/v)=20/100) to obtain 67F (0.8 g, yield 81.9%).

LC-MS (ESI): m/z=316.1 [M+H]⁺.

Step 6: 67F (0.3 g, 0.95 mmol), triethylamine (1.9 g, 19 mmol), and iodotrimethylsilane (1.9 g, 9.5 mmol) were successively added to a 50 mL single-mouth flask and reacted at room temperature for 2 hours. The reaction product was concentrated under reduced pressure to obtain crude 67G, which was directly used in the next step.

LC-MS (ESI): m/z=216.1 [M+H]⁺.

Step 7: 1E (273 mg, 0.95 mmol), 67G (crude, 0.8 g, 0.95 mmol), dioxane (5 mL), and DIPEA (774 mg, 6.0 mmol) were successively added to a 50 mL single-mouth flask and reacted at 100° C. for 12 hours. The reaction product was cooled to room temperature, and the reaction liquid was directly concentrated under reduced pressure and then separated and purified by silica gel column chromatography (eluent dichloromethane/methanol (v/v)=100/8) to obtain 400 mg of a mixture.

After chiral preparation, the title compound 67-1 (80 mg, 18.06%, retention time: 1.855 min) and the title compound 67-2 (50 mg, 11.31%, retention time: 2.423 min) were obtained. Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 150 mL/min; back pressure: 100 bar; column temperature: 38° C.; wavelength: 220 nm; elution time: 3.9 min. [00714](Compound 67-1) [00715]¹H NMR (400 MHz, DMSO-d₆) δ 7.90-7.83 (m, 1H), 7.46 (s, 1H), 7.09-7.04 (m, 1H), 4.87-4.79 (m, 1H), 4.81-4.60 (m, 2H), 4.52-4.35 (m, 2H), 4.08 (s, 1H), 4.04-3.95 (m, 1H), 3.78-3.65 (m, 2H), 3.51-3.37 (m, 2H), 3.28-3.15 (m, 1H), 3.07-2.74 (m, 5H), 2.43-2.16 (m, 4H), 1.86-1.70 (m, 2H).

LC-MS (ESI): m/z=467.1 [M+H]⁺. [00717](Compound 67-2) [00718]¹H NMR (400 MHz, DMSO-d₆) δ 7.96-7.71 (m, 1H), 7.47 (s, 1H), 7.25-6.78 (m, 1H), 4.88-4.64 (m, 3H), 4.53-4.36 (m, 2H), 4.08 (s, 1H), 4.04-3.90 (m, 1H), 3.72 (s, 2H), 3.48-3.39 (m, 2H), 3.27-3.21 (m, 1H), 3.06-2.75 (m, 5H), 2.40-2.17 (m, 4H), 1.87-1.72 (m, 2H).

LC-MS (ESI): m/z=467.1 [M+H]⁺.

### Example 63

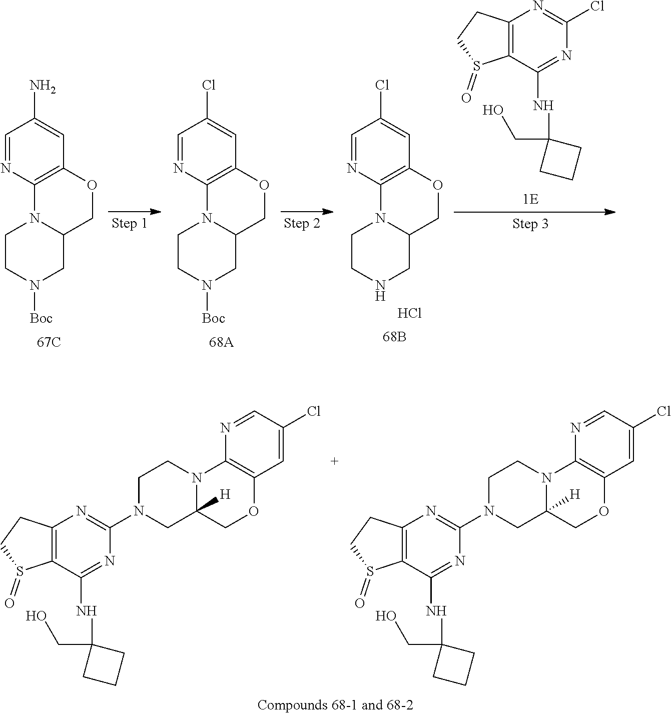

Step 1: Acetonitrile (30 mL), tert-butyl nitrite (1.5 g, 14.7 mmol), and cuprous chloride (1.2 g, 11.8 mmol) were successively added to a 100 mL single-mouth flask and heated to 65° C., and a solution of 67C (3.0 g, 9.8 mmol) in acetonitrile (10 mL) was dropwise added. After the dropwise addition was complete, the mixture was reacted at 65° C. for 4 hours. After heating was turned off, the reaction was continued for 12 hours. The reaction product was concentrated under reduced pressure and then separated and purified by silica gel column chromatography (eluents: ethyl acetate/petroleum ether (v/v)=20/100) to obtain 68A (0.5 g, yield 13.7%).

LC-MS (ESI): m/z=270.1 [M−56+H]⁺.

Step 2: 68A (0.5 g, 1.5 mmol) and a hydrogen chloride-dioxane solution (4N, 10 mL) were successively added to a 50 mL single-mouth flask and reacted at room temperature for 1 hour, and the reaction product was then concentrated under reduced pressure to obtain 68B (0.5 g, yield 100.0%).

LC-MS (ESI): m/z=226.1 [M+H]⁺.

Step 3: 1E (430 mg, 1.5 mmol), 68B (500 mg, 1.5 mmol), dioxane (5 mL), and DIPEA (774 mg, 6.0 mmol) were successively added to a 50 mL single-mouth flask and reacted at 100° C. for 12 hours.

The reaction product was cooled to room temperature, and the reaction liquid was directly concentrated under reduced pressure and then separated and purified by silica gel column chromatography (eluent dichloromethane/methanol (v/v)=100/8) to obtain 500 mg of a mixture.

After chiral preparation, the title compound 68-1 (50 mg, 7.0%, retention time: 1.704 min) and the title compound 68-2 (110 mg, 15.4%, retention time: 2.183 min) were obtained. Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 120 mL/min; back pressure: 100 bar; column temperature: 25° C.; wavelength: 220 nm; elution time: 4 min. [00726](Compound 68-1) [00727]¹H NMR (400 MHz, DMSO-d₆) δ 7.77-7.73 (m, 1H), 7.48 (s, 1H), 7.21-7.15 (m, 1H), 4.88-4.81 (m, 1H), 4.80-4.59 (m, 2H), 4.48-4.33 (m, 2H), 4.09-3.95 (m, 1H), 3.79-3.68 (m, 2H), 3.47-3.34 (m, 2H), 3.28-3.17 (m, 1H), 3.07-2.84 (m, 3H), 2.80-2.67 (m, 2H), 2.42-2.15 (m, 4H), 1.87-1.67 (m, 2H).

LC-MS (ESI): m/z=477.1 [M+H]⁺.

### (Compound 68-2)

¹H NMR (400 MHz, DMSO-d₆) δ7.77-7.73 (m, 1H), 7.48 (s, 1H), 7.19-7.15 (m, 1H), 4.90-4.82 (m, 1H), 4.82-4.63 (m, 2H), 4.47-4.31 (m, 2H), 4.10-3.94 (m, 1H), 3.76-3.67 (m, 2H), 3.48-3.33 (m, 2H), 3.27-3.17 (m, 1H), 3.06-2.84 (m, 3H), 2.80-2.69 (m, 2H), 2.38-2.15 (m, 4H), 1.87-1.68 (m, 2H).

LC-MS (ESI): m/z=477.1 [M+H]⁺.

### Example 64

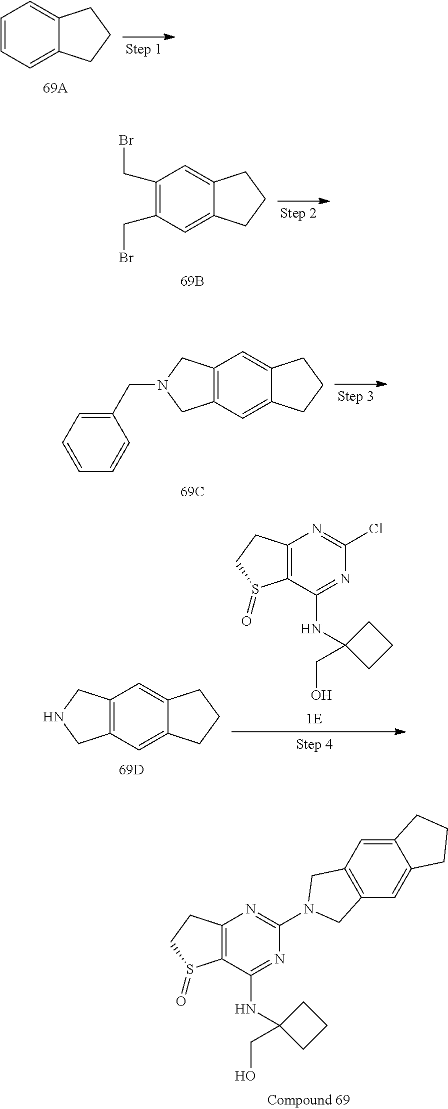 [SMILES: c1ccc2c(c1)CCC2 ; BrCc1cc2c(cc1CBr)CCC2 ; c1ccc(CN2Cc3cc4c(cc3C2)CCC4)cc1 ; c1c2c(cc3c1CNC3)CCC2 ; O=[S@@]1CCc2nc(Cl)nc(NC3(CO)CCC3)c21 ; O=[S@@]1CCc2nc(N3Cc4cc5c(cc4C3)CCC5)nc(NC3(CO)CCC3)c21]

Step 1: Compound 69A (10 g, 84.6 mmol) and paraformaldehyde (25.4 g, 0.85 mol) were dissolved in acetic acid (30 mL), and a 40% hydrobromic acid solution (49.2 mL, 0.85 mol) was then slowly added at 0° C. and stirred for 30 min. The mixture was heated to 80° C., reacted for 12 h, then concentrated, and subjected to column chromatography (pure petroleum ether) to obtain compound 69B (2 g, 8%).

Step 2: Compound 69B (1.0 g, 3.3 mmol) was dissolved in ethanol (30 mL), and benzylamine (0.35 g, 3.3 mmol) was added. The mixture was heated to reflux for 4 h, then passed through a short silica gel column, rinsed with ethyl acetate, concentrated, and subjected to column chromatography (petroleum ether/ethyl acetate=4/1) to obtain compound 69C (0.6 g, 73%).

LC-MS (ESI): m/z=250.4 [M+H]⁺.

Step 3: Compound 69C (0.5 g, 2.0 mmol) was dissolved in methanol (30 mL), and palladium on carbon (50 mg) was then added. After displacement with a hydrogen balloon three times, the mixture was reacted at room temperature for 12 h, filtered and concentrated, and the residue was subjected to column chromatography (methanol/triethylamine=100/1) to obtain compound 69D (0.2 g, 62%).

LC-MS (ESI): m/z=160.2 [M+H]⁺.

Step 4: Compound 69D (0.2 mg, 1.3 mmol) was dissolved in 1,4-dioxane (30.0 mL), compound 1E (0.36 g, 1.3 mmol) and N,N-diisopropylethylamine (0.32 g, 2.6 mmol) were then added, and the mixture was heated back to 90° C. and reacted for 4 h, after which the reaction product was cooled to room temperature, concentrated, and subjected to column chromatography (dichloromethane/methanol=10/1) to obtain compound 69 (0.1 g, 19%).

LC-MS (ESI): m/z=411.2 [M+H]⁺. [00739]¹H NMR (400 MHz, CDCl₃) δ 7.38-7.32 (m, 1H), 7.23-7.12 (m, 2H), 4.91-4.85 (m, 1H), 4.78-4.71 (m, 3H), 3.79 (d, 2H), 3.49-3.41 (m, 1H), 3.27-3.15 (m, 1H), 3.00-2.94 (m, 1H), 2.94-2.83 (m, 5H), 2.44-2.33 (m, 2H), 2.25-2.20 (m, 2H), 2.08-1.99 (m, 2H), 1.87-1.74 (m, 2H).

### Example 65

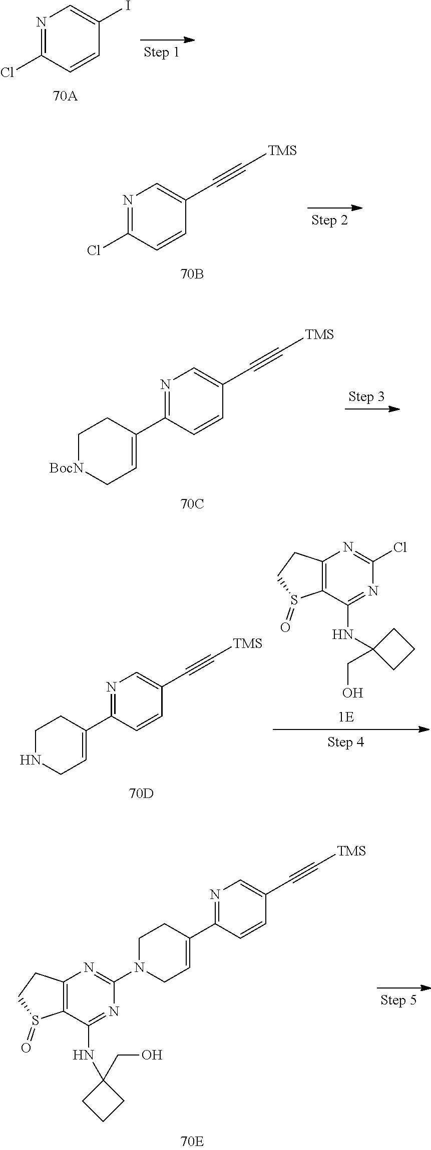 [SMILES: Clc1ccc(I)cn1 ; C[Si](C)(C)C#Cc1ccc(Cl)nc1 ; CC(C)(C)OC(=O)N1CC=C(c2ccc(C#C[Si](C)(C)C)cn2)CC1 ; C[Si](C)(C)C#Cc1ccc(C2=CCNCC2)nc1 ; O=[S@@]1CCc2nc(Cl)nc(NC3(CO)CCC3)c21 ; C[Si](C)(C)C#Cc1ccc(C2=CCN(c3nc4c(c(NC5(CO)CCC5)n3)[S@](=O)CC4)CC2)nc1]

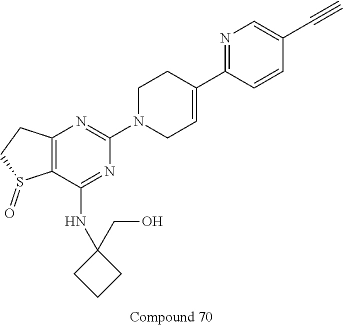 [SMILES: C#Cc1ccc(C2=CCN(c3nc4c(c(NC5(CO)CCC5)n3)[S@](=O)CC4)CC2)nc1]

Step 1: Compound 70A (1.2 g, 5.0 mmol) was dissolved in tetrahydrofuran (50 mL), and bis(triphenylphosphino)palladium dichloride (350.0 mg, 0.5 mmol), triethylamine (1.1 g, 10.0 mmol), trimethylsilylacetylene (650.0 mg, 6.5 mmol), and cuprous iodide (40.0 mg, 0.25 mmol) were then added. After nitrogen displacement three times, the mixture was heated to 50° C. and reacted for 14 h, after which the reaction product was passed through a short silica gel column, rinsed with ethyl acetate, concentrated, and subjected to column chromatography (petroleum ether/ethyl acetate=5/1) to obtain compound 70B (800 mg, 76%).

Step 2: Compound 70B (420.0 mg, 2.0 mmol) was dissolved in 1,4-dioxane (30 mL), 1,1-bis(diphenylphosphino)ferrocene palladium dichloride (144.0 mg, 0.2 mmol), potassium carbonate (560.0 mg, 4.0 mmol), N-Boc-1,2,5,6-tetrahydropyridine-4-boronic acid pinacol ester (720.0 mg, 2.4 mmol), and water (6.0 mL) were then added. After nitrogen displacement three times, the mixture was heated to 45° C. and reacted for 14 h, after which the reaction product was passed through a short silica gel column, rinsed with ethyl acetate, concentrated, and subjected to column chromatography (dichloromethane/methanol=20/1) to obtain compound 70C (400 mg, 56%).

LC-MS (ESI): m/z=357.2 [M+H]⁺.

Step 3: Compound 70C (356.0 mg, 1.0 mmol) was dissolved in dichloromethane (10 mL), trifluoroacetic acid (1.2 mL) was then added, and the mixture was reacted at room temperature for half an hour and then concentrated to obtain compound 70D (150.0 mg, 78%), which was directly used in the next step.

LC-MS (ESI): m/z=257.1 [M+H]⁺.

Step 4: Compound 70D (128.1 mg, 0.5 mmol) was dissolved in 1,4-dioxane (10.0 mL), compound 1E (172.0 mg, 0.6 mmol) was then added, and the mixture was heated back to 90° C. and reacted for 4 h, after which the reaction product was cooled to room temperature, concentrated, and subjected to column chromatography (dichloromethane/methanol=10/1) to obtain compound 70E (200 mg, 79%).

LC-MS (ESI): m/z=508.2 [M+H]⁺.

Step 5: Compound 70E (200.0 mg, 0.4 mmol) was dissolved in methanol (10.0 mL), potassium carbonate (150.0 mg, 1.1 mmol) was then added, and the mixture was then reacted at room temperature for 2 h, concentrated, and then subjected to column chromatography (dichloromethane/methanol=15/1) to obtain compound 70 (110.0 mg, 62%).

LC-MS (ESI): m/z=436.2 [M+H]⁺.

¹H NMR (400 MHz, CDCl₃) δ 8.66 (d, 1H), 7.74-7.72 (m, 1H), 7.35 (d, 1H), 6.75 (t, 1H), 6.57 (s, 1H), 4.47 (s, 2H), 4.06 (s, 2H), 3.87-3.79 (m, 2H), 3.65-3.57 (m, 1H), 3.40-3.32 (m, 1H), 3.23 (s, 1H), 3.08-2.98 (m, 2H), 2.71 (s, 2H), 2.43-2.35 (m, 2H), 2.26-2.18 (m, 2H), 1.96-1.78 (m, 2H).

### Example 66

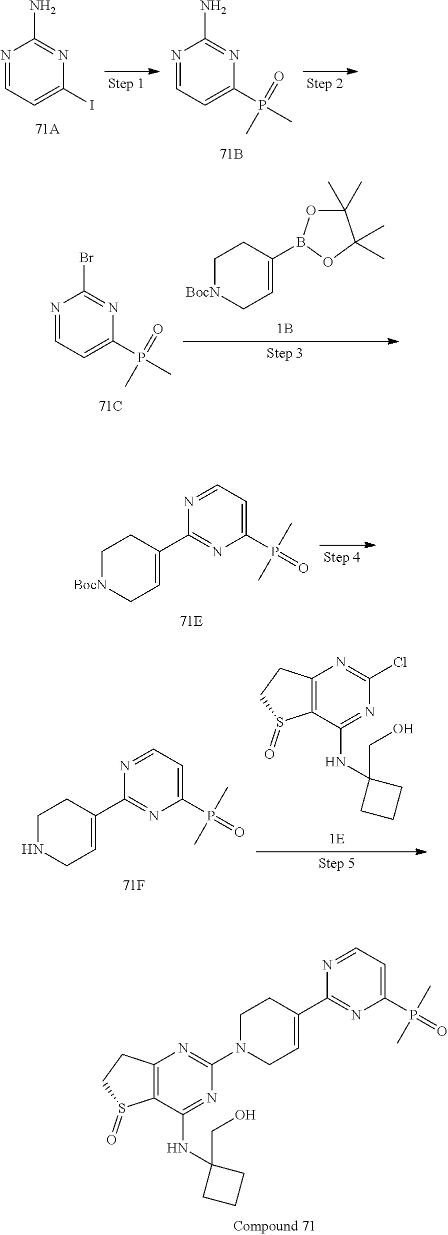

Step 1: Compound 71A (5.00 g, 22.60 mmol) and dimethylphosphine oxide (2.65 g, 33.90 mmol) were weighed into a 250 mL single-mouth flask and dissolved with 1,4-dioxane (50 mL), potassium phosphate (14.39 g, 67.80 mmol), tris(dibenzylideneacetone)dipalladium (1.03 g, 1.13 mmol), and 4,5-bis(diphenylphosphino)-9,9-dimethylxanthene (1.31 g, 2.26 mmol) were added, and after the addition was complete, the mixture was stirred at 90° C. for 16 h. After the completion of the reaction was detected by TLC plate spotting (petroleum ether:ethyl acetate=5:1), water (50 mL) was added and stirred for 5 min, and the mixture was extracted with ethyl acetate (50 mL). The organic phase was separated, dried over anhydrous sodium sulfate, and concentrated under reduced pressure, and the crude product was purified by column chromatography (eluents: petroleum ether:ethyl acetate=1:1) to obtain the target compound 71B (2.2 g, yield: 57%).

LCMS m/z=172.1 [M+1]⁺.

Step 2: Compound 71B (2.2 g, 12.85 mmol) was weighed into a 100 mL single-mouth flask and dissolved with acetonitrile (30 mL). Isoamyl nitrite (3.01 g, 25.70 mmol) and cuprous bromide (4.37 g, 19.22 mmol) were added, and after the addition was complete, the mixture was stirred at 70° C. for 12 h. After the completion of the reaction was detected by TLC plate spotting (petroleum ether: ethyl acetate=5:1), water (50 mL) was added and stirred for 5 min, and ethyl acetate (50 mL) was added for extraction. An organic phase was separated, which was dried over anhydrous sodium sulfate and concentrated under reduced pressure, and the crude product was purified by column chromatography (eluents: petroleum ether:ethyl acetate=3:1) to obtain the target compound 71C (0.43 g, yield 14%).

LCMS m/z=235.0 [M+1]⁺.

Step 3: Compound 71C (0.20 g, 0.85 mmol) and compound 1B (0.32 g, 1.02 mmol) were weighed into a 100 mL single-mouth flask and dissolved with 1,4-dioxane (8 mL), and a 2N sodium carbonate solution (2 mL) and tetrakis(triphenylphosphino)palladium (0.1 g, 0.09 mmol) were added to the reaction liquid. After the addition was complete, the mixture was stirred at 90° C. for 16 h.

After the completion of the reaction was detected by TLC plate spotting (petroleum ether:ethyl acetate=5:1), water (20 mL) was added and stirred for 5 min, ethyl acetate (20 mL) was added for extraction, an organic phase was separated, which was dried over anhydrous sodium sulfate and concentrated under reduced pressure, and the crude product was purified by column chromatography (eluents: petroleum ether:ethyl acetate=5:1) to obtain the target compound 71E (1.0 g, yield 58%).

LCMS m/z=338.1 [M+1]⁺.

Step 4: Compound 71E (0.20 g, 0.59 mmol) was weighed into a 100 mL single-mouth flask and dissolved with dichloromethane (6 mL), trifluoroacetic acid (2 mL) was added, and after the addition was complete, the mixture was stirred for 2 h. The organic phase was concentrated under reduced pressure until no liquid dripped out to obtain the target compound 71F (0.20 g, crude).

LCMS m/z=238.1 [M+1]¹.

Step 5: Compound 1E (150 mg, 0.52 mmol) and compound 71F (150 mg, 0.62 mmol) were added to a 100 mL single-mouth flask and dissolved with 1,4-dioxane (10 mL), and N,N-diisopropylethylamine (0.16 g, 1.56 mmol) was added. After the addition was complete, the system was placed under nitrogen protection and stirred at 90° C. for 16 h, and the reaction liquid was concentrated under reduced pressure to obtain a crude product, which was separated by preparative HPLC to obtain the title compound 71 (110 mg, 43%).

Preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution: mobile phase A: 40-70%; c. flow rate: 15 mL/min; d. elution time: 20 min; retention time: 11.20 min.

LCMS m/z=489.2 [M+1]⁺. [00761]¹H NMR (400 MHZ, DMSO-d₆) δ 8.99 (t, 1H), 7.79 (t, 1H), 7.38 (s, 2H), 4.84 (t, 1H), 4.48 (s, 2H), 3.99 (t, 2H), 3.75 (d, 2H), 3.48-3.39 (m, 1H), 3.26-3.18 (m, 1H), 2.98-2.84 (m, 2H), 2.70 (s, 2H), 2.42-2.30 (m, 2H), 2.24-2.18 (m, 2H), 1.86-1.76 (m, 2H), 1.73 (s, 3H), 1.70 (s, 3H).

### Example 67

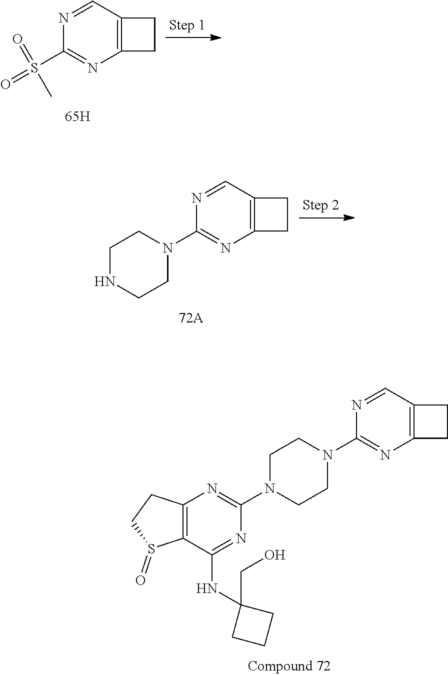 [SMILES: CS(=O)(=O)c1ncc2c(n1)CC2 ; c1nc(N2CCNCC2)nc2c1CC2 ; O=[S@@]1CCc2nc(N3CCN(c4ncc5c(n4)CC5)CC3)nc(NC3(CO)CCC3)c21]

Step 1: 65H (90 mg, 0.49 mmol) was added to a 50 mL single-mouth flask and dissolved with dioxane (6 mL), and piperazine (84 mg, 0.98 mmol) was added and stirred at 90 degrees Celsius for 3 h. Water (5 mL) was added to the reaction liquid, and ethyl acetate (3×10 mL) was added for extraction. The organic phases were combined, dried over anhydrous sodium sulfate, and concentrated to obtain the target compound 72A (60 mg, crude), which was directly used in the next reaction.

LCMS m/z=191.2 [M+1]⁺.

Step 2: 1E (60 mg, 0.21 mmol) was added to a 50 mL single-mouth flask and dissolved with dioxane (6 mL), and 72A (60 mg, 0.32 mmol) and N,N-diisopropylethylamine (68 mg, 0.53 mmol) were successively added and stirred at 95 degrees Celsius overnight. The reaction liquid was concentrated and purified by SFC to obtain the target compound 72 (9 mg, yield: 10%).

Preparation method: instrument: SFC Prep 150; chromatographic column: SunFire silica (19 mm×250 mm; the sample was dissolved with MeOH and filtered with a 0.45 m filter to prepare a sample liquid; preparative chromatography conditions: a. composition of mobile phases A and B: mobile phase A: CO2, mobile phase B: methanol/acetonitrile (7:3); b. isocratic elution, mobile phase B: 35%; c. flow rate: 40 mL/min; d. elution time: 15 min.

LCMS m/z=442.31 [M+1]⁺. [00767]¹H NMR (400 MHz, DMSO-d6) δ 7.95 (s, 1H), 7.36 (s, 1H), 4.81 (s, 1H), 3.84-3.69 (m, 10H), 3.26-3.20 (m, 4H), 3.04-3.02 (m, 2H), 2.98-2.83 (m, 2H), 2.40-2.26 (m, 2H), 2.22-2.16 (m, 2H), 1.83-1.71 (m, 2H).

### Example 68

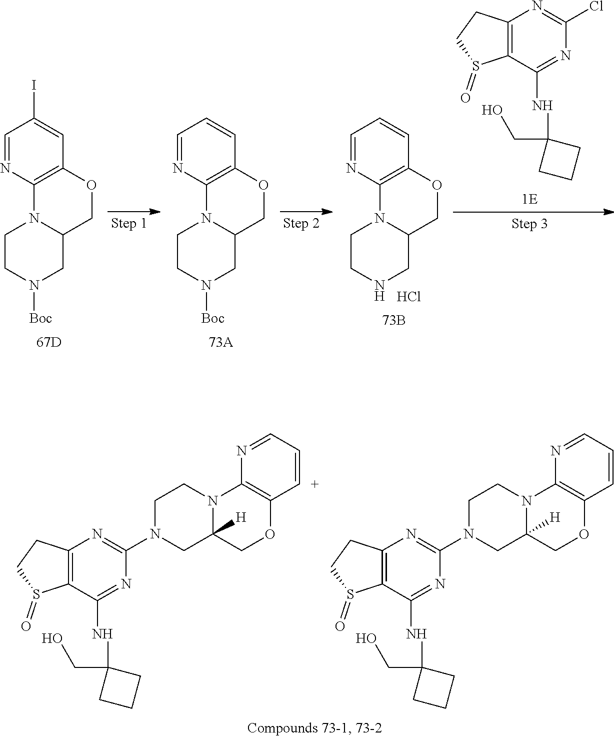

Step 1: Methanol (10 mL), 67D (0.5 g, 1.20 mmol), and palladium on carbon (100 mg) were successively added to a 50 mL single-mouth flask, and the mixture was subjected to a hydrogenation reaction at room temperature for 16 hours, followed by filtration. The filtrate was concentrated under reduced pressure to obtain 73A (0.31 g, yield 88.9%).

LC-MS (ESI): m/z=292.1 [M+H]⁺.

Step 2: 73A (0.31 g, 1.07 mmol) and a hydrogen chloride-dioxane solution (4N, 15 mL) were successively added to a 50 mL single-mouth flask and reacted at room temperature for 1 hour, and the reaction product was then concentrated under reduced pressure to obtain 73B (0.25 g, yield 100.0%).

LC-MS (ESI): m/z=192.1 [M+H]⁺.

Step 3: 1E (287 mg, 1.0 mmol), 73B (250 mg, 1.07 mmol), dioxane (10 mL), and DIPEA (774 mg, 6.0 mmol) were successively added to a 50 mL single-mouth flask and reacted at 100° C. for 12 hours. The reaction product was cooled to room temperature, and the reaction liquid was directly concentrated under reduced pressure and then separated and purified by silica gel column chromatography (eluent dichloromethane/methanol (v/v)=100/8) to obtain 350 mg of a mixture.

After chiral preparation, the title compound 73-1 (91 mg, 20.6%, retention time: 1.472 min) and the title compound 73-2 (63 mg, 14.3%, retention time: 1.770 min) were obtained. Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 100 mL/min; back pressure: 100 bar; column temperature: 25° C.; wavelength: 220 nm; elution time: 4.2 min. [00774](Compound 73-1) [00775]¹H NMR (400 MHz, DMSO-d₆) δ 7.79-7.67 (m, 1H), 7.44 (s, 1H), 7.06-6.94 (m, 1H), 6.68-6.55 (m, 1H), 4.89-4.80 (m, 1H), 4.79-4.62 (m, 2H), 4.50-4.34 (m, 2H), 4.06-3.92 (m, 1H), 3.79-3.65 (m, 2H), 3.50-3.37 (m, 1H), 3.36-3.32 (m, 1H), 3.28-3.16 (m, 1H), 3.06-2.81 (m, 3H), 2.79-2.63 (m, 2H), 2.41-2.25 (m, 2H), 2.25-2.14 (m, 2H), 1.89-1.71 (m, 2H).

LC-MS (ESI): m/z=443.2 [M+H]⁺. [00777](Compound 73-2) [00778]¹H NMR (400 MHz, DMSO-d₆) δ 7.84-7.64 (m, 1H), 7.44 (s, 1H), 7.15-6.83 (m, 1H), 6.80-6.46 (m, 1H), 4.87-4.81 (m, 1H), 4.80-4.62 (m, 2H), 4.50-4.31 (m, 2H), 4.04-3.94 (m, 1H), 3.76-3.68 (m, 2H), 3.50-3.38 (m, 1H), 3.36-3.31 (m, 1H), 3.28-3.16 (m, 1H), 3.05-2.82 (m, 3H), 2.81-2.64 (m, 2H), 2.43-2.25 (m, 2H), 2.25-2.14 (m, 2H), 1.85-1.71 (m, 2H).

LC-MS (ESI): m/z=443.2 [M+H]⁺.

### Example 69

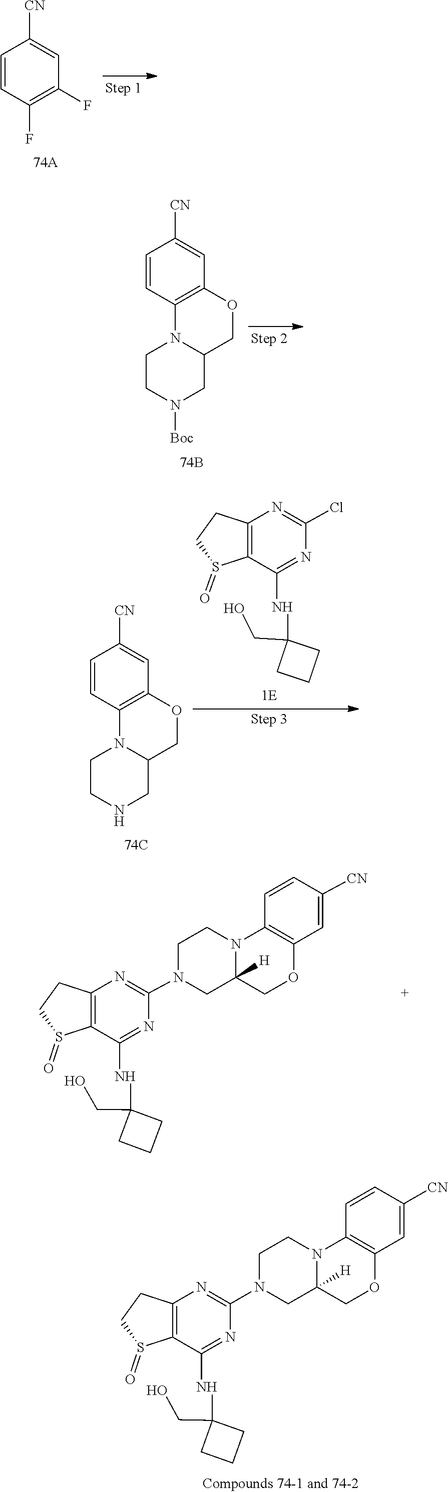

Step 1: N,N-dimethylformamide (50 mL), 74A (5.0 g, 35.97 mmol), tert-butyl 3-(hydroxymethyl)piperazine-1-carboxylate (7.77 g, 35.97 mmol), and potassium hydroxide (6.05 g, 107.85 mmol) were successively added to a 250 mL single-mouth flask and reacted at 100° C. for 16 hours, followed by filtration, after which ethyl acetate (100 mL) was added to the filtrate, which was washed with water (50 mL×3) and with a saturated sodium chloride aqueous solution (100 mL×1).

The organic phase was dried over anhydrous sodium sulfate and filtered, and the filtrate was concentrated under reduced pressure and then separated and purified by silica gel column chromatography (eluents: ethyl acetate/petroleum ether (v/v)=20/100) to obtain 74B (0.5 g, yield 4.4%).

LC-MS (ESI): m/z=316.2 [M+H]⁺.

Step 2: 74B (0.5 g, 1.59 mmol), dichloromethane (15 mL), and triethylamine (3.21 g, 31.8 mmol) were successively added to a 50 mL single-mouth flask, and iodotrimethylsilane (3.18 g, 15.9 mmol) was dropwise added at 0° C. The mixture was reacted at room temperature for 1 hour, and the reaction product was concentrated under reduced pressure to obtain crude product 74C (4.5 g, yield 100.0%).

LC-MS (ESI): m/z=216.2 [M+H]⁺.

Step 3: 1E (456 mg, 1.59 mmol), crude product 74C (4.5 g, 1.59 mmol), dioxane (10 mL), and DIPEA (1.03 g, 8.0 mmol) were successively added to a 50 mL single-mouth flask and reacted at 100° C. for 12 hours. The reaction product was cooled to room temperature, and the reaction liquid was directly concentrated under reduced pressure and then separated and purified by silica gel column chromatography (eluent dichloromethane/methanol (v/v)=100/10) to obtain 450 mg of a mixture.

After chiral preparation, the title compound 74-1 (112 mg, 15.1%, retention time: 0.925 min) and the title compound 74-2 (86 mg, 11.6%, retention time: 1.896 min) were obtained. Chiral preparation method: instrument: Waters 150 MGM; chromatographic column: Chiralpak Column; mobile phase: A: carbon dioxide, and B: isopropanol (0.1% aqueous ammonia); isocratic elution: 70% mobile phase B; flow rate: 100 mL/min; back pressure: 100 bar; column temperature: 25° C.; wavelength: 220 nm; elution time: 5.1 min. [00786](Compound 74-1) [00787]¹H NMR (400 MHz, DMSO-d₆) δ 7.46 (s, 1H), 7.35-7.31 (m, 1H), 7.14-7.09 (m, 1H), 6.89-6.84 (m, 1H), 4.88-4.79 (m, 1H), 4.78-4.60 (m, 2H), 4.51-4.39 (m, 1H), 4.10-4.00 (m, 1H), 3.97-3.88 (m, 1H), 3.77-3.66 (m, 2H), 3.52-3.36 (m, 1H), 3.29-3.16 (m, 1H), 3.16-3.02 (m, 2H), 3.00-2.81 (m, 2H), 2.79-2.62 (m, 2H), 2.44-2.25 (m, 2H), 2.25-2.14 (m, 2H), 1.88-1.69 (m, 2H).

LC-MS (ESI): m/z=467.1 [M+H]⁺.

### (Compound 74-2)

¹H NMR (400 MHz, DMSO-d₆) δ 7.46 (s, 1H), 7.40-7.30 (m, 1H), 7.14-7.10 (m, 1H), 6.88-6.84 (m, 1H), 4.88-4.79 (m, 1H), 4.79-4.60 (m, 2H), 4.51-4.38 (m, 1H), 4.12-3.98 (m, 1H), 3.95-3.87 (m, 1H), 3.79-3.65 (m, 2H), 3.52-3.38 (m, 1H), 3.28-3.17 (m, 1H), 3.15-3.02 (m, 2H), 3.01-2.83 (m, 2H), 2.79-2.61 (m, 2H), 2.45-2.25 (m, 2H), 2.24-2.13 (m, 2H), 1.87-1.67 (m, 2H).

LC-MS (ESI): m/z=467.1 [M+H]⁺.

### Example 70

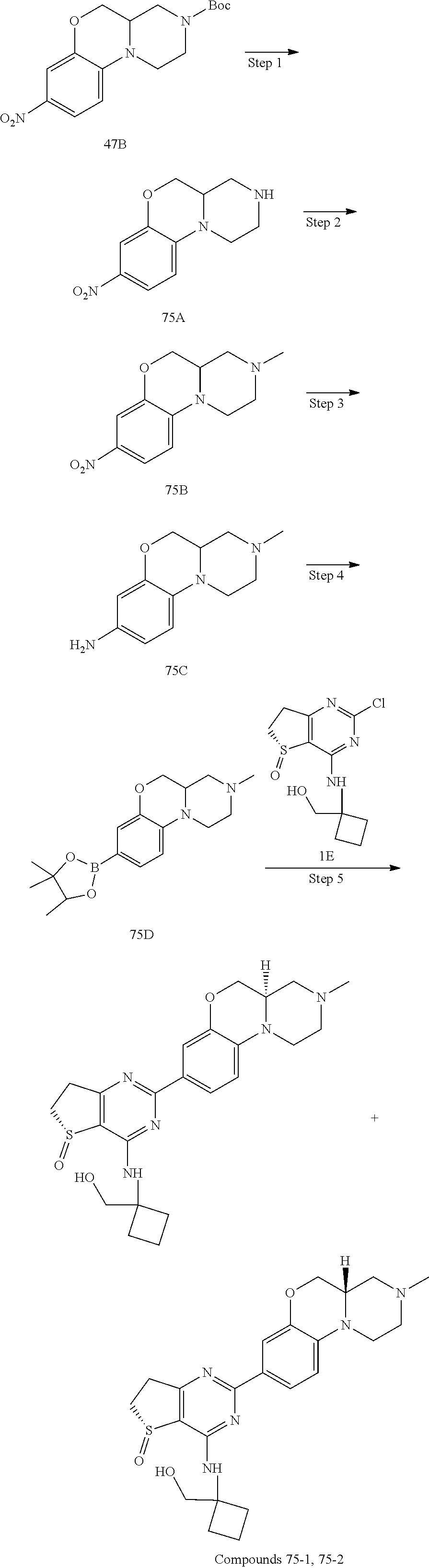

Step 1: 47B (10.0 g, 29.8 mmol) and hydrogen chloride-1,4-dioxane (100 mL) were successively added to a 250 mL single-mouth flask and reacted at room temperature for 1 hour. After concentration, a saturated sodium bicarbonate aqueous solution (100 mL) was added to adjust the pH to >7, and dichloromethane (100 mL×2) was used for extraction. The organic phase was dried over anhydrous sodium sulfate and filtered, and the filtrate was concentrated under reduced pressure to obtain 75A (7.0 g, 99.8%).

LC-MS (ESI): m/z=236.1 [M+H]⁺.

Step 2: 75A (3.5 g, 14.9 mmol), anhydrous ethanol (40 mL), paraformaldehyde (3.5 g), and sodium cyanoborohydride (1.88 g, 29.8 mmol) were successively added to a 50 mL single-mouth flask and reacted at room temperature for 12 hours. The reaction product was concentrated under reduced pressure and then separated and purified by silica gel column chromatography (dichloromethane/methanol (v/v)=90/10) to obtain 75B (2.5 g, 67.6%).

LC-MS (ESI): m/z=250.2 [M+H]⁺.

Step 3: 75B (2.5 g, 10.0 mmol), ethyl acetate (50 mL), and palladium on carbon (10%, 0.5 g) were successively added to a 100 mL single-mouth flask, and the mixture was subjected to a hydrogenation reduction reaction at room temperature for 12 hours. After filtration, the filtrate was concentrated under reduced pressure to obtain 75C (2.2 g, 100%).

LC-MS (ESI): m/z=220.2 [M+H]⁺.

Step 4: 75C (2.2 g, 10.0 mmol), methanol (20 mL), and a hydrogen chloride aqueous solution (3 M, 10 mL) were successively added to a 100 mL single-mouth flask, and sodium nitrite (693 mg, 10.0 mmol) was added at −5° C. Pinacol diborate (5.1 g, 20.1 mmol) was immediately added. The mixture was reacted at room temperature for 1 hour, the reaction product was adjusted to pH >7 by adding saturated sodium bicarbonate and extracted with dichloromethane (100 mL×2), and the organic phase was dried over anhydrous sodium sulfate and filtered. The filtrate was concentrated under reduced pressure and then separated and purified by silica gel column chromatography (dichloromethane/methanol (v/v)=90/10) to obtain 75D (2.0 g, 63.1%).

LC-MS (ESI): m/z=317.2 [M+H]⁺.

Step 5: Under nitrogen protection, 1E (907 mg, 3.16 mmol), 75D (1.0 g, 3.16 mmol), water (4 mL), 1,1′-bis(diphenylphosphino)ferrocene palladium dichloride (224 mg, 0.32 mmol), and potassium carbonate (1.3 g, 9.48 mmol) were successively added to a 100 mL single-mouth flask, dissolved by adding 1,4-dioxane (20 mL), warmed to 100° C., and reacted for 5 hours. The reaction product was cooled to room temperature, and the reaction liquid was directly concentrated under reduced pressure and then separated and purified by silica gel column chromatography (dichloromethane/methanol (v/v)=90/10) to obtain a mixture of compounds 75-1 and 75-2 (200 mg, 13.8%).

After chiral preparation, the title compound 75-1 (81 mg, 5.6%, retention time: 0.885 min) and the title compound 75-2 (74 mg, 5.1%, retention time: 1.795 min) were obtained.

Chiral preparation method: instrument: Waters 150 MGM; chromatographic column: Chiralpak Column; mobile phase: A: carbon dioxide, and B: isopropanol (0.1% aqueous ammonia); isocratic elution: 70% mobile phase B; flow rate: 100 mL/min; back pressure: 100 bar; column temperature: 25° C.; wavelength: 220 nm; elution time: 5.1 min.

### (Compound 75-1)

¹H NMR (400 MHz, DMSO-d₆) δ 7.84-7.79 (m, 1H), 7.75 (s, 1H), 7.64-7.61 (m, 1H), 6.98-6.89 (m, 1H), 4.93-4.83 (m, 1H), 4.31-4.23 (m, 1H), 3.96-3.89 (m, 1H), 3.84-3.73 (m, 3H), 3.66-3.51 (m, 1H), 3.40-3.31 (m, 1H), 3.22-3.09 (m, 2H), 3.02-2.94 (m, 1H), 2.91-2.71 (m, 3H), 2.47-2.32 (m, 2H), 2.31-2.24 (m, 2H), 2.23 (s, 3H), 2.11-2.02 (m, 1H), 1.90-1.77 (m, 2H), 1.75-1.66 (m, 1H).

LC-MS (ESI): m/z=456.2 [M+H]⁺.

### (Compound 75-2)

¹H NMR (400 MHz, DMSO-d₆) δ 7.83-7.80 (m, 1H), 7.75 (s, 1H), 7.64-7.61 (m, 1H), 6.98-6.91 (m, 1H), 4.93-4.86 (m, 1H), 4.30-4.23 (m, 1H), 3.99-3.87 (m, 1H), 3.85-3.73 (m, 3H), 3.63-3.52 (m, 1H), 3.38-3.31 (m, 1H), 3.21-3.11 (m, 2H), 3.02-2.93 (m, 1H), 2.91-2.71 (m, 3H), 2.46-2.32 (m, 2H), 2.31-2.24 (m, 2H), 2.23 (s, 3H), 2.14-1.99 (m, 1H), 1.89-1.78 (m, 2H), 1.75-1.66 (m, 1H).

LC-MS (ESI): m/z=456.2 [M+H]⁺.

### Example 71

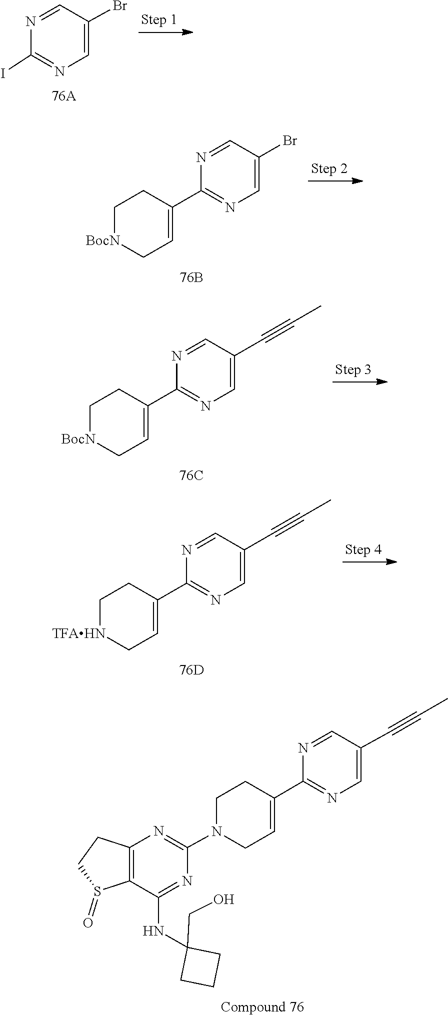

Step 1: Under nitrogen protection, compound 76A (2.00 g, 7.02 mmol), [1-(tert-butoxycarbonyl)-1,2,3,6-tetrahydropyridin-4-yl]boronic acid (2.28 g, 7.37 mmol), potassium carbonate (1.94 g, 14.04 mmol), and [1,1′-bis(diphenylphosphino)ferrocene]palladium dichloride (770 mg, 1.05 mmol) were added to a reaction flask. Tetrahydrofuran (40 mL) and water (8 mL) were added, and after nitrogen displacement, the mixture was warmed to 75° C. and reacted for 16 h. After the disappearance of raw materials was detected by TLC, the reaction product was diluted by adding water (100 mL) and extracted with ethyl acetate (100 mL) three times. The organic phases were combined, dried over anhydrous sodium sulfate, and then concentrated in vacuum to obtain a crude product. The crude product was purified by column chromatography (petroleum ether/ethyl acetate (v/v)=100/0 to 95/5) to obtain the target compound 76B (1.00 g, 41.9%).

LC-MS (ESI): m/z=254.60 [M−56+H]⁺. [00811]¹H NMR (400 MHz, CDCl₃) δ 8.71 (s, 2H), 7.21-7.20 (m, 1H), 4.16-4.15 (m, 2H), 3.64-3.61 (m, 2H), 2.69-2.68 (m, 2H), 1.49 (s, 9H).

Step 2: Compound 76B (1.00 g, 2.94 mmol), 1-(trimethylsilyl)propyne (660 mg, 5.88 mmol), potassium carbonate (1.02 g, 7.35 mmol), cuprous iodide (112 mg, 0.59 mmol), and [1,1′-bis(diphenylphosphino)ferrocene]palladium dichloride (160 mg, 0.44 mmol) were added to a reaction flask, and a solution of 1,4-dioxane (20 mL) was added. After nitrogen displacement, the mixture was warmed to 85° C. and reacted for 16 h. After the disappearance of raw materials was detected by TLC, the reaction product was diluted by adding water (100 mL) and extracted with ethyl acetate (100 mL) three times. The organic phases were combined, dried over anhydrous sodium sulfate, and then concentrated in vacuum to obtain a crude product. The crude product was purified by column chromatography (petroleum ether/ethyl acetate (v/v)=100/0 to 95/5) three times to obtain the target compound 76C (200 mg, 22.7%).

LC-MS (ESI): m/z=244.10 [M−56+H]⁺.

Step 3: Compound 76C (200 mg, 0.67 mmol) was dissolved in dichloromethane (8 mL), and trifluoroacetic acid (2 mL) was added and then stirred at 0° C. for 30 min. After the disappearance of raw materials was detected by TLC, the reaction liquid was concentrated at room temperature to obtain crude 76D (230 mg), which was directly used in the next reaction.

LC-MS (ESI): m/z=200.20 [M+H]⁺.

Step 4: Compound 1E (130 mg, 0.45 mmol) and compound 76D (230 mg, 0.63 mmol) were dissolved in 1,4-dioxane (6 mL), and N,N-diisopropylethylamine (350 mg, 2.71 mmol) was then added. After the addition was complete, the mixture was warmed to 85° C. under nitrogen protection and stirred for 16 h. After the reaction was complete, the reaction liquid was cooled to room temperature and concentrated under reduced pressure to obtain a crude product, which was purified by column chromatography (dichloromethane/methanol (v/v)=100/0 to 90/10) and then further by prep.HPLC (preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution: mobile phase A: 30-95%; c. flow rate: 15 mL/min; d. elution time: 15 min; retention time: 6.0 min) to obtain the target compound 76 (48 mg, 23.6%). [00817]¹H NMR (400 MHz, DMSO-d6) δ 8.80 (s, 2H), 7.34 (s, 1H), 7.29 (s, 1H), 4.85-4.82 (m, 1H), 4.46 (s, 2H), 3.98-3.95 (m, 2H), 3.75-3.74 (m, 2H), 3.47-3.39 (m, 1H), 3.26-3.19 (m, 1H), 2.98-2.92 (m, 1H), 2.89-2.84 (m, 1H), 2.65 (s, 2H), 2.43-2.30 (m, 2H), 2.22-2.18 (m, 2H), 2.12 (s, 3H), 1.83-1.73 (m, 2H).

LCMS m/z=451.2 [M+H]⁺.

### Example 72

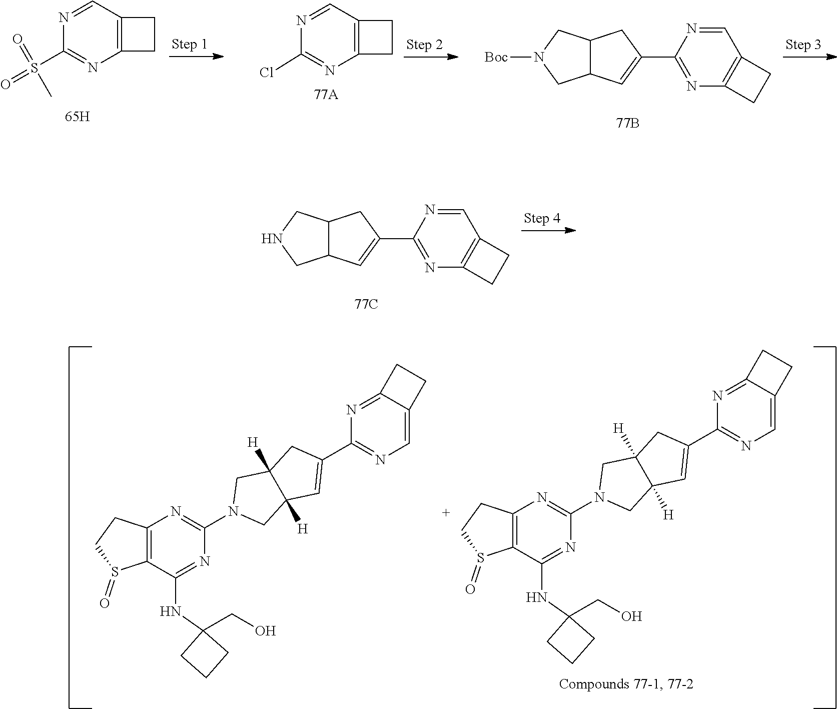

Step 1: Compound 65H (0.5 g, 2.72 mmol) was dissolved in glacial acetic acid (20 mL), and thionyl dichloride (10 mL) was added and stirred at 60° C. overnight. After the reaction was complete, the reaction product was cooled to room temperature, and the reaction system was diluted by adding ethyl acetate (50 mL). The pH of the reaction system was adjusted to neutrality with a saturated sodium bicarbonate aqueous solution and extracted with ethyl acetate (50 mL×3). The organic phase was dried over anhydrous sodium sulfate, filtered, and concentrated, and the residue was subjected to silica gel column chromatography (petroleum ether/ethyl acetate (v/v)=100/0 to 80/20) for fast separation and purification to obtain compound 77A (100 mg, 26%).

LCMS (ESI): m/z=141.1 [M+H]⁺.

Step 2: Under nitrogen protection, compound 42C (238 mg, 0.71 mmol), 77A (100 mg, 0.71 mmol), 1,1-bis(diphenylphosphino)ferrocene palladium dichloride (51 mg, 0.07 mmol), and sodium carbonate (226 mg, 2.13 mmol) were all dissolved in a mixed solvent of 1,4-dioxane/water (10 mL/2 mL), warmed to 90° C., and reacted for 18 h. After the reaction was complete, the reaction product was cooled to room temperature, filtered with diatomite, and washed with ethyl acetate. After the filtrate was concentrated, the obtained residue was separated and purified by column chromatography (petroleum ether/ethyl acetate (v/v)=100/0 to 90/10) to obtain the target compound 77B (102 mg, 45%).

LCMS (ESI): m/z=258.1 [M+H−56]+.

Step 3: Compound 77B (102 mg, 0.33 mmol) was dissolved in a mixed solvent of dichloromethane/trifluoroacetic acid (6 mL/2 mL), stirred at room temperature for 1 h, and directly spin-dried to obtain compound 77C trifluoroacetate, which was directly used in the next reaction (111 mg).

LCMS (ESI): m/z=214.1 [M+H]⁺.

Step 4: Compound 77C trifluoroacetate compound (111 mg) was dissolved in 1,4-dioxane (5 ml), 1E (95 mg, 0.33 mmol) and diisopropylethylamine (128 mg, 0.99 mmol) were added, and the reaction system was placed in an oil bath at 100° C. and reacted for 15 h. After the reaction was complete, the reaction product was cooled to room temperature, and the reaction was monitored by LCMS. After the reaction was complete, the reaction product was directly spin-dried. The crude product was separated and purified by silica gel column chromatography (dichloromethane/methanol (v/v)=100/0 to 80/20) to obtain a mixture of compounds 77-1 and 77-2 (50 mg, 33%). After further chiral resolution, compound 77-1 (26 mg, retention time: 1.891 s) and compound 77-2 (21.6 mg, retention time: 2.901 s) were obtained.

Chiral preparation method: instrument: Waters 150 Prep-SFC E; chromatographic column: Chiralcel Cellulose-2 column; mobile phase: A: carbon dioxide, and B: methanol (0.1% aqueous ammonia); gradient elution: B 40%; flow rate: 100 mL/min; back pressure: 100 bar; column temperature: 25° C.; wavelength: 220 nm; elution time: 7.0 min.

### (Compound 77-1)

¹H NMR (400 MHz, CDCl₃) δ 8.23 (s, 1H), 6.78 (s, 1H), 5.67 (s, 1H), 5.09 (d, 1H), 4.07-3.92 (m, 1H), 3.89-3.73 (m, 4H), 3.67 (s, 1H), 3.61-3.53 (m, 1H), 3.49-3.44 (m, 2H), 3.40 (d, 1H), 3.31 (d, 1H), 3.23 (t, 2H), 3.16 (s, 1H), 3.11-2.92 (m, 3H), 2.85 (d, 1H), 2.36 (d, 2H), 2.23-2.15 (m, 2H), 1.97-1.89 (m, 2H).

LCMS (ESI): m/z=465.2 [M+H]⁺.

### (Compound 77-2)

¹H NMR (400 MHz, CDCl₃) δ 8.23 (s, 1H), 6.78 (s, 1H), 5.83 (s, 1H), 5.09 (d, 1H), 4.06-3.90 (m, 1H), 3.89-3.73 (m, 4H), 3.67 (s, 1H), 3.57 (d, 1H), 3.46 (s, 2H), 3.43-3.28 (m, 2H), 3.23 (s, 2H), 3.18-2.96 (m, 4H), 2.87 (d, 1H), 2.41-2.31 (m, 1H), 2.28-2.15 (m, 3H), 1.99-1.83 (m, 2H).

LCMS (ESI): m/z=465.2 [M+H]⁺.

### Example 73

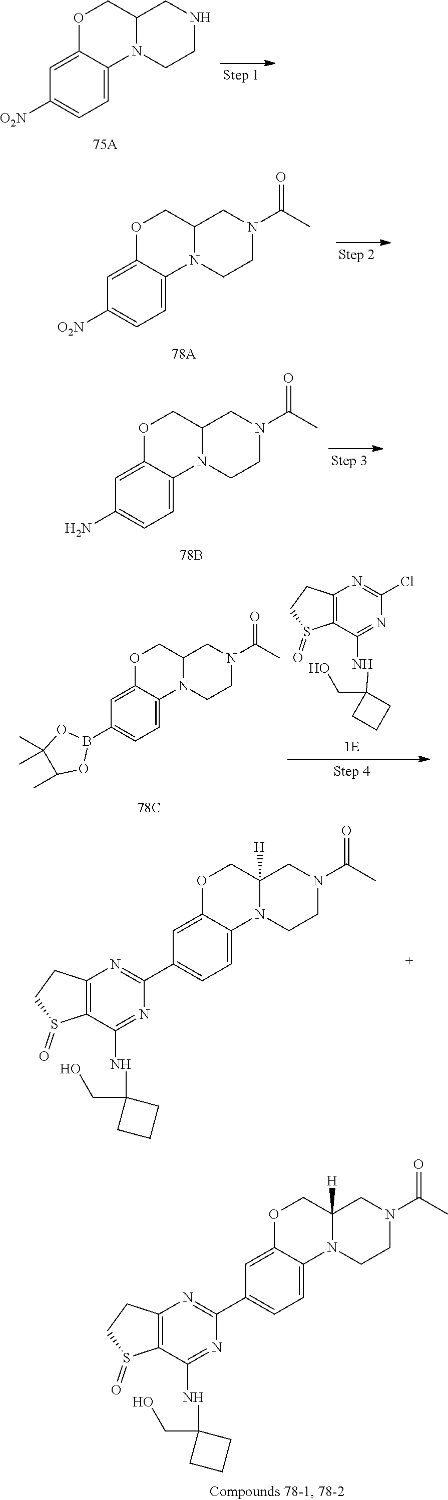

Step 1: 75A (3.5 g, 14.89 mmol) was dissolved in dichloromethane (35 mL), and triethylamine (4.5 g, 44.67 mmol) and acetic anhydride (1.5 g, 14.89 mmol) were added and reacted at room temperature for 1 hour, after which water (50 mL) was added, and the mixture was extracted with dichloromethane (50 mL×2). The organic phase was dried over anhydrous sodium sulfate and filtered, and the filtrate was concentrated under reduced pressure and then separated and purified by silica gel column chromatography (dichloromethane/methanol (v/v)=91/9) to obtain 78A (3.2 g, 77.6%).

LC-MS (ESI): m/z=278.1 [M+H]⁺.

Step 2: Compound 78A (3.2 g, 11.55 mmol) was dissolved in ethyl acetate (50 mL), palladium on carbon (10%, 0.5 g) was added, and the mixture was warmed to 50° C. in a hydrogen atmosphere and reacted for 12 hours. After the reaction was complete, the reaction product was directly filtered and the filtrate was concentrated under reduced pressure to obtain 78B (2.8 g, 98.2%).

LC-MS (ESI): m/z=248.2 [M+H]⁺.

Step 3: 78B (2.5 g, 10.12 mmol), methanol (20 mL), and a hydrogen chloride aqueous solution (3 M, 10 mL) were successively added to a 100 mL single-mouth flask, and sodium nitrite (698 mg, 10.12 mmol) was added at −5° C. Pinacol diboronate (5.1 g, 20.24 mmol) was immediately added, and the mixture was reacted at room temperature for 1 hour. After the completion of the reaction was monitored by TLC, the reaction product was adjusted to pH >7 by adding saturated sodium bicarbonate and extracted with dichloromethane (100 mL×2), and the organic phase was dried over anhydrous sodium sulfate and filtered. The filtrate was concentrated under reduced pressure and then separated and purified by silica gel column chromatography (dichloromethane/methanol (v/v)=91/9) to obtain 78C (2.5 g, 71.8%).

LC-MS (ESI): m/z=345.2 [M+H]⁺.

Step 4: 1E (907 mg, 3.16 mmol), 78C (1.1 g, 3.16 mmol), 1,1′-bis(diphenylphosphino)ferrocene palladium dichloride (224 mg, 0.32 mmol), and potassium carbonate (1.3 g, 9.48 mmol) were successively added to a 100 mL single-mouth flask, dissolved by adding a mixed solvent of 1,4-dioxane (20 mL) and water (4 mL), warmed to 100° C. under nitrogen protection, and reacted for 5 hours. The reaction product was cooled to room temperature, and the reaction liquid was directly concentrated under reduced pressure and then separated and purified by silica gel column chromatography (dichloromethane/methanol (v/v)=91/9) to obtain a mixture of 78-1 and 78-2 (250 mg).

After chiral preparation, the title compound 78-1 (78 mg, 5.1%, retention time: 0.960 min) and the title compound 78-2 (69 mg, 4.5%, retention time: 1.256 min) were obtained. Chiral preparation method: instrument: Waters 150 MGM; chromatographic column: Chiralpak Column; mobile phase: A: carbon dioxide, and B: ethanol (0.1% aqueous ammonia); isocratic elution: 70% mobile phase B; flow rate: 100 mL/min; back pressure: 100 bar; column temperature: 25° C.; wavelength: 220 nm; elution time: 4.0 min. [00841](Compound 78-1) [00842]¹H NMR (400 MHz, DMSO-d₆) δ 7.92-7.75 (m, 2H), 7.69-7.55 (m, 1H), 7.05-6.93 (m, 1H), 4.97-4.82 (m, 1H), 4.50-4.31 (m, 2H), 4.06-3.85 (m, 3H), 3.85-3.73 (m, 2H), 3.64-3.52 (m, 1H), 3.38-3.31 (m, 1H), 3.30-2.60 (m, 6H), 2.46-2.34 (m, 2H), 2.32-2.21 (m, 2H), 2.12-1.98 (m, 3H), 1.94-1.71 (m, 2H).

LC-MS (ESI): m/z=484.4 [M+H]⁺.

### (Compound 78-2)

¹H NMR (400 MHz, DMSO-d₆) δ 7.94-7.74 (m, 2H), 7.70-7.56 (m, 1H), 7.05-6.82 (m, 1H), 5.00-4.84 (m, 1H), 4.51-4.29 (m, 2H), 4.02-3.87 (m, 3H), 3.84-3.75 (m, 2H), 3.64-3.52 (m, 1H), 3.39-3.32 (m, 1H), 3.29-2.62 (m, 6H), 2.46-2.35 (m, 2H), 2.32-2.20 (m, 2H), 2.09-2.02 (m, 3H), 1.91-1.77 (m, 2H).

LC-MS (ESI): m/z=484.4 [M+H]⁺.

### Example 74

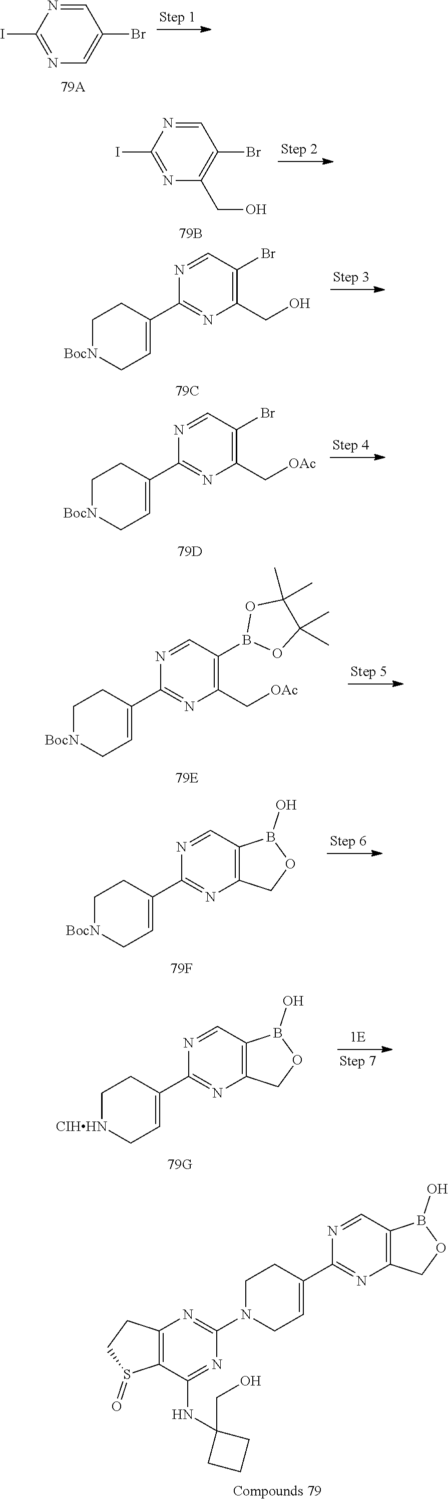

Step 1: Under nitrogen protection, compound 79A (20.00 g, 70.20 mmol) was dissolved in methanol (200 mL). Trifluoroacetic acid (8.17 g, 71.61 mmol) and benzoyl peroxide (19.05 g, 78.63 mmol) were successively and separately added while stirring at room temperature. After the addition was complete, the reaction liquid was warmed to 65° C. and stirred for 16 h. After the disappearance of raw materials was detected by TLC, the reaction product was cooled to room temperature, concentrated under reduced pressure into a small volume, then diluted by adding a saturated sodium carbonate aqueous solution (400 mL), and extracted with ethyl acetate (100 mL) three times. The organic phases were combined, dried over anhydrous sodium sulfate, and then concentrated in vacuum to obtain a crude product. The crude product was purified by column chromatography (petroleum ether/ethyl acetate (v/v)=100/0 to 80/20) to obtain the target compound 79B (2.00 g, 9.05%).

¹H NMR (400 MHz, DMSO-d6) δ 8.67 (s, 1H), 5.58-5.55 (m, 1H), 4.56-4.54 (m, 2H).

LC-MS (ESI): m/z=314.9 [M+H]⁺.

Step 2: Under nitrogen protection, compound 79B (2.00 g, 6.35 mmol) was added to a mixed solution of 1,4-dioxane (20 mL) and water (4 mL). [1-(tert-butoxycarbonyl)-1,2,3,6-tetrahydropyridin-4-yl]boronic acid (2.00 g, 6.48 mmol), [1,1′-bis(diphenylphosphino)ferrocene]palladium dichloride (0.68 g, 0.93 mmol), and potassium carbonate (1.76 g, 12.70 mmol) were added successively and separately with stirring at room temperature.

After the addition was complete, the reaction liquid was warmed to 85° C. and stirred for 16 h. After the disappearance of raw materials was detected by TLC, the reaction product was cooled to room temperature, concentrated under reduced pressure into a small volume, then diluted by adding water (100 mL), and extracted with ethyl acetate (50 mL) three times. The organic phases were combined, dried over anhydrous sodium sulfate, and then concentrated in vacuum to obtain a crude product. The crude product was purified by column chromatography (petroleum ether/ethyl acetate (v/v)=100/0 to 80/20) to obtain the target compound 79C (950 mg, 40.40%). [00851]¹H NMR (400 MHz, CDCl₃) δ 8.67 (s, 1H), 7.27-7.26 (m, 1H), 4.74 (s, 2H), 4.19-4.18 (m, 2H), 3.66-3.63 (m, 2H), 2.73 (s, 2H), 1.49 (s, 9H).

LC-MS (ESI): m/z=314.9 [M+H−56]+.

Step 3: Under nitrogen protection, compound 79C (950 mg, 2.57 mmol) was dissolved in dichloromethane (20 mL), triethylamine (650 mg, 6.41 mmol) was added with stirring in ice bath, and acetyl chloride (300 mg, 3.85 mmol) was slowly added dropwise. After the addition was complete, the reaction liquid was stirred at 0° C. for 1 h. After the disappearance of raw materials was detected by TLC, the reaction was quenched by adding a saturated ammonium chloride aqueous solution (50 mL), and the reaction product was extracted with dichloromethane (40 mL) three times.

The organic phases were combined, dried over anhydrous sodium sulfate, and then concentrated in vacuum to obtain a crude product. The crude product was further purified by column chromatography (petroleum ether/ethyl acetate (v/v)=100/0 to 80/20) to obtain the target compound 79D (750 mg, 70.90%). [00854]¹H NMR (400 MHz, CDCl₃) δ 8.67 (s, 1H), 7.19 (br s, 1H), 5.25 (s, 2H), 4.16-4.15 (m, 2H), 3.63-3.60 (m, 2H), 2.66 (s, 2H), 2.22 (s, 3H), 1.49 (s, 9H).

LC-MS (ESI): m/z=356.3 [M+H−56]+.

Step 4: Under nitrogen protection, compound 79D (750 mg, 1.82 mmol) was dissolved in 1,4-dioxane (20 mL). Pinacol diboronate (530 mg, 2.08 mmol), [1,1′-bis(diphenylphosphino)ferrocene]palladium dichloride (110 mg, 0.20 mmol), and potassium acetate (540 mg, 5.48 mmol) were successively added with stirring at room temperature. After the addition was complete, the reaction liquid was warmed to 80° C. and stirred for 5 h. After the disappearance of raw materials was detected by TLC, the reaction product was cooled to room temperature, concentrated under reduced pressure into a small volume, then diluted by adding water (50 mL), and extracted with ethyl acetate (50 mL) three times. The organic phases were combined, dried by anhydrous sodium sulfate, and then concentrated in vacuum to obtain crude product 79E (1.2 g), which was directly used in the next reaction.

Step 5: Crude compound 79E (1.2 g) was dissolved in methanol (20 mL), and a sodium hydroxide aqueous solution (240 mg, 6.01 mmol, 1 M) was added, and after the addition was complete, the mixture was stirred at room temperature for 16 h under nitrogen protection. The reaction liquid was concentrated under reduced pressure into a small volume, diluted by adding water (20 mL), and extracted twice with ethyl acetate (50 mL). The aqueous phase was further concentrated and then purified by a reverse-phase column (water/acetonitrile (v/v)=100/0 to 10/90) to obtain compound 79F (270 mg, 42.51%).

LCMS m/z=262.2 [M−56+H]¹.

Step 6: Compound 79F (270 mg, 0.85 mmol) was dissolved in dichloromethane (4 mL), and a hydrogen chloride dioxane solution (310 mg, 8.51 mmol, 4 M) was added in ice bath. After the addition was complete, the mixture was stirred for 1.5 h under nitrogen protection. The reaction liquid was concentrated under reduced pressure to obtain crude compound 79G (200 mg), which was directly used in the next reaction.

LCMS m/z=218.2 [M+H]⁺.

Step 7: Compound 1E (100 mg, 0.35 mmol) and crude compound 79G (200 mg) were dissolved in 1,4-dioxane (6 mL), and N,N-diisopropylethylamine (135 mg, 1.04 mmol) was then added. After the addition was complete, the mixture was warmed to 85° C. under nitrogen protection and stirred for 16 h. After the reaction was complete, the reaction liquid was concentrated under reduced pressure to obtain a crude product, which was purified by column chromatography (dichloromethane/methanol (v/v)=100/0 to 91/9) and then further purified by a reverse-phase column (water/acetonitrile (v/v)=100/1 to 1/85) to obtain the target compound 79 (21 mg, 12.90%). [00862]¹H NMR (400 MHz, DMSO-d6) δ 8.92 (s, 1H), 7.33-7.27 (m, 1H), 4.84 (s, 1H), 4.67-4.55 (m, 1H), 4.43 (br s, 1H), 3.97 (s, 1H), 3.76-3.75 (m, 2H), 3.51-3.40 (m, 1H), 3.24-3.19 (m, 3H), 2.99-2.83 (m, 2H), 2.67 (s, 2H), 2.42-2.34 (m, 2H), 2.22-2.21 (m, 2H), 1.81-1.76 (m, 2H), 1.53-1.47 (m, 1H), 1.27-1.21 (m, 1H), 1.10-0.95 (m, 1H).

LCMS m/z=469.40 [M+H]⁺.

### Example 75

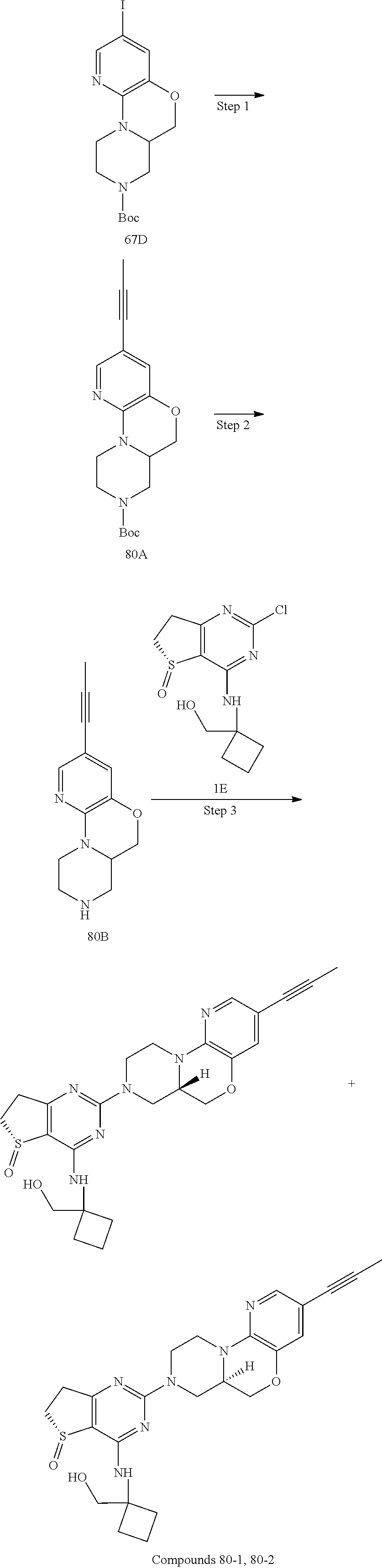

Step 1: 67D (0.8 g, 1.92 mmol), tetrahydrofuran (15 mL), cuprous iodide (72 mg, 0.38 mmol), triethylamine (582 mg, 5.76 mmol), bis(triphenylphosphino)palladium dichloride (139 mg, 0.19 mmol), and a propyne-tetrahydrofuran solution (1 M, 19.2 mL, 19.2 mmol) were successively added to a 100 mL single-mouth flask and reacted for 6 hours at room temperature under nitrogen protection, and the reaction product was concentrated under reduced pressure and then separated and purified by silica gel column chromatography (eluents: ethyl acetate/petroleum ether (v/v)=20/100) to obtain 80A (500 mg, 79.2%).

LC-MS (ESI): m/z=330.2 [M+H]⁺.

Step 2: 80A (0.5 g, 1.5 mmol), triethylamine (3.0 g, 30 mmol), and iodotrimethylsilane (3.0 g, 15 mmol) were successively added to a 50 mL single-mouth flask and reacted at room temperature for 2 hours. The reaction product was concentrated under reduced pressure to obtain crude 80B, which was directly used in the next reaction.

LC-MS (ESI): m/z=230.1 [M+H]⁺.

Step 3: 1E (431 mg, 1.5 mmol), 80B (crude, 0.8 g, 1.5 mmol), dioxane (5 mL), and N,N-diisopropylethylamine (1.16 g, 9.0 mmol) were successively added to a 50 mL single-mouth flask and reacted at 100° C. for 6 hours. The reaction product was cooled to room temperature, and the reaction liquid was directly concentrated under reduced pressure and then separated and purified by silica gel column chromatography (dichloromethane/methanol (v/v)=100/8) to obtain 450 mg of a mixture.

After chiral preparation, the title compound 80-1 (110 mg, 15.3%, retention time: 0.983 min) and the title compound 80-2 (106 mg, 14.7%, retention time: 1.247 min) were obtained. Chiral preparation method: instrument: Waters 150 MGM; chromatographic column: Chiralpak Column; mobile phase: A: carbon dioxide, and B: ethanol (0.1% aqueous ammonia); isocratic elution: 70% mobile phase B; flow rate: 100 mL/min; back pressure: 100 bar; column temperature: 25° C.; wavelength: 220 nm; elution time: 4.0 min.

### (Compound 80-1)

¹H NMR (400 MHz, DMSO-d₆) δ 7.80-7.75 (m, 1H), 7.43 (s, 1H), 7.03-6.94 (m, 1H), 4.89-4.79 (m, 1H), 4.79-4.60 (m, 2H), 4.52-4.31 (m, 2H), 4.01-3.92 (m, 1H), 3.78-3.65 (m, 2H), 3.49-3.35 (m, 2H), 3.27-3.16 (m, 1H), 3.05-2.83 (m, 3H), 2.81-2.69 (m, 2H), 2.42-2.26 (m, 2H), 2.24-2.14 (m, 2H), 2.00 (s, 3H), 1.87-1.71 (m, 2H).

LC-MS (ESI): m/z=481.2 [M+H]⁺.

### (Compound 80-2)

¹H NMR (400 MHz, DMSO-d₆) δ 7.85-7.70 (m, 1H), 7.43 (s, 1H), 7.12-6.77 (m, 1H), 4.93-4.80 (m, 1H), 4.79-4.59 (m, 2H), 4.50-4.32 (m, 2H), 4.10-3.89 (m, 1H), 3.78-3.62 (m, 2H), 3.49-3.34 (m, 2H), 3.28-3.16 (m, 1H), 3.06-2.83 (m, 3H), 2.81-2.71 (m, 2H), 2.40-2.25 (m, 2H), 2.24-2.14 (m, 2H), 2.00 (s, 3H), 1.86-1.72 (m, 2H).

LC-MS (ESI): m/z=481.2 [M+H]⁺.

### Example 76

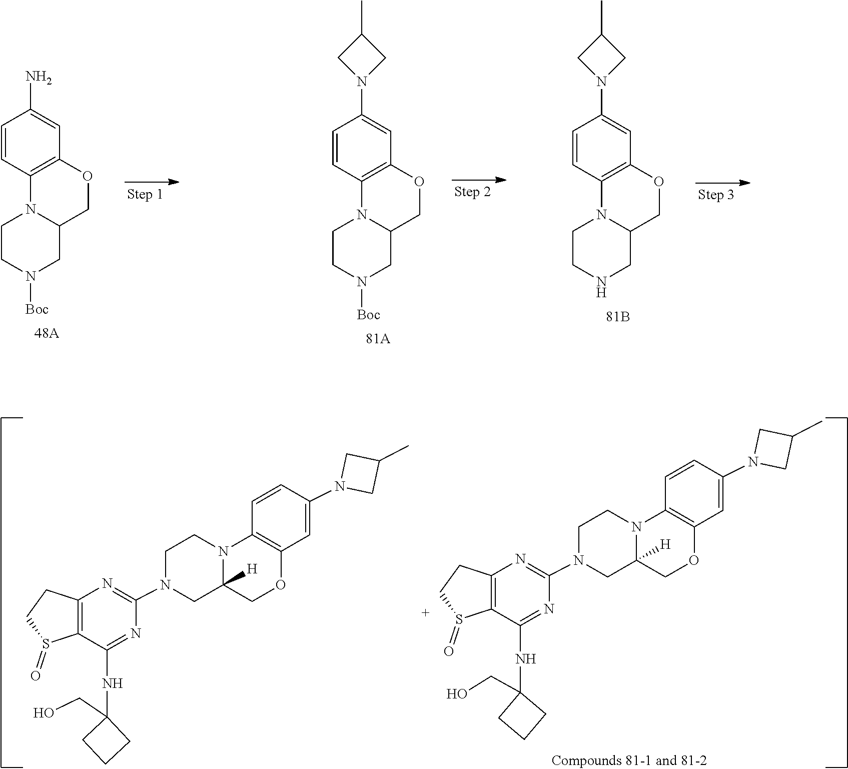

Step 1: At room temperature, compound 48A (1.0 g, 3.27 mmol), 1,3-dibromo-2-methylpropane (0.78 g, 3.60 mmol), and N,N-dimethylformamide (15 mL) were added to a 50 mL round-bottomed flask, and cesium carbonate (3.20 g, 9.82 mmol) was then added. The mixture was heated to 70° C. and reacted for 14 h. After the reaction was complete, the reaction liquid was cooled to room temperature, concentrated under reduced pressure, and then separated and purified by silica gel column chromatography (eluents: EA/PE=0-35%) to obtain the target compound 81A (0.3 g, 25%).

LC-MS (ESI): m/z=360.5 [M+H]⁺.

Step 2: At room temperature, 81A (0.30 g, 0.83 mmol), dichloromethane (3 mL), and trifluoroacetic acid (1 mL) were added to a 25 mL round-bottomed flask and continuously stirred for 2 h at this temperature. After the reaction was complete, the reaction liquid was concentrated under reduced pressure to obtain a crude product of target compound 81B trifluoroacetate (0.20 g, 92%).

LC-MS (ESI): m/z=260.3 [M+H]⁺.

Step 3: At room temperature, the crude product of compound 81B trifluoroacetate (0.20 g, 0.77 mmol), compound 1E (0.27 g, 0.92 mmol), and 1,4-dioxane (5 mL) were added to a 25 mL round-bottomed flask, and N,N-diisopropylethylamine (0.30 g, 2.31 mmol) was added at room temperature.

The mixture was then heated to 100° C. and reacted for 12 h. After the reaction was complete, the reaction liquid was cooled to room temperature, concentrated under reduced pressure, and then separated and purified by silica gel column chromatography (eluents: MeOH/DCM=0-10%) to obtain 0.26 g of a racemate. The racemate was separated by SFC to obtain two isomers, compound 81-1 (50 mg, retention time 1.94 min, 13%) and compound 81-2 (50 mg, retention time 2.18 min, 13%). Separation conditions of preparative chromatography: 1. Instrument: SHIMADZU LC-20AP; 2. chromatographic column: C18; 3. mobile phase system: A for 0.1% TFA in H₂O; B for ACN; 4. gradient: B 3-33%; 5. flow rate: 75 mL/min.

### Compound 81-1:

¹H NMR (400 MHz, Chloroform-d) δ 6.76-6.66 (m, 1H), 6.09-5.95 (m, 3H), 4.83-4.57 (m, 2H), 4.27-4.21 (m, 1H), 4.07-4.01 (m, 1H), 3.98-3.90 (m, 2H), 3.85 (s, 2H), 3.70-3.62 (m, 1H), 3.62-3.54 (m, 1H), 3.44-3.32 (m, 3H), 3.25-3.17 (m, 1H), 3.08-2.96 (m, 3H), 2.84-2.74 (m, 2H), 2.70-2.61 (m, 1H), 2.36-2.20 (m, 4H), 1.98-1.84 (m, 2H), 1.24 (d, 3H).

LC-MS (ESI): m/z=511.3 [M+H]⁺.

Compound 81-2: ¹H NMR (400 MHz, Chloroform-d) δ 6.76-6.68 (m, 1H), 6.19-6.08 (m, 2H), 6.04 (s, 1H), 4.80-4.58 (m, 2H), 4.31-4.22 (m, 1H), 4.09-3.98 (m, 3H), 3.92-3.81 (m, 2H), 3.70-3.54 (m, 2H), 3.47-3.36 (m, 3H), 3.26-3.16 (m, 1H), 3.09-2.95 (m, 3H), 2.89-2.75 (m, 2H), 2.72-2.60 (m, 1H), 2.32-2.26 (m, 4H), 1.97-1.84 (m, 2H), 1.25 (d, 3H).

LC-MS (ESI): m/z=511.3 [M+H]⁺.

### Example 77

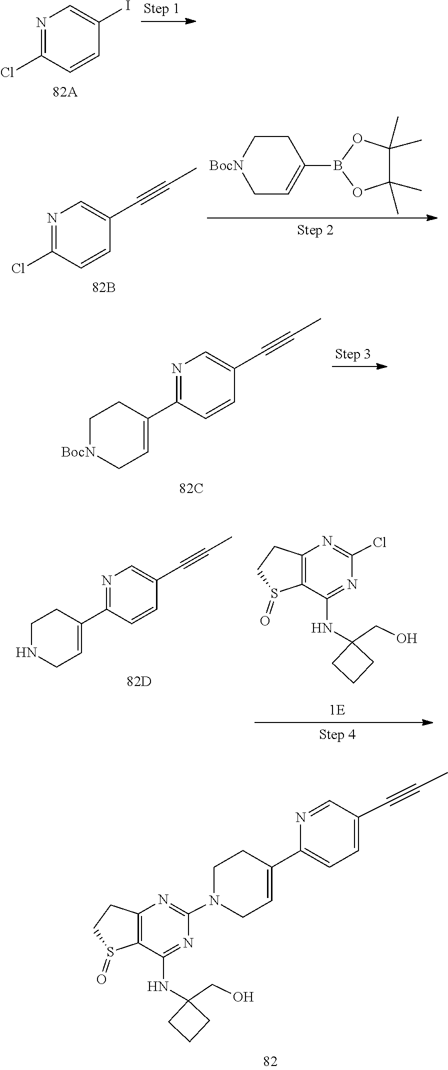

Step 1: Compound 82A (2.0 g, 8.4 mmol) was dissolved in toluene (50 mL), tributylpropynylstannane (3.1 g, 9.2 mmol) and tetrakis(triphenylphosphino)palladium (0.1 g, 0.8 mmol) were successively added, and after nitrogen displacement three times, the mixture was heated to 100° C. and reacted for 14 h. After the completion of the reaction was monitored by a TLC plate, the reaction product was cooled to room temperature, concentrated, and purified by a silica gel column (PE/EA=10/1) to obtain product 82B (1.2 g, 94.4%).

¹H NMR (400 MHz, CDCl₃) δ 8.37 (s, 1H), 7.62 (d, 1H), 7.25 (d, 1H), 2.07 (s, 3H).

Step 2: Compound 82B (1.2 g, 7.9 mmol) was dissolved in 1,4-dioxane (20 mL), and 1,1-bis(diphenylphosphino)ferrocene palladium dichloride (0.6 g, 0.8 mmol), cesium carbonate (5.2 g, 15.8 mmol), N-Boc-1,2,5,6-tetrahydropyridine-4-boronic acid pinacol ester (2.9 g, 9.5 mmol), and water (4 mL) were then added. After nitrogen displacement three times, the mixture was heated to 90° C. and reacted for 4 h, after which the reaction product was passed through a short silica gel column, rinsed with ethyl acetate, concentrated, and subjected to column chromatography (petroleum ether/ethyl acetate=9/1) to obtain compound 82C (2.0 g, 84.6%).

LC-MS (ESI): m/z=299.4 [M+H]⁺.

Step 3: Compound 82C (1.5 g, 5.0 mmol) was dissolved in dichloromethane (10 mL), and trifluoroacetic acid (3.8 mL) was then added. The mixture was reacted at room temperature for half an hour and then concentrated to obtain compound 82D trifluoroacetate (0.9 g, 90.3%), which was directly used in the next step.

LC-MS (ESI): m/z=199.4 [M+H]⁺.

Step 4: Compound 1E (1.68 g, 5.8 mmol) was dissolved in 1,4-dioxane (20.0 mL), and compound 82D trifluoroacetate (1.4 g, 7.0 mmol) and diisopropylethylamine (3.0 g, 23.3 mmol) were then added. The mixture was heated to 90° C. and reacted for 12 h, after which the reaction product was cooled to room temperature, concentrated, and subjected to column chromatography (dichloromethane/methanol=10/1) to obtain the target product 82 (2.5 g, 95%).

¹H NMR (400 MHz, DMSO-d6) δ 8.54 (d, 1H), 7.78-7.76 (m, 1H), 7.57 (d, 1H), 7.42 (s, 1H), 6.84 (s, 1H), 4.87 (s, 1H), 4.41 (s, 2H), 3.96 (t, 2H), 3.75 (s, 2H), 3.51-3.38 (m, 1H), 3.25-2.17 (m, 1H), 2.98-2.84 (m, 2H), 2.61 (s, 2H), 2.39-2.28 (m, 2H), 2.22-2.18 (m, 2H), 2.08 (s, 3H), 1.87-1.72 (m, 2H).

LC-MS (ESI): m/z=450.1 [M+H]⁺.

### Example 78

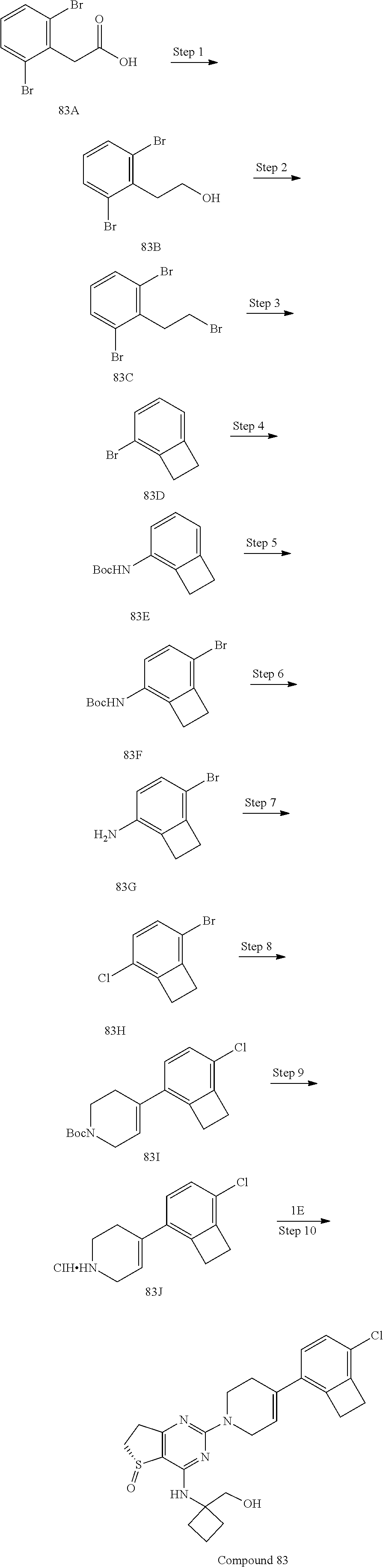

Step 1: Under nitrogen protection, tetrahydrofuran (50 mL) was added to a reaction flask containing compound 83A (5.00 g, 17.01 mmol), and the mixture was stirred at 0° C. for 15 min. A borane tetrahydrofuran complex (4.39 g, 51.03 mmol, 1 M) was slowly added dropwise, and after the addition was complete, the reaction liquid was warmed to 80° C. and stirred for 16 h. After the disappearance of raw materials was detected by TLC, the reaction product was cooled to room temperature, and the reaction was quenched by adding water (20 mL), after which the reaction liquid was concentrated under reduced pressure into a small volume, then diluted by adding water (100 mL), and extracted with ethyl acetate (100 mL) three times. The organic phases were combined, dried by anhydrous sodium sulfate, and then concentrated in vacuum to obtain crude product 83B (4.50 g, 94.50%), which was directly used in the next reaction.

¹H NMR (400 MHz, DMSO-d6) δ 7.65-7.62 (m, 2H), 7.11-7.07 (m, 1H), 3.57-3.53 (m, 2H), 3.16-3.12 (m, 2H).

Step 2: Under nitrogen protection, compound 83B (4.50 g, 16.07 mmol) was dissolved in dichloromethane (45 mL). N-bromosuccinimide (3.43 g, 19.29 mmol) and triphenylphosphine (5.86 g, 22.34 mmol) were added while stirring in ice bath. After the addition was complete, the reaction liquid was warmed to room temperature and stirred for 16 h. After the disappearance of raw materials was monitored by TLC, tert-butyl hydroperoxide (2 mL) was added and stirred for 10 min, the reaction was quenched by adding a saturated sodium bisulfite aqueous solution (100 mL), and the reaction product was extracted with dichloromethane (50 mL) three times. The organic phases were combined, dried over anhydrous sodium sulfate, and then concentrated in vacuum to obtain a crude product. The crude product was purified by column chromatography (eluents: PE) to obtain the target compound 83C (3.20 g, 58.07%).

¹H NMR (400 MHz, CDCl₃) δ 7.53-7.51 (m, 2H), 6.98-6.94 (m, 1H), 3.58-3.47 (m, 4H).

Step 3: Under nitrogen protection, compound 83C (3.20 g, 9.33 mmol) was dissolved in anhydrous tetrahydrofuran (100 mL), and n-butyl lithium (3.6 mL, 2.5 M) was slowly added dropwise with stirring at −70° C. After the addition was complete, the reaction liquid was stirred at −70° C. for 5 h. The reaction was quenched by slowly dropwise adding water (20 mL) and the reaction liquid was extracted with ethyl acetate (100 mL) three times. The organic phases were combined, dried by anhydrous sodium sulfate, and then concentrated in vacuum to obtain 83D (2.10 g, 86.04%).

¹H NMR (400 MHz, CDCl₃) δ 7.29-7.25 (m, 1H), 7.08-7.04 (m, 1H), 6.99-6.98 (m, 1H), 3.13 (s, 4H).

Step 4: Compound 83D (1.00 g, 5.46 mmol) was dissolved in 1,4-dioxane (20 mL), and tert-butyl carbamate (960 mg, 8.19 mmol), 2-dicyclohexylphosphine-2′,4′,6′-triisopropyl biphenyl (260 mg, 0.55 mmol), cesium carbonate (3.56 g, 10.93 mmol), and palladium acetate (62 mg, 0.27 mmol) were successively added. After the addition was complete, the mixture was stirred at 100° C. for 4 h under nitrogen protection. After the disappearance of raw materials was monitored by TLC, the reaction liquid was cooled to room temperature, the reaction was quenched by adding a saturated sodium bicarbonate solution (30 mL), and the reaction product was extracted with ethyl acetate (50 mL) three times. The organic phases were combined, dried over anhydrous sodium sulfate, and then concentrated in vacuum to obtain a crude product. The crude product was purified by column chromatography (eluents: PE:EA=100:1 to 50:1) to obtain the target compound 83E (1.00 g, 93.27%).

¹H NMR (400 MHz, CDCl₃) δ 7.28-7.26 (m, 1H), 7.15-7.11 (m, 1H), 6.77-6.75 (m, 1H), 6.30 (s, 1H), 3.20-3.18 (m, 2H), 3.12-3.10 (m, 2H), 1.52 (s, 9H).

LCMS m/z=164.1 [M−56+H]⁺.

Step 5: Compound 83E (880 mg, 3.65 mmol) was dissolved in acetic acid (6 mL), N-bromosuccinimide (715 mg, 4.01 mmol) was then added, and after the addition was complete, the mixture was stirred at room temperature for 16 h under nitrogen protection. The reaction was diluted by adding water (30 mL) and extracted with ethyl acetate (50 mL) three times. The organic phases were combined, dried over anhydrous sodium sulfate, and then concentrated in vacuum to obtain a crude product. The crude product was purified by column chromatography (eluents: PE:EA=100:1 to 50:1) to obtain the target compound 83F (700 mg, 64.35%).

¹H NMR (400 MHz, CDCl₃) δ 7.48-7.47 (m, 1H), 7.27-7.26 (m, 1H), 6.77 (s, 1H), 3.36-3.33 (m, 2H), 2.98-2.96 (m, 2H), 1.51 (s, 9H).

Step 6: Compound 83F (700 mg, 2.35 mmol) was dissolved in 6 mL of dichloromethane, hydrogen chloride dioxane (6 mL) was added and stirred at 0° C. for 30 min. After the disappearance of raw materials was detected by TLC, the reaction liquid was concentrated at room temperature to obtain crude 83G (750 mg), which was directly used in the next reaction.

Step 7: Crude compound 83G (750 mg, 2.34 mmol) was dissolved in a 15% wt hydrogen chloride aqueous solution (5 mL) in ice bath, and sodium nitrite (265 mg, 3.84 mmol) was added. After stirring for 20 min, it was slowly dropwise added to a reaction flask containing cuprous chloride (1.14 g, 11.51 mmol) and concentrated hydrochloric acid (10 mL), and after the addition was complete, the reaction liquid was stirred for 16 h at room temperature. The mixture was diluted by adding water (20 mL) and extracted with ethyl acetate (50 mL) three times. The organic phases were combined, dried over anhydrous sodium sulfate, and then concentrated in vacuum to obtain a crude product. The crude product was purified by column chromatography (eluent: PE) to obtain the target compound 83H (300 mg, 43.13%).

¹H NMR (400 MHz, CDCl₃) δ 7.60-7.59 (m, 1H), 7.48-7.47 (m, 1H), 3.16-3.15 (m, 2H), 3.04-3.02 (m, 2H).

Step 8: Under nitrogen protection, compound 83H (300 mg, 1.38 mmol) was dissolved in tetrahydrofuran (12 mL) and water (2 mL), and [1-(tert-butoxycarbonyl)-1,2,3,6-tetrahydropyridin-4-yl]boronic acid (435 mg, 1.41 mmol), potassium carbonate (480 mg, 3.45 mmol), and [1,1′-bis(diphenylphosphino)ferrocene]palladium dichloride (150 mg, 0.21 mmol) were successively added. After the addition was complete, the reaction liquid was reacted at 75° C. for 16 h under nitrogen protection. After the disappearance of raw materials was monitored by TLC, the reaction product was diluted by adding water (50 mL) and extracted with ethyl acetate (50 mL) three times. The organic phases were combined, dried over anhydrous sodium sulfate, and then concentrated in vacuum to obtain a crude product. The crude product was purified by column chromatography (eluents: PE:EA=100:1 to 10:1) to obtain the target compound 831 (140 mg, 31.37%).

¹H NMR (400 MHz, CDCl₃) δ 7.43-7.42 (m, 2H), 6.07-6.05 (m, 1H), 4.08-4.07 (m, 2H), 3.62-3.59 (m, 2H), 3.20-3.16 (m, 4H), 2.48-2.42 (m, 2H), 1.49 (s, 9H).

LC-MS (ESI): m/z=282.0 [M−56+H+18]⁺.

Step 9: Compound 831 (130 mg, 0.41 mmol) was dissolved in 2 mL of dichloromethane, and hydrogen chloride dioxane (2 mL) was added and stirred at 0° C. for 30 min. After the disappearance of raw materials was monitored by TLC, the reaction liquid was concentrated at room temperature to obtain crude 83J (110 mg), which was directly used in the next reaction.

Step 10: 1E (80 mg, 0.28 mmol) was dissolved in 1,4-dioxane (4 mL), and 83J (110 mg, 0.42 mmol) and N,N-diisopropylethylamine (110 mg, 0.84 mmol) were successively added. After the addition was complete, the reaction liquid was warmed to 85° C. under nitrogen protection and stirred for 16 h. After the reaction liquid naturally returned to room temperature, the reaction was diluted by adding water (50 mL) and extracted with ethyl acetate (50 mL) three times. The organic phases were combined, dried over anhydrous sodium sulfate, and then concentrated in vacuum to obtain a crude product. The crude product was purified by column chromatography (eluents: DCM:MeOH=100:1 to 10:1) twice and then further by prep.HPLC (preparation method: instrument: waters 2767 preparative liquid chromatographic instrument; chromatographic column: SunFire@Prep C18 (19 mm×250 mm); the sample was dissolved with DMF and filtered with a 0.45 m filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A: 35-90%; c. flow rate: 15 mL/min; d. elution time: 15 min; retention time: 6.0 min) to obtain the target compound 83 (2.5 mg, 1.91%).

¹H NMR (400 MHz, CDCl₃) δ 7.16-7.15 (m, 1H), 7.12-7.11 (m, 1H), 6.98-6.97 (m, 1H), 4.36 (s, 2H), 4.02 (s, 2H), 3.91-3.90 (m, 2H), 3.65-3.56 (m, 1H), 3.45-3.38 (m, 1H), 3.19-3.16 (m, 2H), 3.15-3.13 (m, 2H), 2.57-2.53 (m, 3H), 2.34-2.27 (m, 6H), 2.00-1.89 (m, 3H).

LCMS m/z=471.4 [M+H]⁺.

### Biological Test:

### 1. Effect of Compounds on PDE4B2 Activity

The effect of compounds on PDE4B2 activity was detected by a fluorescence polarization kit (BPS Bioscience, Catalog #60343). According to the instructions of the kit, FAM-Cyclic-3′,5′-AMP at a final concentration of 0.1 M, 1 ng/well of PDE4B2 (PDE buffer was added to the negative control well), and a compound diluted in a gradient manner (PDE buffer containing 10% DMSO was added to the positive control well) were added to each well, fully mixed, and then reacted at room temperature for 1 h. The Binding Agent was diluted with the Binding Agent Diluent (cAMP) at a ratio of 1:100 for later use, the Binding Agent dilution was taken and added to the test plate at 50 μl/well and incubated at room temperature with slow shaking for 20 minutes. After the incubation was complete, Envision was used for FP detection, with Excitation at 480 nm and Emission at 535 nm. FP was usually represented by mP value.

$$mP = ( \frac{{I}_{II} - G ( {I}_{⊥} )}{{I}_{II} + G ( {I}_{⊥} )} ) \times 1000$$

I_{∥} (S535): fluorescence intensity in parallel direction
I_{⊥} (P535): fluorescence intensity in perpendicular direction
G: G factor=1

### Calculation of Inhibition Rate (% Inhibition):

$$\% Inhibition = [ 1 - ( {mP}_{( s a m p l e )} - m {P}_{( n e g ative control )} ) / ( {mP}_{( positive control )} - m {P}_{( n e g ative control )} ) ] \times 100 \%$$

mP₍ₛₐₘₚₗₑ₎: mP of the test compound in a reaction well
mP_{(negative control)}: mP in the negative control well
mP_{(positive control)}: mP in the positive control well

According to the calculated inhibition rate at each concentration, the IC₅₀ value of each compound was calculated by GraphPad Prism 8 software.

The IC₅₀ values of the compounds of the present invention against PDE412 are less than 300 nM, and the IC₅₀ values of the compounds are preferably less than 100 nM, more preferably less than 50 nM, and further preferably less than 10 nM. The IC₅₀ values of some specific compounds are as shown in Table 3 below, wherein A<10 nM, 10 nM≤B<50 nM, and 50 nM≤C<100 nM.

TABLE 3
PDE4B2 activity

| Compound | PDE4B2 IC₅₀ (nM) |
|---|---|
| Compound 1 | A |
| Compound 4 | B |
| Compound 5 | A |
| Compound 6 | B |
| Compound 8 | B |
| Compound 10 | B |
| Compound 13 | B |
| Compound 14 | B |
| Compound 19 | B |
| Compound 20 | B |
| Compound 21-1 | B |
| Compound 22 | B |
| Compound 25 | B |
| Compound 43 | A |
| Compound 46-1 | A |
| Compound 46-2 | A |
| Compound 47-1 | B |
| Compound 47-2 | A |
| Compound 48-1 | A |
| Compound 48-2 | A |
| Compound 50 | B |
| Compound 51 | A |
| Compound 53 | A |
| Compound 54 | A |
| Compound 55 | A |
| Compound 56 | A |
| Compound 57 | B |
| Compound 58 | A |
| Compound 59 | B |
| Compound 60 | B |
| Compound 61-1 | A |
| Compound 61-2 | B |
| Compound 62-1 | A |
| Compound 62-2 | B |
| Compound 63 | A |
| Compound 64 | A |
| Compound 65 | A |
| Compound 66 | A |
| Compound 67-1 | A |
| Compound 67-2 | B |
| Compound 68-1 | A |
| Compound 68-2 | A |
| Compound 69 | A |
| Compound 70 | A |
| Compound 71 | B |
| Compound 74-1 | A |
| Compound 75-1 | A |
| Compound 75-2 | B |
| Compound 76 | A |
| Compound 77-1 | A |
| Compound 77-2 | B |
| Compound 78-1 | B |
| Compound 78-2 | B |
| Compound 79 | A |
| Compound 80-1 | A |
| Compound 80-2 | A |
| Compound 81-1 | A |
| Compound 81-2 | A |
| Compound 82 | A |
| Compound 83 | A |

From the above, it can be seen that the compounds of the present invention, such as the examples, have good inhibitory activities, for example, the IC₅₀ values of compound 1 and compound 5 are 2.45 nM and 8.10 nM, respectively.

### 2. Effect of Compounds on PDE4D3 Activity

The effect of compounds on PDE4D3 activity was detected by a fluorescence polarization kit (BPS Bioscience, Catalog #60346). According to the instructions of the kit, FAM-Cyclic-3′,5′-AMP at a final concentration of 0.1 M, 0.05 ng/well of PDE4D3 (PDE buffer was added to the negative control well), and a compound diluted in a gradient manner (PDE buffer containing 10% DMSO was added to the positive control well) were added to each well, fully mixed, and then reacted at room temperature for 1 h. The Binding Agent was diluted with the Binding Agent Diluent (cAMP) at a ratio of 1:100 for later use, the Binding Agent dilution was taken and added to the test plate at 100 l/well and incubated at room temperature with slow shaking for 1 h. After the incubation was complete, Envision was used for FP detection, with Excitation at 480 nm and Emission at 535 nm. FP was usually represented by mP value.

$$mP = ( \frac{{I}_{II} - G ( {I}_{⊥} )}{{I}_{II} + G ( {I}_{⊥} )} ) \times 1000$$

I_{∥} (S535): fluorescence intensity in parallel direction
I⊥ (P535): fluorescence intensity in perpendicular direction
G: G factor=1

### Calculation of Inhibition Rate (% Inhibition):

$$\% Inhibition = [ 1 - ( {mP}_{( s a m p l e )} - m {P}_{( n e g ative control )} ) / ( {mP}_{( positive control )} - m {P}_{( n e g ative control )} ) ] \times 100 \%$$

mP₍ₛₐₘₚₗₑ₎: mP of the test compound in a reaction well
mP_{(negative control)}: mP in the negative control well
mP_{(positive control)}: mP in the positive control well

According to the calculated inhibition rate at each concentration, the IC₅₀ value of each compound was calculated by GraphPad Prism 8 software.

Conclusion: The compounds of the present invention, such as the examples, have good selectivity.

### 3. Effect of Compounds on PDE4B1 Activity

The effect of compounds on PDE4B1 activity was detected by a fluorescence polarization kit (IMAP FP IPP Explorer KIT, Molecular Device, Cat #R8124). The IMAP Reaction Buffer containing 0.1% BSA (5×) in the kit was diluted into a 1-fold reaction buffer containing 1 mM DTT. 0.12 nM PDE4B1 (BPS, Cat #60041) was added to the 1-fold reaction buffer to form a 2-fold enzyme solution. 10 μL of the 2-fold enzyme solution was added to wells of a 384-well reaction plate. For the negative control well, 10 μL of the 1-fold reaction buffer was used instead of the enzyme solution. Centrifugation at 1000 rpm for 1 minute and incubation at room temperature for 15 minutes were carried out. 0.2 uM FAM-labeled cAMP (FAM-cAMP, Molecular Device, Cat #R7506) was added to the 1-fold reaction buffer to form a 2-fold substrate solution. 10 μL of the 2-fold substrate solution was added to each well of the 384-well reaction plate. Centrifugation at 1000 rpm for 1 minute was carried out. A reaction was carried out at room temperature for 20 minutes. IMAP Progressive Binding Buffer A (5×), IMAP Progressive Binding Buffer B (5×), and IMAP Progressive Binding Reagent (provided by IMAP FP IPP Explorer Kit) were prepared into a reaction stop solution according to the instructions for use. 60 μL of an 80/60-fold reaction stop solution was added to each well of the 384-well reaction plate to stop the reaction, and the reaction product was centrifuged at 1000 rpm for 1 minute. Incubation at room temperature in the dark for 60 minutes was carried out. The mP values (Ex480/Em535 (s), Em535 (p)) were read by a microplate reader (Envision).

FP was usually represented by mP value. mP value for FP measurement=1000*(S−G*P)/(S+G*P)
S: fluorescence intensity in parallel direction
P: fluorescence intensity in perpendicular direction
G: G factor=1

### Calculation of Inhibition Rate (% Inhibition):

$$\% Inhibition = ( m {P}_{( p o s itive control )} - m {P}_{( s a m p l e )} ) / ( {mP}_{( p o s itive control )} - {mP}_{( n e g ative control )} ) \times 100 \%$$

mP₍ₛₐₘₚₗₑ₎: mP of the test compound in a reaction well
mP_{(negative control)}: mP in the negative control well
mP_{(positive control)}: mP in the positive control well

According to the calculated inhibition rate at each concentration, the IC₅₀ value of each compound was calculated by XLFit excel add-in version 5.4.0.8 software.

Y=Bottom+(Top−Bottom)/(1+(IC₅₀/X)\hat{ }HillSlope) Fitting formula:

The IC₅₀ values of the compounds of the present invention against PDE4B1 are less than 300 nM, and the IC₅₀ values of the compounds are preferably less than 100 nM, more preferably less than 50 nM, and further preferably less than 10 nM. The IC₅₀ values of some specific compounds are as shown in Table 4 below, wherein A<10 nM, 10 nM≤B<50 nM, and 50 nM≤C<100 nM.

TABLE 4
PDE4B1 activity

| Compound | PDE4B1 IC₅₀ (nM) |
|---|---|
| Compound 20 | A |
| Compound 22 | B |
| Compound 25 | A |
| Compound 26 | A |
| Compound 27 | A |
| Compound 28 | A |
| Compound 29 | A |
| Compound 30 | A |
| Compound 31 | A |
| Compound 34 | A |
| Compound 35 | A |
| Compound 36 | A |
| Compound 37 | A |
| Compound 40-1 | A |
| Compound 40-2 | A |
| Compound 42-1 | A |
| Compound 42-2 | A |

### 4. Effect of Compounds on PDE4D2 Activity

The effect of compounds on PDE4D2 activity was detected by a fluorescence polarization kit (BPS Bioscience, Catalog #60345). During the reaction, 12.5 μL of 0.272 ng/well of an enzyme (final concentration: 0.068 ng/well) and 12.5 μL of the compound diluted in a gradient manner (DMSO concentration: 4%) were first pre-incubated at room temperature for 15 minutes, the same volume of the enzyme at the same concentration and 12.5 μL of PDE buffer containing 4% DMSO were added to the positive control well, and 25 μL of PDE buffer containing 2% DMSO was added to the negative control well. After completion, 25 μL of 0.2 M FAM-Cyclic-3′,5′-AMP (final concentration: 0.1 M) was added to each well, fully mixed, and then incubated at room temperature with slow shaking for 30 minutes. The Binding Agent was diluted with the Binding Agent Diluent (cAMP) at a ratio of 1:100 for later use, 100 μl/well of the Binding Agent dilution was taken and added to all wells of the test plate and incubated at room temperature with slow shaking for 1 hour.

After the incubation was complete, BMG LRBTECH microplate reader was used for FP detection, with Excitation at 485 nm and Emission at 520 nm. FP was usually represented by mP value.

$$mP = ( \frac{{I}_{II} - G ( {I}_{⊥} )}{{I}_{II} + G ( {I}_{⊥} )} ) \times 1000$$

I_{∥} (S520): fluorescence intensity in parallel direction
I_{⊥} (P520): fluorescence intensity in perpendicular direction
G: G factor=1

### Calculation of Inhibition Rate (% Inhibition):

$$\% Inhibition = [ 1 - ( {mP}_{( s a m p l e )} - m {P}_{( n e g ative control )} ) / ( {mP}_{( positive control )} - m {P}_{( n e g ative control )} ) ] \times 100 \%$$

mP₍ₛₐₘₚₗₑ₎: mP of the test compound in a reaction well
mP_{(negative control)}: mP in the negative control well
mP_{(positive control)}: mP in the positive control well

According to the calculated inhibition rate at each concentration, the IC₅₀ value of each compound was calculated by GraphPad Prism 8 software.

The IC₅₀ values of some specific compounds of the present invention are as shown in Table 5 below, wherein A<10 nM, 10 nM≤B<50 nM, and 50 nM≤C<100 nM.

TABLE 5
PDE4D2 activity

| Compound | PDE4D2 IC₅₀ (nM) |
|---|---|
| Compound 1 | A |
| Compound 4 | B |
| Compound 5 | A |
| Compound 6 | A |
| Compound 8 | B |
| Compound 9 | B |
| Compound 10 | B |
| Compound 13 | A |
| Compound 14 | A |
| Compound 19 | B |
| Compound 20 | A |
| Compound 22 | B |
| Compound 25 | A |
| Compound 26 | A |
| Compound 27 | A |
| Compound 28 | A |
| Compound 29 | A |
| Compound 30 | A |
| Compound 31 | A |
| Compound 32 | B |
| Compound 34 | A |
| Compound 35 | A |
| Compound 37 | A |
| Compound 40-1 | A |
| Compound 40-2 | A |
| Compound 42-1 | A |
| Compound 42-2 | A |
| Compound 43 | B |
| Compound 46-1 | A |
| Compound 46-2 | A |
| Compound 47-2 | A |
| Compound 48-1 | A |
| Compound 48-2 | B |
| Compound 50 | B |
| Compound 51 | B |
| Compound 52 | B |
| Compound 53 | A |
| Compound 54 | A |
| Compound 55 | A |
| Compound 56 | A |
| Compound 57 | B |
| Compound 58 | A |
| Compound 59 | B |
| Compound 60 | B |
| Compound 61-1 | A |
| Compound 61-2 | B |
| Compound 62-1 | A |
| Compound 62-2 | B |
| Compound 63 | A |
| Compound 64 | B |
| Compound 65 | A |
| Compound 66 | A |
| Compound 67-1 | B |
| Compound 67-2 | C |
| Compound 68-1 | B |
| Compound 68-2 | B |
| Compound 69 | B |
| Compound 70 | A |
| Compound 71 | B |
| Compound 74-1 | A |
| Compound 75-1 | B |
| Compound 75-2 | C |
| Compound 76 | A |
| Compound 77-1 | A |
| Compound 77-2 | B |
| Compound 79 | B |
| Compound 80-1 | B |
| Compound 80-2 | B |
| Compound 81-1 | A |
| Compound 81-2 | A |
| Compound 82 | A |
| Compound 83 | A |

### 5. Pharmacokinetic Test in Mice

5.1 Experimental animals: Male ICR mice, 20-25 g, 6 mice/compound. Purchased from Chengdu Ddossy Experimental Animals Co., Ltd.

5.2 Experimental design: On the day of the experiment, 42 ICR mice were randomly grouped according to their body weights. The animals were fasted with water available for 12-14 h one day before administration and fed 4 h after administration.

TABLE 6
Administration information

| | | Administration information | | | | | |
|---|---|---|---|---|---|---|---|
| | Num- | | Administration | Administration | Administration | | |
| | ber | Test | dosage | concentration | volume | Collected | Mode of |
| Group | Male | compound | (mg/kg) | (mg/mL) | (mL/kg) | sample | administration |
| G1 | 3 | Compound | 5 | 1 | 5 | Plasma | Intravenous |
| G2 | 3 | 37 | 10 | 1 | 10 | Plasma | Intragastric |
| G3 | 3 | Compound | 5 | 1 | 5 | Plasma | Intravenous |
| G4 | 3 | 28 | 10 | 1 | 10 | Plasma | Intragastric |
| G5 | 3 | Compound | 5 | 1 | 5 | Plasma | Intravenous |
| G6 | 3 | 42-1 | 10 | 1 | 10 | Plasma | Intragastric |
| G7 | 3 | Compound | 5 | 1 | 5 | Plasma | Intravenous |
| G8 | 3 | 5 | 10 | 1 | 10 | Plasma | Intragastric |
| G9 | 3 | Compound | 5 | 1 | 5 | Plasma | Intravenous |
| G10 | 3 | 1 | 10 | 1 | 10 | Plasma | Intragastric |
| G11 | 3 | Compound | 5 | 1 | 5 | Plasma | Intravenous |
| G12 | 3 | 67-1 | 10 | 1 | 10 | Plasma | Intragastric |
| G13 | 3 | Compound | 5 | 1 | 5 | Plasma | Intravenous |
| G14 | 3 | 80-1 | 10 | 1 | 10 | Plasma | Intragastric |

Note:
Vehicle I for intravenous administration: 5% DMA + 5% Solutol + 90% Saline, and vehicle II for intravenous administration: 10% DMA + 10% Solutol + 80% Saline; vehicle for intragastric administration: 0.5% MC
(DMA: dimethylacetamide; Solutol: polyethylene glycol-15-hydroxystearate; Saline: physiological saline; MC: methylcellulose)

Before and after administration, 0.06 mL of blood was taken from the orbit under isoflurane anesthesia and placed in an EDTAK2 centrifuge tube for centrifugation at 5000 rpm at 4-C for 10 min, followed by plasma collection. The blood collection time points for the intravenous group and intragastric group were both 0 m, 5 mi, 15 mi, 30 mi, 1 h, 2 h, 4 h, 6 h, 8 h, and 24 h. Before analysis and detection, all the samples were stored at −80° C., and the samples were subjected to quantitative analysis using LC-MS/MS.

TABLE 7
Pharmacokinetic parameters of test compounds in plasma of mice

| Test | Mode of | CL | Vdₛₛ | AUC₀₋ₜ | T1/2 | |
|---|---|---|---|---|---|---|
| compound | administration | (mL/min/kg) | (L/kg) | (hr*ng/mL) | (h) | F (%) |
| Compound | i.v. (5 mg/kg) | 7.03 ± 1.1 | 1.30 ± 0.086 | 6000 ± 1086 | 3.34 ± 0.30 | — |
| 37 | i.g. (10 mg/kg) | — | — | 26929 ± 3912 | 4.30 ± 0.69 | ≥98 |
| Compound | i.v. (5 mg/kg) | 4.67 ± 1.2 | 1.31 ± 0.28 | 9211 ± 2066 | 3.59 ± 0.19 | — |
| 28 | i.g. (10 mg/kg) | — | — | 26287 ± 4150 | 7.04 ± 1.8 | 71.4 ± 11 |
| Compound | i.v. (5 mg/kg) | 7.00 ± 0.89 | 1.08 ± 0.22 | 2760 ± 350 | 2.06 ± 0.59 | — |
| 42-1 | i.g. (10 mg/kg) | — | — | 37722 ± 3009 | 3.44 ± 0.65 | ≥98 |
| Compound | i.v. (5 mg/kg) | 5.71 ± 0.86 | 1.31 ± 0.12 | 7395 ± 1110 | 2.72 ± 0.23 | — |
| 5 | i.g. (10 mg/kg) | — | — | 27184 ± 2232 | 3.42 ± 0.16 | 91.9 ± 7.5 |
| Compound | i.v. (5 mg/kg) | 4.55 ± 0.46 | 2.07 ± 0.20 | 8655 ± 759 | 6.17 ± 0.71 | |
| 1 | i.g. (10 mg/kg) | — | — | 26389 ± 1635 | 15.7 ± 4.1 | 76.2 ± 4.7 |
| Compound | i.v. (5 mg/kg) | 9.04 ± 0.75 | 0.986 ± 0.076 | 4552 ± 393 | 1.27 ± 0.053 | |
| 67-1 | i.g. (10 mg/kg) | — | — | 41862 ± 676 | 2.66 ± 0.14 | ≥98 |
| Compound | i.v. (5 mg/kg) | 6.34 ± 0.27 | 0.968 ± 0.076 | 6156 ± 249 | 1.78 ± 0.17 | — |
| 80-1 | i.g. (10 mg/kg) | — | — | 35989 ± 3265 | 2.67 ± 0.62 | ≥98 |

—: not applicable.

Conclusion: The compounds of the present invention, such as the examples, exhibit excellent pharmacokinetic properties in the PK test in mice.

### 6. Pharmacokinetic Test in Beagle Dogs

6.1 Experimental animals: male beagle dogs, about 8-11 kg, 6 beagle dogs/compound, purchased from Beijing Marshall Biotechnology Co., Ltd.

6.2 Experimental method: On the day of the experiment, 18 beagle dogs were randomly grouped according to their body weights. The animals were fasted with water available for 12-14 h one day before administration and fed 4 h after administration. Administration was carried out according to Table 8.

TABLE 8
Administration information

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | sample | administration |
| G1 | 3 | Compound | 1 | 1 | 1 | Plasma | Intravenous |
| G2 | 3 | 31 | 5 | 1 | 5 | Plasma | Intragastric |
| G3 | 3 | Compound | 1 | 1 | 1 | Plasma | Intravenous |
| G4 | 3 | 5 | 5 | 1 | 5 | Plasma | Intragastric |
| G5 | | Compound | 1 | 1 | 1 | Plasma | Intravenous |
| G6 | | 67-1 | 5 | 1 | 5 | Plasma | Intragastric |

Note:
Vehicle I for intravenous administration: 5% DMA + 5% Solutol + 90% Saline, and vehicle II for intravenous administration: 10% DMA + 10% Solutol + 80% Saline; vehicle for intragastric administration: 0.5% MC
(DMA: dimethylacetamide; Solutol: polyethylene glycol-15-hydroxystearate; Saline: physiological saline; MC: Methylcellulose solution)

Before and after administration, 1 ml of blood was taken from the jugular vein or limb vein and placed in an EDTAK2 centrifuge tube. Centrifugation was carried out at 5000 rpm at 4° C. for 10 min, and plasma was collected. The blood collection time points for the intravenous group and intragastric group were both 0 min, 5 min, 15 min, 30 min, 1 h, 2 h, 4 h, 6 h, 8 h, 10 h, 12 h, 24 h, 48 h, and 72 h. Before analysis and detection, all the samples were stored at −80° C., and the samples were subjected to quantitative analysis using LC-MS/MS.

TABLE 9
Pharmacokinetic parameters of test compounds in plasma of beagle dogs

| Test | Mode of | CL | | AUC₀₋ₜ | | |
|---|---|---|---|---|---|---|
| compound | administration | (mL/min/kg) | Vdₛₛ (L/kg) | (hr*ng/ml) | T1/2 (h) | F (%) |
| Compound | i.v. (1 mg/kg) | 2.69 ± 1.7 | 0.957 ± 0.37 | 7070 ± 3250 | 5.85 ± 0.63 | — |
| 31 | i.g. (5 mg/kg) | — | — | 26113 ± 11945 | 7.38 ± 1.2 | ≥98 |
| Compound | i.v. (1 mg/kg) | 13.8 ± 2.3 | 4.16 ± 0.76 | 1202 ± 210 | 3.91 ± 0.037 | — |
| 5 | i.g. (5 mg/kg) | — | — | 5022 ± 1696 | 6.32 ± 2.1 | ≥98 |
| Compound | i.v. (1 mg/kg) | 3.24 ± 0.27 | 1.09 ± 0.091 | 5154 ± 436 | 6.20 ± 0.35 | |
| 67-1 | i.g. (5 mg/kg) | — | — | 9080 ± 1987 | 6.39 ± 0.54 | 58.7 ± 13 |

—: not applicable.

Conclusion: The compounds of the present invention, such as the examples, exhibit excellent pharmacokinetic properties in the PK test in beagle dogs.

### 7. Pharmacokinetic Test in Monkeys

Experimental animals: Male cynomolgus monkeys, 3-5 kg, 3-6 years old, 6 monkeys/compound. Purchased from Suzhou Xishan Biotechnology Inc.

Experimental method: On the day of the experiment, 12 monkeys were randomly grouped according to their body weights. The animals were fasted with water available for 14-18 h one day before administration and fed 4 h after administration.

TABLE 10
Administration information

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | sample | administration |
| G1 | 3 | Compound 31 | 1 | 0.5 | 2 | Plasma | Intravenous |
| G2 | 3 | Compound 31 | 3 | 1 | 3 | Plasma | Intragastric |
| G3 | 3 | Compound | 1 | 0.5 | 2 | Plasma | Intravenous |
| | | 67-1 | | | | | |
| G4 | 3 | Compound | 5 | 1 | 5 | Plasma | Intragastric |
| | | 67-1 | | | | | |

Note:
Vehicle for intravenous administration: 5% DMA + 5% Solutol + 90% Saline;
* Dosage is calculated based on free base.

Before and after administration, 1.0 mL of blood sample was taken from the limb vein and placed in an EDTAK2 centrifuge tube. Centrifugation was carried out at 5000 rpm at 4° C. for 10 min, and plasma was collected. The blood collection time points for the intravenous group were 0 min, 5 min, 15 min, 30 min, 1 h, 2 h, 4 h, 6 h, 8 h, 10 h, 12 h, and 24 h. Before analysis and detection, all the samples were stored at −80° C., and the samples were subjected to quantitative analysis using LC-MS/MS.

TABLE 11
Pharmacokinetic parameters of test compounds in plasma of monkeys

| | Mode of | CL | Vdₛₛ | AUC₀₋ₜ | F |
|---|---|---|---|---|---|
| Test compound | administration | (mL/min/kg) | (L/kg) | (hr*ng/mL) | (%) |
| Compound 31 | i.v. (1 mg/kg) | 1.95 ± 0.15 | 0.888 ± 0.16 | 8546 ± 664 | — |
| Compound 31 | i.g. (3 mg/kg) | — | — | 7928 ± 3236 | 30.9 ± 13 |
| Compound 67-1 | i.v. (1 mg/kg) | 5.70 ± 0.63 | 1.06 ± 0.024 | 4391 ± 592 | — |
| Compound 67-1 | i.g. (5 mg/kg) | — | — | 7662 ± 1168 | 34.9 ± 5.3 |

Conclusion: The compounds of the present invention, such as the examples, exhibit excellent pharmacokinetic properties in the PK test in cynomolgus monkeys.

8. Test for hERG Potassium Ion Channel

Experimental Platform: Electrophysiological Manual Patch-Clamp System

Cell line: Chinese hamster ovary (CHO) cell line stably expressing hERG potassium ion channel

Experimental method: CHO (Chinese Hamster Ovary) cells stably expressing hERG potassium channel were subjected to a whole cell patch-clamp technique to record the hERG potassium channel current at room temperature. The glass microelectrode was made of a glass electrode blank (BF150-86-10, Sutter) by a puller. The tip resistance after filling the liquid in the electrode was about 2-5 MQ. The glass microelectrode can be connected to the patch-clamp amplifier by inserting the glass microelectrode into an amplifier probe. The clamping voltage and data recording were controlled and recorded by the pClamp 10 software through a computer. The sampling frequency was 10 kHz, and the filtering frequency was 2 kHz. After the whole cell records were obtained, the cells were clamped at −80 mV, and the step voltage that induced the hERG potassium current (I_{hERG}) was depolarized from −80 mV to +20 mV for 2 s, then repolarized to −50 mV, and returned to −80 mV after 1 s. This voltage stimulation was given every 10 s, and the administration process was started after the hERG potassium current was confirmed to be stable (at least 1 minute). The compound was administered for at least 1 minute at each test concentration, and at least 2 cells (n: 2) were tested at each concentration.

Data processing: Data analysis processing was carried out by using pClamp 10, GraphPad Prism 5, and Excel software. The degree of inhibition on the hERG potassium current (peak value of hERG tail current induced at −50 mV) at different compound concentrations was calculated by the following formula:

$$Inhibition \% = [ 1 - ( I / Io ) ] \times 100 \%$$

where Inhibition % represents the percentage inhibition of the compound on the hERG potassium current, and I and Io represent the amplitude of the hERG potassium current after and before administration, respectively.

Compound IC₅₀ was calculated using GraphPad Prism 5 software by fitting according to the following equation:

Y=Bottom+(Top−Bottom)/(1+10\hat{ }((LogIC50−X)*HillSlope))
where X represents the Log value of the tested concentration of the test sample, Y represents the percentage inhibition at the corresponding concentration, and Bottom and Top represent the minimum and maximum percentage inhibitions, respectively.

Experimental results: The IC₅₀ values for the inhibitory effect of the test compounds on the hERG potassium channel current are shown in Table 12 below:

TABLE 12
Inhibitory activity of test compounds
on hERG potassium channel current

| Test compound | IC₅₀ (μM) |
|---|---|
| Compound 37 | 13.3 |
| Compound 31 | 12.2 |
| Compound 40-2 | >20 |
| Compound 34 | 13.6 |
| Compound 80-1 | 18.7 |

Conclusion: The compounds of the present invention, such as the examples, have no obvious inhibitory effect on the hERG potassium channel current.

9. Detection of Inhibitory Activity of Compounds on the Release of Tumor Necrosis Factor-α (TNF-α) from Human Peripheral Blood Mononuclear Cells Induced by Lipopolysaccharide (LPS) In Vitro

Normal human peripheral blood which was anticoagulated (citric acid anticoagulation) was collected, and hPBMCs were prepared by Ficoll-Paque PLUS (Cytiva, Cat #17144002, density 1.077 g/mL). The concentration of hPBMC cells was adjusted to 0.25×10⁶ cells/mL with RPMI1640 medium and inoculated into a 96-well plate in an amount of 50000 cells per well. Subsequently, different concentrations of drugs were added for pre-incubation for 1 h (the DMSO final concentration was 0.1%, and the positive and negative control wells had the same volume of RPMI1640 containing 0.1% DMSO). After the pre-incubation was complete, 100 ng/mL LPS (SIGMA, L₂₆₃₀) was added to the compound and positive control wells, and the negative control well had the same volume of RPMI1640 containing 0.1% DMSO. Incubation was carried out for 4 h in an incubator at 37° C. and 5% CO₂. The cell supernatant was collected, and the content of TNF-α in the supernatant sample was detected by human TNF-α Elisa quantitative test kit (Sino Biological, Cat #KIT10602). The IC₅₀ value was calculated using GraphPad Prism software. The IC₅₀ values of some specific compounds of the present invention are as shown in Table 13 below, wherein A<10 nM, 10 nM≤B<50 nM, and 50 nM≤C<100 nM.

TABLE 13
Inhibitory activity of test compounds
on TNF-α induced by LPS in vitro

| Test compound | Re IC₅₀ (nM) | Max inhibition (%) |
|---|---|---|
| Compound 31 | C | 75.37 |
| Compound 37 | A | / |
| Compound 54 | B | 78.4 |
| Compound 55 | B | 67.23 |
| Compound 62-1 | B | 75.83 |
| Compound 64 | A | 70.83 |
| Compound 65 | B | 82 |
| Compound 66 | B | 79.33 |
| Compound 67-1 | B | / |
| Compound 67-2 | B | / |
| Compound 68-1 | B | / |
| Compound 68-2 | C | / |
| Compound 69 | C | 74.38 |
| Compound 70 | A | 70.54 |
| Compound 73-1 | C | 66.38 |
| Compound 74-1 | B | 72.39 |
| Compound 75-1 | A | 63.52 |
| Compound 75-2 | B | 72.44 |
| Compound 76 | A | 73.75 |
| Compound 77-1 | A | 75.70 |
| Compound 77-2 | A | 73.99 |
| Compound 78-1 | A | 61.16 |
| Compound 78-2 | B | 58.51 |
| Compound 79 | A | 61.49 |
| Compound 80-1 | A | 70.26 |
| Compound 80-2 | B | 63.03 |
| Compound 83 | A | 77.65 |

Re IC₅₀: relative IC₅₀.

1. A compound as represented by formula (I) or a stereoisomer or pharmaceutically acceptable salt thereof, 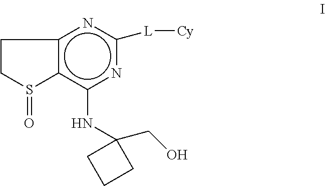
wherein Cy is selected from Cy1, Cy2, a 5-membered monocyclic heteroaryl, or an 8- to 10-membered bicyclic heteroaryl, wherein the monocyclic heteroaryl and the bicyclic heteroaryl are optionally further substituted with 1-3 R; 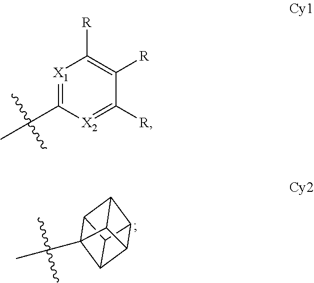
Cy2 is optionally substituted with 1-3 R;
L is -(L₁)n-(L₂)m-;
L₁ is —NR₁—, —CR₂═N—, —CR₂═CR₂—, or —CR₂R₂—;
L₂ is 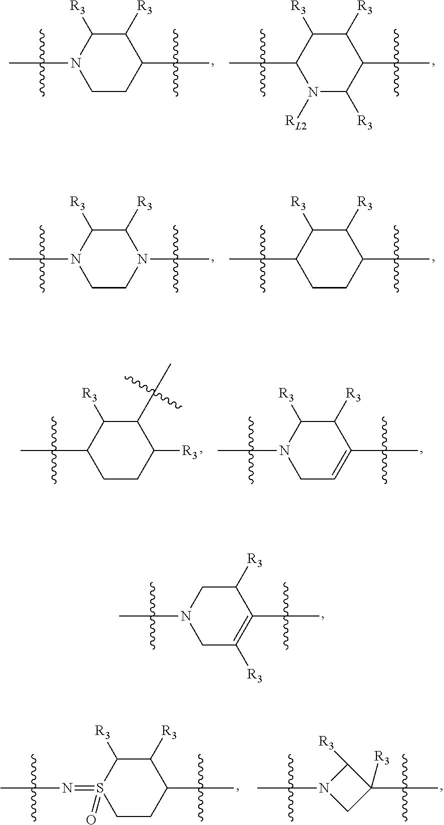
C₁₋₄ alkylene, C₂₋₄ alkenylene, C₂₋₄ alkynylene, CO, O, NR_{L2}, C₃₋₅ cycloalkylene, 6-9-membered arylene, 5- or 6-membered heteroarylene containing 1-3 heteroatoms selected from N, S and O, 5-membered monoheterocyclic alkylene containing 1-3 heteroatoms selected from N, S and O, 7- or 8-membered monoheterocyclic alkylene containing 1-3 heteroatoms selected from N, S and O, 5- to 10-membered fused-heterocyclic alkylene containing 1-3 heteroatoms selected from N, S and O, 6- to 10-membered bridged-heterocyclic alkylene containing 1-3 heteroatoms selected from N, S and O, and 7- to 12-membered spiro-heterocyclic alkylene containing 1-3 heteroatoms selected from N, S and O, wherein the alkylene, alkenylene, alkynylene, cycloalkylene, arylene, heteroarylene, monoheterocyclic alkylene, fused-heterocyclic alkylene, bridged-heterocyclic alkylene, and spiro-heterocyclic alkylene are optionally substituted with 1-3 groups selected from halogen, ═O, CN, C₁₋₄ alkyl, C₁₋₄ alkoxy, OH, and NH₂;
R_{L2} is H, C₁₋₄ alkyl, or C₃₋₆ cycloalkyl;
X₁ and X₂ are independently CRx_{X1} or N;
each R_{X1} is independently H, halogen, C₁₋₄ alkyl, C₁₋₄ haloalkyl, C₂₋₄ alkenyl, C₂₋₄ alkynyl, or —SO₂NH₂, or R_{X1} and R₁, R_{X1} and R₂, or R_{X1} and R₃, together with the atoms to which they are attached, form C₃₋₈ cycloalkyl, 5- to 10-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
each R is independently H, halogen, cyano, C₁₋₄ alkyl, C₂₋₄ alkenyl, C₂₋₄ alkynyl, C₃₋₈ cycloalkyl, 4-to 7-membered heterocycloalkyl containing 1-3 heteroatoms selected from N, S, O, Si and P, —SO₂NH₂, —NHSO₂NH₂, —NHCONH₂, —NHCOC₁₋₄ alkyl, —CONHC₁₋₄ alkyl, —NHSO₂C₁₋₄ alkyl, —SO₂NHC₁₋₄ alkyl, —SO₂C₁₋₄ alkyl, —P(O)(C₁₋₄ alkyl)₂, —SCF₃, —SF₅, or —C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, —C(O)C₁₋₄ alkyl, and NH₂;
or two adjacent R, or R and R_{X1} together with the atoms to which they are attached form C₃₋₈ cycloalkyl, 5- to 10-membered heterocycloalkyl containing 1-3 heteroatoms selected from N, S, O, P and B, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, —C(O)C₁₋₄ alkyl, and NH₂;
R₁ is H, C₁₋₄ alkyl, C₁₋₄ haloalkyl, or C₃₋₆ cycloalkyl;
R₂ is H, C₁₋₄ alkyl, C₁₋₄ haloalkyl, C₁₋₄ alkoxy, —NHC₁₋₄ alkyl, or C₃₋₆ cycloalkyl;
R₃ is H, halogen, ═O, CN, C₁₋₄ alkyl, or C₁₋₄ alkoxy;
n is 0, 1, 2, 3, or 4;
m is 0, 1, 2, or 3;
provided that the compound is not

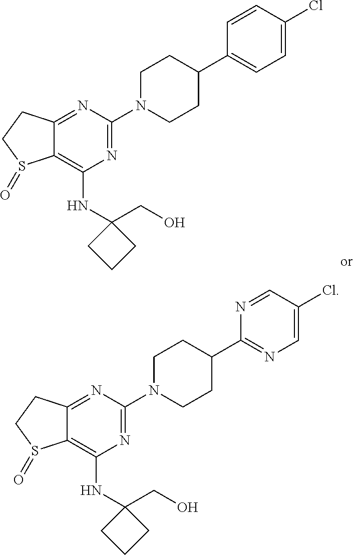 [SMILES: O=S1CCc2nc(N3CCC(c4ccc(Cl)cc4)CC3)nc(NC3(CO)CCC3)c21 ; O=S1CCc2nc(N3CCC(c4ncc(Cl)cn4)CC3)nc(NC3(CO)CCC3)c21]

2. The compound or the stereoisomer or pharmaceutically acceptable salt thereof according to claim 1,
wherein each L₂ is independently selected from CO, O, NR_{L2}, C₁₋₄ alkylene, C₂₋₄ alkenylene, C₂₋₄ alkynylene 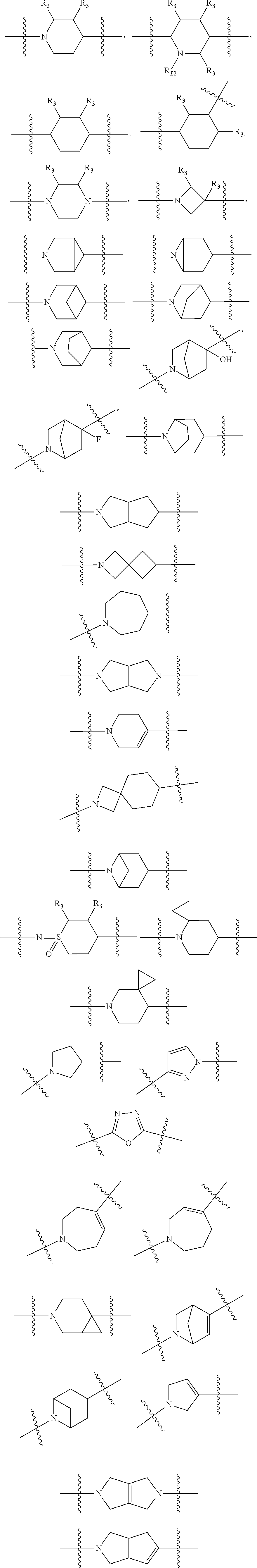 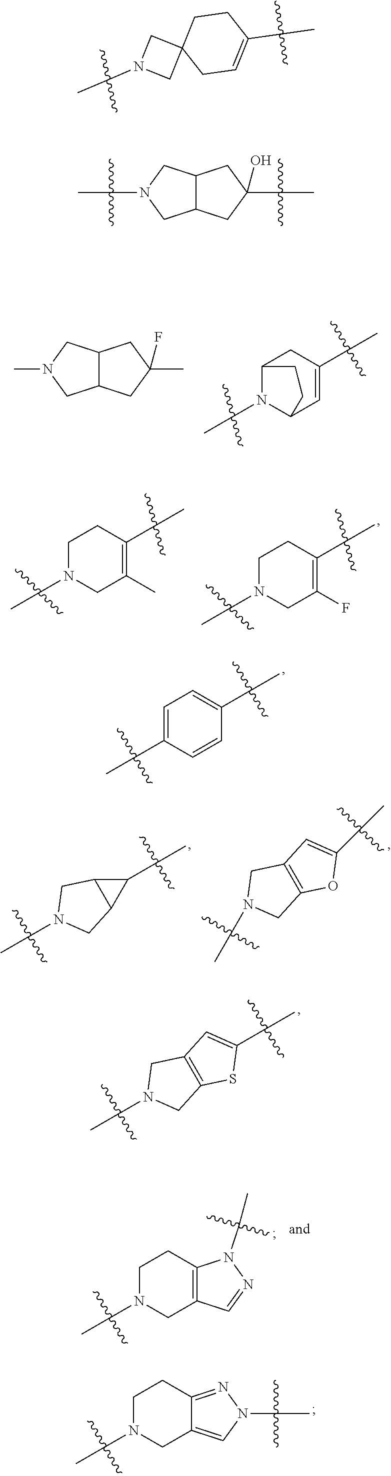
R_{L2} is H, C₁₋₄ alkyl, or C₃₋₆ cycloalkyl;
each R_{X1} is independently H, halogen, C₁₋₄ alkyl, or C₁₋₄ haloalkyl, or R_{X1} and R₁, R_{X1} and R₂, or R_{X1} and R₃ together with the atoms to which they are attached form C₃₋₆ cycloalkyl, 5- to 7-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
each R is independently H, halogen, cyano, C₁₋₄ alkyl, C₂₋₄ alkenyl, C₂₋₄ alkynyl, C₃₋₆ cycloalkyl, 4- to 6-membered heterocycloalkyl containing 1-3 heteroatoms selected from N, S, O, Si and P, —SO₂NH₂, —NHSO₂NH₂, —NHSO₂C₁₋₄ alkyl, —SO₂NHC₁₋₄ alkyl, SO₂C₁₋₄ alkyl, —P(O)(C₁₋₂ alkyl)₂, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, —C(O)C₁₋₄ alkyl, and NH₂; or two adjacent R, or R and R_{X1} together with the atoms to which they are attached form C₃₋₆ cycloalkyl, or 5- to 10-membered heterocycloalkyl containing 1-3 heteroatoms selected from N, S, O, P and B, wherein the cycloalkyl and heterocycloalkyl are optionally substituted with 1-3 groups selected from halogen, ═O, CN, C₁₋₄ alkyl, C₁₋₄ alkoxy, OH, —C(O)C₁₋₄ alkyl and NH₂;
R₁ is H, C₁₋₄ alkyl, or C₁₋₄ haloalkyl;
R₂ is H, C₁₋₄ alkyl, C₁₋₄ haloalkyl, or C₁₋₄ alkoxy;
R₃ is H, halogen, ═O, CN, or C₁₋₄ alkyl;
n is 0, 1, 2, 3, or 4;
m is 0, 1, or 2.

3. The compound or the stereoisomer or pharmaceutically acceptable salt thereof according to claim 2, wherein the compound has a structure of the following formula II: 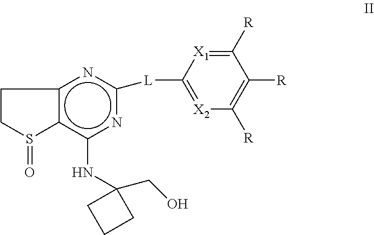
wherein L is selected from one of the following structures: 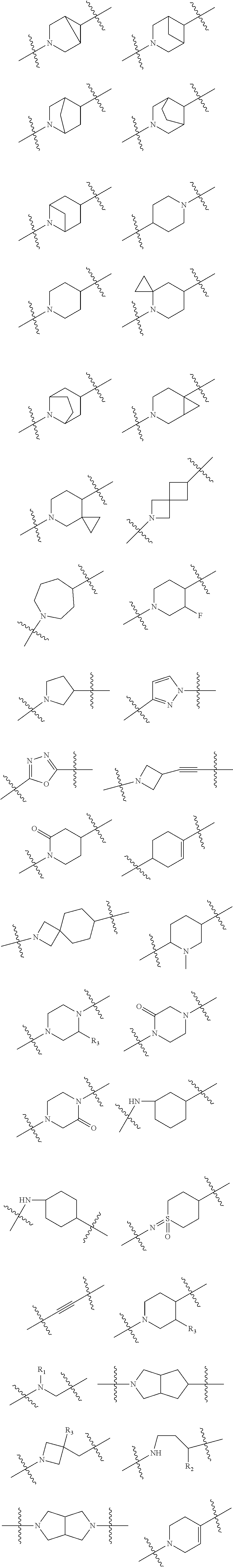 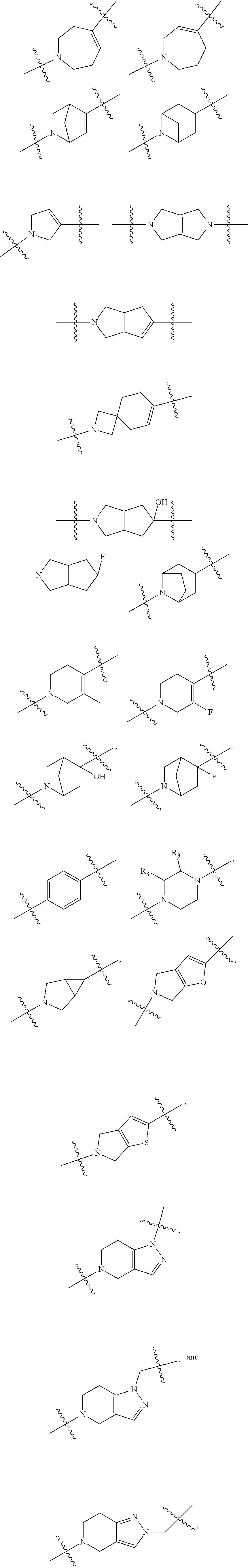
X₁ and X₂ are independently CR_{X1} or N;
each R_{X1} is independently H, halogen, C₁₋₄ alkyl, or C₁₋₄ haloalkyl, or R_{X1} and R₁, R_{X1} and R₂, or R_{X1} and R₃ together with the atoms to which they are attached form C₃ 6 cycloalkyl, 5- to 7-membered heterocycloalkyl containing 1-3 heteroatoms selected from N, S and O, or 5- or 6-membered heteroaryl containing 1-3 heteroatoms selected from N, S and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
each R is independently H, halogen, cyano, C₁₋₄ alkyl, C₂₋₄ alkynyl, C₃ 6 cycloalkyl, 4- to 6-membered heterocycloalkyl containing 1-3 heteroatoms selected from N, S, O, Si and P, —SO₂NH₂, —NHSO₂C₁₋₄ alkyl, —SO₂NHC₁₋₄ alkyl, SO₂C₁₋₄ alkyl, —P(O)(CH₃)₂, SCF₃, SF₅, or C₁₋₄ haloalkyl, wherein the cycloalkyl and heterocycloalkyl are optionally substituted with 1-3 groups selected from halogen, ═O, CN, C₁₋₄ alkyl, C₁₋₄ alkoxy, OH, —C(O)C₁₋₄ alkyl, and NH₂; or two adjacent R, or R and R_{X1} together with the atoms to which they are attached form C₃₋₆ cycloalkyl, or 5- to 10-membered heterocycloalkyl containing 1-3 heteroatoms selected from N, S, O, P and B, wherein the cycloalkyl and heterocycloalkyl are optionally substituted with 1-3 groups selected from halogen, ═O, CN, C₁₋₄ alkyl, C₁₋₄ alkoxy, OH, —C(O)C₁₋₄ alkyl and NH₂;
R₁ is H, or C₁₋₄ alkyl, or C₁₋₄ haloalkyl;
R₂ is H, C₁₋₄ alkyl, or C₁₋₄ haloalkyl; and
R₃ is H, halogen, ═O, CN, or C₁₋₄ alkyl.

4. The compound or the stereoisomer or pharmaceutically acceptable salt thereof according to claim 1, wherein
Cy is selected from the following groups that are optionally further substituted with 1-3 R: 5-membered monocyclic heteroaryl, 5-membered heteroaryl fused benzene ring, 5-membered heteroaryl fused 5-membered heteroaryl, 5-membered heteroaryl fused 6-membered heteroaryl, 5-membered heteroaryl fused 4-membered cycloalkyl, 5-membered heteroaryl fused 5-membered cycloalkyl, 5-membered heteroaryl fused 6-membered cycloalkyl, 5-membered heteroaryl fused 4-membered heterocycloalkyl, 5-membered heteroaryl fused 5-membered heterocycloalkyl, 5-membered heteroaryl fused 6-membered heterocycloalkyl, 6-membered heteroaryl fused benzene ring, 6-membered heteroaryl fused 5-membered heteroaryl, 6-membered heteroaryl fused 6-membered heteroaryl, 6-membered heteroaryl fused 4-membered cycloalkyl, 6-membered heteroaryl fused 5-membered cycloalkyl, 6-membered heteroaryl fused 6-membered cycloalkyl, 6-membered heteroaryl fused 4-membered heterocycloalkyl, 6-membered heteroaryl fused 5-membered heterocycloalkyl, and 6-membered heteroaryl fused 6-membered heterocycloalkyl;
or Cy is selected from the following structures that are optionally further substituted with 1-3 R:

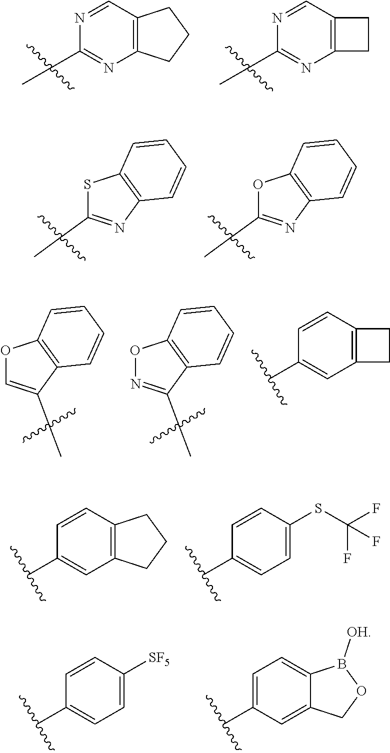

5. The compound or the stereoisomer or pharmaceutically acceptable salt thereof according to claim 3, wherein
L is selected from 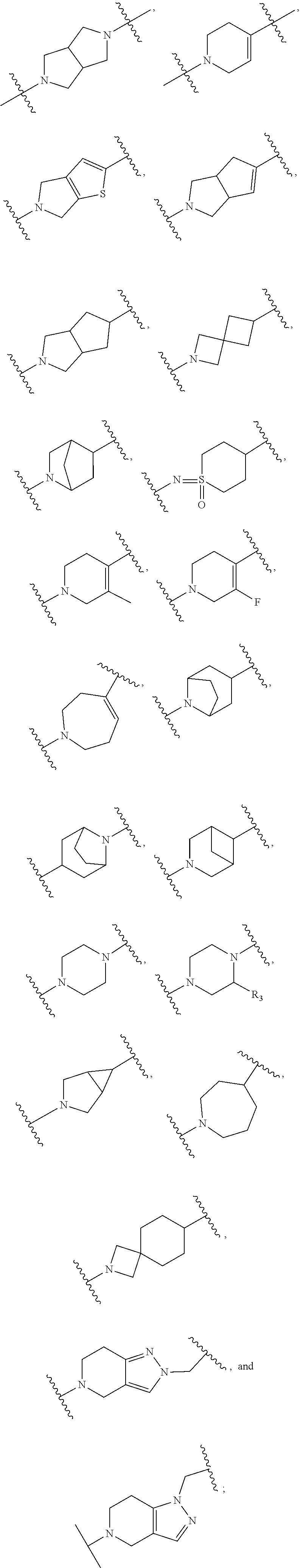
X₁ and X₂ are independently CRx_{X1} or N;
each R_{X1} is independently H, F, Cl, methyl, or ethyl, or R_{X1} and R₃ together with the atoms to which they are attached form C₅ cycloalkyl, C₆ cycloalkyl, 5-membered heterocycloalkyl containing 1-3 heteroatoms selected from N, S and O, or 6-membered heterocycloalkyl containing 1-3 heteroatoms selected from N, S and O;
R₃ is H, F, Cl, ═O, CN, or C₁₋₃ alkyl; and
each R is independently H, F, Cl, methyl, ethyl, acetylene, or propyne, or two adjacent R together with the atoms to which they are attached form cyclopropenyl, cyclobutenyl, cyclopentenyl, or cyclohexenyl, wherein the cyclopropenyl, cyclobutenyl, cyclopentenyl, and cyclohexenyl are optionally substituted with 1-3 groups selected from F, Cl, ═O, CN, methyl, ethyl, methoxy, ethoxy, OH, and NH₂.

6. The compound or the stereoisomer or pharmaceutically acceptable salt thereof according to claim 1, wherein 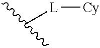
is selected from the following structures:

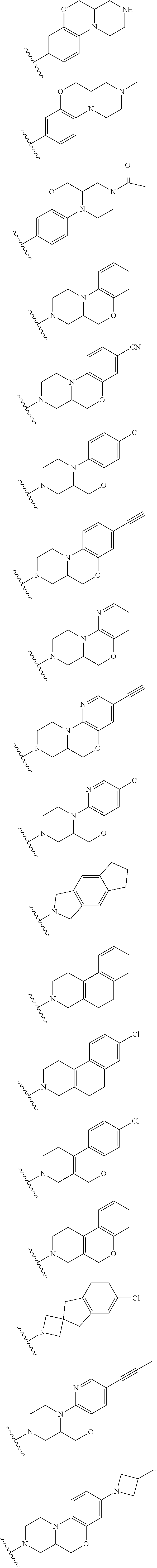

7. The compound or the stereoisomer or pharmaceutically acceptable salt thereof according to claim 1, wherein the compound is selected from one of structures.

8. The compound or the stereoisomer or pharmaceutically acceptable salt thereof according to claim 7, wherein the compound is selected from one of structures in Table 2.

9. A pharmaceutical composition or pharmaceutical preparation, wherein the pharmaceutical composition or pharmaceutical preparation comprises the compound or the stereoisomer or pharmaceutically acceptable salt thereof according to claim 1, and a pharmaceutically acceptable carrier and/or auxiliary material.

10. The pharmaceutical composition or pharmaceutical preparation according to claim 9, wherein the pharmaceutical composition or pharmaceutical preparation comprises 1-1500 mg of the compound or the stereoisomer or pharmaceutically acceptable salt thereof, and a pharmaceutically acceptable carrier and/or auxiliary material.

11. (canceled)

12. A method for treating/preventing a disease in a mammal, wherein the method comprises administering to a subject a therapeutically effective amount of the compound or the stereoisomer or pharmaceutically acceptable salt thereof according to claim 1.

13. The method according to claim 12, wherein the therapeutically effective amount is 1-1500 mg.

14. The method according to claim 12, wherein the disease is cancer, COPD, idiopathic pulmonary fibrosis, or interstitial lung disease.

15. The method according to claim 12, wherein the disease is a PDE4B-mediated disease.
